(19) United States
(12) Patent Application Publication
(10) Pub. No.: US 2025/0160204 A1
(43) Pub. Date: May 15, 2025

(54) MATERIALS FOR ELECTRONIC DEVICES

(71) Applicant: Merck Patent GmbH, Darmstadt (DE)

(72) Inventor: Philipp STOESSEL, Darmstadt (DE)

(21) Appl. No.: 18/838,008

(22) PCT Filed: Feb. 13, 2023
(86) PCT No.: PCT/EP2023/053406

§ 371 Date: Aug. 13, 2024

(30) Foreign Application Priority Data
Feb. 14, 2022 (EP) 22156647.4

(51) Int. Cl. H10K 85/60; C07D 221/18; C07D 401/04; C07D 401/10; C07D 401/14; C07D 405/04; C07D 405/10; C07D 405/14; C07D 409/04; C07D 471/08; C07D 498/08; C09K 11/02; H10K 50/11; H10K 50/15; H10K 50/16; H10K 50/18

(52) U.S. Cl. CPC H10K 85/6572; C07D 221/18; C07D 401/04; C07D 401/10; C07D 401/14; C07D 405/04; C07D 405/10; C07D 405/14; C07D 409/04; C07D 471/08; C07D 498/08; C09K 11/02; H10K 85/615; H10K 85/622; H10K 85/626; H10K 85/654; H10K 85/657; H10K 85/6574; H10K 50/11; H10K 50/15; H10K 50/16; H10K 50/18; H10K 50/181

(57) ABSTRACT

The present invention relates to compounds that are suitable for use in electronic devices, and to electronic devices, more particularly organic electroluminescent devices, containing these compounds.

The present invention relates to materials for use in electronic devices, especially in organic electroluminescent devices, and to electronic devices, especially organic electroluminescent devices comprising these materials.

Electronic devices containing organic, organometallic and/or polymeric semiconductors are becoming increasingly important, and are being used in many commercial products for reasons of cost and because of their performance. Examples here include organic-based charge transport materials (for example triarylamine-based hole transporters) in photocopiers, organic or polymeric light-emitting diodes (OLEDs or PLEDs) in readout and display devices or organic photoreceptors in photocopiers. Organic solar cells (O-SCs), organic field-effect transistors (O-FETs), organic thin-film transistors (O-TFTs), organic integrated circuits (O-ICs), organic optical amplifiers and organic laser diodes (O-lasers) are at an advanced stage of development and may have great future significance.

Electronic devices in the context of this invention are understood to mean organic electronic devices containing organic semiconductor materials as functional materials. In particular, the electronic devices represent electroluminescent devices such as OLEDs.

The construction of OLEDs in which organic compounds are used as functional materials is known to the person skilled in the art from the prior art. In general, OLEDs are understood to mean electronic devices having one or more layers which comprise organic compounds and emit light on application of a voltage.

In electronic devices, especially OLEDs, there is a great need to improve performance data, especially lifetime, efficiency and operating voltage. For these aspects, it has not been possible to date to find a satisfactory solution.

Electronic devices typically include cathode, anode and at least one functional, preferably emitting, layer. Apart from these layers, they may also contain further layers, for example in each case one or more hole injection layers, hole transport layers, hole blocker layers, electron transport layers, electron injection layers, exciton blocker layers, electron blocker layers and/or charge generation layers.

A major influence on the performance data of electronic devices is possessed by the hole transport layers and electron transport layers.

It is an object of the present invention to provide compounds that are suitable for use in an electronic device, especially an OLED, especially as material of hole transport layers or material of electron transport layers, where they lead to good properties.

It has been found that, surprisingly, this object is achieved by particular triptycenes described in detail hereinafter that are of good suitability for use in electronic devices, especially OLEDs. These OLEDs especially have a long lifetime, high efficiency and relatively low operating voltage. The present invention therefore provides these compounds and electronic devices, especially organic electroluminescent devices, comprising these compounds.

The present invention provides a compound of one of the formulae (1) and (2):

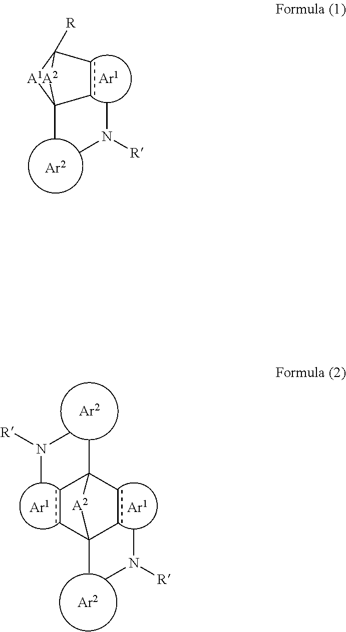

where the symbols used are as follows:
Ar¹, Ar² are the same or different at each instance and are an aromatic or heteroaromatic ring system which has 5 to 60 aromatic ring atoms and may be substituted by one or more R radicals, where Ar¹ and/or Ar² may each be joined to R′ via an R radical or a single bond;
A¹, A² are the same or different at each instance and are a divalent alkylene group having 1 to 4 carbon atoms, a divalent alkenylene group having 2 to 4 carbon atoms or a divalent arylene or heteroarylene group having 5 to 60 ring atoms, where the alkylene, alkenylene, arylene and heteroarylene groups may be substituted by one or more R groups;
R′ is the same or different at each instance and is H, D, F, Cl, Br, I, OAr′, SAr′, B(R¹)₂, B(OR¹)₂, CHO, C(═O)R, CR═C(R)₂, CN, C(═O)OR, C(═O)NR, Si(R)₃, NO₂, P(═O)(R)₂, OSO₂R, OR, S(═O)R, S(═O)₂R, SR, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R radicals, where one or more nonadjacent CH₂ groups may be replaced by —RC═CR—, —C≡C—, Si(R)₂, NR, CONR, C═O, C═S, —C(═O)O—, P(═O)(R), —O—, —S—, SO or SO₂, or an aromatic or heteroaromatic ring system which has 5 to 60 aromatic ring atoms, preferably 5 to 40 aromatic ring atoms, and may be substituted in each case by one or more R radicals, where two or more R radicals may together form an aliphatic, heteroaliphatic, aromatic or heteroaromatic ring system which may be substituted by one or more R¹ radicals;
R is the same or different at each instance and is H, D, F, Cl, Br, I, N(Ar′)₂, N(R¹)₂, OAr′, SAr′, B(R¹)₂, B(OR¹)₂, CHO, C(═O)R¹, CR¹=C(R¹)₂, CN, C(═O)OR¹, C(═O)NR¹, Si(R¹)₃, NO₂, P(R¹)₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C═C—, Si(R¹)₂, NR¹, CONR¹, C═O, C═S, —C(═O)O—, P(═O)(R¹), —O—, —S—, SO or SO₂, or an aromatic or heteroaromatic ring system which has 5 to 60 aromatic ring atoms, preferably 5 to 40 aromatic ring atoms, and may be substituted in each case by one or more R¹ radicals, where two or more R radicals preferably bonded to the same cycle or the same carbon atom may together and/or with R′ form an aliphatic, heteroaliphatic, aromatic or heteroaromatic ring system which may be substituted by one or more R¹ radicals;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, I, B(R²)₂, B(OR²)₂, N(R²)₂, CHO, C(═O)R², CR²=C(R²)₂, CN, C(═O)OR², Si(R²)₃, NO₂, P(R²)₂, P(═O)(R²)₂, OSO₂R², SR², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C═C—, Si(R²)₂, C═O, C═S, —C(═O)O—, NR², CONR², P(═O)(R²), —O—, —S—, SO or SO₂, and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R¹ radicals together may form an aliphatic, heteroaliphatic, aromatic or heteroaromatic ring system;
R² is the same or different at each instance and is H, D, F, CN or an aliphatic, aromatic or heteroaromatic organic radical having 1 to 20 carbon atoms, in which one or more hydrogen atoms may also be replaced by D or F; at the same time, two or more R² substituents may be joined to one another and may form a ring.

An aryl group in the context of this invention contains 6 to 40 carbon atoms; a heteroaryl group in the context of this invention contains 5 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An aromatic ring system in the context of this invention contains 6 to 60 carbon atoms, preferably 6 to 40 carbon atoms, in the ring system. A heteroaromatic ring system in the context of this invention contains 1 to 60 carbon atoms, preferably 1 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit (preferably less than 10% of the atoms other than H), for example a carbon, nitrogen or oxygen atom or carbonyl group. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are joined, for example, by a linear or cyclic alkyl group or by a silyl group. Preferred aromatic or heteroaromatic ring systems are simple aryl or heteroaryl groups and groups in which two or more aryl or heteroaryl groups are joined directly to one another, for example biphenyl, terphenyl, quaterphenyl or bipyridine, and also fluorene or spirobifluorene.

An electron-rich heteroaromatic ring system is characterized in that it is a heteroaromatic ring system containing no electron-deficient heteroaryl groups. An electron-deficient heteroaryl group is a six-membered heteroaryl group having at least one having at least one nitrogen atom or a five-membered heteroaryl group having at least two heteroatoms, one of which is a nitrogen atom and the other is oxygen, sulfur or a substituted nitrogen atom, where further aryl or heteroaryl groups may also be fused onto these groups in each case. By contrast, electron-rich heteroaryl groups our five-membered heteroaryl groups having exactly one heteroatom selected from oxygen, sulfur and substituted nitrogen, to which may be fused further aryl groups and/or further electron-rich five-membered heteroaryl groups. Thus, examples of electron-rich heteroaryl groups are pyrrole, furan, thiophene, indole, benzofuran, benzothiophene, carbazole, dibenzofuran, dibenzothiophene or indenocarbazole. An electron-rich heteroaryl group is also referred to as an electron-rich heteroaromatic radical.

An electron-deficient heteroaromatic ring system is characterized in that it contains at least one electron-deficient heteroaryl group, and especially preferably no electron-rich heteroaryl groups.

In the context of the present invention, the term “alkyl group” is used as an umbrella term both for linear and branched alkyl groups and for cyclic alkyl groups. Analogously, the terms “alkenyl group” and “alkynyl group” are used as umbrella terms both for linear or branched alkenyl or alkynyl groups and for cyclic alkenyl or alkynyl groups.

A cyclic alkyl, alkoxy or thioalkoxy group in the context of this invention is understood to mean a monocyclic, bicyclic or polycyclic group.

The term “divalent” in a divalent alkylene, alkenylene, arylene or heteroarylene group, as defined for A¹ and A², is intended to make clear that these groups are each bonded to the two carbon atoms shown explicitly in the formula (1) or formula (2). As set out in the definition of A¹ and A², these groups may still be substituted by one or more R radicals. Even in the presence of such substitution by R, these groups are still referred to as divalent groups in the context of the present invention.

In the context of the present invention, an aliphatic hydrocarbyl radical or an alkyl group or an alkenyl or alkynyl group which may contain 1 to 40 carbon atoms and in which individual hydrogen atoms or CH₂ groups may also be replaced by the abovementioned groups are preferably understood to mean the following radicals: methyl, ethyl, n-propyl, i-propyl, n-butyl, i-butyl, s-butyl, t-butyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, cyclooctyl, 2-ethylhexyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)-cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl, ethenyl, propenyl, butenyl, pentenyl, cyclopentenyl, hexenyl, cyclohexenyl, heptenyl, cycloheptenyl, octenyl, cyclooctenyl, cyclooctadienyl, ethynyl, propynyl, butynyl, pentynyl, hexynyl, heptynyl or octynyl. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms, preferably 5-40 aromatic ring atoms, and may also be substituted in each case by the abovementioned radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring system, in the context of the present description, should be understood to mean, inter alia, that the two radicals are joined to one another by a chemical bond with formal elimination of two hydrogen atoms. This is illustrated by the following scheme:

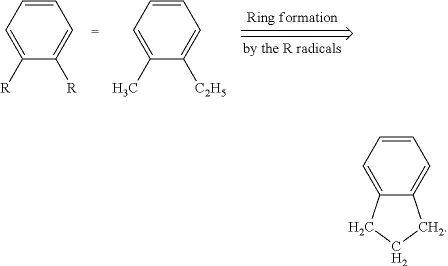

In addition, however, the abovementioned wording shall also be understood to mean that, if one of the two radicals is hydrogen, the second radical binds to the position to which the hydrogen atom was bonded, forming a ring. This will be illustrated by the following scheme:

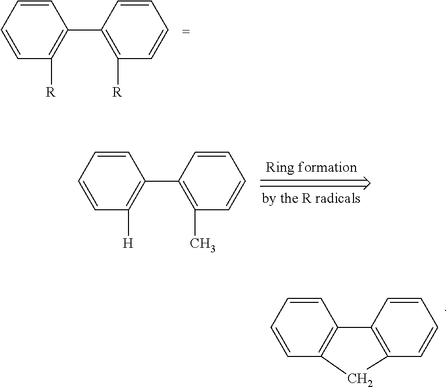

In a preferred embodiment, the compound is a compound of the formula (3) or (4):

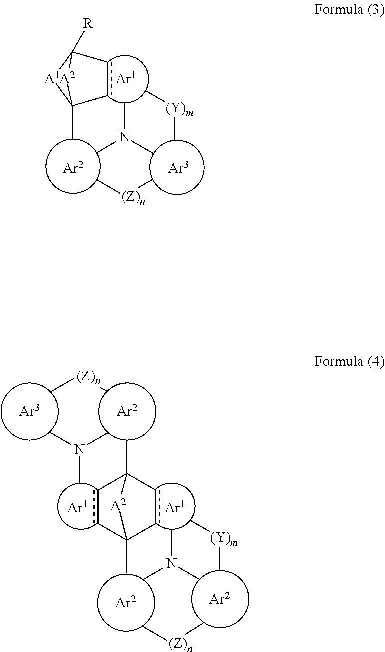

where the symbols used have the definitions given above for formula (1), and in addition:
Ar³ is the same or different at each instance and is an aromatic or heteroaromatic ring system which has 5 to 60 aromatic ring atoms and may be substituted by one or more R radicals;
m, n are the same or different at each instance and are 0 or 1;
Y, Z are the same or different at each instance and are a single bond, BR, BAr′, C═O, C(R)₂, —RC═CR—, o-arylene, NR, NAr′, PR, SO₂, SiR₂, SiAr′₂, P(O)R, P(O)Ar′, O or S.

In a preferred embodiment, A¹ and A² are the same or different at each instance and are CR₂, —CR₂CR₂—, —CR₂CR₂CR₂—, CR═CR—, o-arylene or o-heteroarylene, preferably CR₂, —CR₂CR₂—, o-arylene or o-heteroarylene, where the o-arylene or o-heteroarylene groups may be substituted by one or more R radicals.

Further preferred embodiments of the invention are shown by the formulae (5) and (6):

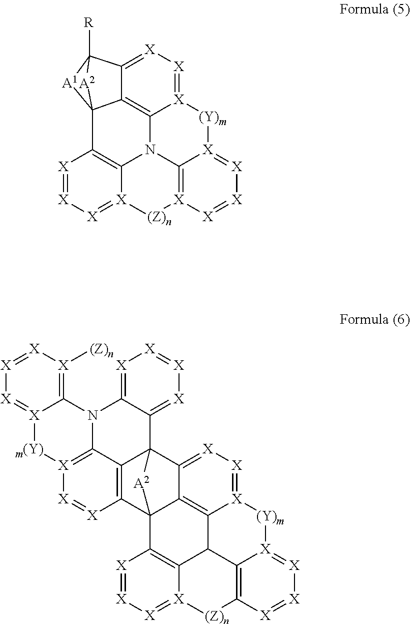

where the symbols used have the definitions given above for formula (3) or formula (4), and in addition:
X is the same or different at each instance and is C when Z or Y is bonded there, CR, CH, CD or N, with the proviso that not more than three X groups per cycle are N.

Preferred embodiments of the formulae (5) and (6) are shown by the following formulae (5-1) to (5-4) and (6-1) to (6-4):

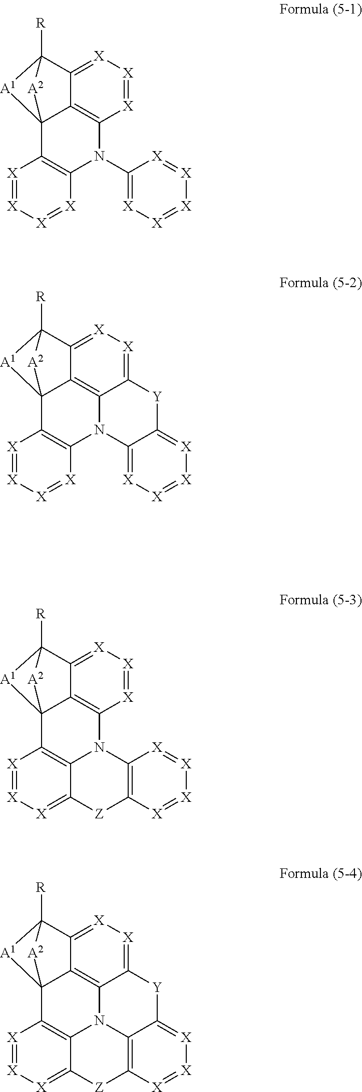

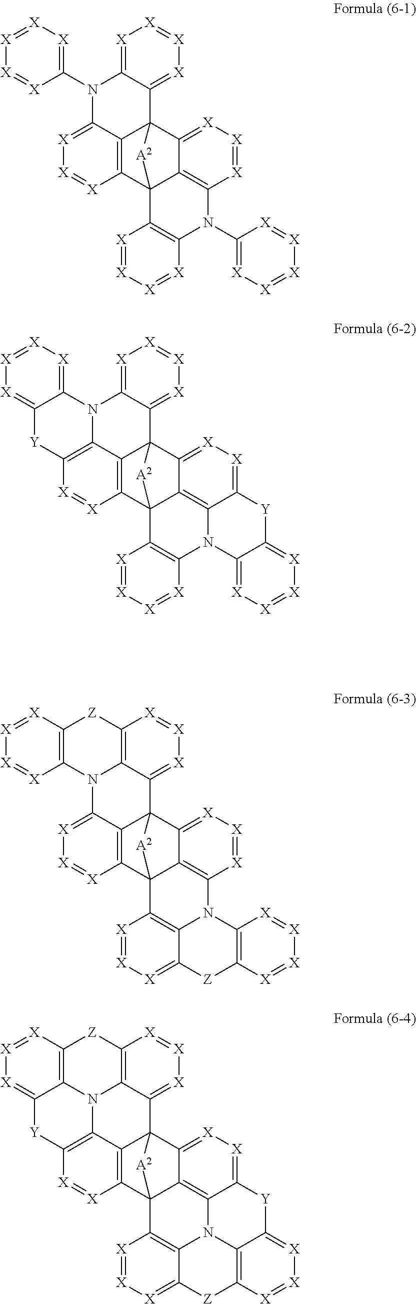

where the symbols used have the definitions given above for formula (5) or formula (6).

Preferred embodiments of the formula (5) or (6) are shown by the formulae (7-1) to (7-4), (8-1) to (8-4), (9-1) to (9-4), (10-1) to (10-4), (11-1) to (11-4) and (12-1) to (12-4):

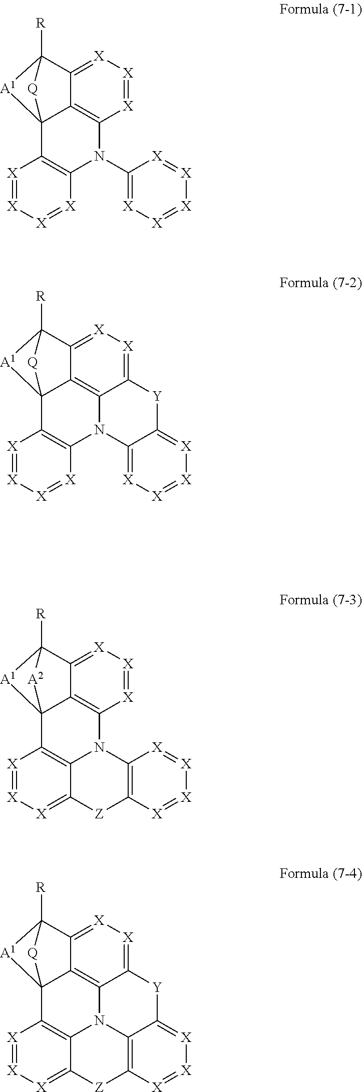

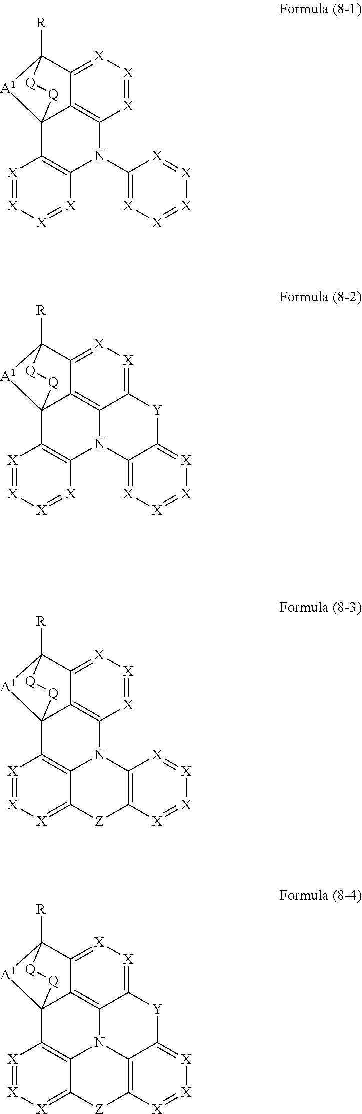

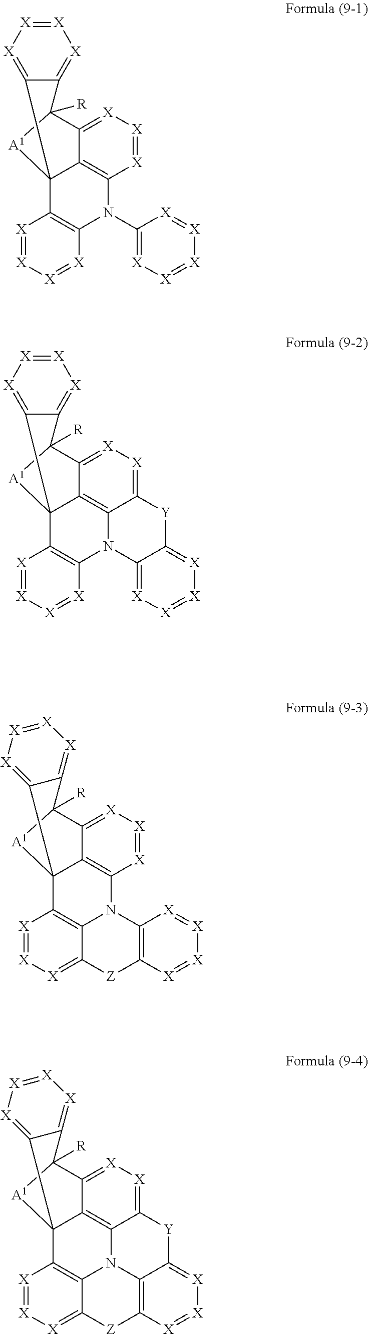

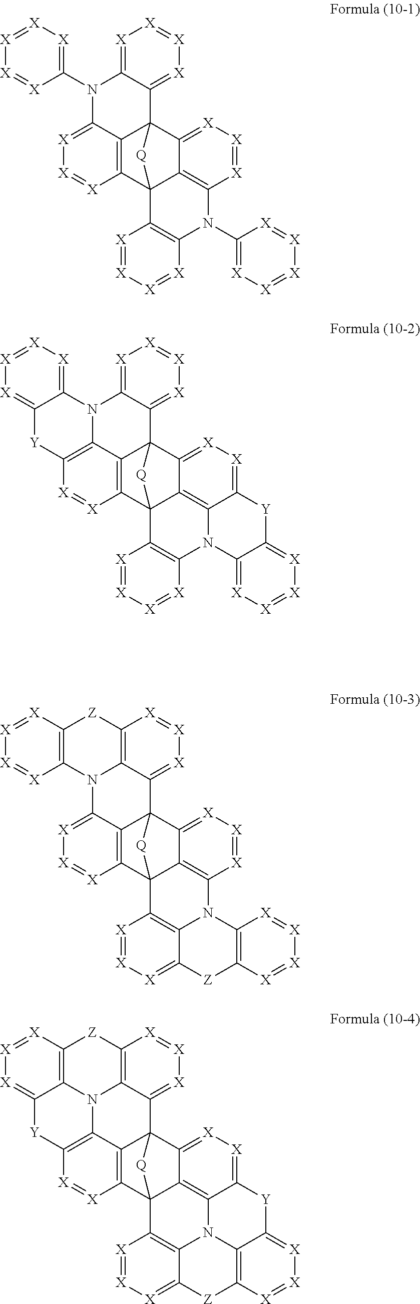

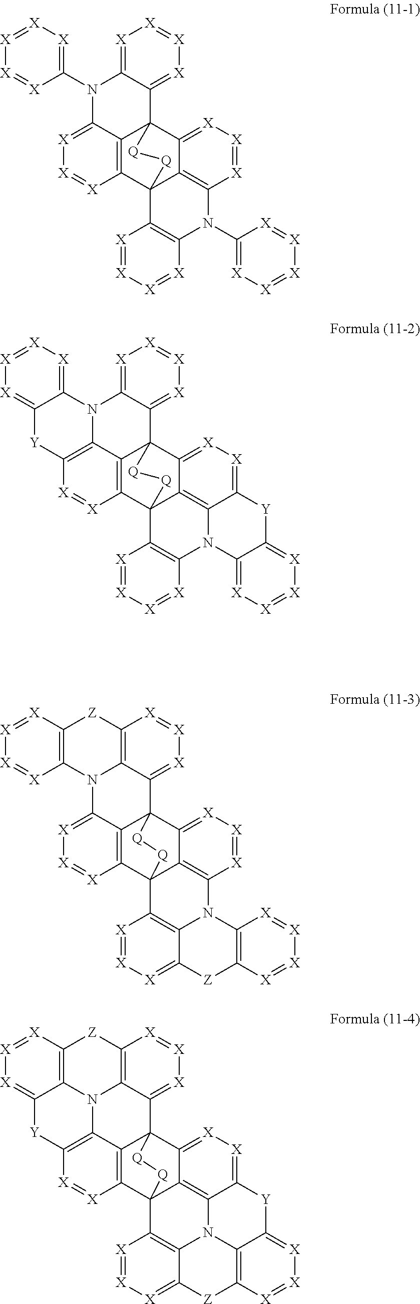

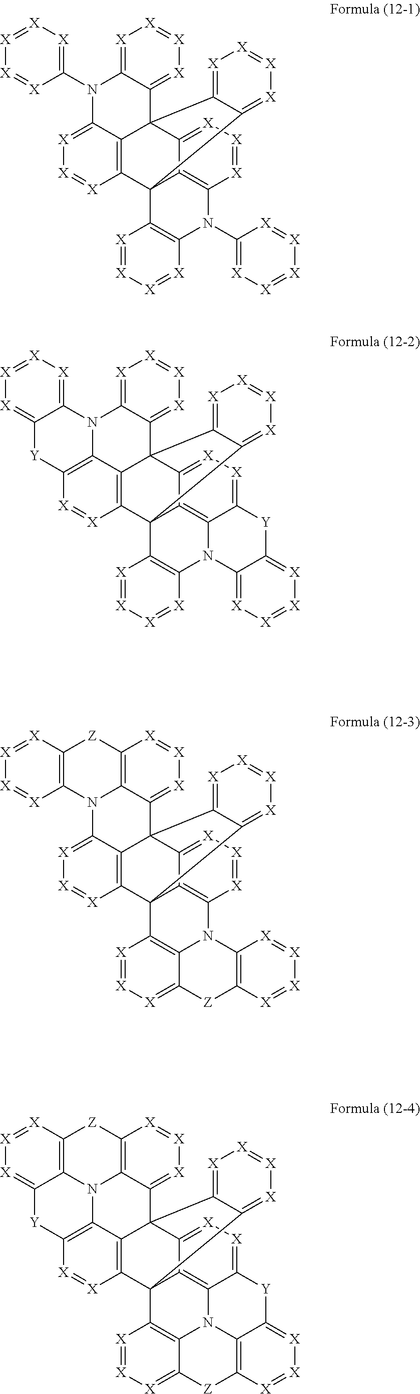

where the symbols used have the definitions given above for formula (5) or formula (6), and in addition:
X is the same or different at each instance and is CR, CH, CD or N, with the proviso that not more than three X groups per cycle are N;
Q is the same or different at each instance and is CH, CD or CR.

Further preferred embodiments are shown by the following formulae (7-1-1) to (7-4-1), (7-1-2) to (7-4-2), (8-1-1) to (8-4-1), (8-1-2) to (8-4-2), (9-1-1) to (9-4-1) and (9-1-2) to (9-4-2):

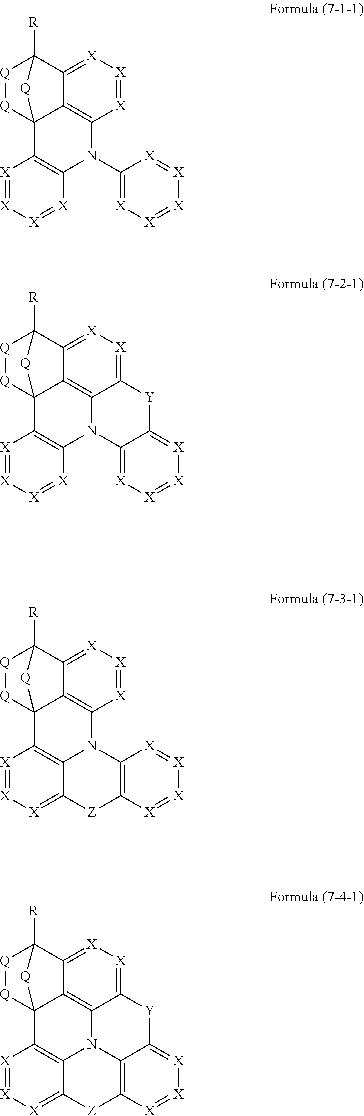

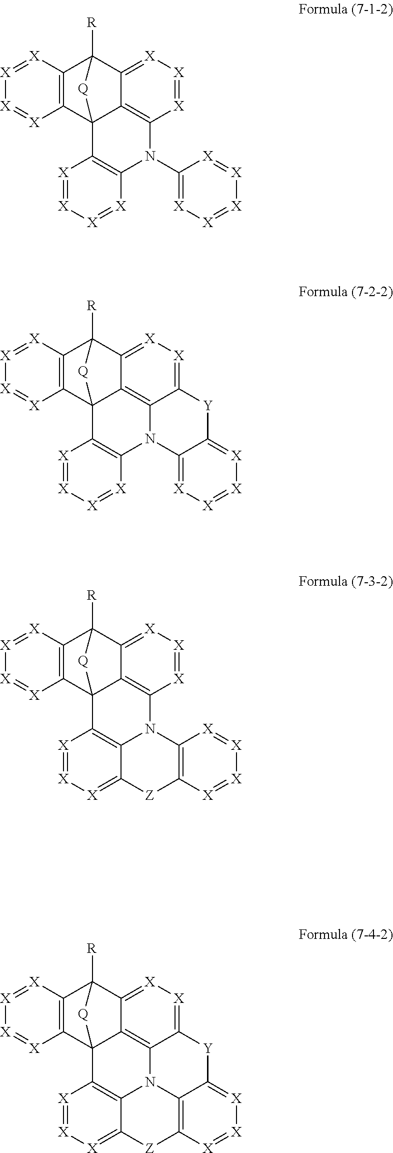

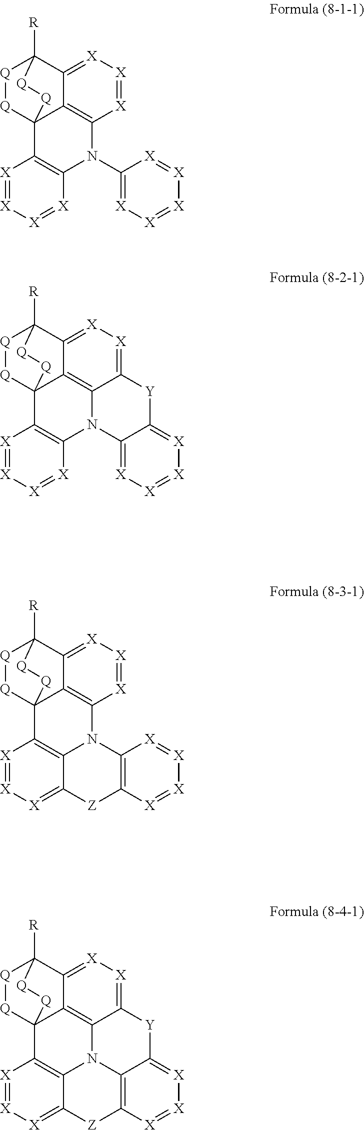

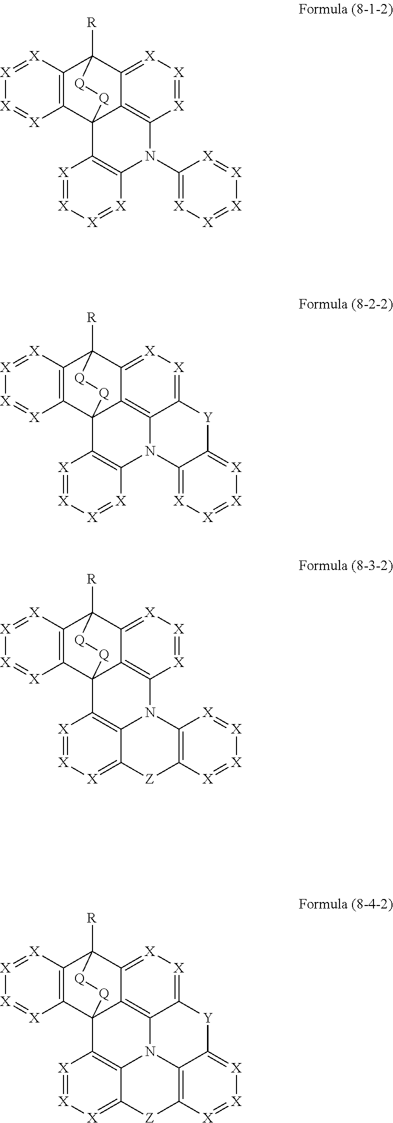

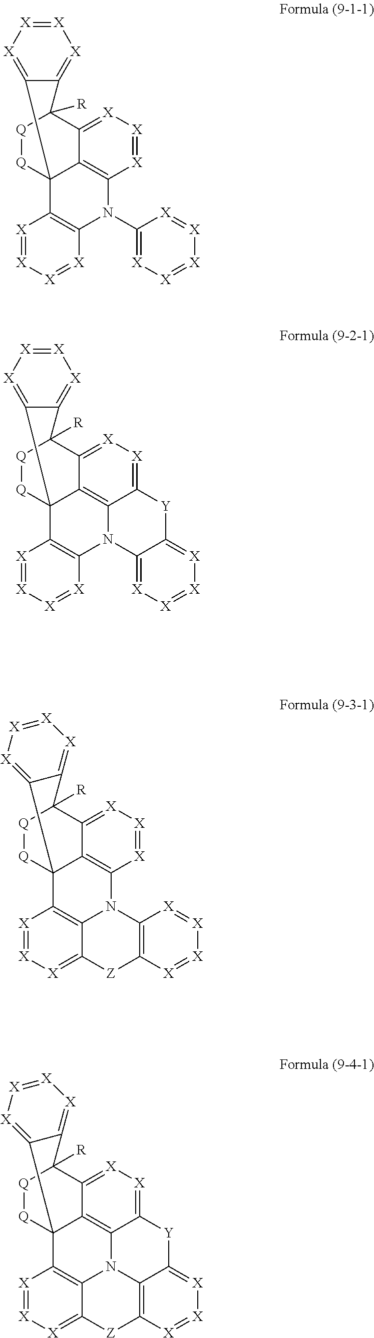

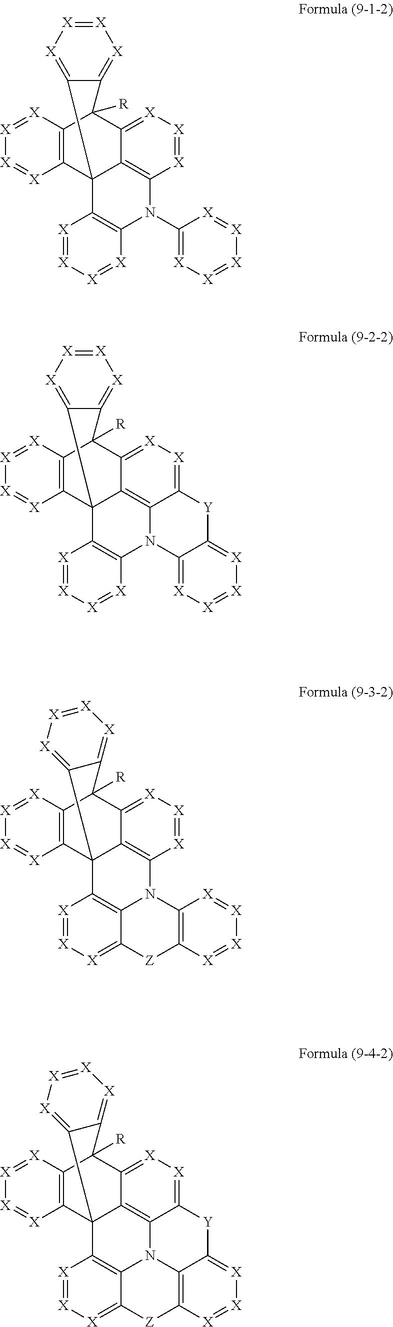

where the symbols used have the definitions given above for formulae (7-1) to (7-4).

In a preferred embodiment, X is the same or different at each instance and is CH, CD, CF or N, with the proviso that not more than three X groups per cycle are N, where the N are not adjacent. Preferably, not more than two X groups are N, more preferably not more than one X group is N, and most preferably no X group is N.

Further preferred embodiments are shown by the following formulae (7-1-1-1) to (7-1-1-8), (7-1-2-1) to (7-1-2-8), (7-2-1-1) to (7-2-1-8), (7-2-2-1) to (7-2-2-8), (7-3-1-1) to (7-3-1-8), (7-3-2-1) to (7-3-2-8), (7-4-1-1) to (7-4-1-8), (7-4-2-1) to (7-4-2-8), (8-1-1-1) to (8-1-1-8), (8-1-2-1) to (8-1-2-8), (8-2-1-1) to (8-2-1-8), (8-2-2-1) to (8-2-2-8), (8-3-1-1) to (8-3-1-8), (8-3-2-1) to (8-3-2-8), (8-4-1-1) to (8-4-1-8), (8-4-2-1) to (8-4-2-8), (9-1-1-1) to (9-1-1-8), (9-1-2-1) to (9-1-2-8), (9-2-1-1) to (9-2-1-8), (9-2-2-1) to (9-2-2-8), (9-3-1-1) to (9-3-1-8), (9-3-2-1) to (9-3-2-8), (9-4-1-1) to (9-4-1-8), (9-4-2-1) to (9-4-2-8), (10-1-1) to (10-1-8), (10-2-1) to (10-2-8), (10-3-1) to (10-3-8), (10-4-1) to (10-4-8), (11-1-1) to (11-1-8), (11-2-1) to (11-2-8), (11-3-1) to (11-3-8), (11-4-1) to (11-4-8), (12-1-1) to (12-1-8), (12-2-1) to (12-2-8), (12-3-1) to (12-3-8) and (12-4-1) to (12-4-8):

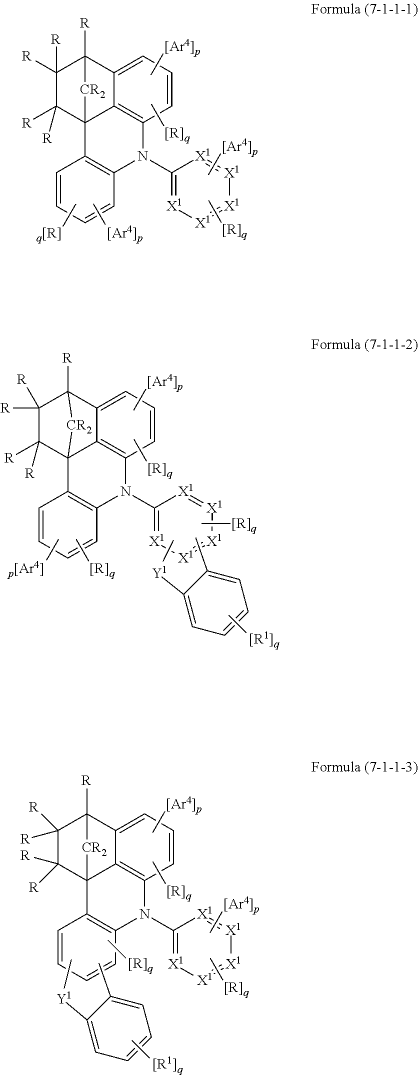

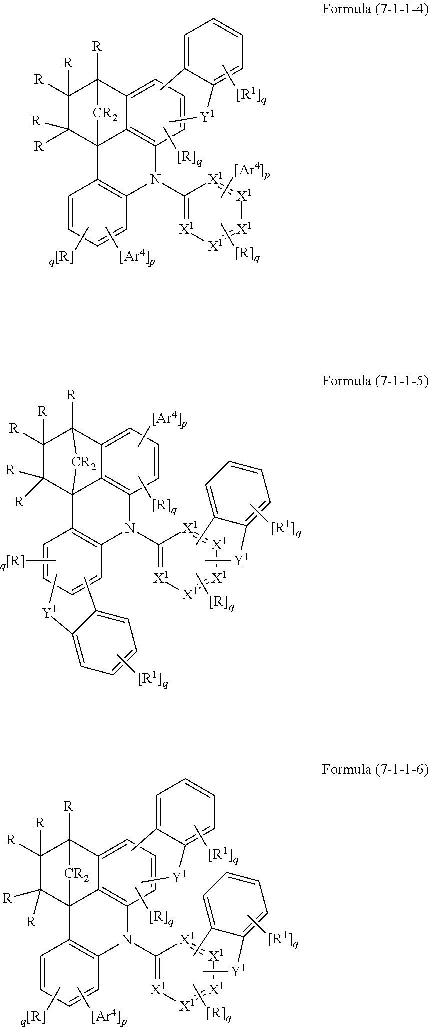

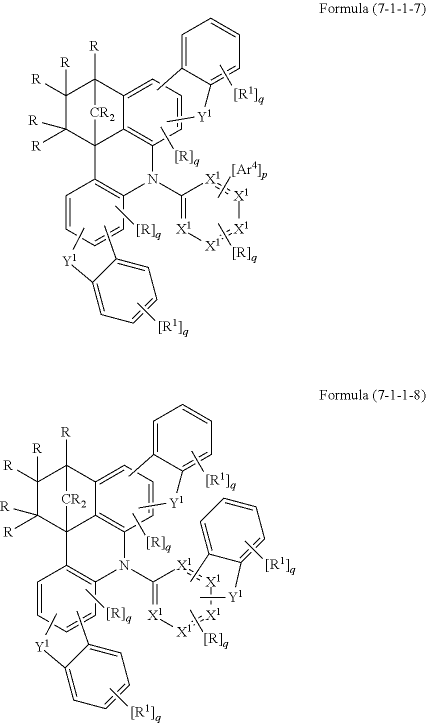

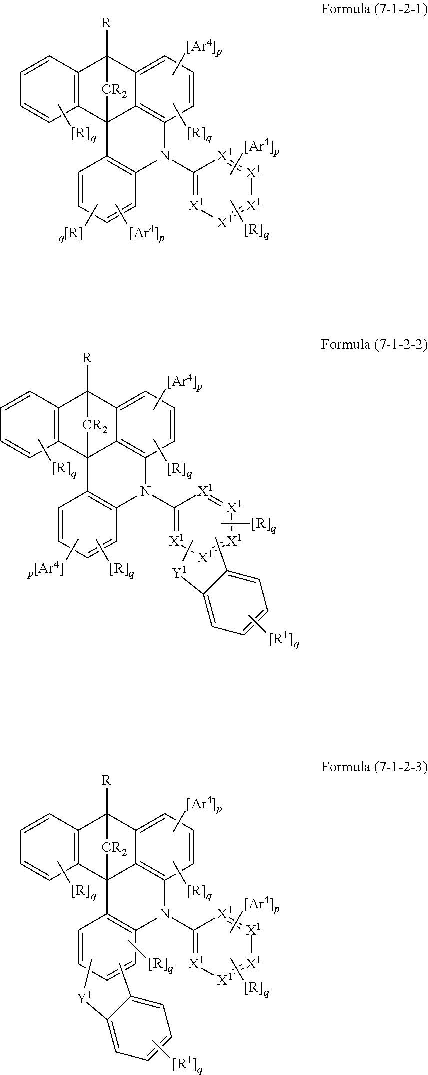

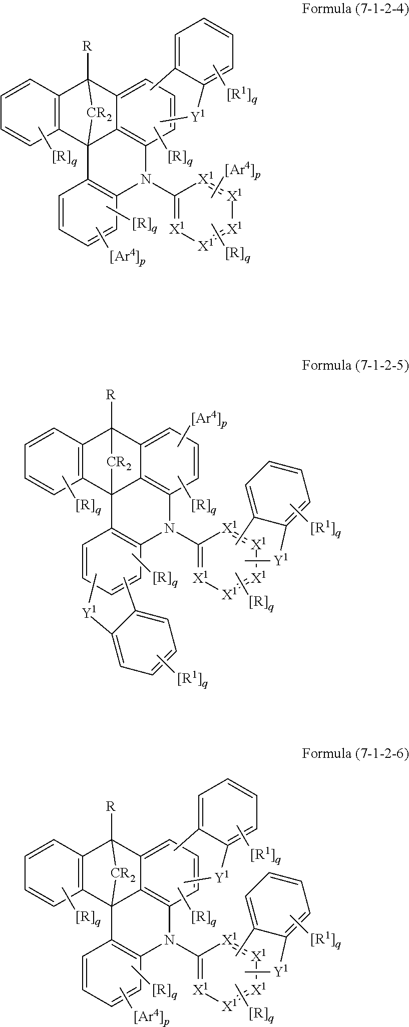

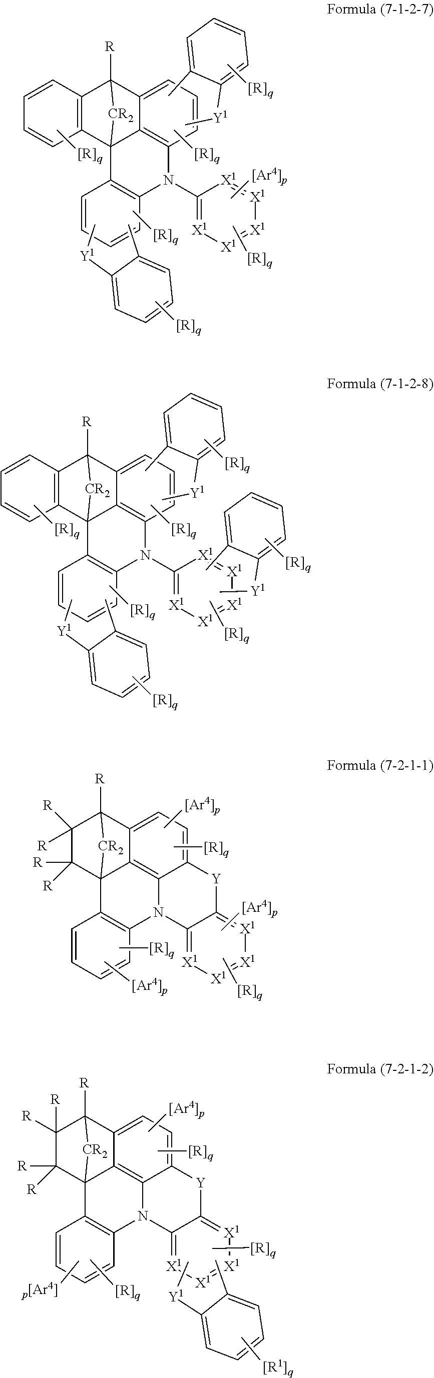

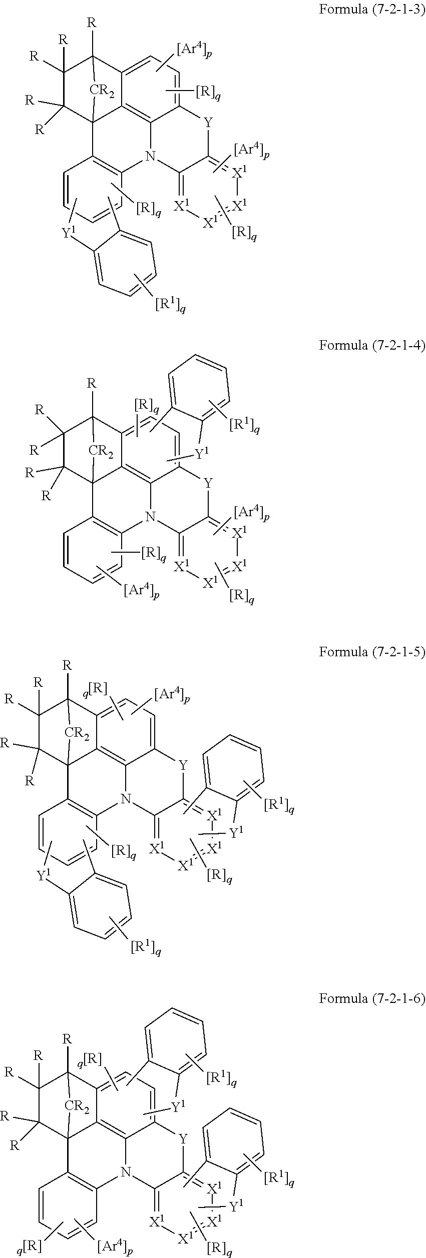

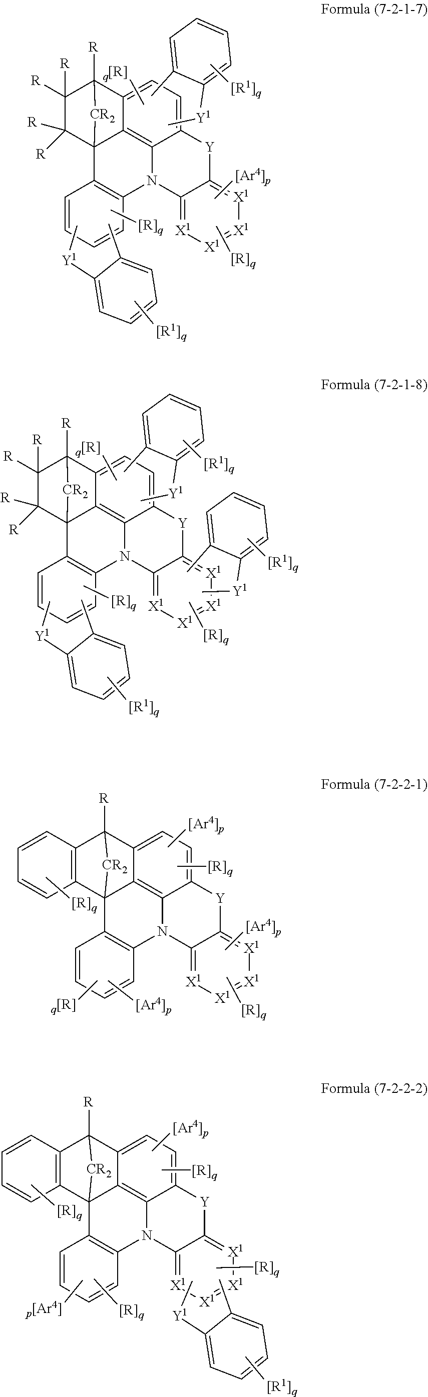

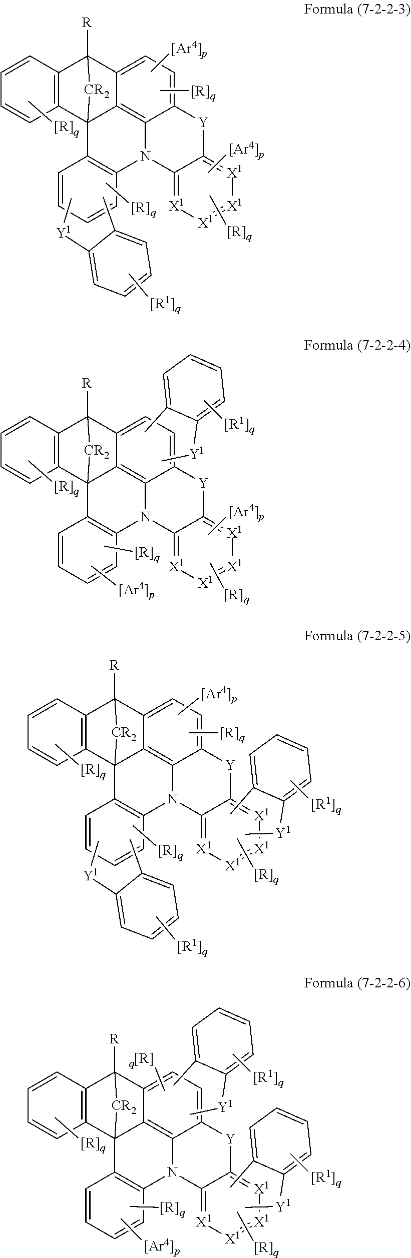

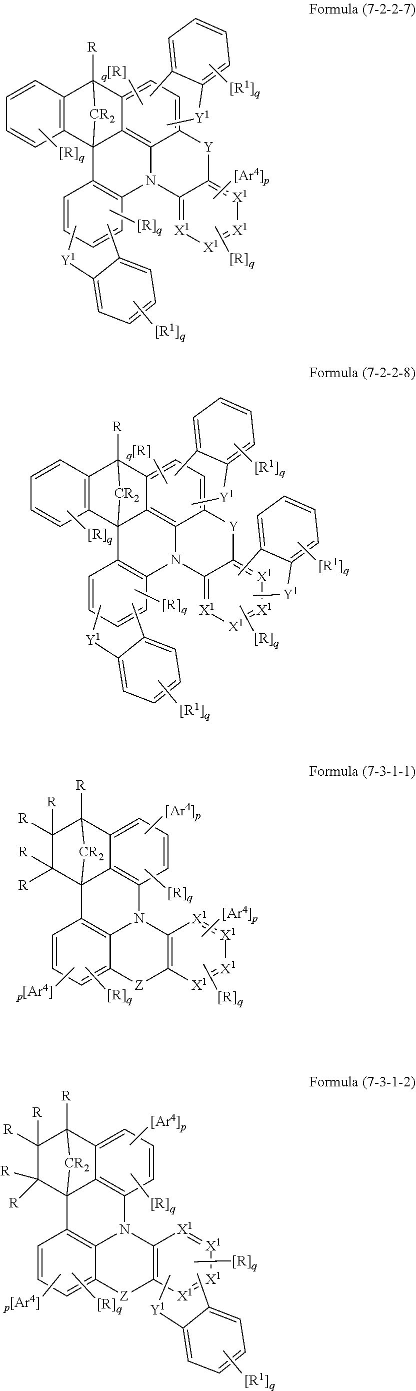

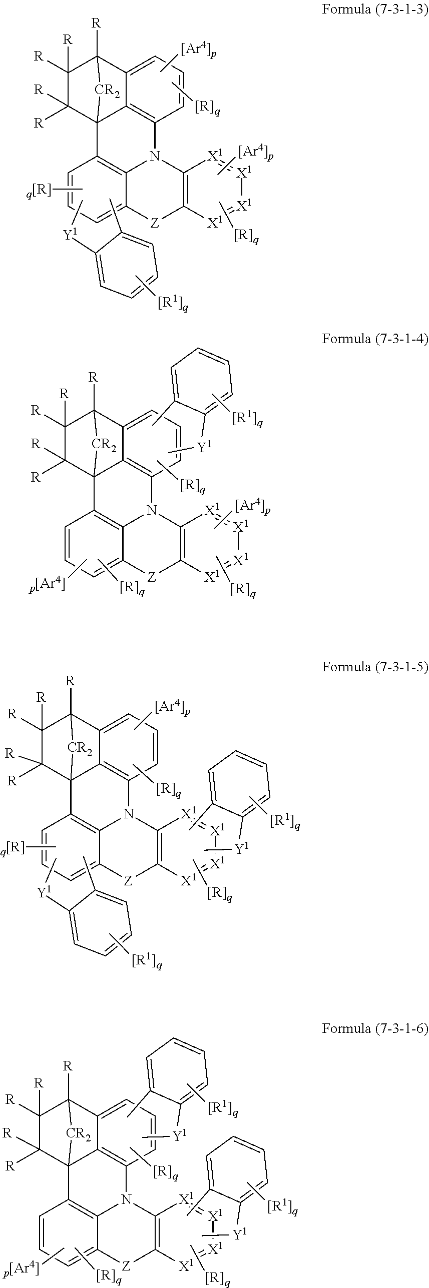

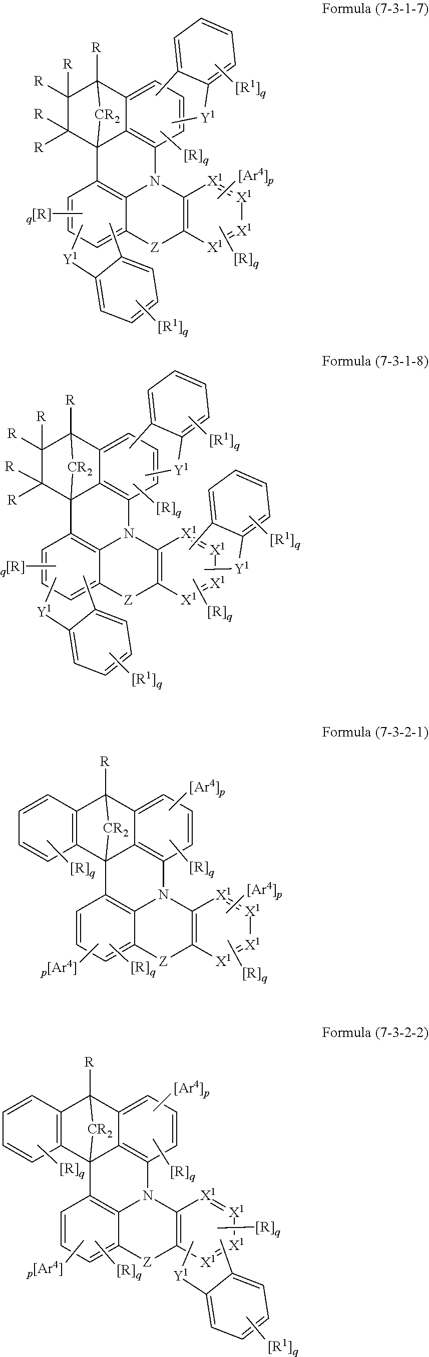

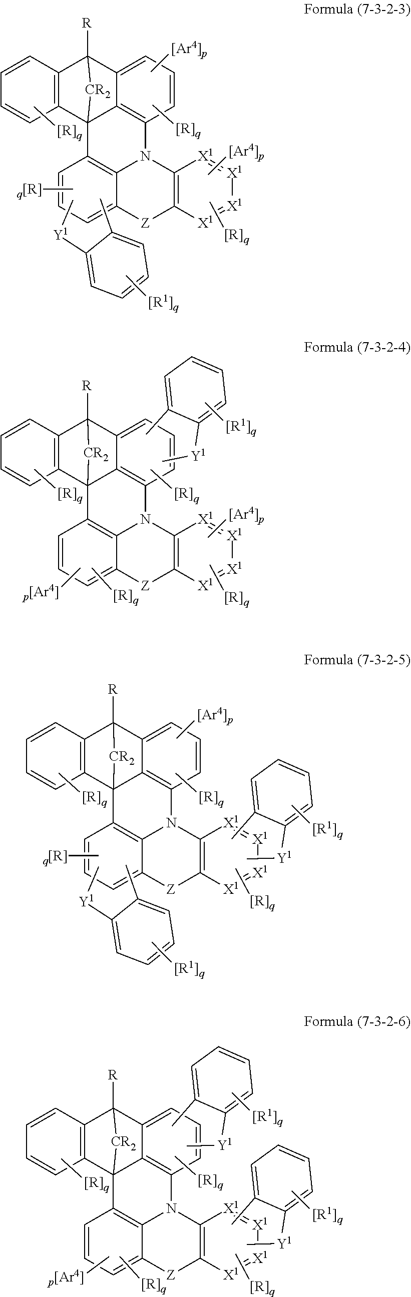

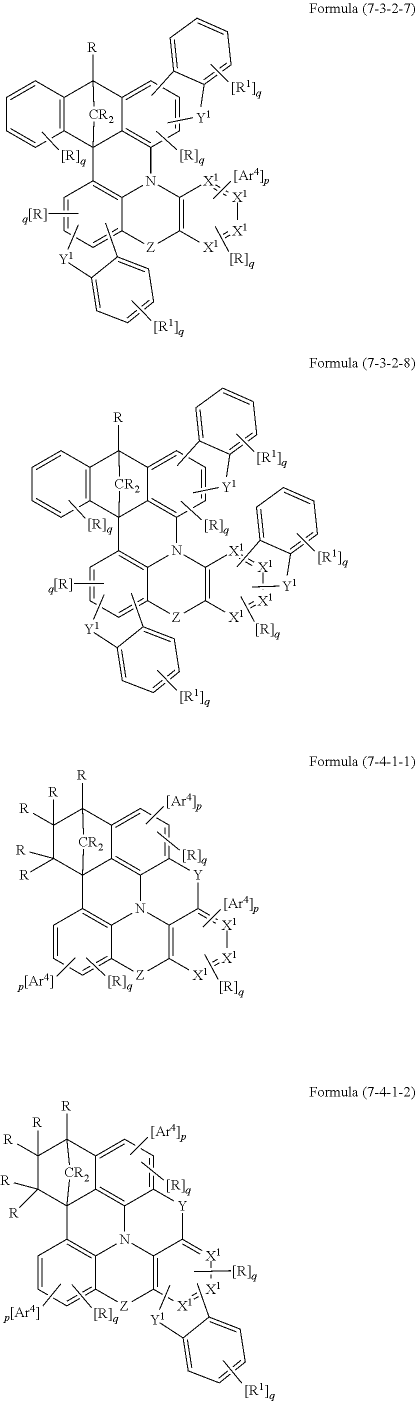

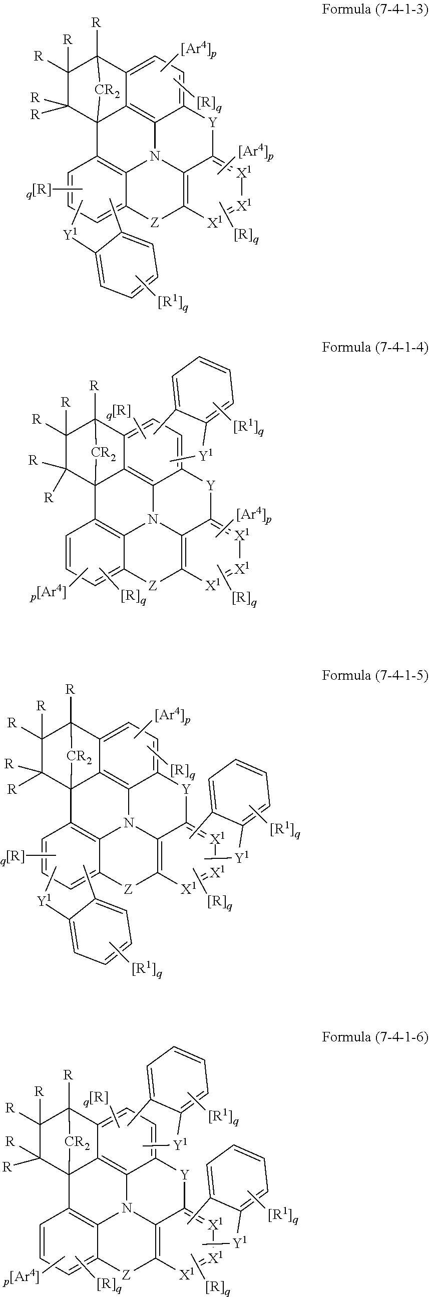

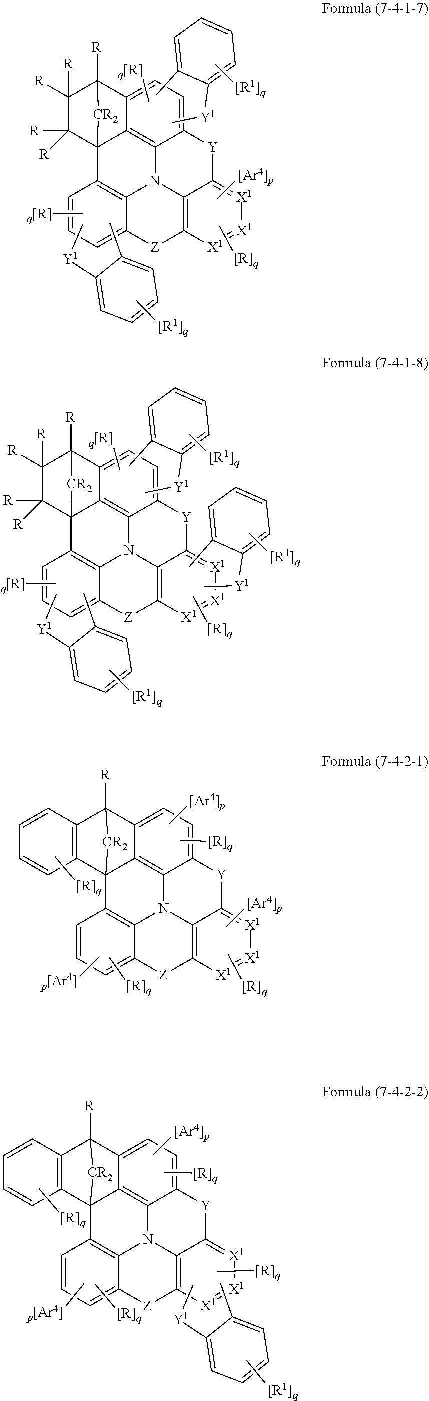

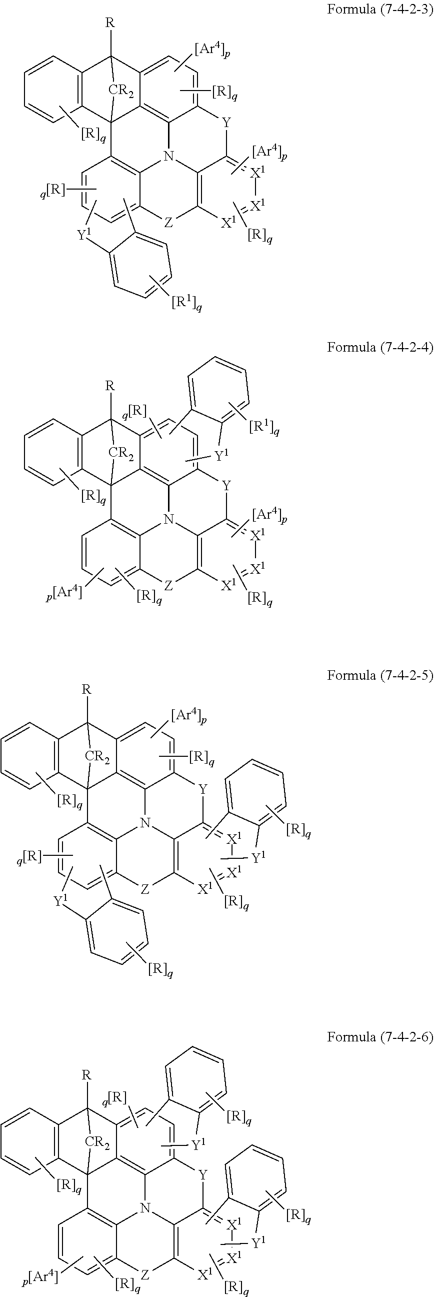

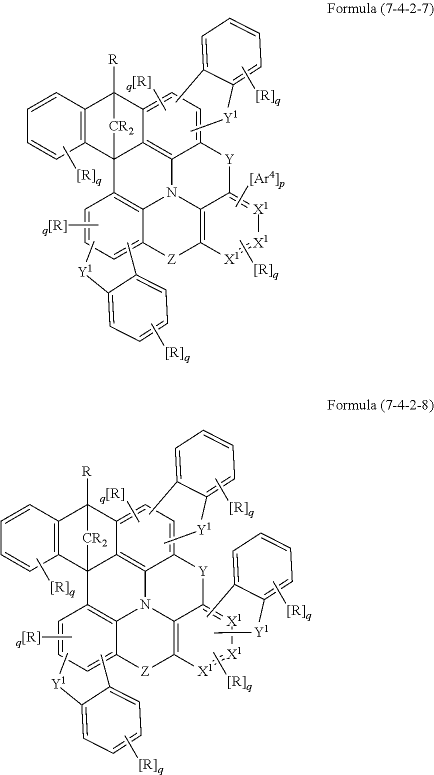

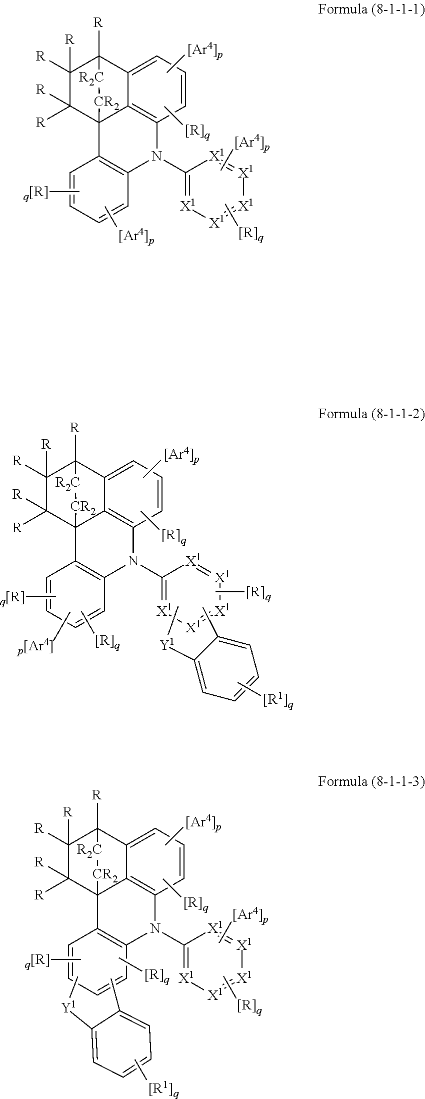

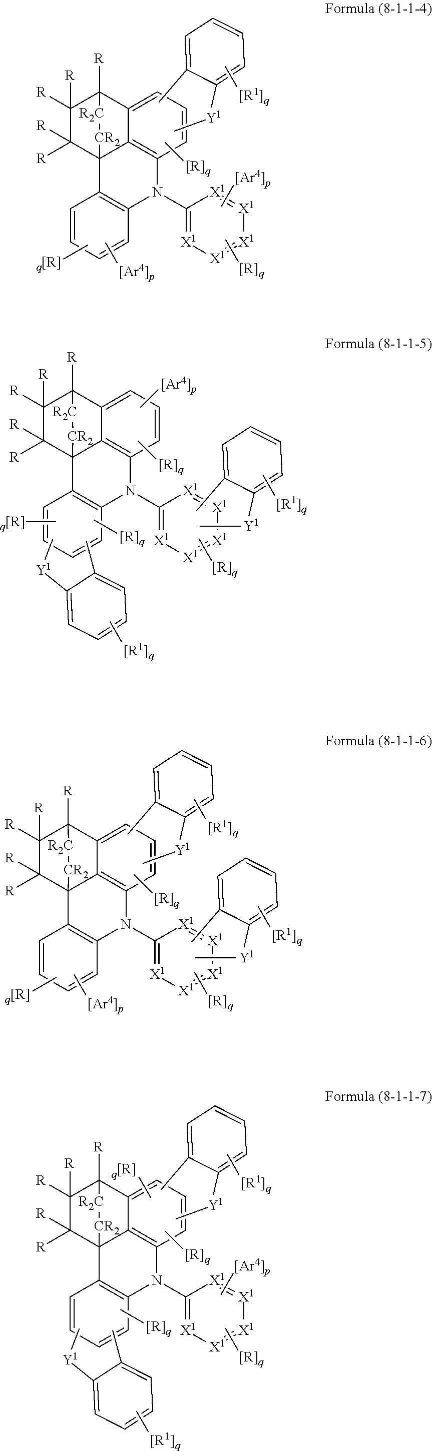

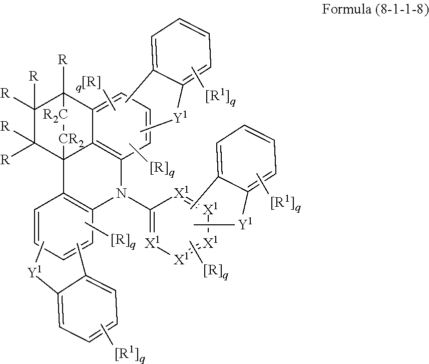

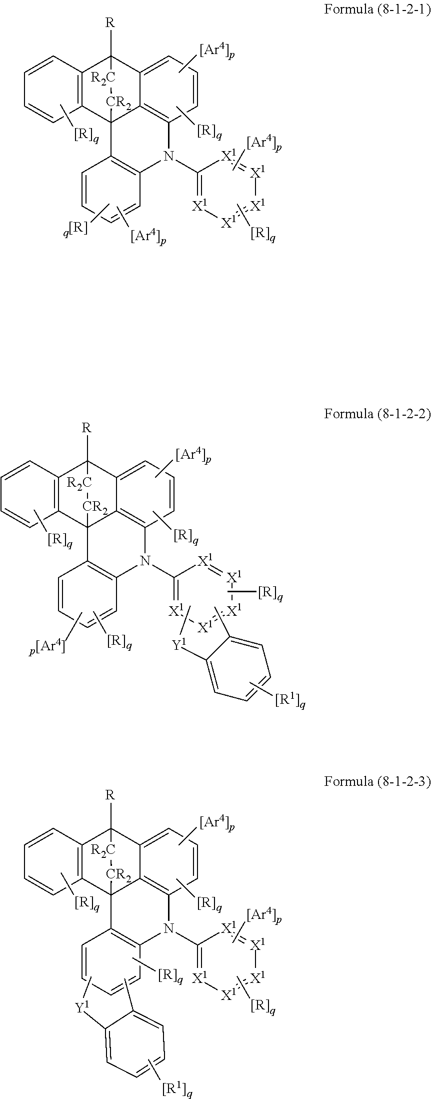

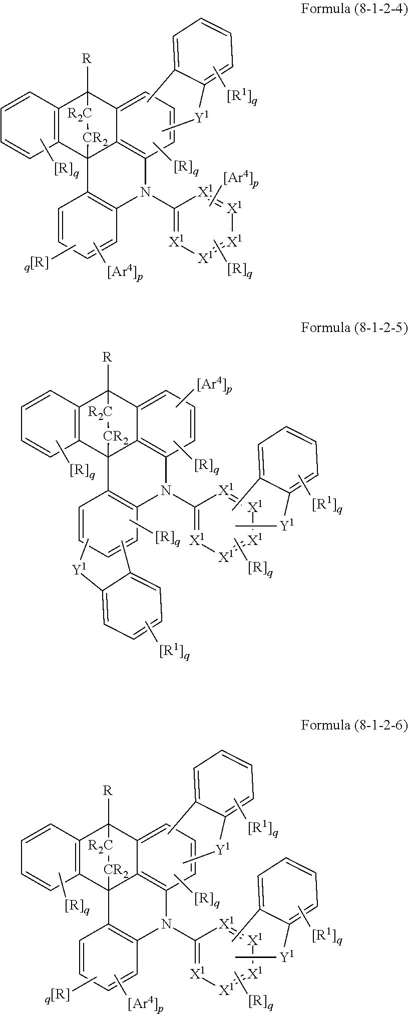

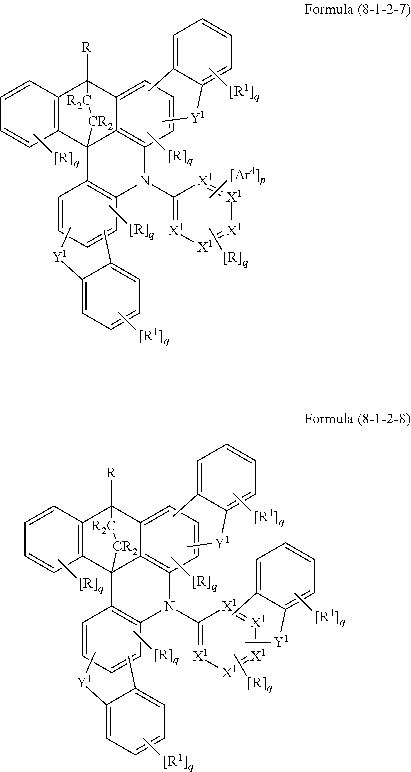

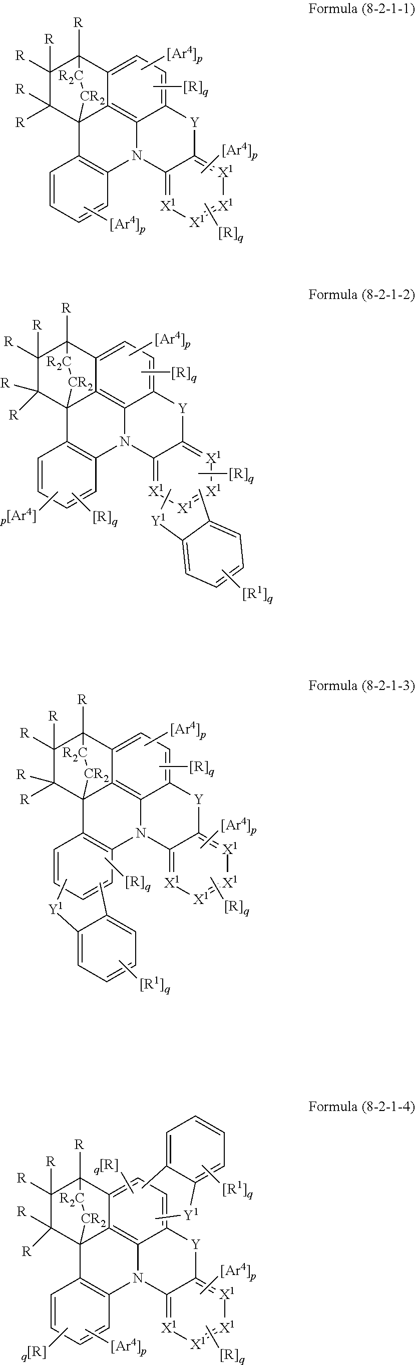

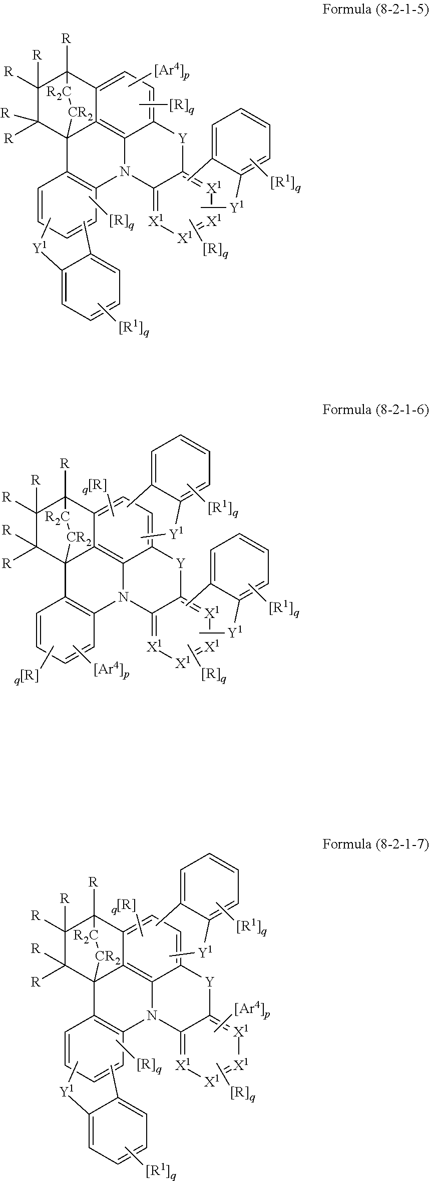

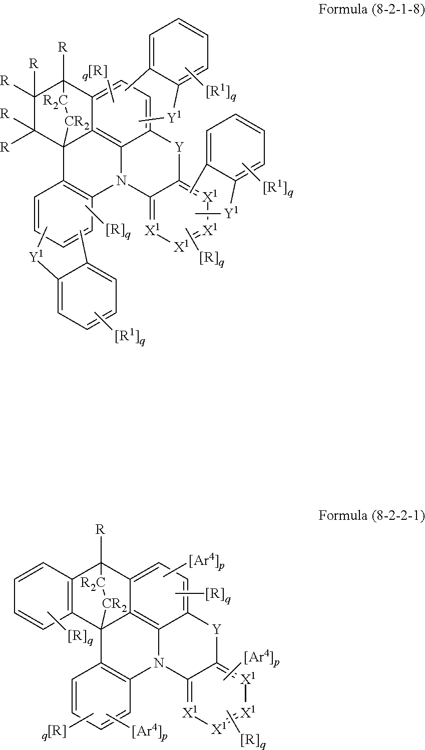

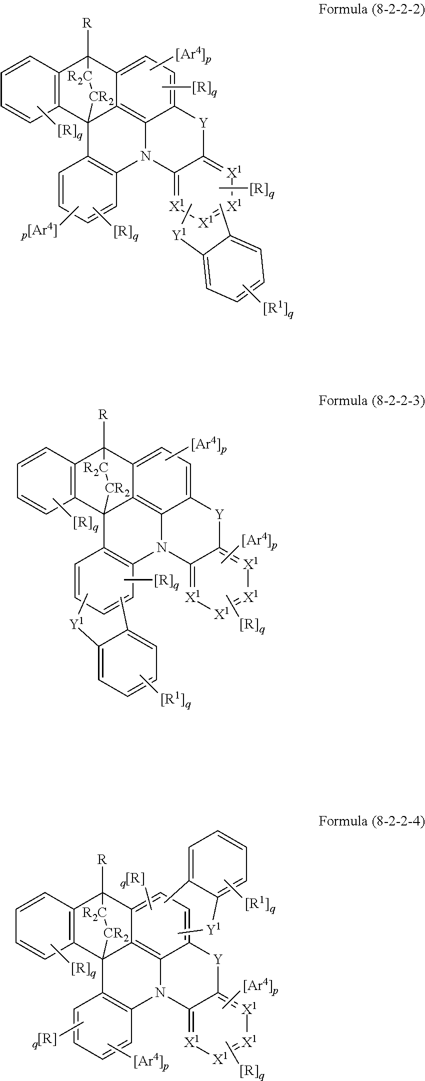

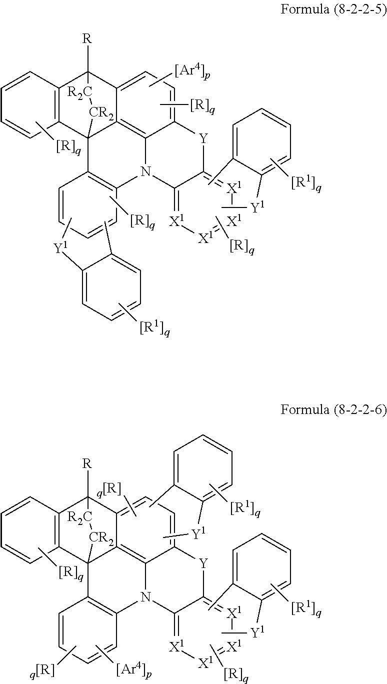

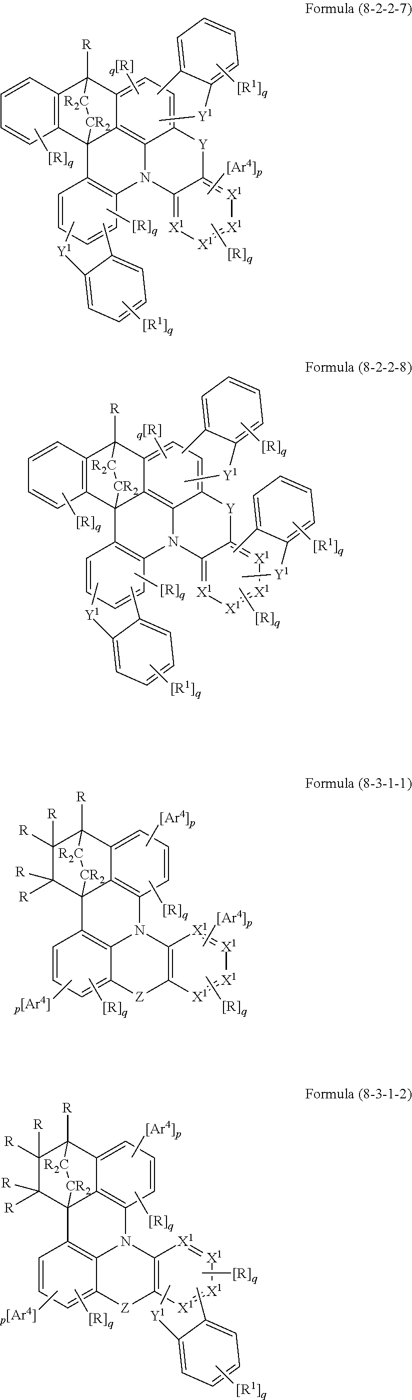

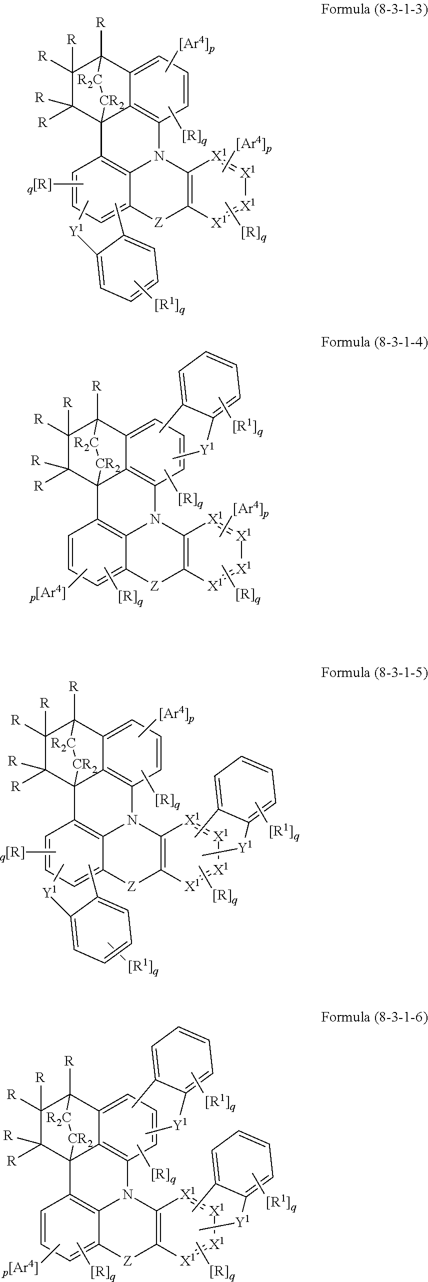

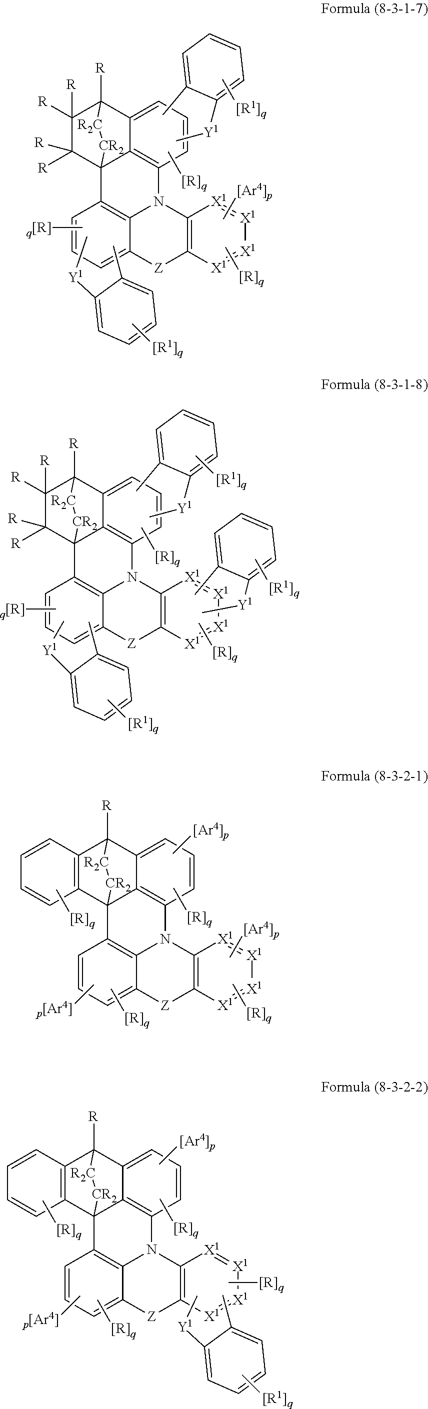

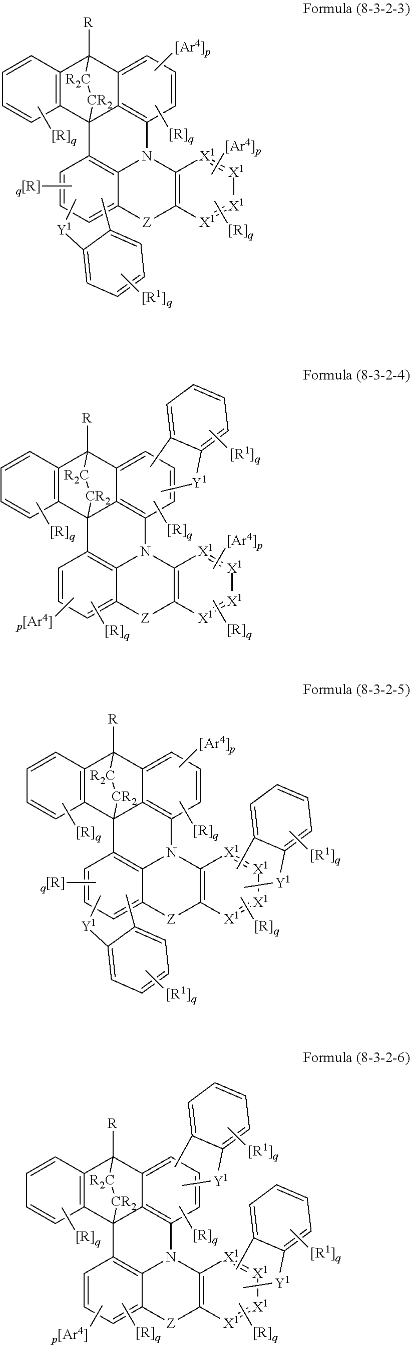

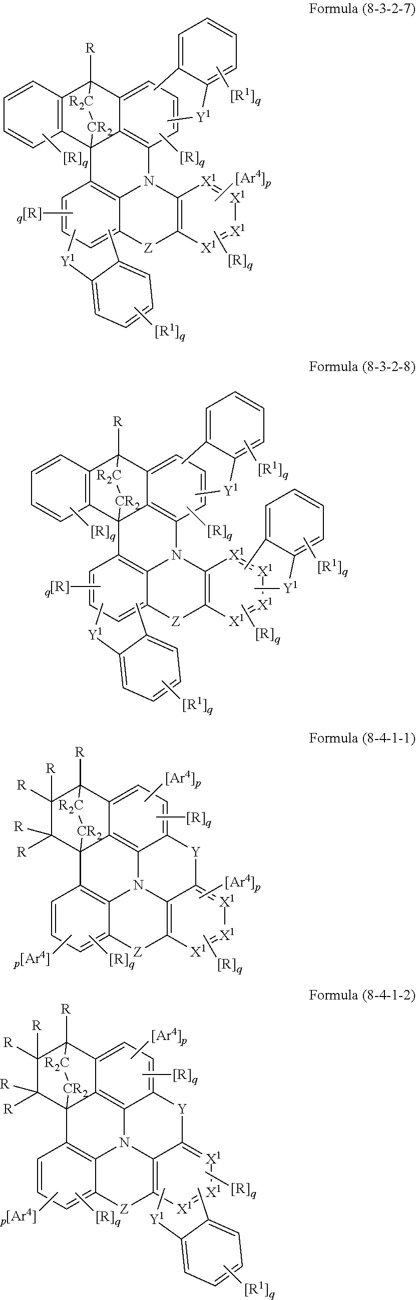

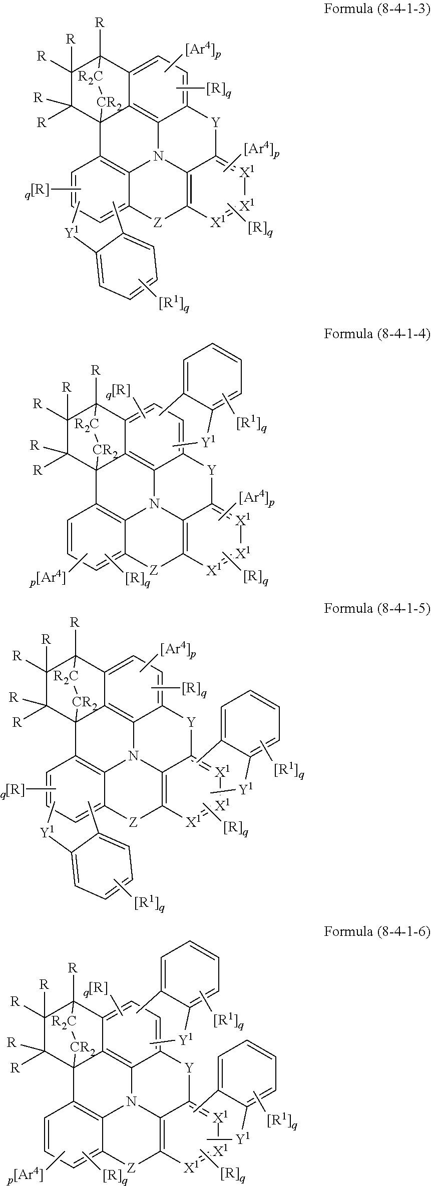

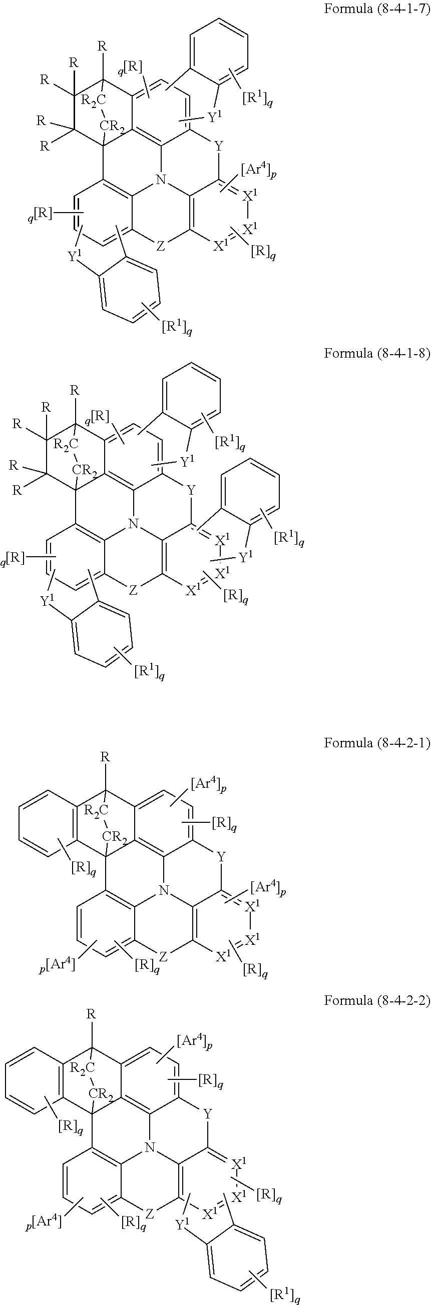

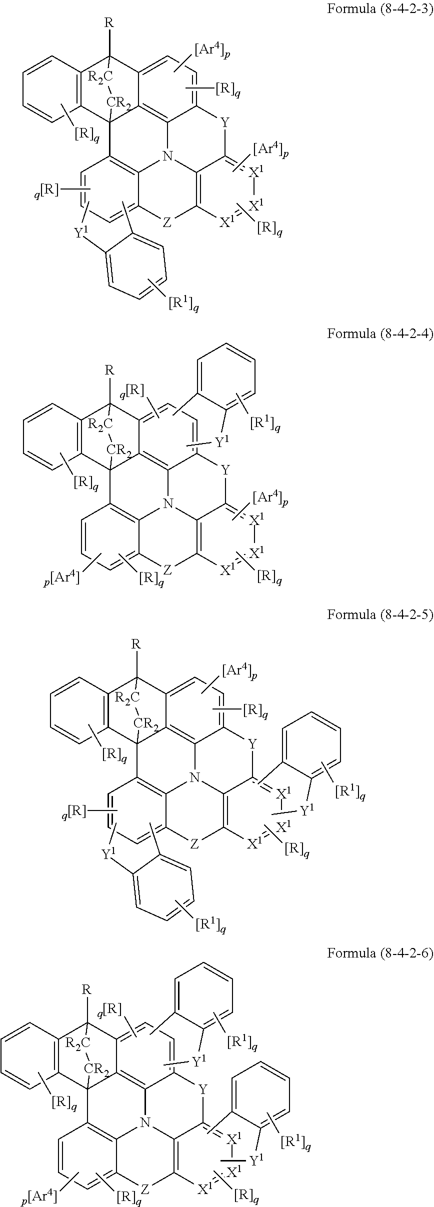

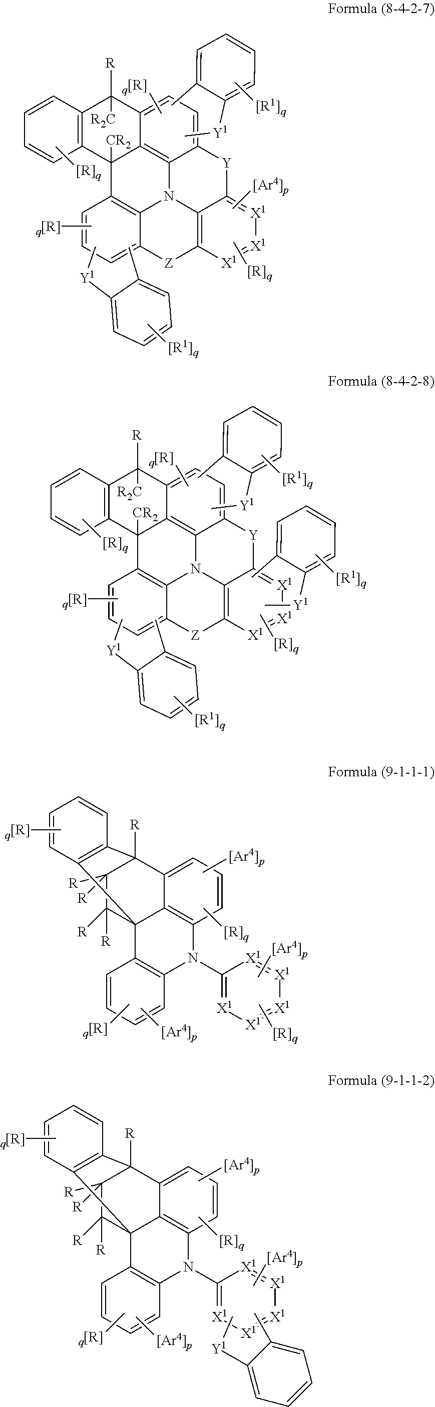

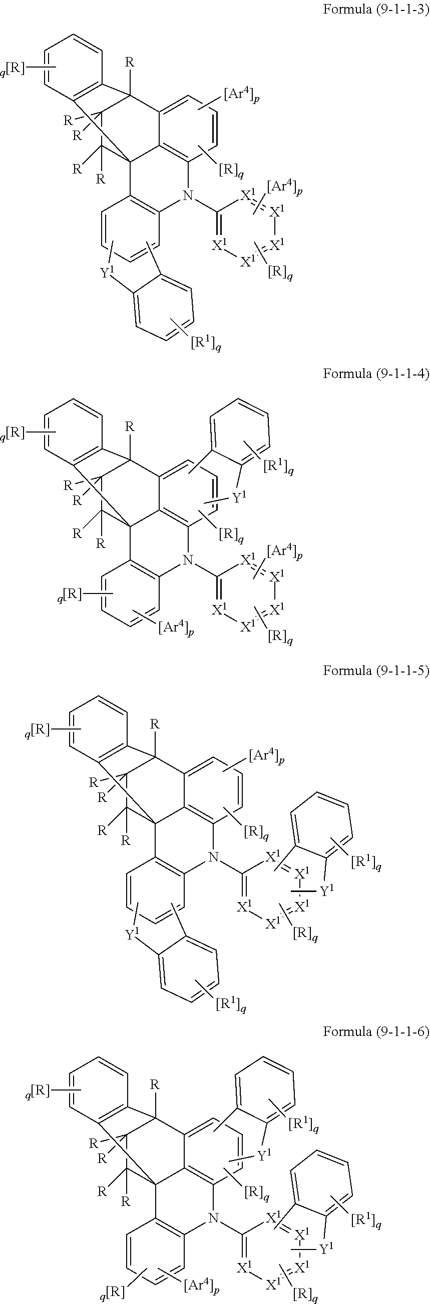

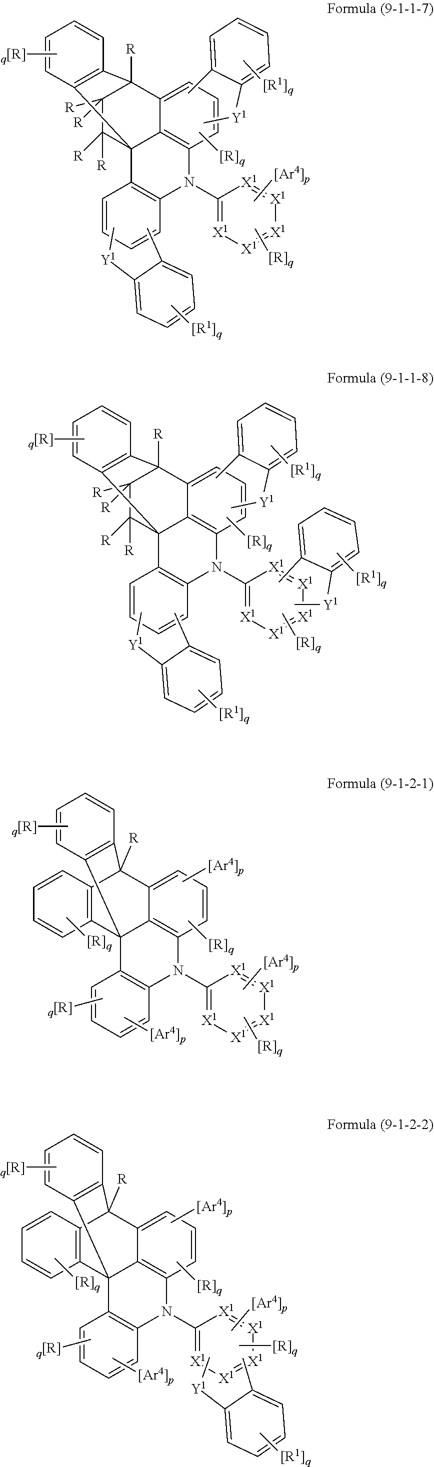

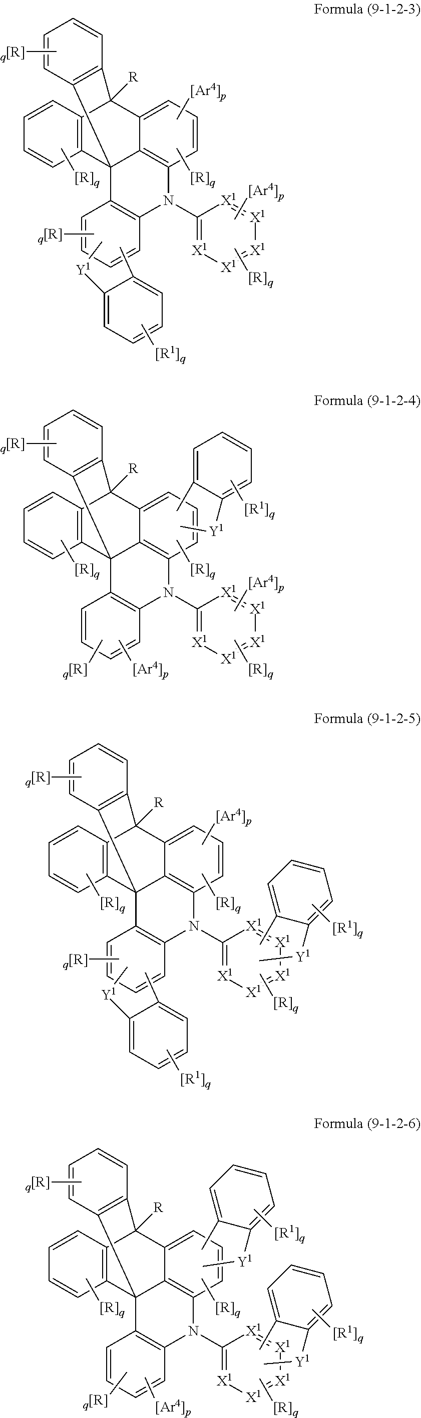

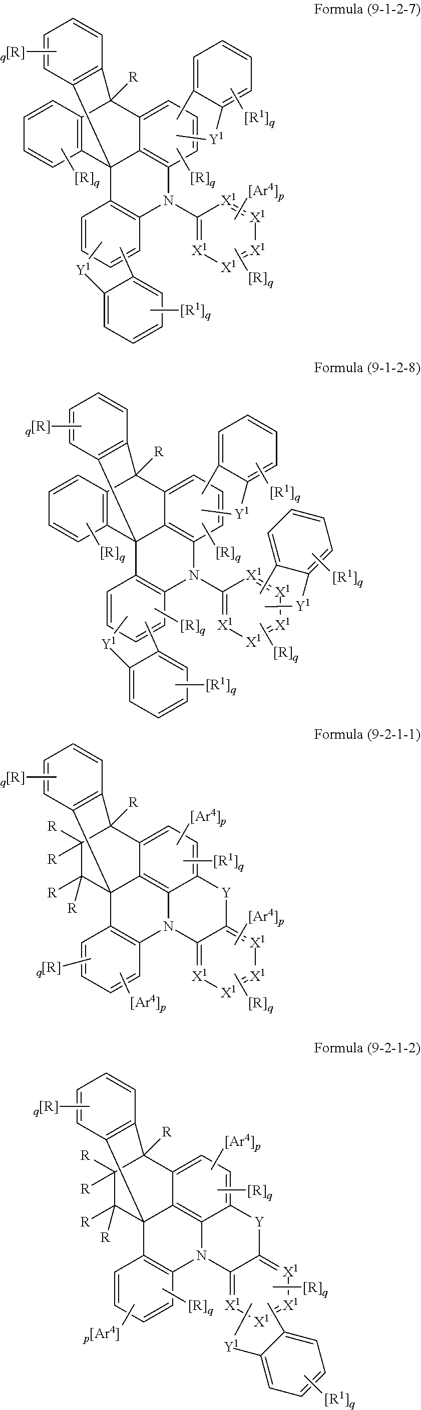

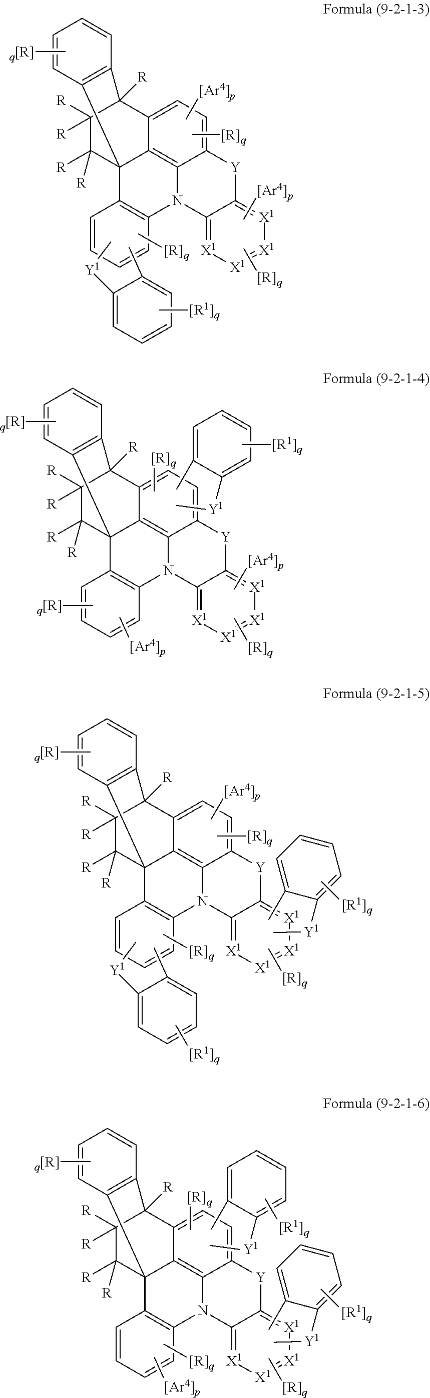

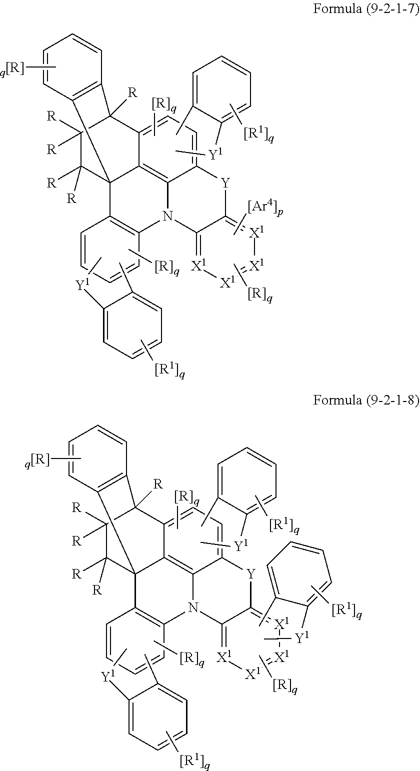

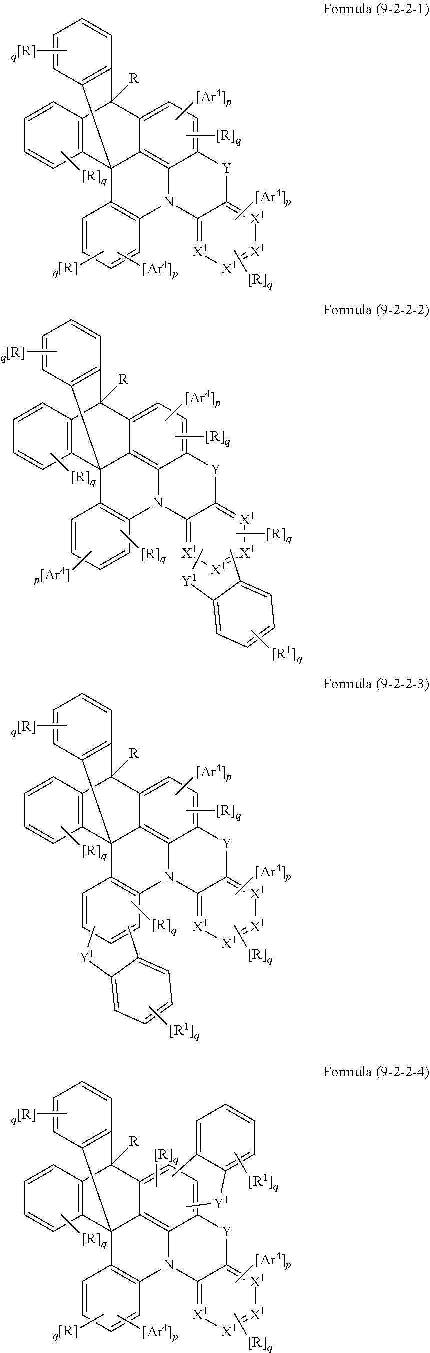

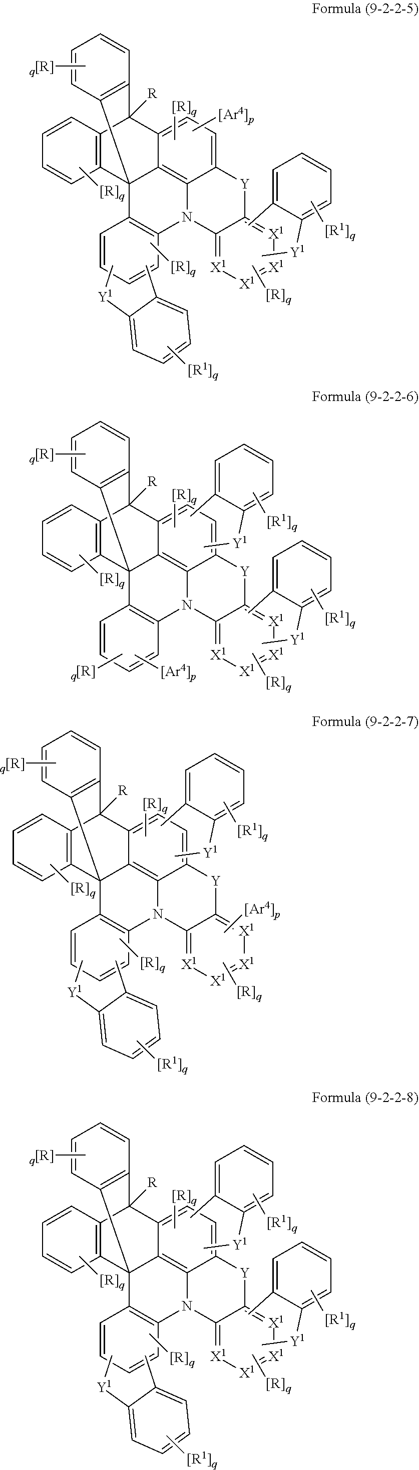

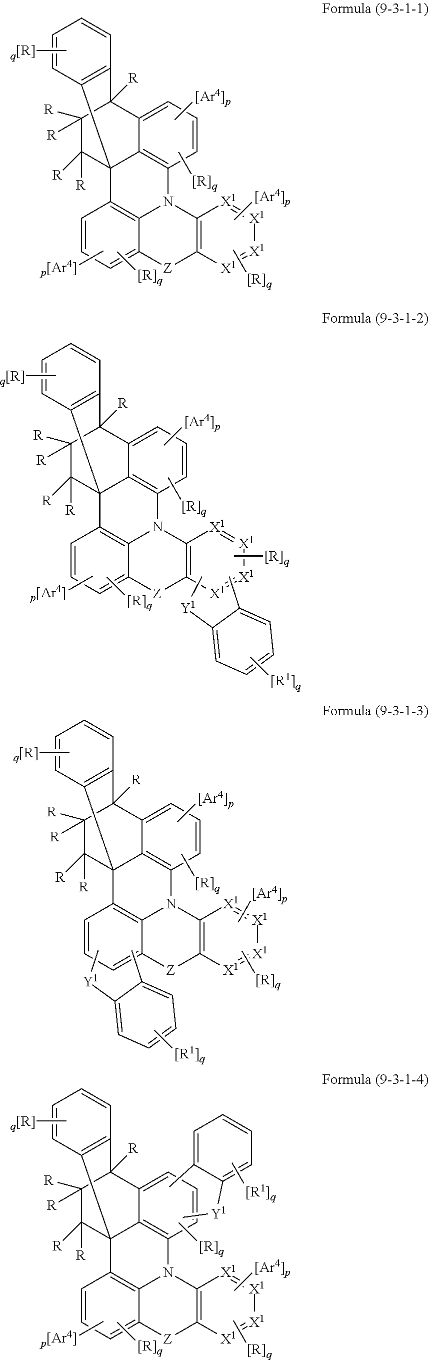

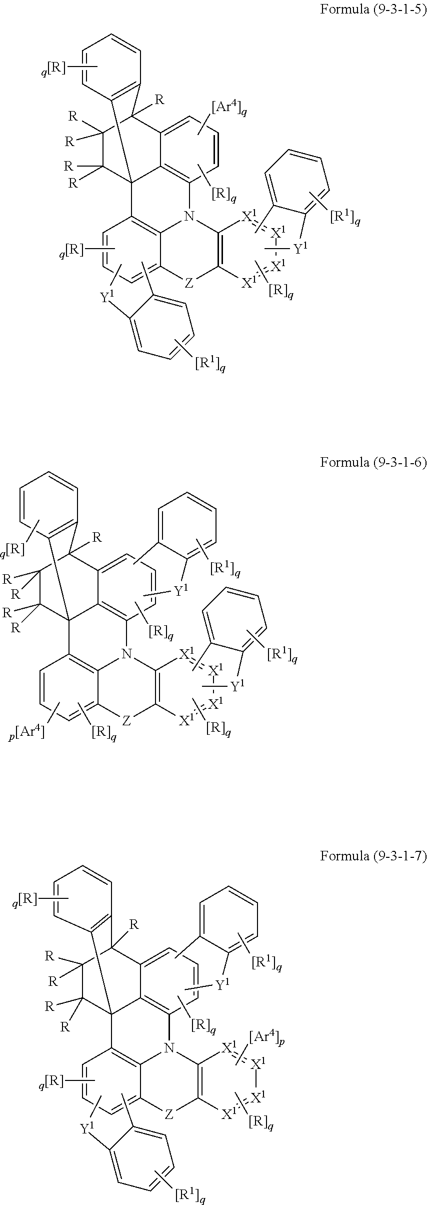

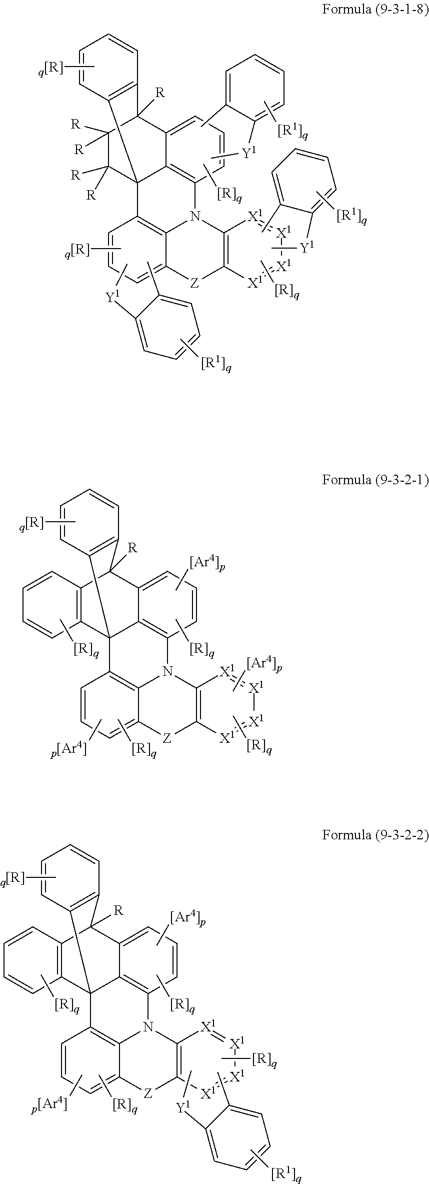

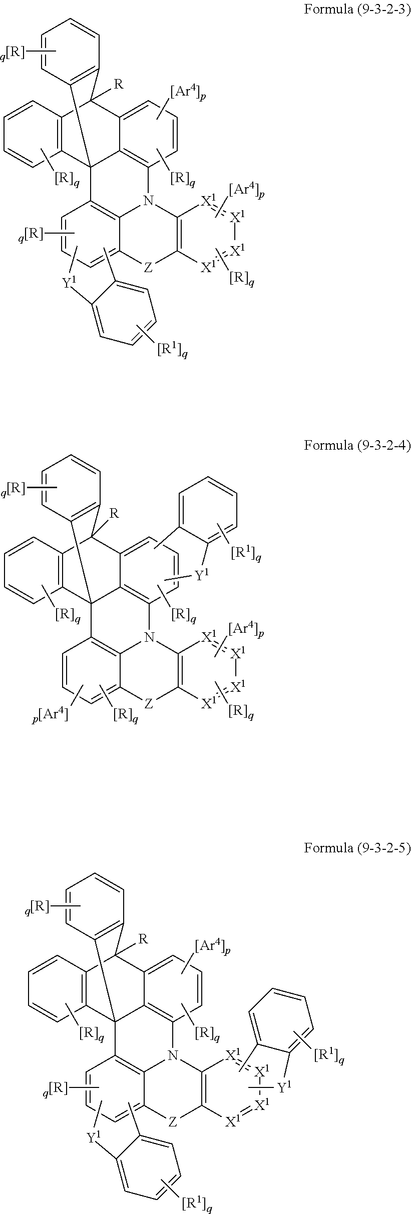

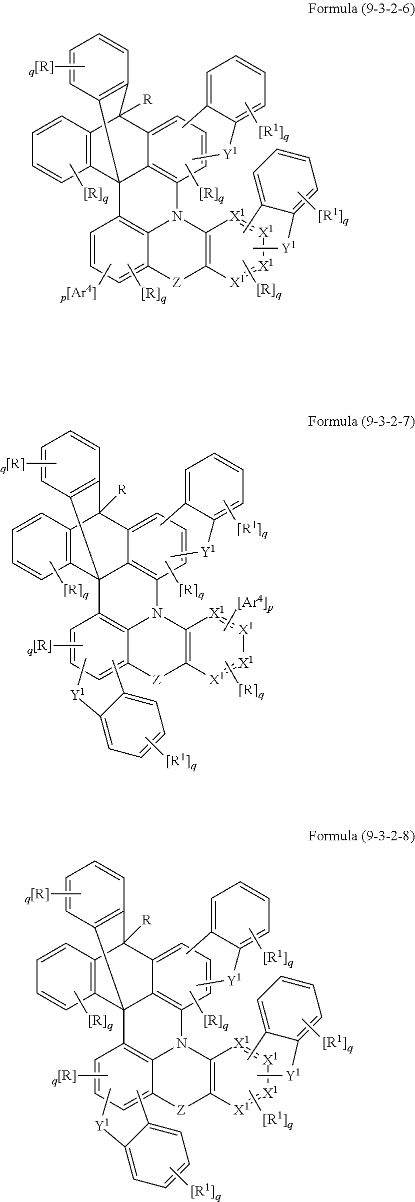

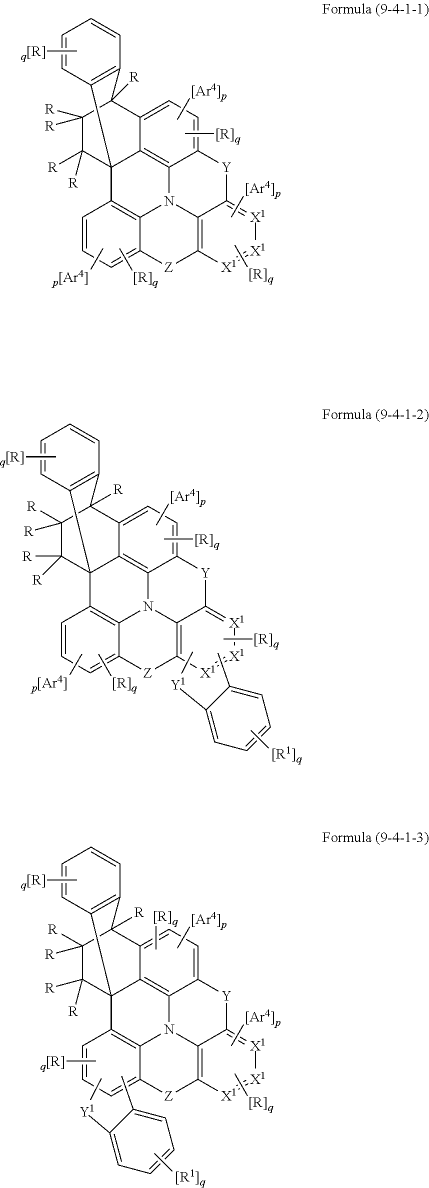

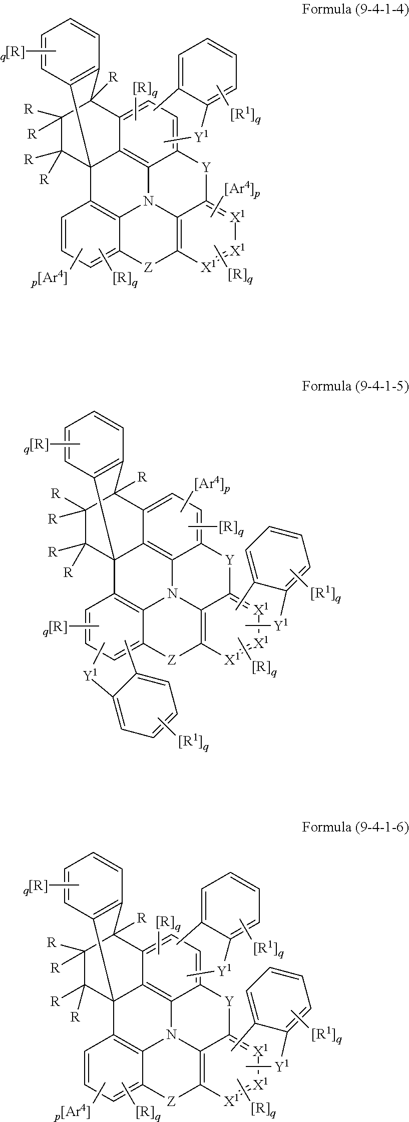

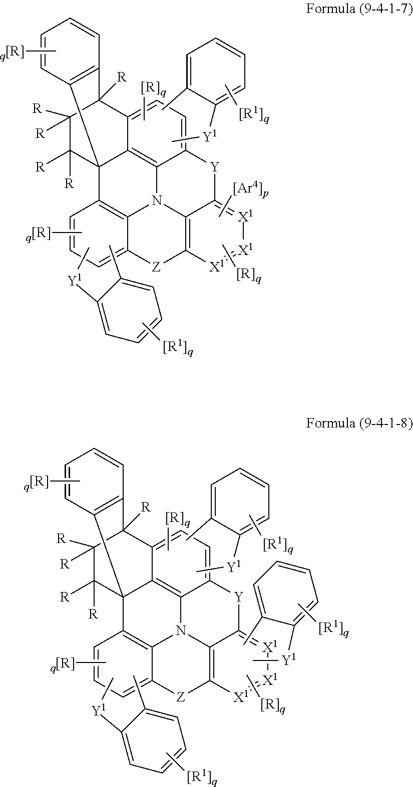

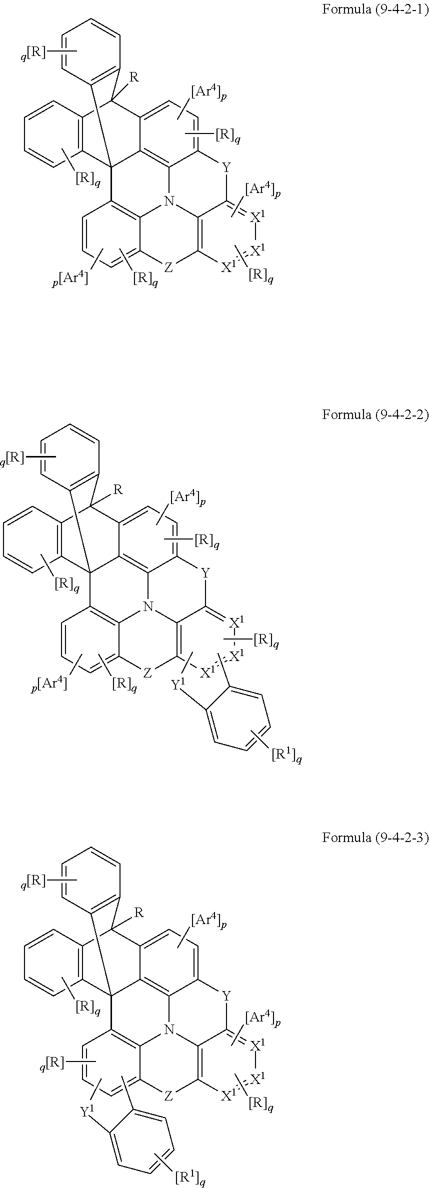

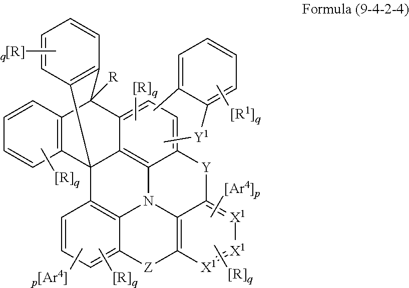

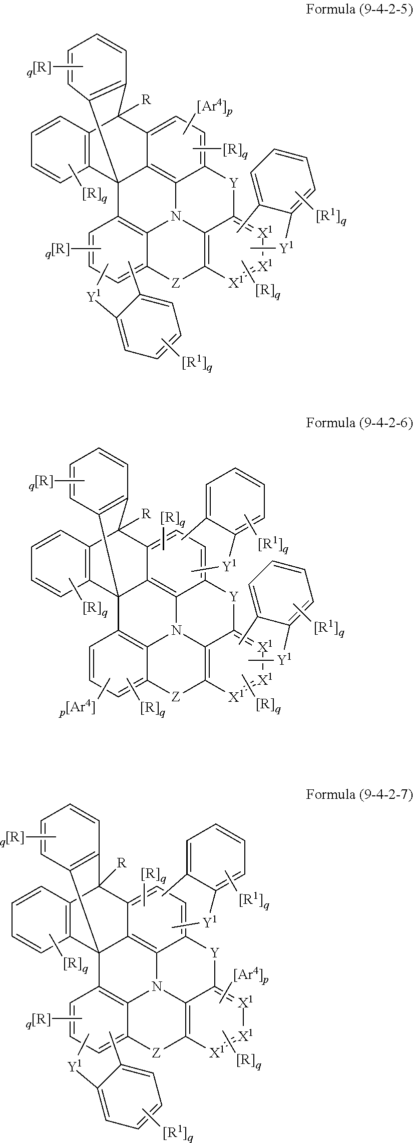

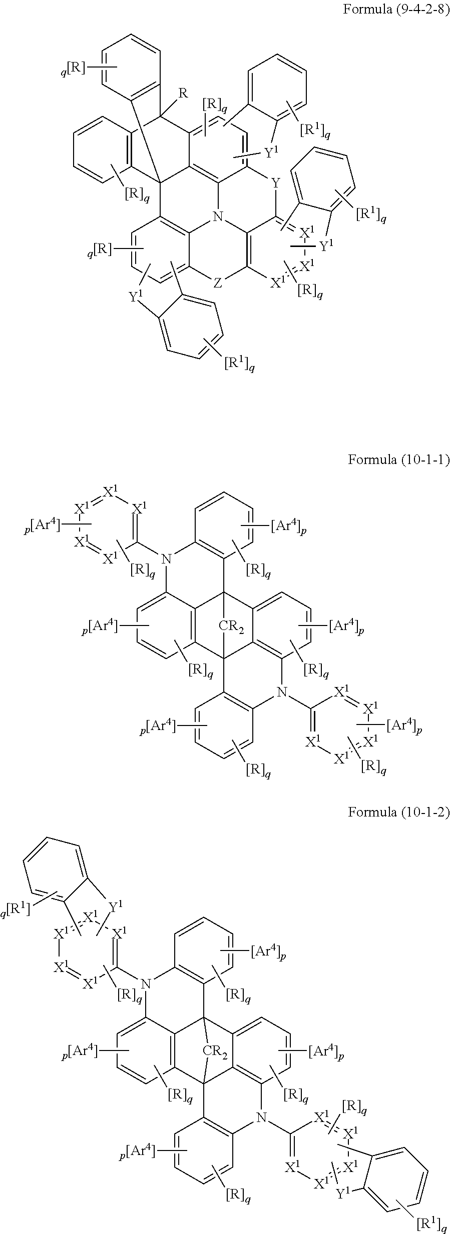

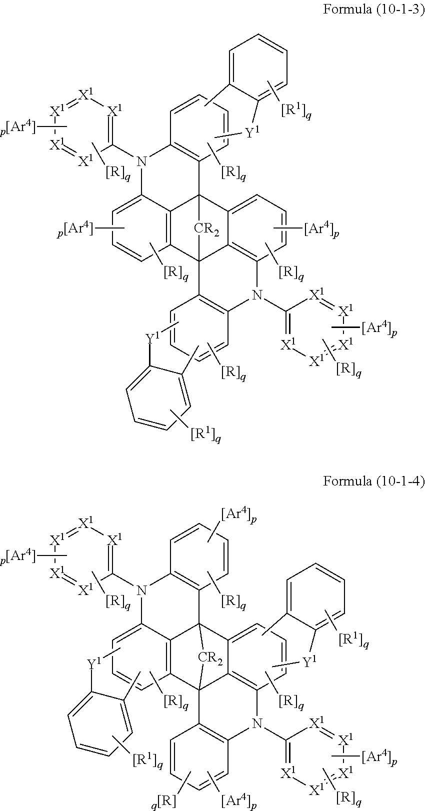

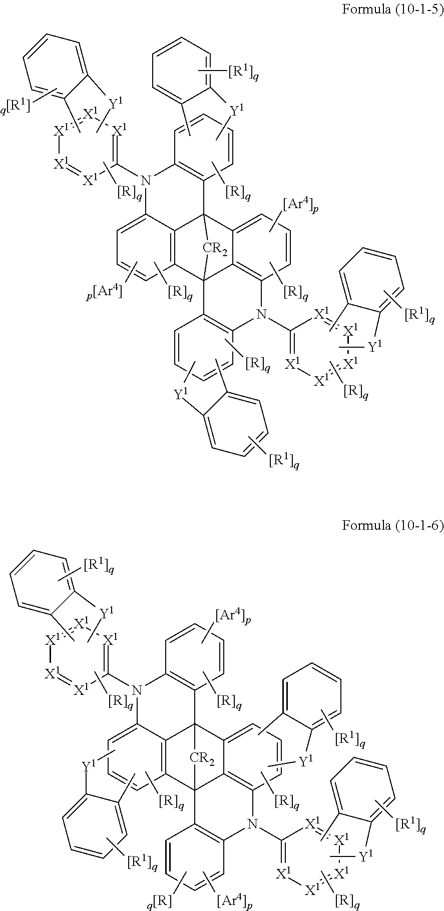

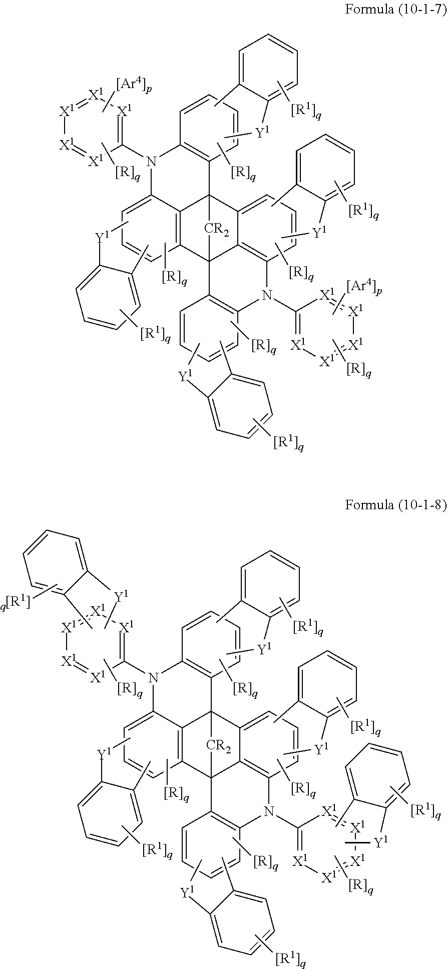

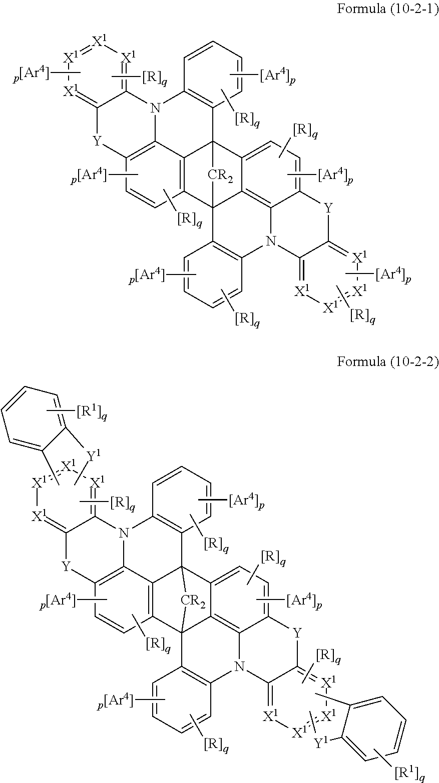

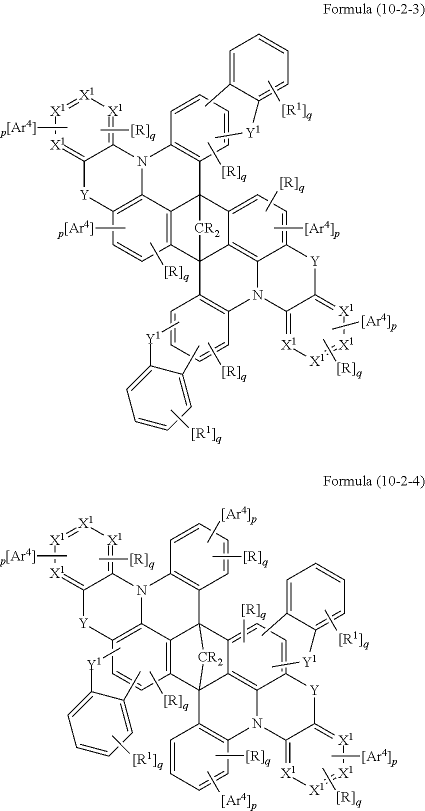

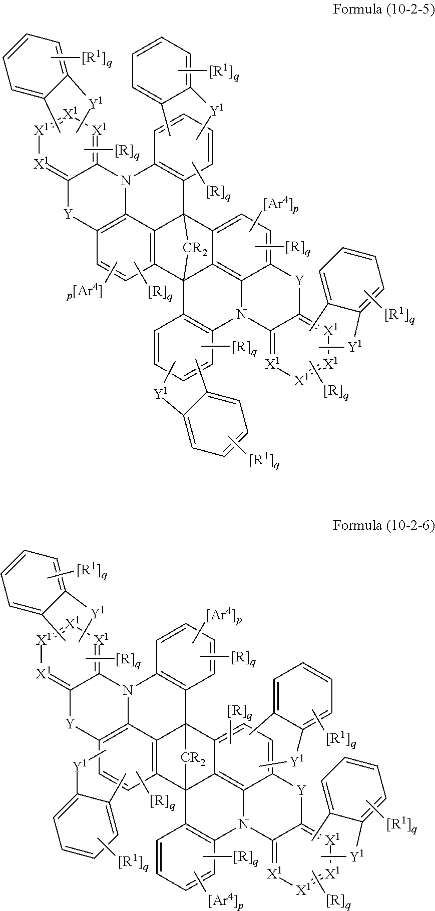

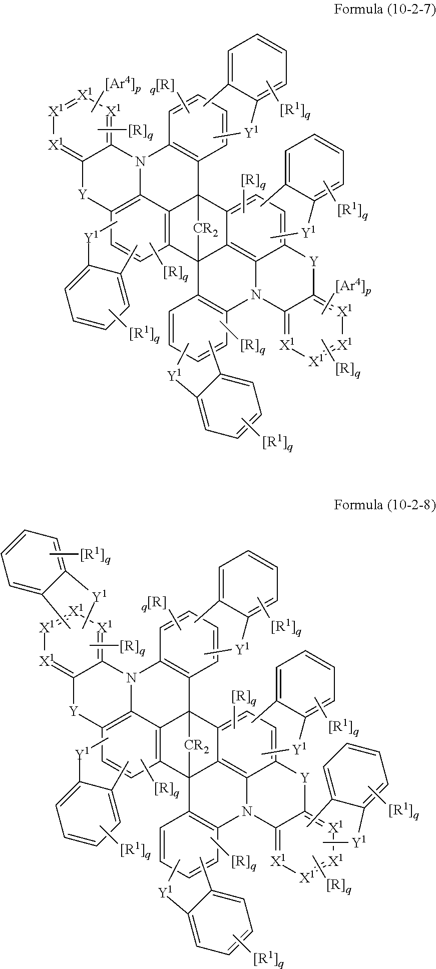

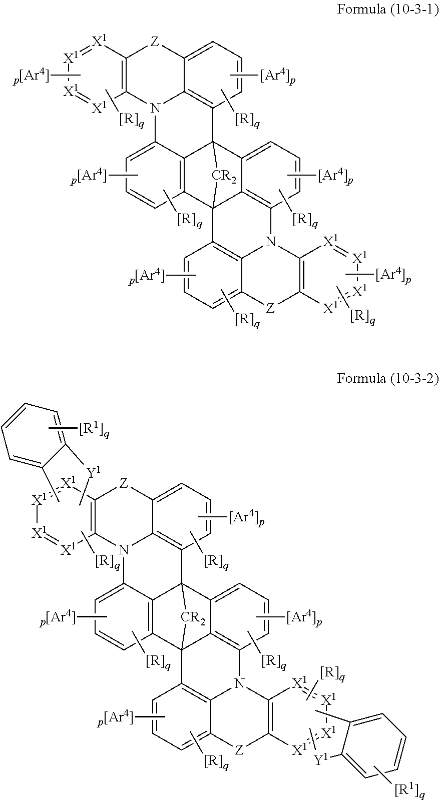

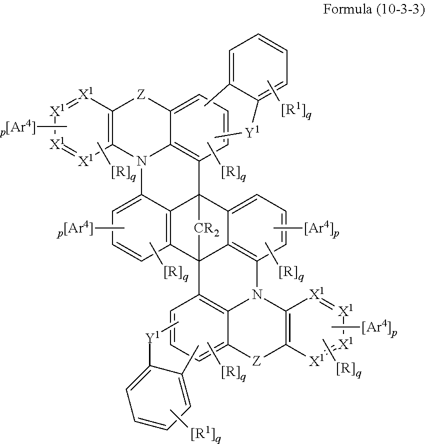

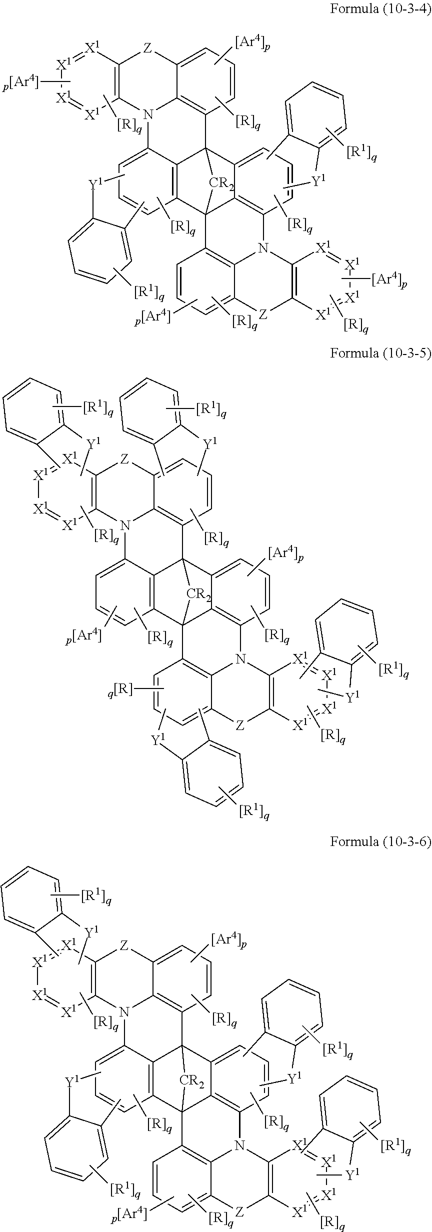

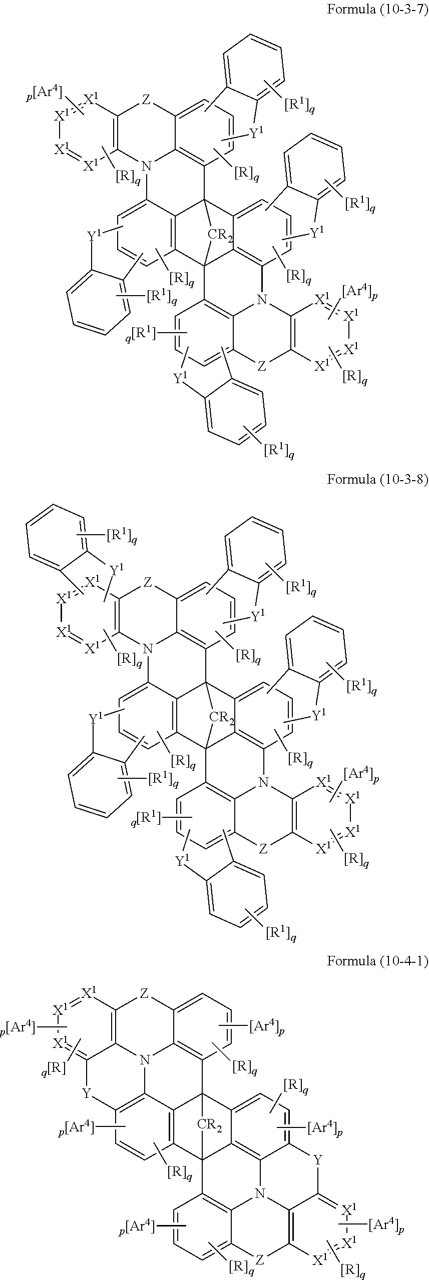

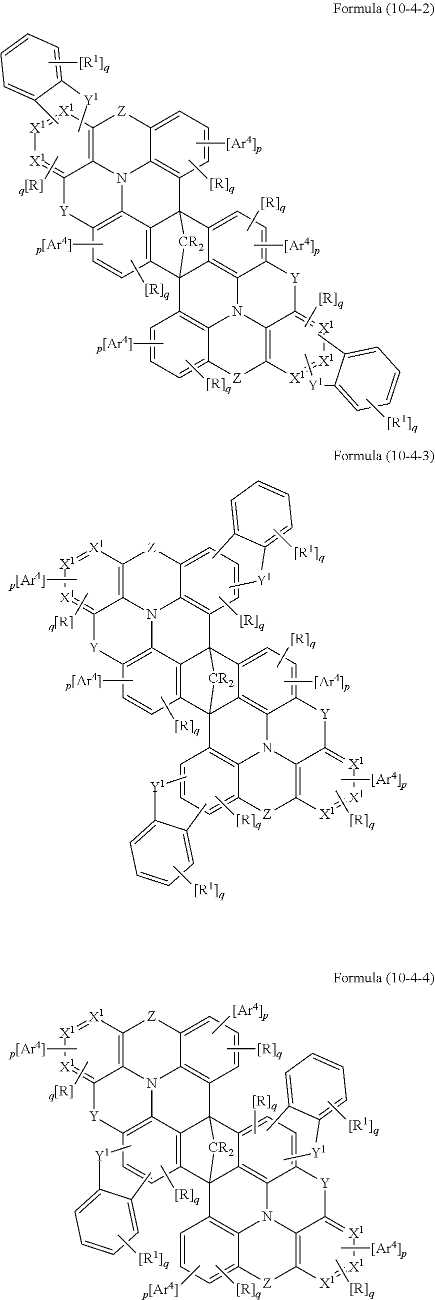

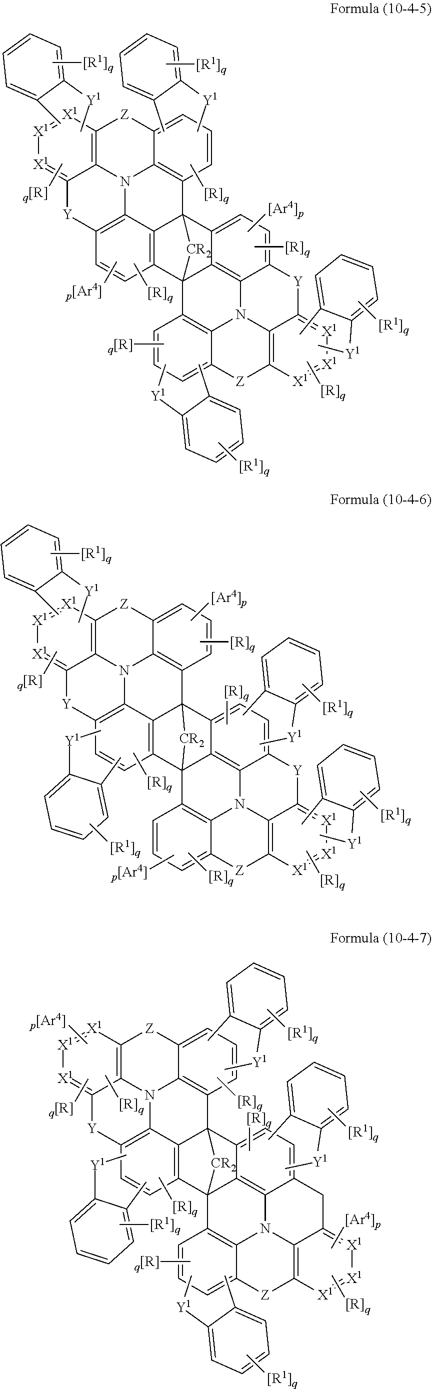

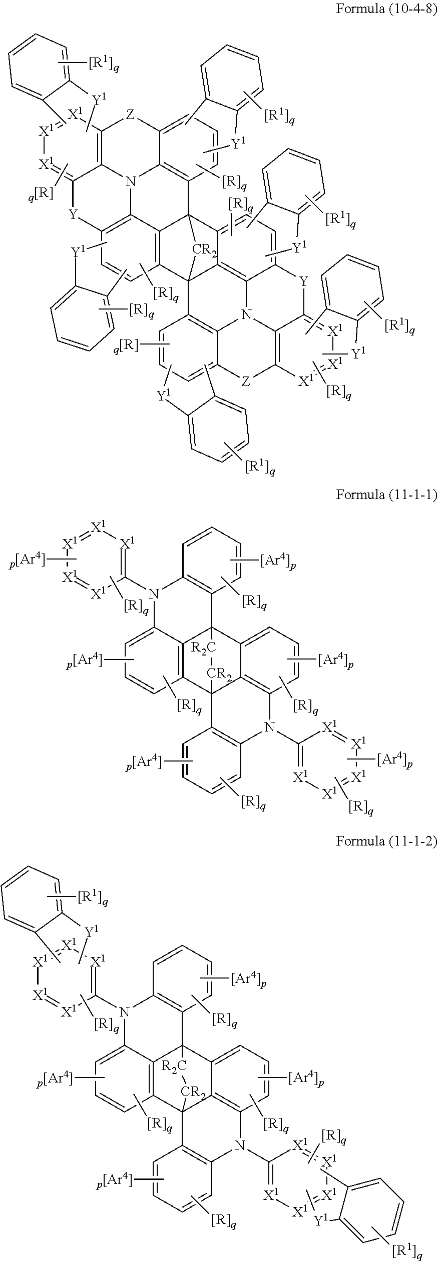

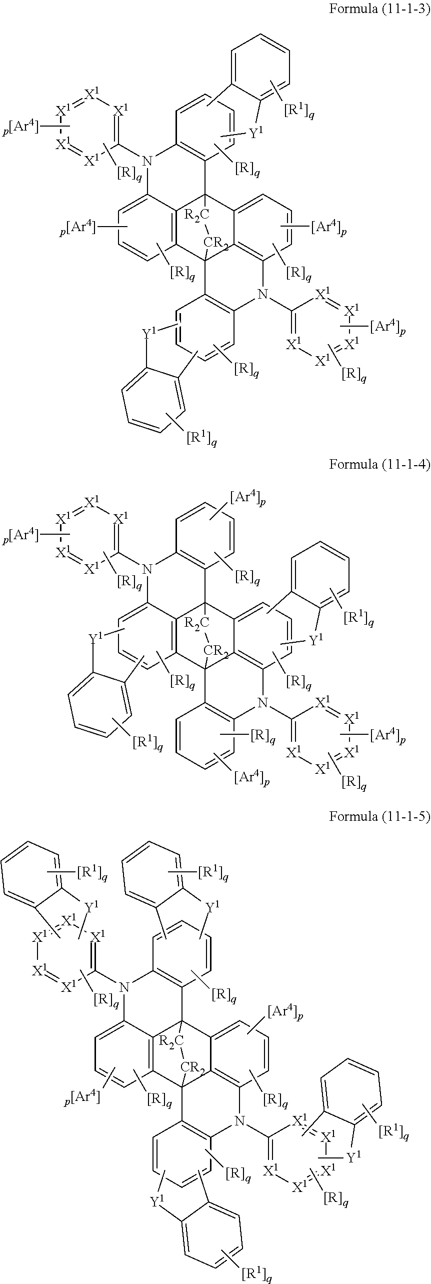

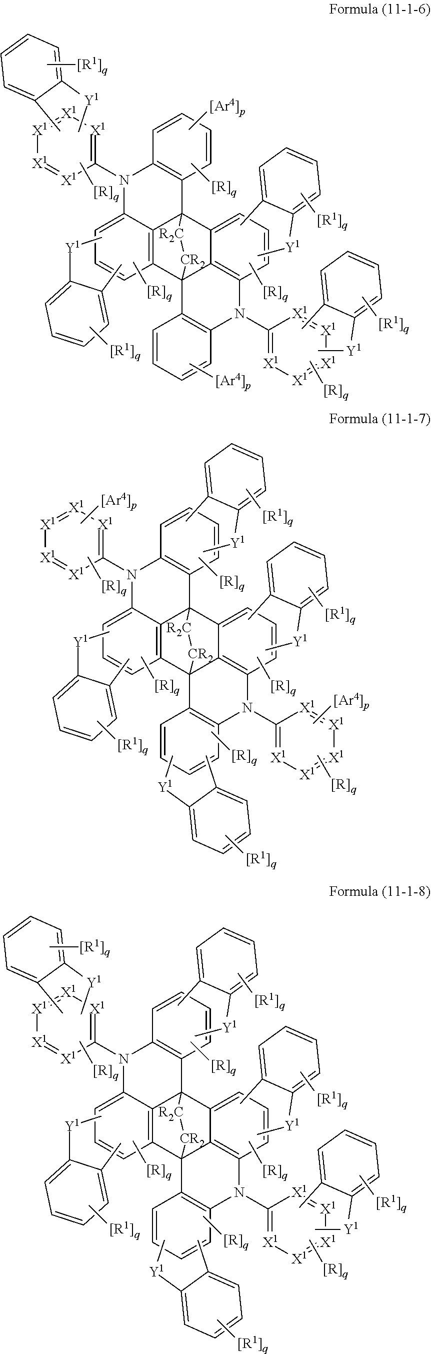

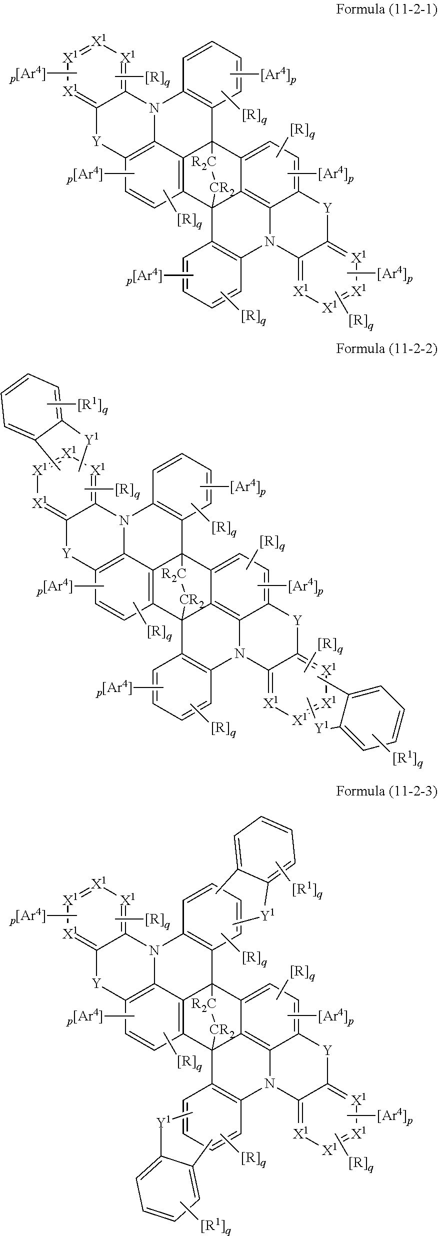

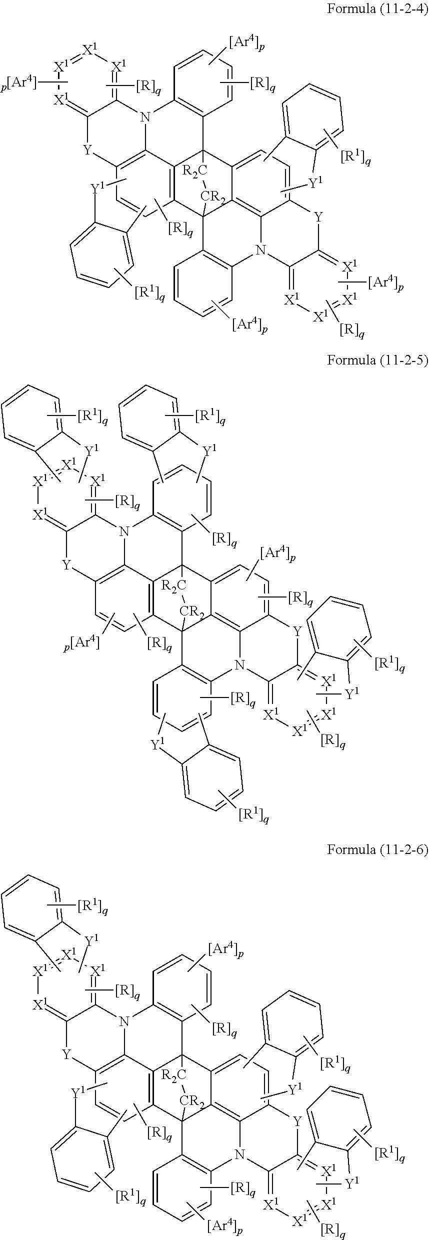

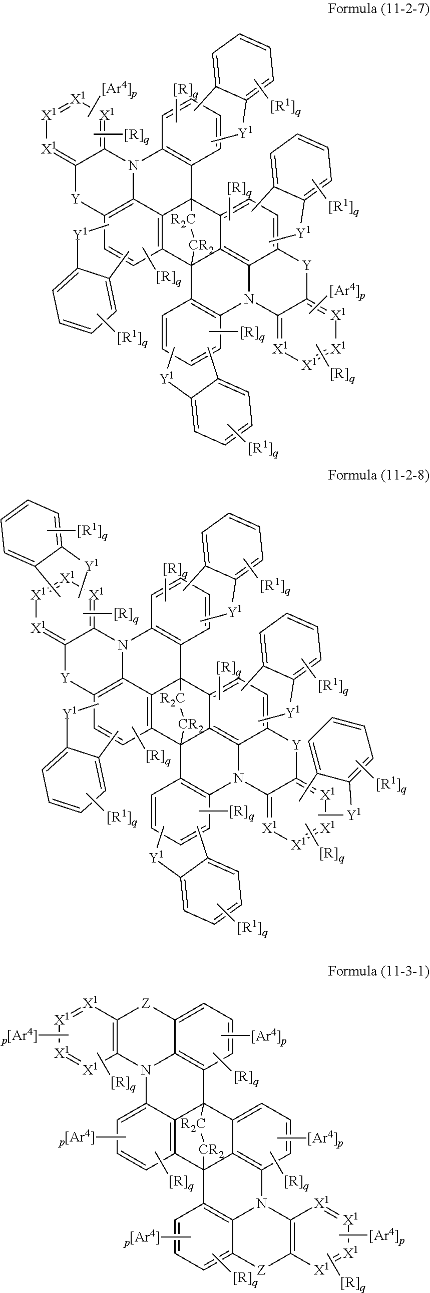

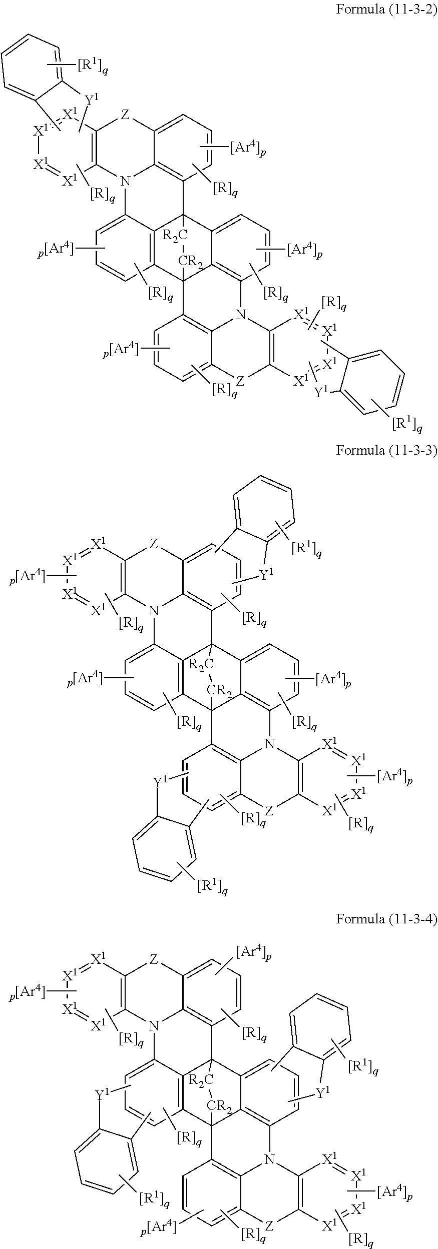

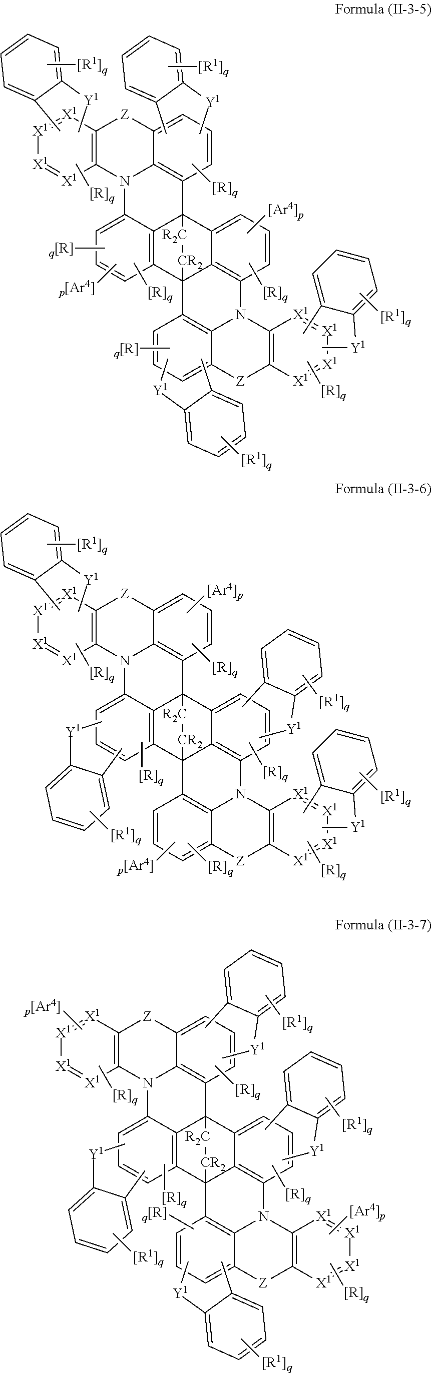

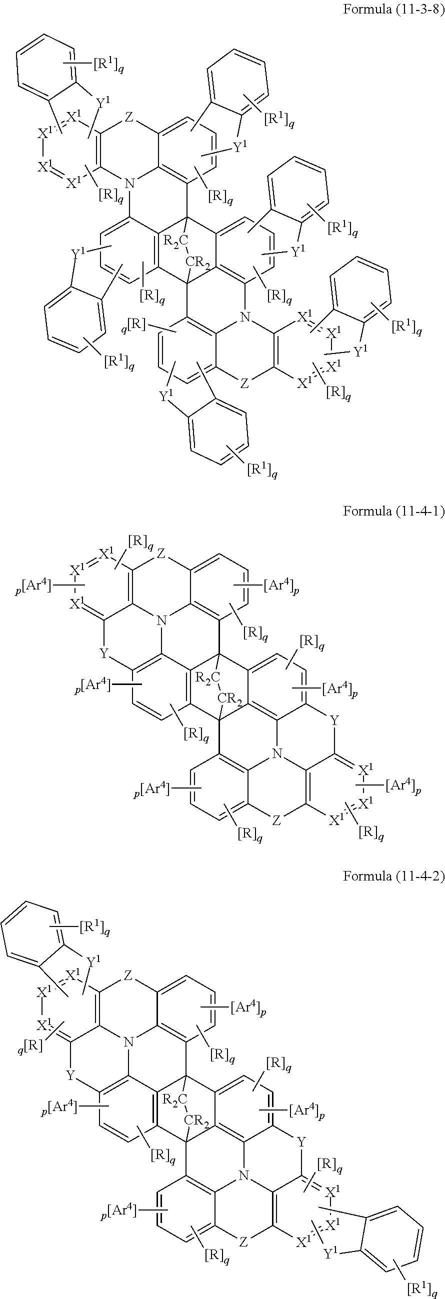

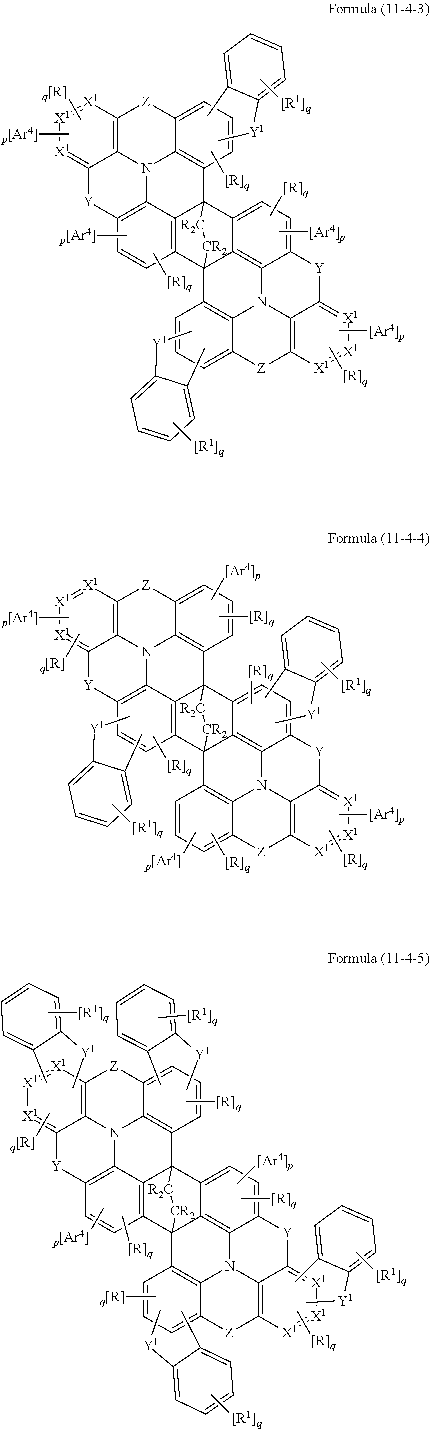

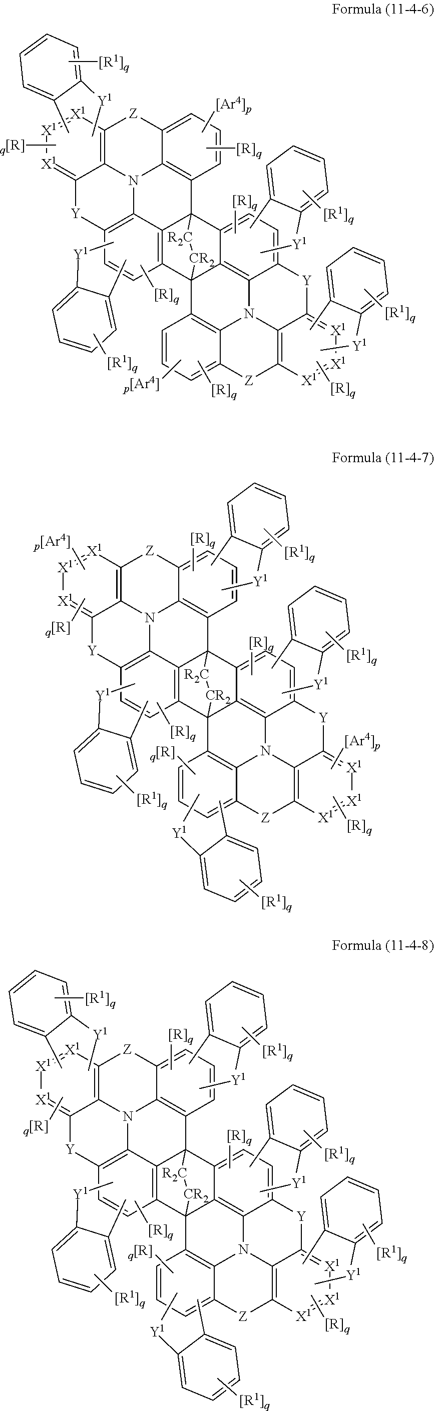

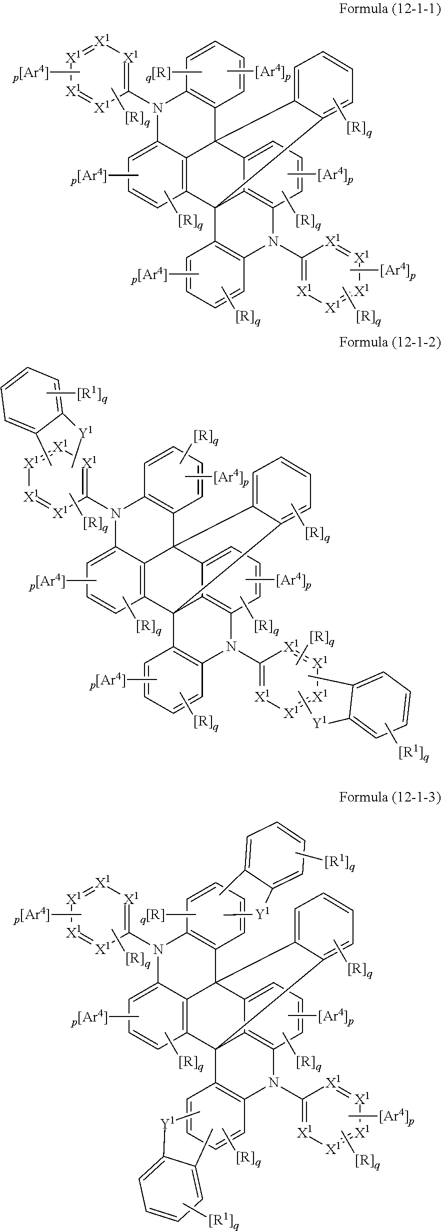

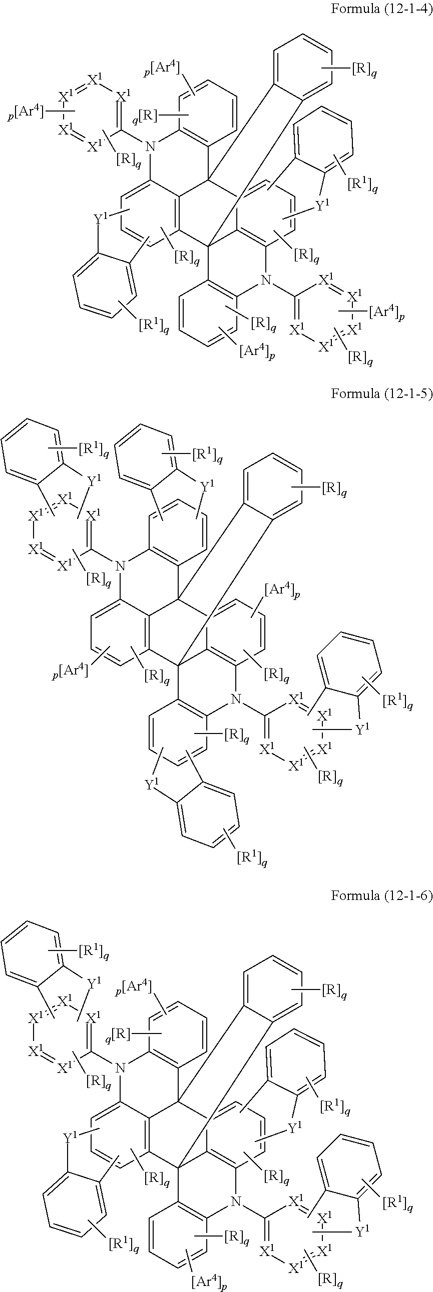

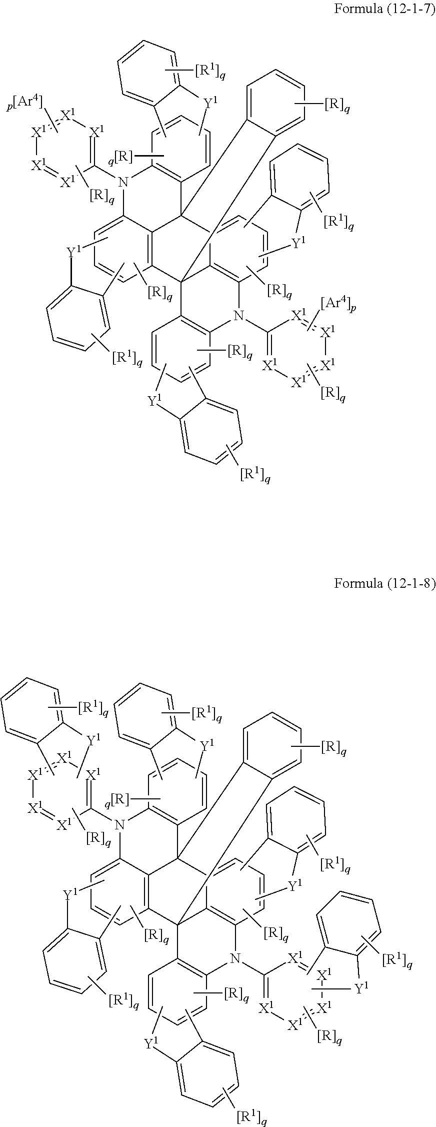

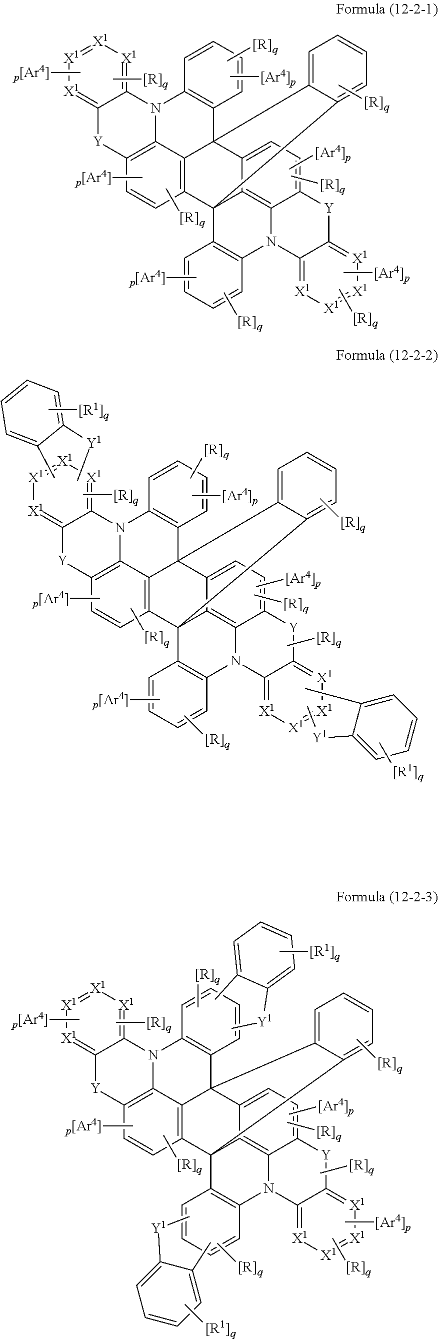

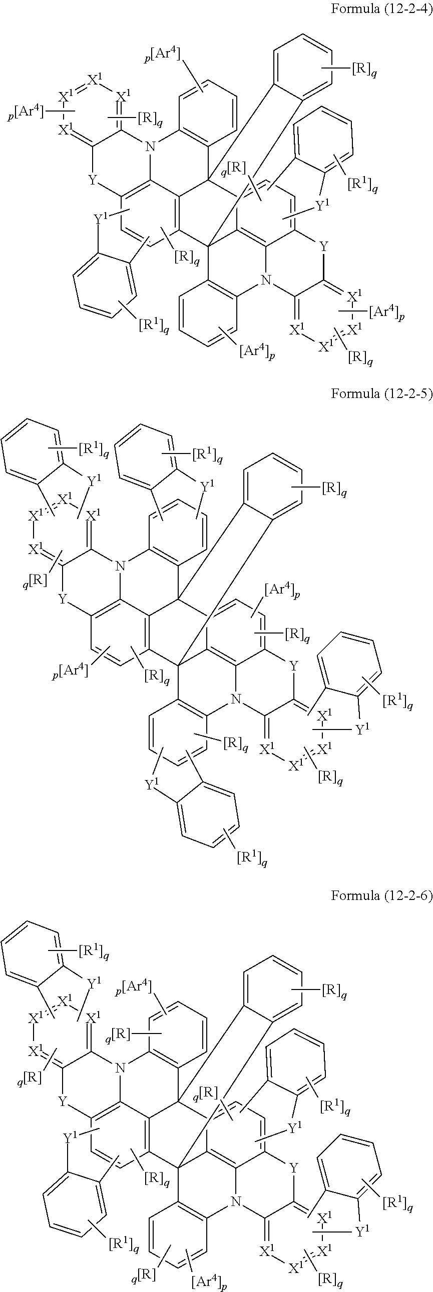

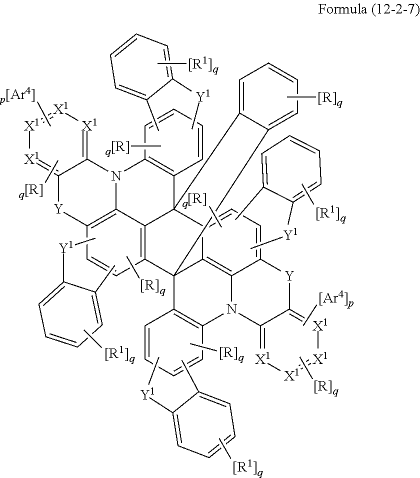

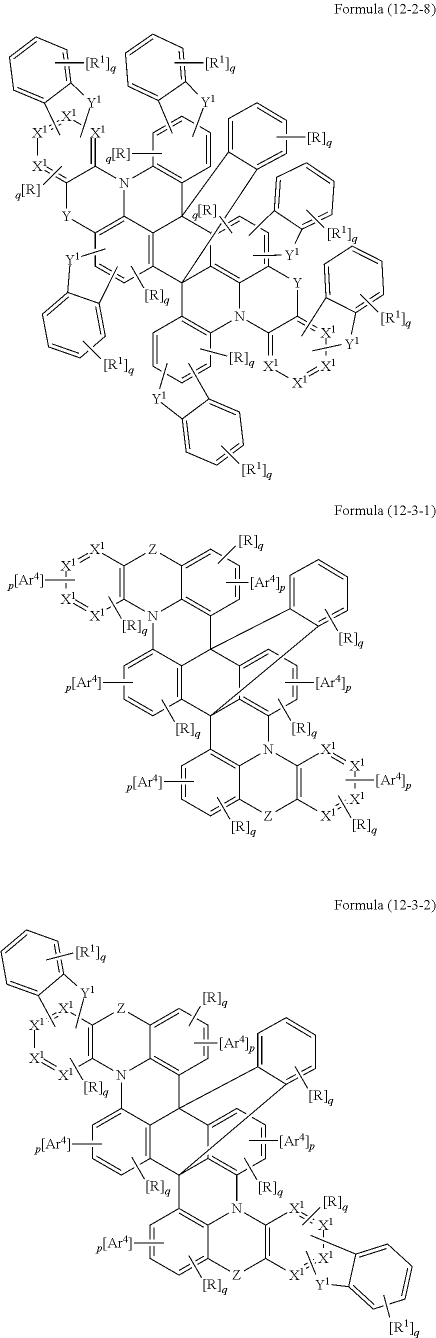

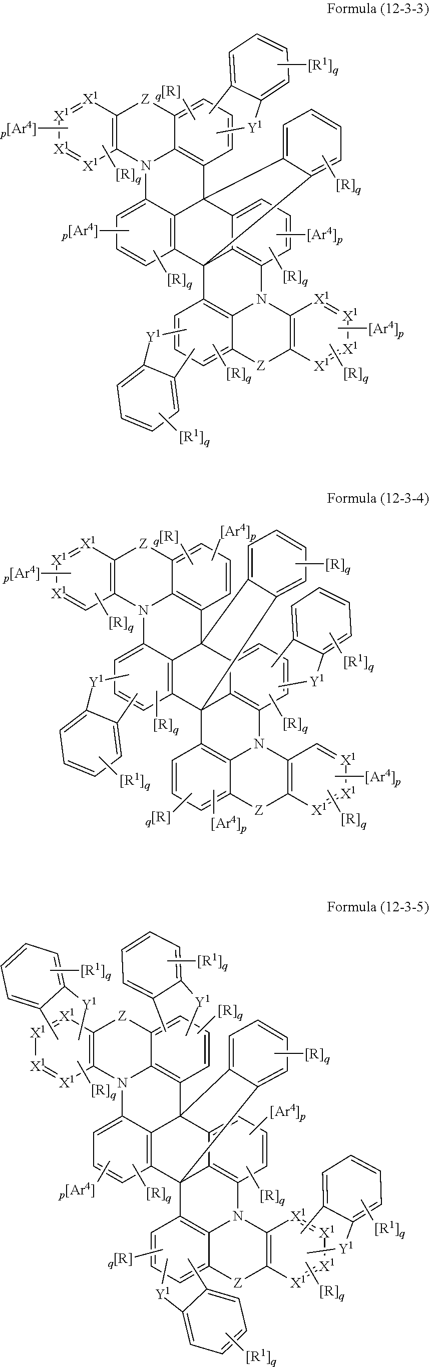

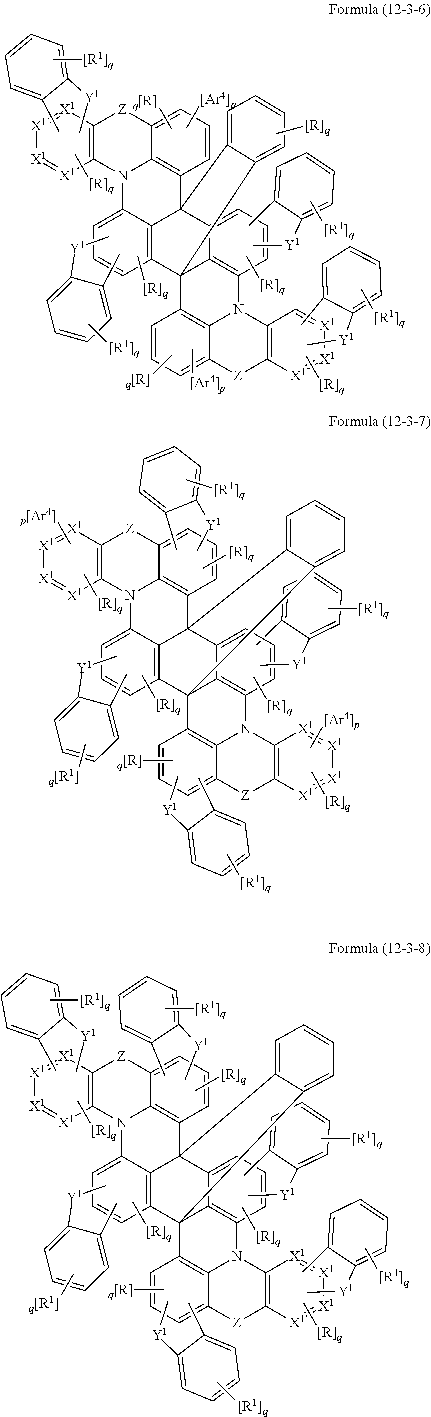

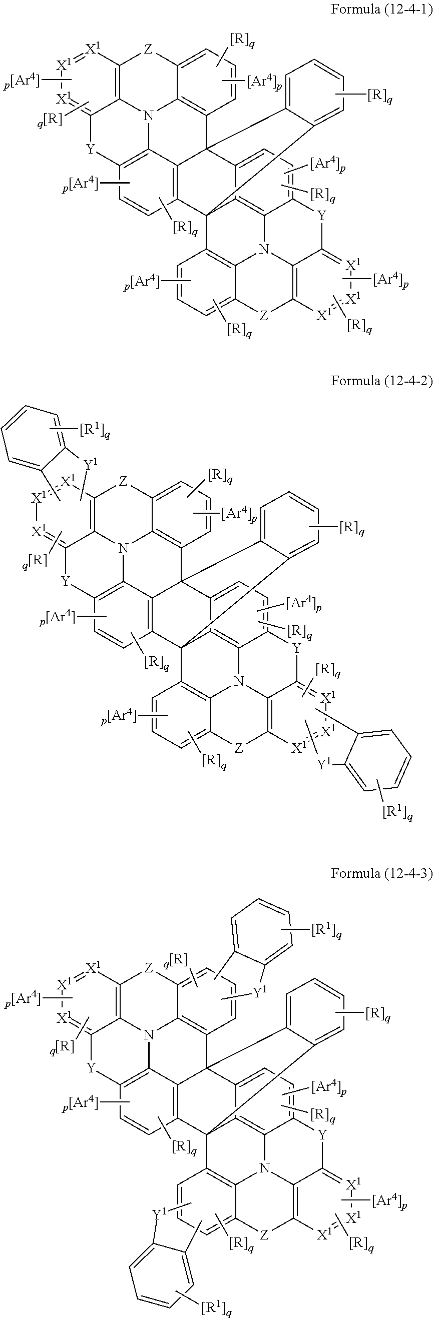

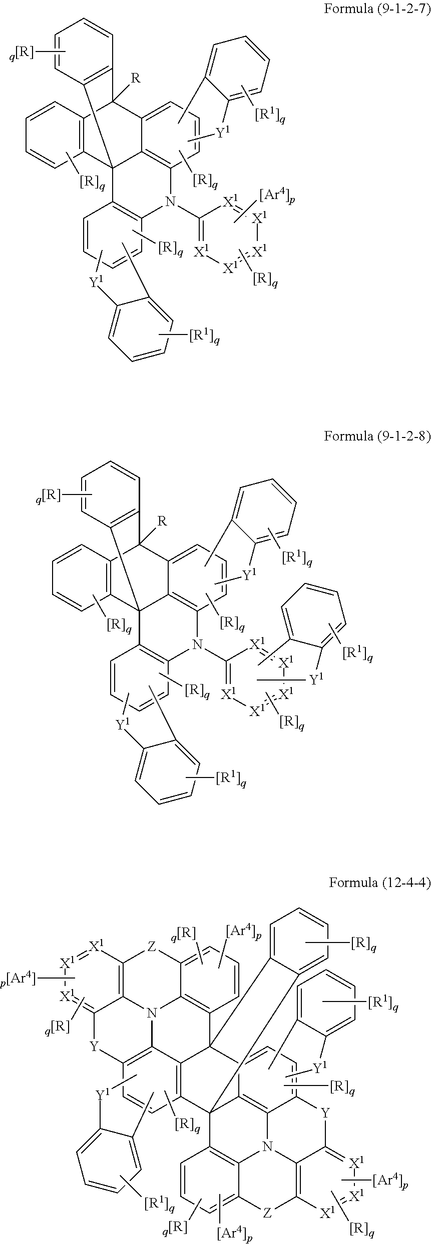

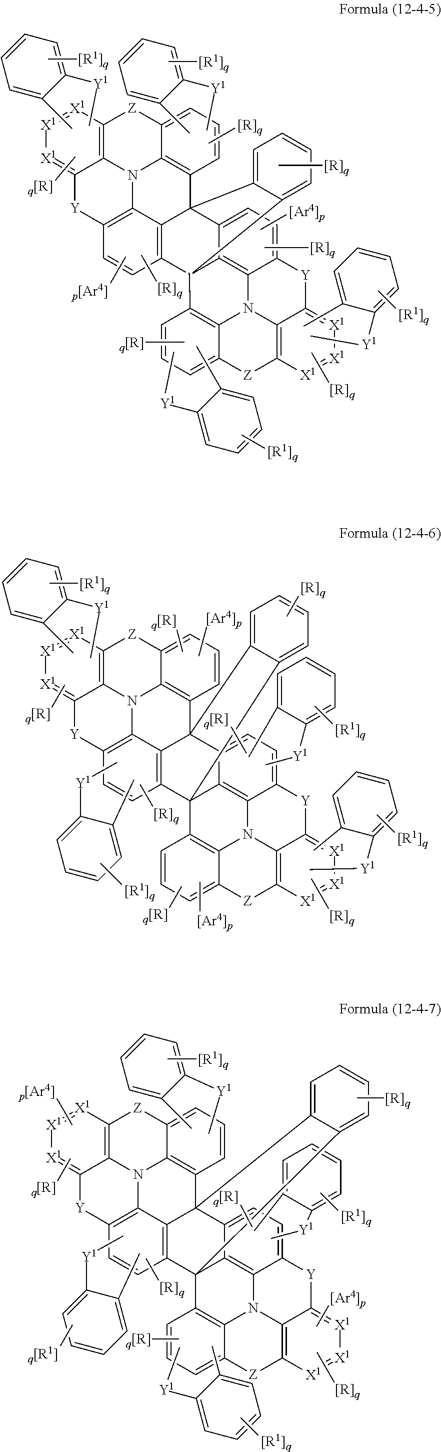

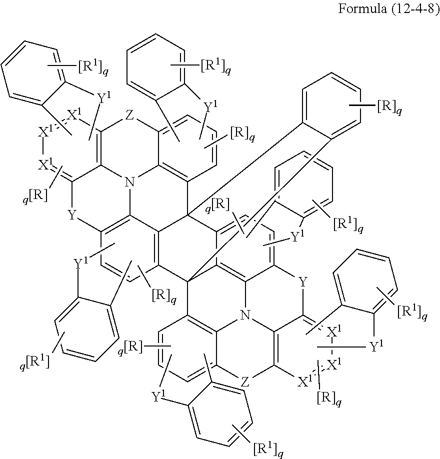

where the symbols used have the definitions given above for formulae (7-1) to (7-4), where, in addition:
X¹ is the same or different at each instance and is CH, CD, CF or N, with the proviso that not more than three X¹ groups per cycle are N;
q, p are the same or different at each instance and, depending on the substitutable positions on the respective cycle, are 0, 1, 2, 3, 4 or 5;
Y¹ is the same or different at each instance and is BR, BAr′, C═O, C(R)₂, —RC═CR—, o-arylene, NR, NAr′, PR, SO₂, SiR₂, SiAr′₂, P(O)R, P(O)Ar′, O or S;
Ar⁴ is the same or different at each instance and is an aromatic or heteroaromatic ring system which has 5 to 60 aromatic ring atoms and may be substituted by one or more R¹ radicals.

In a preferred embodiment, X¹ is the same or different at each instance and is CH, CD, CF or N, with the proviso that not more than three X¹ groups per cycle are N, where the N are not adjacent.

The maximum value of p+q for each cycle is found from the substitutable positions. When there is already a ring system fused onto this cycle via Y¹, as a result of this system, there are already 2 positions that are no longer available for further substitution.

The systems fusible with Y¹ in the formulae may be fused in any desired manner onto adjacent carbon atoms in the respective cycles to form a five-membered ring. Together with the cycle, an aromatic or heteroaromatic system is then formed.

In a preferred embodiment, Y¹ is the same or different at each instance and is BR, BAr′, C═O, C(R)₂, NR, NAr′, PR, SO₂, SiR₂, SiAr′₂, P(O)R, P(O)Ar′, O or S, more preferably BAr′, C═O, C(R)₂, NAr′, PR, SO₂, SiR₂, SiAr′₂, P(O)Ar′, O or S, especially BAr′, C═O, C(R)₂, NAr′, PR, SO₂, SiR₂, SiAr′₂, P(O)R, P(O)Ar′, O or S.

In a preferred embodiment, p is 0, 1 or 2, preferably 0 or 1.

In a preferred embodiment, p is 0, 1 or 2, preferably 0 or 1, while R, if present, is not an aromatic or heteroaromatic ring system; in that case, R is preferably the same or different at each instance and is H, D, F, CN or a straight-chain alkyl group having 1 to 20 carbon atoms.

As well as the substituents, suitability of the compound for different uses can be controlled via the presence or absence of Y and Z.

In a preferred embodiment, m, n are 0 for use as HTM (hole transport material). When m+n=1, and Y or Z is a single bond, the compounds are preferentially suitable for hTMMs (hole-transporting triplet matrix materials).

For FE (fluorescence emitters), it is preferable that n and/or m=1, and Z and/or Y is BR, preferably BAr′ (CABNA type).

In a preferred embodiment, Y and/or Z is the same or different at each instance and is a single bond, BAr′, C═O, C(R)z, NAr, PR, SO₂, SiR₂, SiAr′₂, P(O)Ar′, O or S, more preferably a single bond, BAr′, C═O, C(R)₂, SiR₂, SiAr′z, P(O)Ar′, O or S, especially a single bond, BAr′ or O.

Particular preference is given to the following embodiments:

| Formula | Y | Z |
|---|---|---|
| 7-2 | single bond | — |
| 7-2 | BAr′ | — |
| 7-2 | O | — |
| 7-3 | — | single bond |
| 7-3 | — | BAr′ |
| 7-3 | — | O |
| 7-4 | single bond | single bond |
| 7-4 | BAr′ | BAr′ |
| 7-4 | single bond | BAr′ |
| 7-4 | BAr′ | single bond |
| 7-4 | BAr′ | O |
| 7-4 | O | BAr′ |
| 8-2 | single bond | — |
| 8-2 | BAr′ | — |
| 8-2 | O | — |
| 8-3 | — | single bond |
| 8-3 | — | BAr′ |
| 8-3 | — | O |
| 8-4 | single bond | single bond |
| 8-4 | BAr′ | BAr′ |
| 8-4 | single bond | BAr′ |
| 8-4 | BAr′ | single bond |
| 8-4 | BAr′ | O |
| 8-4 | O | BAr′ |
| 9-2 | single bond | — |
| 9-2 | BAr′ | — |
| 9-2 | O | — |
| 9-3 | — | single bond |
| 9-3 | — | BAr′ |
| 9-3 | — | O |
| 9-4 | single bond | single bond |
| 9-4 | BAr′ | BAr′ |
| 9-4 | single bond | BAr′ |
| 9-4 | BAr′ | single bond |
| 9-4 | BAr′ | O |
| 9-4 | O | BAr′ |
| 10-2 | single bond | — |
| 10-2 | BAr′ | — |
| 10-2 | O | — |
| 10-3 | — | single bond |
| 10-3 | — | BAr′ |
| 10-3 | — | O |
| 10-4 | single bond | single bond |
| 10-4 | BAr′ | BAr′ |
| 10-4 | single bond | BAr′ |
| 10-4 | BAr′ | single bond |
| 10-4 | BAr | O |
| 10-4 | O | BAr′ |
| 11-2 | single bond | — |
| 11-2 | BAr′ | — |
| 11-2 | O | — |
| 11-3 | — | single bond |
| 11-3 | — | BAr′ |
| 11-3 | — | O |
| 11-4 | single bond | single bond |
| 11-4 | BAr′ | BAr′ |
| 11-4 | single bond | BAr′ |
| 11-4 | BAr′ | single bond |
| 11-4 | BAr′ | O |
| 11-4 | O | BAr′ |
| 12-2 | single bond | — |
| 12-2 | BAr′ | — |
| 12-2 | O | — |
| 12-3 | — | single bond |
| 12-3 | — | BAr′ |
| 12-3 | — | O |
| 12-4 | single bond | single bond |
| 12-4 | BAr′ | BAr′ |
| 12-4 | single bond | BAr′ |
| 12-4 | BAr′ | single bond |
| 12-4 | BAr′ | O |
| 12-4 | O | BAr′ |
| 7-2-1 | single bond | — |
| 7-2-1 | BAr′ | — |
| 7-2-1 | O | — |
| 7-3-1 | — | single bond |
| 7-3-1 | — | BAr′ |
| 7-3-1 | — | O |
| 7-4-1 | single bond | single bond |
| 7-4-1 | BAr′ | BAr′ |
| 7-4-1 | single bond | BAr′ |
| 7-4-1 | BAr′ | single bond |
| 7-4-1 | BAr′ | O |
| 7-4-1 | O | BAr′ |
| 7-2-2 | single bond | — |
| 7-2-2 | BAr′ | — |
| 7-2-2 | O | — |
| 7-3-2 | — | single bond |
| 7-3-2 | — | BAr′ |
| 7-3-2 | — | O |
| 7-4-2 | single bond | single bond |
| 7-4-2 | BAr′ | BAr′ |
| 7-4-2 | single bond | BAr′ |
| 7-4-2 | BAr′ | single bond |
| 7-4-2 | BAr′ | O |
| 7-4-2 | O | BAr′ |
| 8-2-1 | single bond | — |
| 8-2-1 | BAr′ | — |
| 8-2-1 | O | — |
| 8-3-1 | — | single bond |
| 8-3-1 | — | BAr′ |
| 8-3-1 | — | O |
| 8-4-1 | single bond | single bond |
| 8-4-1 | BAr′ | BAr′ |
| 8-4-1 | single bond | BAr′ |
| 8-4-1 | BAr′ | single bond |
| 8-4-1 | BAr′ | O |
| 8-4-1 | O | BAr′ |
| 8-2-2 | single bond | — |
| 8-2-2 | BAr′ | — |
| 8-2-2 | O | — |
| 8-3-2 | — | single bond |
| 8-3-2 | — | BAr′ |
| 8-3-2 | — | O |
| 8-4-2 | single bond | single bond |
| 8-4-2 | BAr′ | BAr′ |
| 8-4-2 | single bond | BAr′ |
| 8-4-2 | BAr′ | single bond |
| 8-4-2 | BAr′ | O |
| 8-4-2 | O | BAr′ |
| 9-2-1 | single bond | — |
| 9-2-1 | BAr′ | — |
| 9-2-1 | O | — |
| 9-3-1 | — | single bond |
| 9-3-1 | — | BAr′ |
| 9-3-1 | — | O |
| 9-4-1 | single bond | single bond |
| 9-4-1 | BAr′ | BAr′ |
| 9-4-1 | single bond | BAr′ |
| 9-4-1 | BAr′ | single bond |
| 9-4-1 | BAr′ | O |
| 9-4-1 | O | BAr′ |
| 9-2-2 | single bond | — |
| 9-2-2 | BAr′ | — |
| 9-2-2 | O | — |
| 9-3-2 | — | single bond |
| 9-3-2 | — | BAr′ |
| 9-3-2 | — | O |
| 9-4-2 | single bond | single bond |
| 9-4-2 | BAr′ | BAr′ |
| 9-4-2 | single bond | BAr′ |
| 9-4-2 | BAr′ | single bond |
| 9-4-2 | BAr′ | O |
| 9-4-2 | O | BAr′ |
| 7-2-1-1 | single bond | — |
| 7-2-1-1 | BAr′ | — |
| 7-2-1-1 | O | — |
| 7-3-1-1 | — | single bond |
| 7-3-1-1 | — | BAr′ |
| 7-3-1-1 | — | O |
| 7-4-1-1 | single bond | single bond |
| 7-4-1-1 | BAr′ | BAr′ |
| 7-4-1-1 | single bond | BAr′ |
| 7-4-1-1 | BAr′ | single bond |
| 7-4-1-1 | BAr′ | O |
| 7-4-1-1 | O | BAr′ |
| 7-2-2-1 | single bond | — |
| 7-2-2-1 | BAr′ | — |
| 7-2-2-1 | O | — |
| 7-3-2-1 | — | single bond |
| 7-3-2-1 | — | BAr′ |
| 7-3-2-1 | — | O |
| 7-4-2-1 | single bond | single bond |
| 7-4-2-1 | BAr′ | BAr′ |
| 7-4-2-1 | single bond | BAr′ |
| 7-4-2-1 | BAr′ | single bond |
| 7-4-2-1 | BAr′ | O |
| 7-4-2-1 | O | BAr′ |
| 7-2-1-2 | single bond | — |
| 7-2-1-2 | BAr′ | — |
| 7-2-1-2 | O | — |
| 7-3-1-2 | — | single bond |
| 7-3-1-2 | — | BAr′ |
| 7-3-1-2 | — | O |
| 7-4-1-2 | single bond | single bond |
| 7-4-1-2 | BAr′ | BAr′ |
| 7-4-1-2 | single bond | BAr′ |
| 7-4-1-2 | BAr′ | single bond |
| 7-4-1-2 | BAr′ | O |
| 7-4-1-2 | O | BAr′ |
| 7-2-2-2 | single bond | — |
| 7-2-2-2 | BAr′ | — |
| 7-2-2-2 | O | — |
| 7-3-2-2 | — | single bond |
| 7-3-2-2 | — | BAr′ |
| 7-3-2-2 | — | O |
| 7-4-2-2 | single bond | single bond |
| 7-4-2-2 | BAr′ | BAr′ |
| 7-4-2-2 | single bond | BAr′ |
| 7-4-2-2 | BAr′ | single bond |
| 7-4-2-2 | BAr′ | O |
| 7-4-2-2 | O | BAr′ |
| 7-2-1-3 | single bond | — |
| 7-2-1-3 | BAr′ | — |
| 7-2-1-3 | O | — |
| 7-3-1-3 | — | single bond |
| 7-3-1-3 | — | BAr′ |
| 7-3-1-3 | — | O |
| 7-4-1-3 | single bond | single bond |
| 7-4-1-3 | BAr′ | BAr′ |
| 7-4-1-3 | single bond | BAr′ |
| 7-4-1-3 | BAr′ | single bond |
| 7-4-1-3 | BAr′ | O |
| 7-4-1-3 | O | BAr′ |
| 7-2-2-3 | single bond | — |
| 7-2-2-3 | BAr′ | — |
| 7-2-2-3 | O | — |
| 7-3-2-3 | — | single bond |
| 7-3-2-3 | — | BAr′ |
| 7-3-2-3 | — | O |
| 7-4-2-3 | single bond | single bond |
| 7-4-2-3 | BAr′ | BAr′ |
| 7-4-2-3 | single bond | BAr′ |
| 7-4-2-3 | BAr′ | single bond |
| 7-4-2-3 | BAr′ | O |
| 7-4-2-3 | O | BAr′ |
| 7-2-1-4 | single bond | — |
| 7-2-1-4 | BAr′ | — |
| 7-2-1-4 | O | — |
| 7-3-1-4 | — | single bond |
| 7-3-1-4 | — | BAr′ |
| 7-3-1-4 | — | O |
| 7-4-1-4 | single bond | single bond |
| 7-4-1-4 | BAr′ | BAr′ |
| 7-4-1-4 | single bond | BAr′ |
| 7-4-1-4 | BAr′ | single bond |
| 7-4-1-4 | BAr′ | O |
| 7-4-1-4 | O | BAr′ |
| 7-2-2-4 | single bond | — |
| 7-2-2-4 | BAr′ | — |
| 7-2-2-4 | O | — |
| 7-3-2-4 | — | single bond |
| 7-3-2-4 | — | BAr′ |
| 7-3-2-4 | — | O |
| 7-4-2-4 | single bond | single bond |
| 7-4-2-4 | BAr′ | BAr′ |
| 7-4-2-4 | single bond | BAr′ |
| 7-4-2-4 | BAr′ | single bond |
| 7-4-2-4 | BAr′ | O |
| 7-4-2-4 | O | BAr′ |
| 7-2-1-5 | single bond | — |
| 7-2-1-5 | BAr′ | — |
| 7-2-1-5 | O | — |
| 7-3-1-5 | — | single bond |
| 7-3-1-5 | — | BAr′ |
| 7-3-1-5 | — | O |
| 7-4-1-5 | single bond | single bond |
| 7-4-1-5 | BAr′ | BAr′ |
| 7-4-1-5 | single bond | BAr′ |
| 7-4-1-5 | BAr′ | single bond |
| 7-4-1-5 | BAr′ | O |
| 7-4-1-5 | O | BAr′ |
| 7-2-2-5 | single bond | — |
| 7-2-2-5 | BAr′ | — |
| 7-2-2-5 | O | — |
| 7-3-2-5 | — | single bond |
| 7-3-2-5 | — | BAr′ |
| 7-3-2-5 | — | O |
| 7-4-2-5 | single bond | single bond |
| 7-4-2-5 | BAr′ | BAr′ |
| 7-4-2-5 | single bond | BAr′ |
| 7-4-2-5 | BAr′ | single bond |
| 7-4-2-5 | BAr′ | O |
| 7-4-2-5 | O | BAr′ |
| 7-2-1-6 | single bond | — |
| 7-2-1-6 | BAr′ | — |
| 7-2-1-6 | O | — |
| 7-3-1-6 | — | single bond |
| 7-3-1-6 | — | BAr′ |
| 7-3-1-6 | — | O |
| 7-4-1-6 | single bond | single bond |
| 7-4-1-6 | BAr′ | BAr′ |
| 7-4-1-6 | single bond | BAr′ |
| 7-4-1-6 | BAr′ | single bond |
| 7-4-1-6 | BAr′ | O |
| 7-4-1-6 | O | BAr′ |
| 7-2-2-6 | single bond | — |
| 7-2-2-6 | BAr′ | — |
| 7-2-2-6 | O | — |
| 7-3-2-6 | — | single bond |
| 7-3-2-6 | — | BAr′ |
| 7-3-2-6 | — | O |
| 7-4-2-6 | single bond | single bond |
| 7-4-2-6 | BAr′ | BAr′ |
| 7-4-2-6 | single bond | BAr′ |
| 7-4-2-6 | BAr′ | single bond |
| 7-4-2-6 | BAr′ | O |
| 7-4-2-6 | O | BAr′ |
| 7-2-1-7 | single bond | — |
| 7-2-1-7 | BAr′ | — |
| 7-2-1-7 | O | — |
| 7-3-1-7 | — | single bond |
| 7-3-1-7 | — | BAr′ |
| 7-3-1-7 | — | O |
| 7-4-1-7 | single bond | single bond |
| 7-4-1-7 | BAr′ | BAr′ |
| 7-4-1-7 | single bond | BAr′ |
| 7-4-1-7 | BAr′ | single bond |
| 7-4-1-7 | BAr′ | O |
| 7-4-1-7 | O | BAr′ |
| 7-2-2-7 | single bond | — |
| 7-2-2-7 | BAr′ | — |
| 7-2-2-7 | O | — |
| 7-3-2-7 | — | single bond |
| 7-3-2-7 | — | BAr′ |
| 7-3-2-7 | — | O |
| 7-4-2-7 | single bond | single bond |
| 7-4-2-7 | BAr′ | BAr′ |
| 7-4-2-7 | single bond | BAr′ |
| 7-4-2-7 | BAr′ | single bond |
| 7-4-2-7 | BAr′ | O |
| 7-4-2-7 | O | BAr′ |
| 7-2-1-8 | single bond | — |
| 7-2-1-8 | BAr′ | — |
| 7-2-1-8 | O | — |
| 7-3-1-8 | — | single bond |
| 7-3-1-8 | — | BAr′ |
| 7-3-1-8 | — | O |
| 7-4-1-8 | single bond | single bond |
| 7-4-1-8 | BAr′ | BAr′ |
| 7-4-1-8 | single bond | BAr′ |
| 7-4-1-8 | BAr′ | single bond |
| 7-4-1-8 | BAr′ | O |
| 7-4-1-8 | O | BAr′ |
| 7-2-2-8 | single bond | — |
| 7-2-2-8 | BAr′ | — |
| 7-2-2-8 | O | — |
| 7-3-2-8 | — | single bond |
| 7-3-2-8 | — | BAr′ |
| 7-3-2-8 | — | O |
| 7-4-2-8 | single bond | single bond |
| 7-4-2-8 | BAr′ | BAr′ |
| 7-4-2-8 | single bond | BAr′ |
| 7-4-2-8 | BAr′ | single bond |
| 7-4-2-8 | BAr′ | O |
| 7-4-2-8 | O | BAr′ |
| 8-2-1-1 | single bond | — |
| 8-2-1-1 | BAr′ | — |
| 8-2-1-1 | O | — |
| 8-3-1-1 | — | single bond |
| 8-3-1-1 | — | BAr′ |
| 8-3-1-1 | — | O |
| 8-4-1-1 | single bond | single bond |
| 8-4-1-1 | BAr′ | BAr′ |
| 8-4-1-1 | single bond | BAr′ |
| 8-4-1-1 | BAr′ | single bond |
| 8-4-1-1 | BAr′ | O |
| 8-4-1-1 | O | BAr′ |
| 8-2-2-1 | single bond | — |
| 8-2-2-1 | BAr′ | — |
| 8-2-2-1 | O | — |
| 8-3-2-1 | — | single bond |
| 8-3-2-1 | — | BAr′ |
| 8-3-2-1 | — | O |
| 8-4-2-1 | single bond | single bond |
| 8-4-2-1 | BAr′ | BAr′ |
| 8-4-2-1 | single bond | BAr′ |
| 8-4-2-1 | BAr′ | single bond |
| 8-4-2-1 | BAr′ | O |
| 8-4-2-1 | O | BAr′ |
| 8-2-1-2 | single bond | — |
| 8-2-1-2 | BAr′ | — |
| 8-2-1-2 | O | — |
| 8-3-1-2 | — | single bond |
| 8-3-1-2 | — | BAr′ |
| 8-3-1-2 | — | O |
| 8-4-1-2 | single bond | single bond |
| 8-4-1-2 | BAr′ | BAr′ |
| 8-4-1-2 | single bond | BAr′ |
| 8-4-1-2 | BAr′ | single bond |
| 8-4-1-2 | BAr′ | O |
| 8-4-1-2 | O | BAr′ |
| 8-2-2-2 | single bond | — |
| 8-2-2-2 | BAr′ | — |
| 8-2-2-2 | O | — |
| 8-3-2-2 | — | single bond |
| 8-3-2-2 | — | BAr′ |
| 8-3-2-2 | — | O |
| 8-4-2-2 | single bond | single bond |
| 8-4-2-2 | BAr′ | BAr′ |
| 8-4-2-2 | single bond | BAr′ |
| 8-4-2-2 | BAr′ | single bond |
| 8-4-2-2 | BAr′ | O |
| 8-4-2-2 | O | BAr′ |
| 8-2-1-3 | single bond | — |
| 8-2-1-3 | BAr′ | — |
| 8-2-1-3 | O | — |
| 8-3-1-3 | — | single bond |
| 8-3-1-3 | — | BAr′ |
| 8-3-1-3 | — | O |
| 8-4-1-3 | single bond | single bond |
| 8-4-1-3 | BAr′ | BAr′ |
| 8-4-1-3 | single bond | BAr |
| 8-4-1-3 | BAr′ | single bond |
| 8-4-1-3 | BAr′ | O |
| 8-4-1-3 | O | BAr′ |
| 8-2-2-3 | single bond | — |
| 8-2-2-3 | BAr′ | — |
| 8-2-2-3 | O | — |
| 8-3-2-3 | — | single bond |
| 8-3-2-3 | — | BAr′ |
| 8-3-2-3 | — | O |
| 8-4-2-3 | single bond | single bond |
| 8-4-2-3 | BAr′ | BAr′ |
| 8-4-2-3 | single bond | BAr′ |
| 8-4-2-3 | BAr′ | single bond |
| 8-4-2-3 | BAr′ | O |
| 8-4-2-3 | O | BAr′ |
| 8-2-1-4 | single bond | — |
| 8-2-1-4 | BAr′ | — |
| 8-2-1-4 | O | — |
| 8-3-1-4 | — | single bond |
| 8-3-1-4 | — | BAr′ |
| 8-3-1-4 | — | O |
| 8-4-1-4 | single bond | single bond |
| 8-4-1-4 | BAr′ | BAr′ |
| 8-4-1-4 | single bond | BAr′ |
| 8-4-1-4 | BAr′ | single bond |
| 8-4-1-4 | BAr′ | O |
| 8-4-1-4 | O | BAr′ |
| 8-2-2-4 | single bond | — |
| 8-2-2-4 | BAr′ | — |
| 8-2-2-4 | O | — |
| 8-3-2-4 | — | single bond |
| 8-3-2-4 | — | BAr′ |
| 8-3-2-4 | — | O |
| 8-4-2-4 | single bond | single bond |
| 8-4-2-4 | BAr′ | BAr′ |
| 8-4-2-4 | single bond | BAr′ |
| 8-4-2-4 | BAr′ | single bond |
| 8-4-2-4 | BAr′ | O |
| 8-4-2-4 | O | BAr′ |
| 8-2-1-5 | single bond | — |
| 8-2-1-5 | BAr′ | — |
| 8-2-1-5 | O | — |
| 8-3-1-5 | — | single bond |
| 8-3-1-5 | — | BAr′ |
| 8-3-1-5 | — | O |
| 8-4-1-5 | single bond | single bond |
| 8-4-1-5 | BAr′ | BAr′ |
| 8-4-1-5 | single bond | BAr′ |
| 8-4-1-5 | BAr′ | single bond |
| 8-4-1-5 | BAr′ | O |
| 8-4-1-5 | O | BAr′ |
| 8-2-2-5 | single bond | — |
| 8-2-2-5 | BAr′ | — |
| 8-2-2-5 | O | — |
| 8-3-2-5 | — | single bond |
| 8-3-2-5 | — | BAr′ |
| 8-3-2-5 | — | O |
| 8-4-2-5 | single bond | single bond |
| 8-4-2-5 | BAr′ | BAr′ |
| 8-4-2-5 | single bond | BAr′ |
| 8-4-2-5 | BAr′ | single bond |
| 8-4-2-5 | BAr′ | O |
| 8-4-2-5 | O | BAr′ |
| 8-2-1-6 | single bond | — |
| 8-2-1-6 | BAr′ | — |
| 8-2-1-6 | O | — |
| 8-3-1-6 | — | single bond |
| 8-3-1-6 | — | BAr′ |
| 8-3-1-6 | — | O |
| 8-4-1-6 | single bond | single bond |
| 8-4-1-6 | BAr′ | BAr′ |
| 8-4-1-6 | single bond | BAr′ |
| 8-4-1-6 | BAr′ | single bond |
| 8-4-1-6 | BAr | O |
| 8-4-1-6 | O | BAr′ |
| 8-2-2-6 | single bond | — |
| 8-2-2-6 | BAr′ | — |
| 8-2-2-6 | O | — |
| 8-3-2-6 | — | single bond |
| 8-3-2-6 | — | BAr′ |
| 8-3-2-6 | — | O |
| 8-4-2-6 | single bond | single bond |
| 8-4-2-6 | BAr′ | BAr′ |
| 8-4-2-6 | single bond | BAr′ |
| 8-4-2-6 | BAr′ | single bond |
| 8-4-2-6 | BAr′ | O |
| 8-4-2-6 | O | BAr′ |
| 8-2-1-7 | single bond | — |
| 8-2-1-7 | BAr′ | — |
| 8-2-1-7 | O | — |
| 8-3-1-7 | — | single bond |
| 8-3-1-7 | — | BAr′ |
| 8-3-1-7 | — | O |
| 8-4-1-7 | single bond | single bond |
| 8-4-1-7 | BAr′ | BAr′ |
| 8-4-1-7 | single bond | BAr′ |
| 8-4-1-7 | BAr′ | single bond |
| 8-4-1-7 | BAr′ | O |
| 8-4-1-7 | O | BAr′ |
| 8-2-2-7 | single bond | — |
| 8-2-2-7 | BAr′ | — |
| 8-2-2-7 | O | — |
| 8-3-2-7 | — | single bond |
| 8-3-2-7 | — | BAr′ |
| 8-3-2-7 | — | O |
| 8-4-2-7 | single bond | single bond |
| 8-4-2-7 | BAr′ | BAr′ |
| 8-4-2-7 | single bond | BAr′ |
| 8-4-2-7 | BAr′ | single bond |
| 8-4-2-7 | BAr′ | O |
| 8-4-2-7 | O | BAr′ |
| 8-2-1-8 | single bond | — |
| 8-2-1-8 | BAr′ | — |
| 8-2-1-8 | O | — |
| 8-3-1-8 | — | single bond |
| 8-3-1-8 | — | BAr′ |
| 8-3-1-8 | — | O |
| 8-4-1-8 | single bond | single bond |
| 8-4-1-8 | BAr′ | BAr′ |
| 8-4-1-8 | single bond | BAr′ |
| 8-4-1-8 | BAr′ | single bond |
| 8-4-1-8 | BAr′ | O |
| 8-4-1-8 | O | BAr′ |
| 8-2-2-8 | single bond | — |
| 8-2-2-8 | BAr′ | — |
| 8-2-2-8 | O | — |
| 8-3-2-8 | — | single bond |
| 8-3-2-8 | — | BAr′ |
| 8-3-2-8 | — | O |
| 8-4-2-8 | single bond | single bond |
| 8-4-2-8 | BAr′ | BAr′ |
| 8-4-2-8 | single bond | BAr′ |
| 8-4-2-8 | BAr′ | single bond |
| 8-4-2-8 | BAr′ | O |
| 8-4-2-8 | O | BAr′ |
| 9-2-1-1 | single bond | — |
| 9-2-1-1 | BAr′ | — |
| 9-2-1-1 | O | — |
| 9-3-1-1 | — | single bond |
| 9-3-1-1 | — | BAr′ |
| 9-3-1-1 | — | O |
| 9-4-1-1 | single bond | single bond |
| 9-4-1-1 | BAr′ | BAr′ |
| 9-4-1-1 | single bond | BAr′ |
| 9-4-1-1 | BAr′ | single bond |
| 9-4-1-1 | BAr′ | O |
| 9-4-1-1 | O | BAr′ |
| 9-2-2-1 | single bond | — |
| 9-2-2-1 | BAr′ | — |
| 9-2-2-1 | O | — |
| 9-3-2-1 | — | single bond |
| 9-3-2-1 | — | BAr′ |
| 9-3-2-1 | — | O |
| 9-4-2-1 | single bond | single bond |
| 9-4-2-1 | BAr′ | BAr′ |
| 9-4-2-1 | single bond | BAr′ |
| 9-4-2-1 | BAr′ | single bond |
| 9-4-2-1 | BAr′ | O |
| 9-4-2-1 | O | BAr′ |
| 9-2-1-2 | single bond | — |
| 9-2-1-2 | BAr′ | — |
| 9-2-1-2 | O | — |
| 9-3-1-2 | — | single bond |
| 9-3-1-2 | — | BAr′ |
| 9-3-1-2 | — | O |
| 9-4-1-2 | single bond | single bond |
| 9-4-1-2 | BAr′ | BAr′ |
| 9-4-1-2 | single bond | BAr′ |
| 9-4-1-2 | BAr′ | single bond |
| 9-4-1-2 | BAr′ | O |
| 9-4-1-2 | O | BAr′ |
| 9-2-2-2 | single bond | — |
| 9-2-2-2 | BAr′ | — |
| 9-2-2-2 | O | — |
| 9-3-2-2 | — | single bond |
| 9-3-2-2 | — | BAr′ |
| 9-3-2-2 | — | O |
| 9-4-2-2 | single bond | single bond |
| 9-4-2-2 | BAr′ | BAr′ |
| 9-4-2-2 | single bond | BAr′ |
| 9-4-2-2 | BAr′ | single bond |
| 9-4-2-2 | BAr′ | O |
| 9-4-2-2 | O | BAr′ |
| 9-2-1-3 | single bond | — |
| 9-2-1-3 | BAr′ | — |
| 9-2-1-3 | O | — |
| 9-3-1-3 | — | single bond |
| 9-3-1-3 | — | BAr′ |
| 9-3-1-3 | — | O |
| 9-4-1-3 | single bond | single bond |
| 9-4-1-3 | BAr′ | BAr′ |
| 9-4-1-3 | single bond | BAr′ |
| 9-4-1-3 | BAr′ | single bond |
| 9-4-1-3 | BAr′ | O |
| 9-4-1-3 | O | BAr′ |
| 9-2-2-3 | single bond | — |
| 9-2-2-3 | BAr′ | — |
| 9-2-2-3 | O | — |
| 9-3-2-3 | — | single bond |
| 9-3-2-3 | — | BAr′ |
| 9-3-2-3 | — | O |
| 9-4-2-3 | single bond | single bond |
| 9-4-2-3 | BAr′ | BAr′ |
| 9-4-2-3 | single bond | BAr′ |
| 9-4-2-3 | BAr′ | single bond |
| 9-4-2-3 | BAr′ | O |
| 9-4-2-3 | O | BAr′ |
| 9-2-1-4 | single bond | — |
| 9-2-1-4 | BAr′ | — |
| 9-2-1-4 | O | — |
| 9-3-1-4 | — | single bond |
| 9-3-1-4 | — | BAr′ |
| 9-3-1-4 | — | O |
| 9-4-1-4 | single bond | single bond |
| 9-4-1-4 | BAr′ | BAr′ |
| 9-4-1-4 | single bond | BAr′ |
| 9-4-1-4 | BAr′ | single bond |
| 9-4-1-4 | BAr′ | O |
| 9-4-1-4 | O | BAr′ |
| 9-2-2-4 | single bond | — |
| 9-2-2-4 | BAr′ | — |
| 9-2-2-4 | O | — |
| 9-3-2-4 | — | single bond |
| 9-3-2-4 | — | BAr′ |
| 9-3-2-4 | — | O |
| 9-4-2-4 | single bond | single bond |
| 9-4-2-4 | BAr′ | BAr′ |
| 9-4-2-4 | single bond | BAr′ |
| 9-4-2-4 | BAr′ | single bond |
| 9-4-2-4 | BAr′ | O |
| 9-4-2-4 | O | BAr′ |
| 9-2-1-5 | single bond | — |
| 9-2-1-5 | BAr′ | — |
| 9-2-1-5 | O | — |
| 9-3-1-5 | — | single bond |
| 9-3-1-5 | — | BAr′ |
| 9-3-1-5 | — | O |
| 9-4-1-5 | single bond | single bond |
| 9-4-1-5 | BAr′ | BAr′ |
| 9-4-1-5 | single bond | BAr′ |
| 9-4-1-5 | BAr′ | single bond |
| 9-4-1-5 | BAr′ | O |
| 9-4-1-5 | O | BAr′ |
| 9-2-2-5 | single bond | — |
| 9-2-2-5 | BAr′ | — |
| 9-2-2-5 | O | — |
| 9-3-2-5 | — | single bond |
| 9-3-2-5 | — | BAr′ |
| 9-3-2-5 | — | O |
| 9-4-2-5 | single bond | single bond |
| 9-4-2-5 | BAr′ | BAr′ |
| 9-4-2-5 | single bond | BAr′ |
| 9-4-2-5 | BAr′ | single bond |
| 9-4-2-5 | BAr′ | O |
| 9-4-2-5 | O | BAr′ |
| 9-2-1-6 | single bond | — |
| 9-2-1-6 | BAr′ | — |
| 9-2-1-6 | O | — |
| 9-3-1-6 | — | single bond |
| 9-3-1-6 | — | BAr′ |
| 9-3-1-6 | — | O |
| 9-4-1-6 | single bond | single bond |
| 9-4-1-6 | BAr′ | BAr′ |
| 9-4-1-6 | single bond | BAr′ |
| 9-4-1-6 | BAr′ | single bond |
| 9-4-1-6 | BAr′ | O |
| 9-4-1-6 | O | BAr′ |
| 9-2-2-6 | single bond | — |
| 9-2-2-6 | BAr′ | — |
| 9-2-2-6 | O | — |
| 9-3-2-6 | — | single bond |
| 9-3-2-6 | — | BAr′ |
| 9-3-2-6 | — | O |
| 9-4-2-6 | single bond | single bond |
| 9-4-2-6 | BAr′ | BAr′ |
| 9-4-2-6 | single bond | BAr′ |
| 9-4-2-6 | BAr′ | single bond |
| 9-4-2-6 | BAr′ | O |
| 9-4-2-6 | O | BAr′ |
| 9-2-1-7 | single bond | — |
| 9-2-1-7 | BAr′ | — |
| 9-2-1-7 | O | — |
| 9-3-1-7 | — | single bond |
| 9-3-1-7 | — | BAr′ |
| 9-3-1-7 | — | O |
| 9-4-1-7 | single bond | single bond |
| 9-4-1-7 | BAr′ | BAr′ |
| 9-4-1-7 | single bond | BAr′ |
| 9-4-1-7 | BAr′ | single bond |
| 9-4-1-7 | BAr′ | O |
| 9-4-1-7 | O | BAr′ |
| 9-2-2-7 | single bond | — |
| 9-2-2-7 | BAr′ | — |
| 9-2-2-7 | O | — |
| 9-3-2-7 | — | single bond |
| 9-3-2-7 | — | BAr′ |
| 9-3-2-7 | — | O |
| 9-4-2-7 | single bond | single bond |
| 9-4-2-7 | BAr′ | BAr′ |
| 9-4-2-7 | single bond | BAr′ |
| 9-4-2-7 | BAr′ | single bond |
| 9-4-2-7 | BAr′ | O |
| 9-4-2-7 | O | BAr′ |
| 9-2-1-8 | single bond | — |
| 9-2-1-8 | BAr′ | — |
| 9-2-1-8 | O | — |
| 9-3-1-8 | — | single bond |
| 9-3-1-8 | — | BAr′ |
| 9-3-1-8 | — | O |
| 9-4-1-8 | single bond | single bond |
| 9-4-1-8 | BAr′ | BAr′ |
| 9-4-1-8 | single bond | BAr′ |
| 9-4-1-8 | BAr′ | single bond |
| 9-4-1-8 | BAr′ | O |
| 9-4-1-8 | O | BAr′ |
| 9-2-2-8 | single bond | — |
| 9-2-2-8 | BAr′ | — |
| 9-2-2-8 | O | — |
| 9-3-2-8 | — | single bond |
| 9-3-2-8 | — | BAr′ |
| 9-3-2-8 | — | O |
| 9-4-2-8 | single bond | single bond |
| 9-4-2-8 | BAr′ | BAr′ |
| 9-4-2-8 | single bond | BAr′ |
| 9-4-2-8 | BAr′ | single bond |
| 9-4-2-8 | BAr′ | O |
| 9-4-2-8 | O | BAr′ |
| 10-2-1 | single bond | — |
| 10-2-1 | BAr′ | — |
| 10-2-1 | O | — |
| 10-3-1 | — | single bond |
| 10-3-1 | — | BAr′ |
| 10-3-1 | — | O |
| 10-4-1 | single bond | single bond |
| 10-4-1 | BAr′ | BAr′ |
| 10-4-1 | single bond | BAr′ |
| 10-4-1 | BAr′ | single bond |
| 10-4-1 | BAr′ | O |
| 10-4-1 | O | BAr′ |
| 10-2-2 | single bond | — |
| 10-2-2 | BAr′ | — |
| 10-2-2 | O | — |
| 10-3-2 | — | single bond |
| 10-3-2 | — | BAr′ |
| 10-3-2 | — | O |
| 10-4-2 | single bond | single bond |
| 10-4-2 | BAr′ | BAr′ |
| 10-4-2 | single bond | BAr′ |
| 10-4-2 | BAr′ | single bond |
| 10-4-2 | BAr′ | O |
| 10-4-2 | O | BAr′ |
| 10-2-3 | single bond | — |
| 10-2-3 | BAr′ | — |
| 10-2-3 | O | — |
| 10-3-3 | — | single bond |
| 10-3-3 | — | BAr′ |
| 10-3-3 | — | O |
| 10-4-3 | single bond | single bond |
| 10-4-3 | BAr′ | BAr′ |
| 10-4-3 | single bond | BAr′ |
| 10-4-3 | BAr′ | single bond |
| 10-4-3 | BAr′ | O |
| 10-4-3 | O | BAr′ |
| 10-2-4 | single bond | — |
| 10-2-4 | BAr′ | — |
| 10-2-4 | O | — |
| 10-3-4 | — | single bond |
| 10-3-4 | — | BAr′ |
| 10-3-4 | — | O |
| 10-4-4 | single bond | single bond |
| 10-4-4 | BAr′ | BAr′ |
| 10-4-4 | single bond | BAr′ |
| 10-4-4 | BAr′ | single bond |
| 10-4-4 | BAr′ | O |
| 10-4-4 | O | BAr′ |
| 10-2-5 | single bond | — |
| 10-2-5 | BAr′ | — |
| 10-2-5 | O | — |
| 10-3-5 | — | single bond |
| 10-3-5 | — | BAr′ |
| 10-3-5 | — | O |
| 10-4-5 | single bond | single bond |
| 10-4-5 | BAr′ | BAr′ |
| 10-4-5 | single bond | BAr′ |
| 10-4-5 | BAr′ | single bond |
| 10-4-5 | BAr′ | O |
| 10-4-5 | O | BAr′ |
| 10-2-6 | single bond | — |
| 10-2-6 | BAr′ | — |
| 10-2-6 | O | — |
| 10-3-6 | — | single bond |
| 10-3-6 | — | BAr′ |
| 10-3-6 | — | O |
| 10-4-6 | single bond | single bond |
| 10-4-6 | BAr′ | BAr′ |
| 10-4-6 | single bond | BAr′ |
| 10-4-6 | BAr′ | single bond |
| 10-4-6 | BAr′ | O |
| 10-4-6 | O | BAr′ |
| 10-2-7 | single bond | — |
| 10-2-7 | BAr′ | — |
| 10-2-7 | O | — |
| 10-3-7 | — | single bond |
| 10-3-7 | — | BAr′ |
| 10-3-7 | — | O |
| 10-4-7 | single bond | single bond |
| 10-4-7 | BAr′ | BAr′ |
| 10-4-7 | single bond | BAr′ |
| 10-4-7 | BAr′ | single bond |
| 10-4-7 | BAr′ | O |
| 10-4-7 | O | BAr′ |
| 10-2-8 | single bond | — |
| 10-2-8 | BAr′ | — |
| 10-2-8 | O | — |
| 10-3-8 | — | single bond |
| 10-3-8 | — | BAr′ |
| 10-3-8 | — | O |
| 10-4-8 | single bond | single bond |
| 10-4-8 | BAr′ | BAr′ |
| 10-4-8 | single bond | BAr′ |
| 10-4-8 | BAr′ | single bond |
| 10-4-8 | BAr′ | O |
| 10-4-8 | O | BAr′ |
| 11-2-1 | single bond | — |
| 11-2-1 | BAr′ | — |
| 11-2-1 | O | — |
| 11-3-1 | — | single bond |
| 11-3-1 | — | BAr′ |
| 11-3-1 | — | O |
| 11-4-1 | single bond | single bond |
| 11-4-1 | BAr′ | BAr′ |
| 11-4-1 | single bond | BAr |
| 11-41 | BAr′ | single bond |
| 11-4-1 | BAr′ | O |
| 11-4-1 | O | BAr′ |
| 11-2-2 | single bond | — |
| 11-2-2 | BAr′ | — |
| 11-2-2 | O | — |
| 11-3-2 | — | single bond |
| 11-3-2 | — | BAr′ |
| 11-3-2 | — | O |
| 11-4-2 | single bond | single bond |
| 11-4-2 | BAr′ | BAr′ |
| 11-4-2 | single bond | BAr′ |
| 11-4-2 | BAr′ | single bond |
| 11-4-2 | BAr′ | O |
| 11-4-2 | O | BAr′ |
| 11-2-3 | single bond | — |
| 11-2-3 | BAr′ | — |
| 11-2-3 | O | — |
| 11-3-3 | — | single bond |
| 11-3-3 | — | BAr′ |
| 11-3-3 | — | O |
| 11-4-3 | single bond | single bond |
| 11-4-3 | BAr′ | BAr′ |
| 11-4-3 | single bond | BAr′ |
| 11-4-3 | BAr′ | single bond |
| 11-4-3 | BAr′ | O |
| 11-4-3 | O | BAr′ |
| 11-2-4 | single bond | — |
| 11-2-4 | BAr′ | — |
| 11-2-4 | O | — |
| 11-3-4 | — | single bond |
| 11-3-4 | — | BAr′ |
| 11-3-4 | — | O |
| 11-4-4 | single bond | single bond |
| 11-4-4 | BAr′ | BAr′ |
| 11-4-4 | single bond | BAr′ |
| 11-4-4 | BAr′ | single bond |
| 11-4-4 | BAr′ | O |
| 11-4-4 | O | BAr′ |
| 11-2-5 | single bond | — |
| 11-2-5 | BAr′ | — |
| 11-2-5 | O | — |
| 11-3-5 | — | single bond |
| 11-3-5 | — | BAr′ |
| 11-3-5 | — | O |
| 11-4-5 | single bond | single bond |
| 11-4-5 | BAr′ | BAr′ |
| 11-4-5 | single bond | BAr′ |
| 11-4-5 | BAr′ | single bond |
| 11-4-5 | BAr′ | O |
| 11-4-5 | O | BAr′ |
| 11-2-6 | single bond | — |
| 11-2-6 | BAr′ | — |
| 11-2-6 | O | — |
| 11-3-6 | — | single bond |
| 11-3-6 | — | BAr′ |
| 11-3-6 | — | O |
| 11-4-6 | single bond | single bond |
| 11-4-6 | BAr′ | BAr′ |
| 11-4-6 | single bond | BAr′ |
| 11-4-6 | BAr′ | single bond |
| 11-4-6 | BAr′ | O |
| 11-4-6 | O | BAr′ |
| 11-2-7 | single bond | — |
| 11-2-7 | BAr′ | — |
| 11-2-7 | O | — |
| 11-3-7 | — | single bond |
| 11-3-7 | — | BAr′ |
| 11-3-7 | — | O |
| 11-4-7 | single bond | single bond |
| 11-4-7 | BAr′ | BAr′ |
| 11-4-7 | single bond | BAr′ |
| 11-4-7 | BAr′ | single bond |
| 11-4-7 | BAr′ | O |
| 11-4-7 | O | BAr′ |
| 11-2-8 | single bond | — |
| 11-2-8 | BAr′ | — |
| 11-2-8 | O | — |
| 11-3-8 | — | single bond |
| 11-3-8 | — | BAr′ |
| 11-3-8 | — | O |
| 11-4-8 | single bond | single bond |
| 11-4-8 | BAr′ | BAr′ |
| 11-4-8 | single bond | BAr′ |
| 11-4-8 | BAr′ | single bond |
| 11-4-8 | BAr′ | O |
| 11-4-8 | O | BAr′ |
| 12-2-1 | single bond | — |
| 12-2-1 | BAr′ | — |
| 12-2-1 | O | — |
| 12-3-1 | — | single bond |
| 12-3-1 | — | BAr′ |
| 12-3-1 | — | O |
| 12-4-1 | single bond | single bond |
| 12-4-1 | BAr′ | BAr′ |
| 12-4-1 | single bond | BAr′ |
| 12-4-1 | BAr′ | single bond |
| 12-4-1 | BAr′ | O |
| 12-4-1 | O | BAr′ |
| 12-2-2 | single bond | — |
| 12-2-2 | BAr′ | — |
| 12-2-2 | O | — |
| 12-3-2 | — | single bond |
| 12-3-2 | — | BAr′ |
| 12-3-2 | — | O |
| 12-4-2 | single bond | single bond |
| 12-4-2 | BAr′ | BAr′ |
| 12-4-2 | single bond | BAr′ |
| 12-4-2 | BAr′ | single bond |
| 12-4-2 | BAr′ | O |
| 12-4-2 | O | BAr′ |
| 12-2-3 | single bond | — |
| 12-2-3 | BAr′ | — |
| 12-2-3 | O | — |
| 12-3-3 | — | single bond |
| 12-3-3 | — | BAr′ |
| 12-3-3 | — | O |
| 12-4-3 | single bond | single bond |
| 12-4-3 | BAr′ | BAr′ |
| 12-4-3 | single bond | BAr′ |
| 12-4-3 | BAr′ | single bond |
| 12-4-3 | BAr′ | O |
| 12-4-3 | O | BAr′ |
| 12-2-4 | single bond | — |
| 12-2-4 | BAr′ | — |
| 12-2-4 | O | — |
| 12-3-4 | — | single bond |
| 12-3-4 | — | BAr′ |
| 12-3-4 | — | O |
| 12-4-4 | single bond | single bond |
| 12-4-4 | BAr′ | BAr′ |
| 12-4-4 | single bond | BAr′ |
| 12-4-4 | BAr′ | single bond |
| 12-4-4 | BAr′ | O |
| 12-4-4 | O | BAr′ |
| 12-2-5 | single bond | — |
| 12-2-5 | BAr′ | — |
| 12-2-5 | O | — |
| 12-3-5 | — | single bond |
| 12-3-5 | — | BAr′ |
| 12-3-5 | — | O |
| 12-4-5 | single bond | single bond |
| 12-4-5 | BAr′ | BAr′ |
| 12-4-5 | single bond | BAr′ |
| 12-4-5 | BAr′ | single bond |
| 12-4-5 | BAr′ | O |
| 12-4-5 | O | BAr′ |
| 12-2-6 | single bond | — |
| 12-2-6 | BAr′ | — |
| 12-2-6 | O | — |
| 12-3-6 | — | single bond |
| 12-3-6 | — | BAr′ |
| 12-3-6 | — | O |
| 12-4-6 | single bond | single bond |
| 12-4-6 | BAr′ | BAr′ |
| 12-4-6 | single bond | BAr′ |
| 12-4-6 | BAr′ | single bond |
| 12-4-6 | BAr′ | O |
| 12-4-6 | O | BAr′ |
| 12-2-7 | single bond | — |
| 12-2-7 | BAr′ | — |
| 12-2-7 | O | — |
| 12-3-7 | — | single bond |
| 12-3-7 | — | BAr′ |
| 12-3-7 | — | O |
| 12-4-7 | single bond | single bond |
| 12-4-7 | BAr′ | BAr′ |
| 12-4-7 | single bond | BAr′ |
| 12-4-7 | BAr′ | single bond |
| 12-4-7 | BAr′ | O |
| 12-4-7 | O | BAr′ |
| 12-2-8 | single bond | — |
| 12-2-8 | BAr′ | — |
| 12-2-8 | O | — |
| 12-3-8 | — | single bond |
| 12-3-8 | — | BAr′ |
| 12-3-8 | — | O |
| 12-4-8 | single bond | single bond |
| 12-4-8 | BAr′ | BAr′ |
| 12-4-8 | single bond | BAr′ |
| 12-4-8 | BAr′ | single bond |
| 12-4-8 | BAr′ | O |
| 12-4-8 | O | BAr′ |

In a preferred embodiment, the compounds based on formula (2) and the preferred embodiments thereof are symmetric, especially C2-symmetric.

In a preferred embodiment, R is the same or different at each instance and is D, F, Cl, Br, I, N(Ar′)₂, N(R¹)₂, OAr′, SAr′, B(R¹)₂, B(OR¹)₂, CHO, C(═O)R¹, CR¹═C(R¹)₂, C(═O)OR¹, C(═O)NR¹, Si(R¹)₃, NO₂, P(R¹)₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C═C—, Si(R¹)₂, NR¹, CONR¹, C═O, C═S, —C(═O)O—, P(═O)(R¹), —O—, —S—, SO or SO₂, or an aromatic or heteroaromatic ring system which has 5 to 60 aromatic ring atoms, preferably 5 to 40 aromatic ring atoms, and may be substituted in each case by one or more R¹ radicals, where two or more R radicals preferably bonded to the same cycle may together form an aliphatic, heteroaliphatic, aromatic or heteroaromatic ring system which may be substituted by one or more R¹ radicals.

In a preferred embodiment, at least one R group is an aromatic or heteroaromatic ring system which has 5 to 60 aromatic ring atoms, preferably 5 to 40 aromatic ring atoms, and may be substituted in each case by one or more R¹ radicals, where two or more R radicals preferably bonded to the same cycle may together form an aliphatic, heteroaliphatic, aromatic or heteroaromatic ring system which may be substituted by one or more R¹ radicals.

There follows a description of preferred substituents R, R′, Ar′, R¹ and R². In a particularly preferred embodiment of the invention, the preferences specified hereinafter for R, R′, Ar′, R¹ and R² occur simultaneously and are applicable to the structures of the formula (1) and to all preferred embodiments detailed above.

In a preferred embodiment of the invention, R is the same or different at each instance and is selected from the group consisting of D, F, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic, aromatic or heteroaromatic ring system. More preferably, R is the same or different at each instance and is selected from the group consisting of D, CN, F, a straight chain alkyl group having 1 to 6 carbon atoms, especially having 1, 2, 3 or 4 carbon atoms, or a branched or cyclic alkyl group having 3 to 6 carbon atoms, where the alkyl group may be substituted in each case by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R is the same or different at each instance and is selected from the group consisting of H or an aromatic or heteroaromatic ring system which has 6 to 24 aromatic ring atoms and may be substituted in each case by one or more R¹ radicals, preferably nonaromatic R¹ radicals.

In a preferred embodiment of the invention, R′ is the same or different at each instance and is selected from the group consisting of H, D, F, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may be substituted in each case by one or more R radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R radicals; at the same time, two R radicals together may also form an aliphatic, aromatic or heteroaromatic ring system. More preferably, R′ is the same or different at each instance and is selected from the group consisting of H, D, F or an aromatic or heteroaromatic ring system which has 6 to 24 aromatic ring atoms and may be substituted in each case by one or more R radicals, preferably nonaromatic R radicals. Most preferably, R′ is the same or different at each instance and is selected from the group consisting of H, D or an aromatic or heteroaromatic ring system which has 6 to 24 aromatic ring atoms and may be substituted in each case by one or more R radicals.

Suitable aromatic or heteroaromatic ring systems R and R′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene which may be joined via the 1, 2, 3 or 4 position, dibenzofuran, carbazole which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R and/or R′ is a heteroaryl group, especially triazine, pyrimidine or quinazoline, preference may also be given to aromatic or heteroaromatic R¹ radicals on this heteroaryl group.

The R and R′ groups here, when they are an aromatic or heteroaromatic ring system, are preferably selected from the groups of the formulae R-1 to R-163 that follow, where, in the case of R′, R¹ is R:

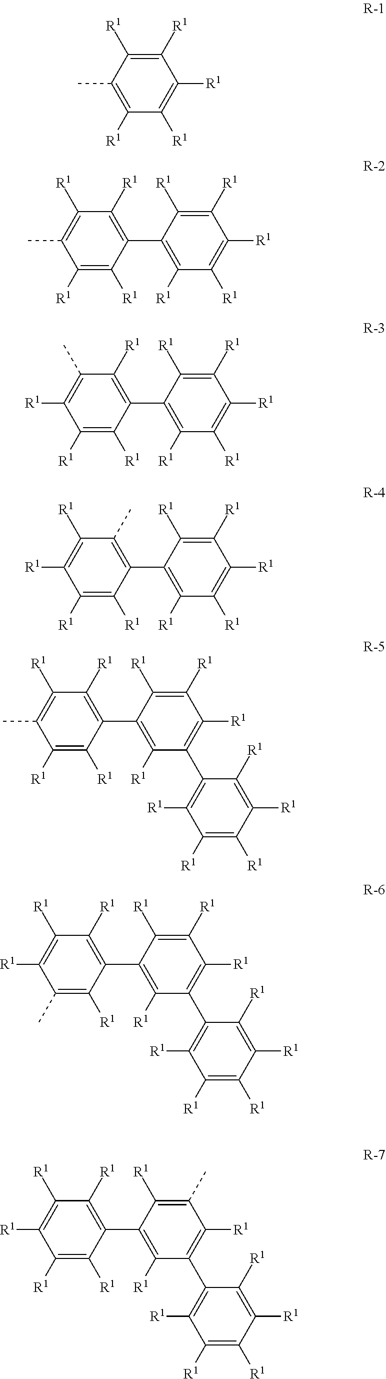

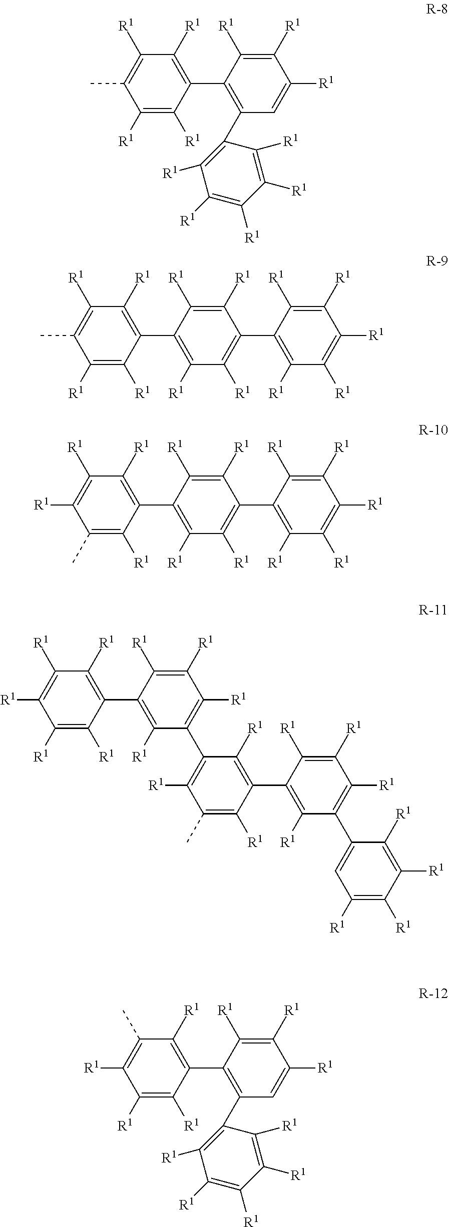

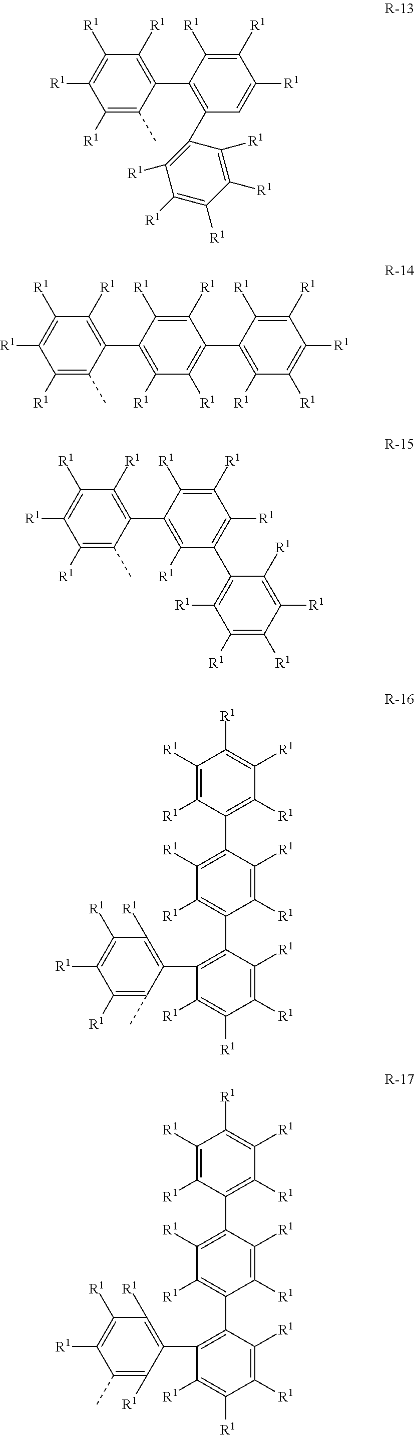

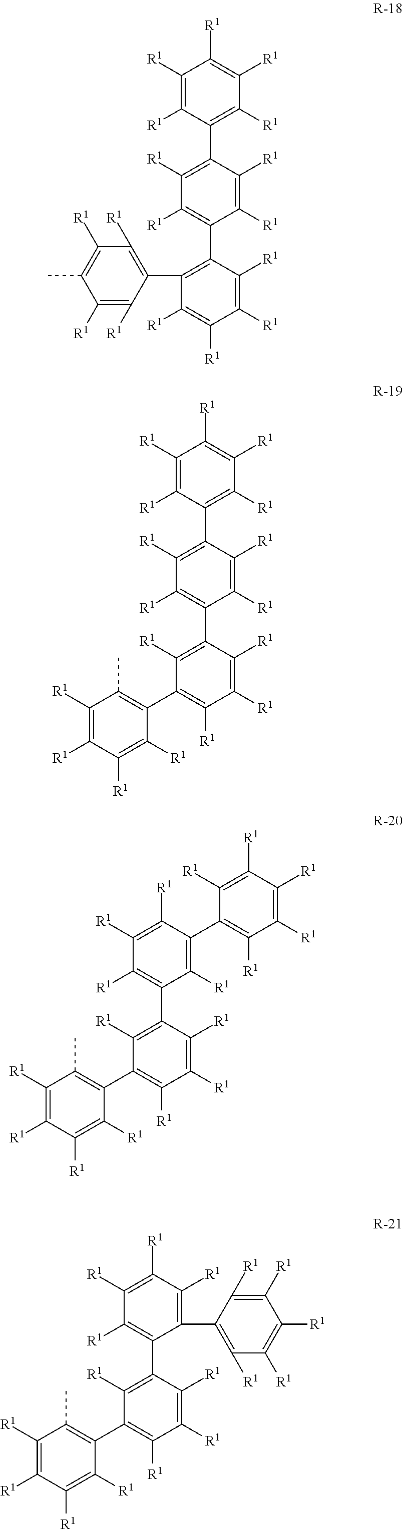

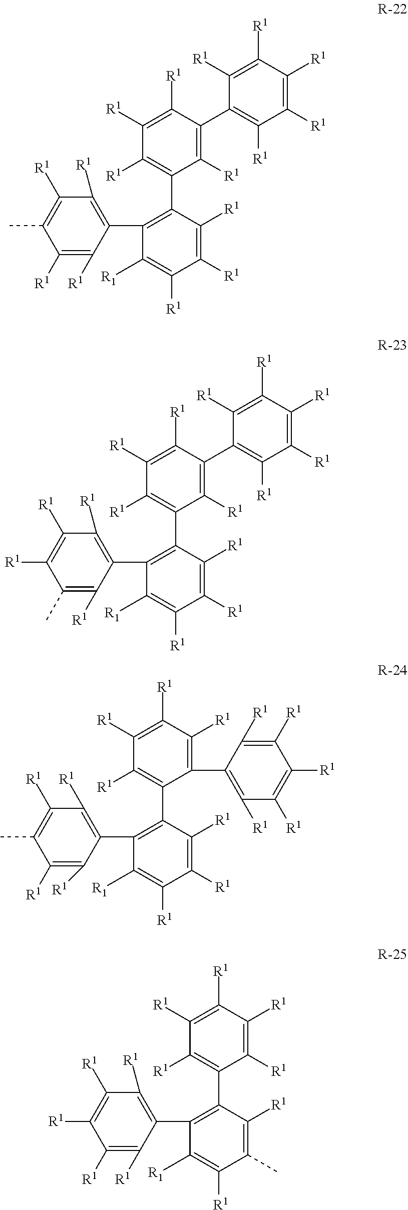

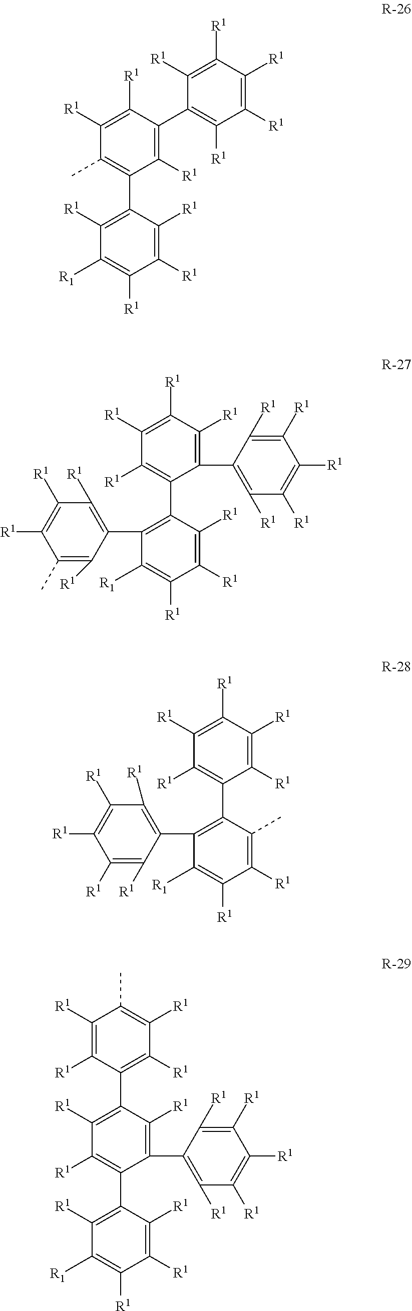

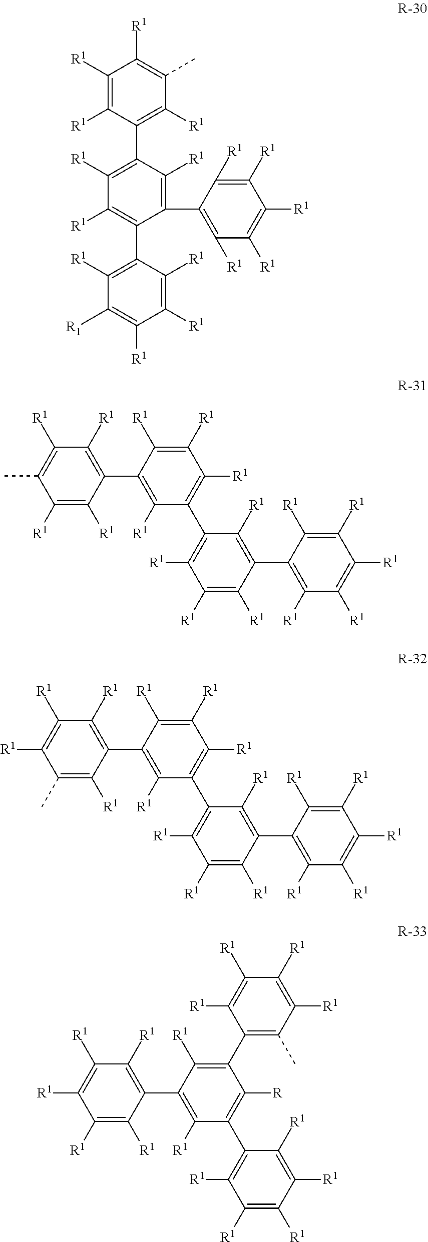

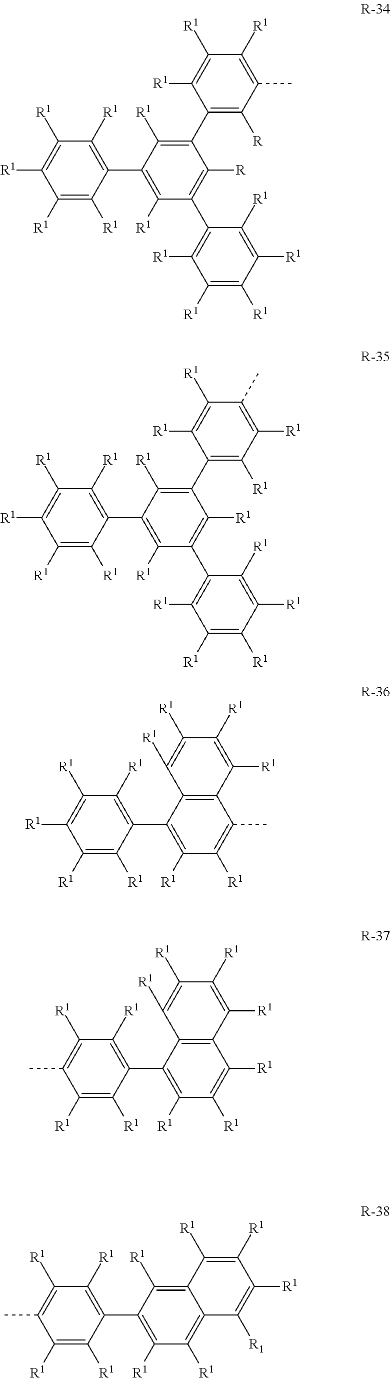

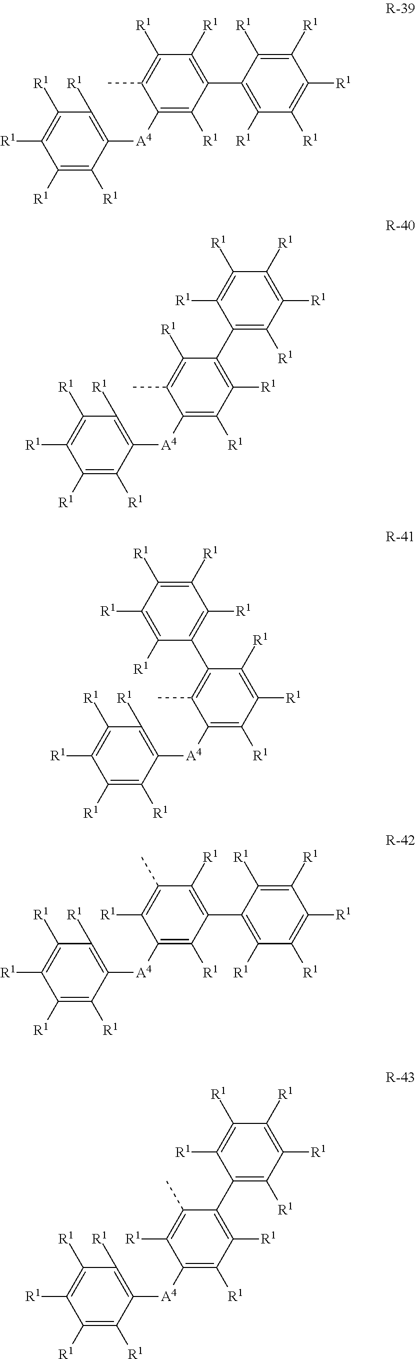

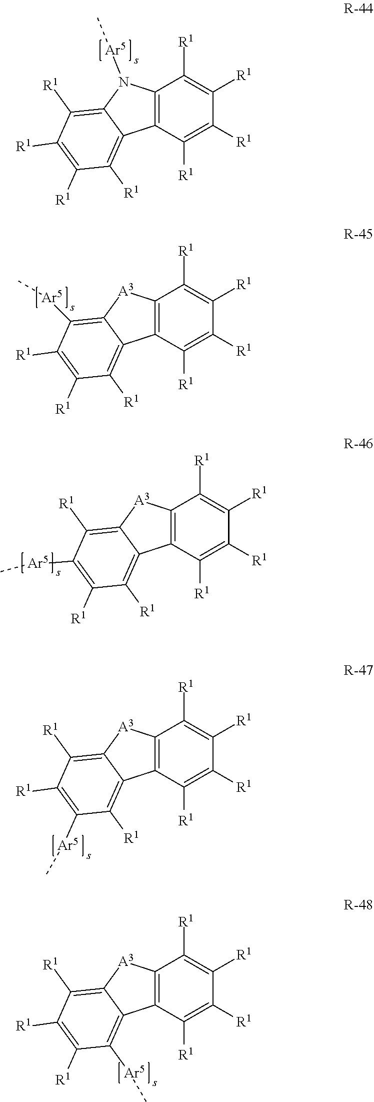

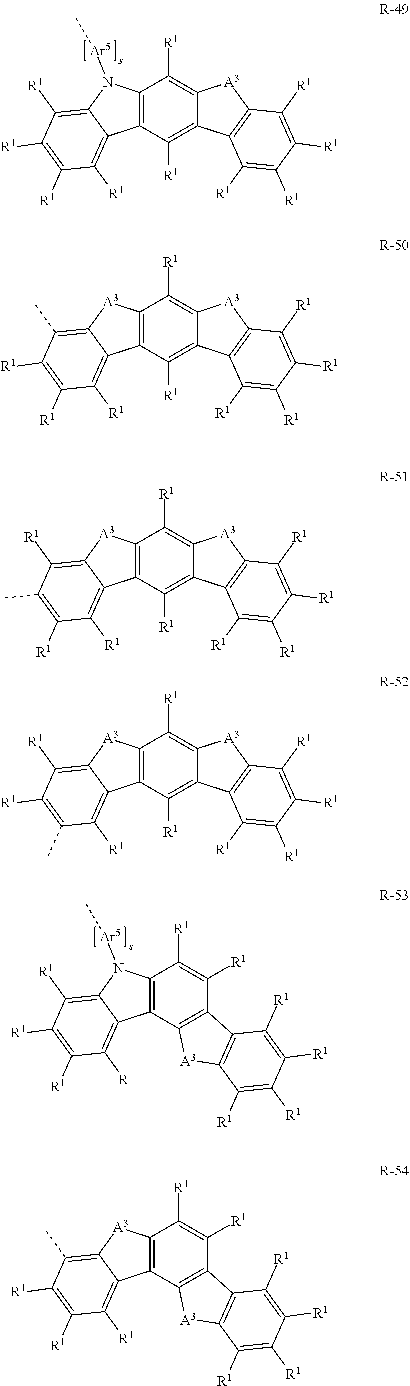

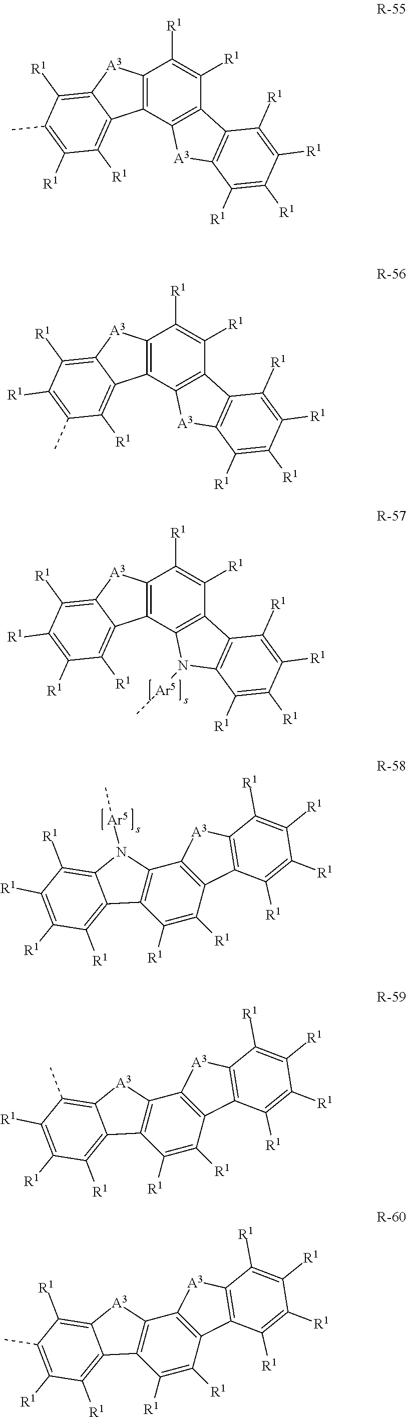

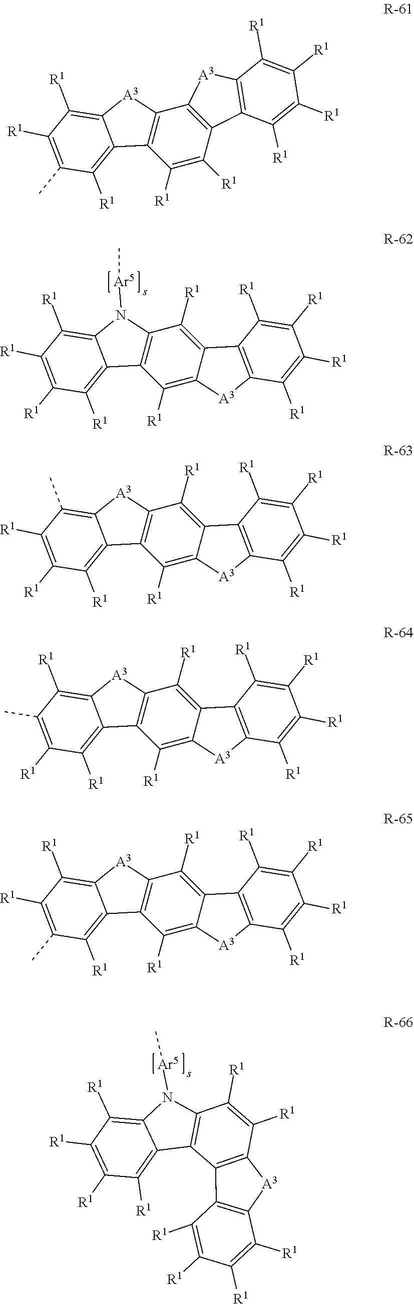

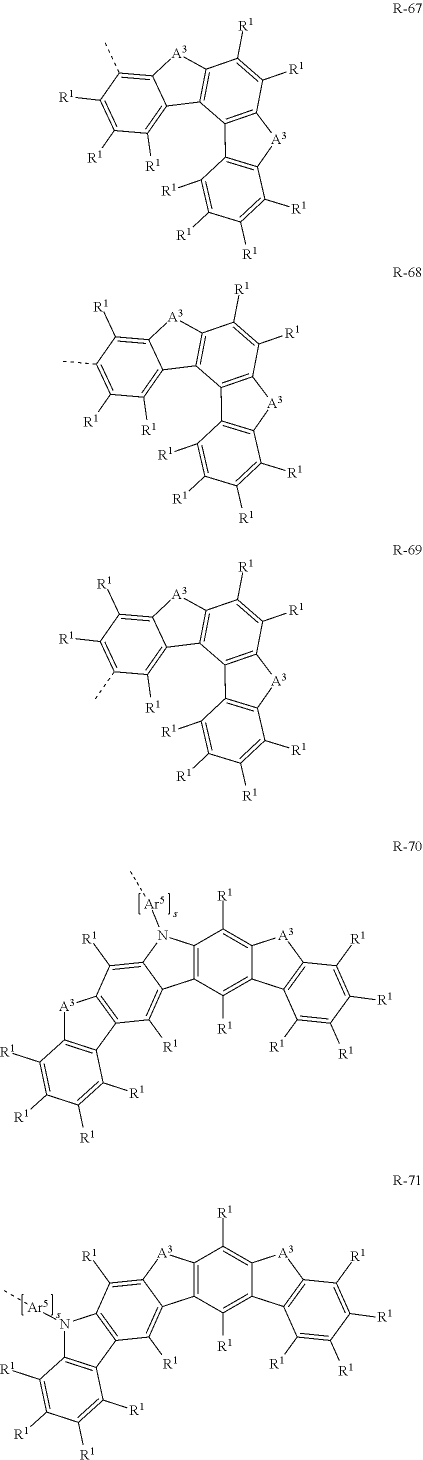

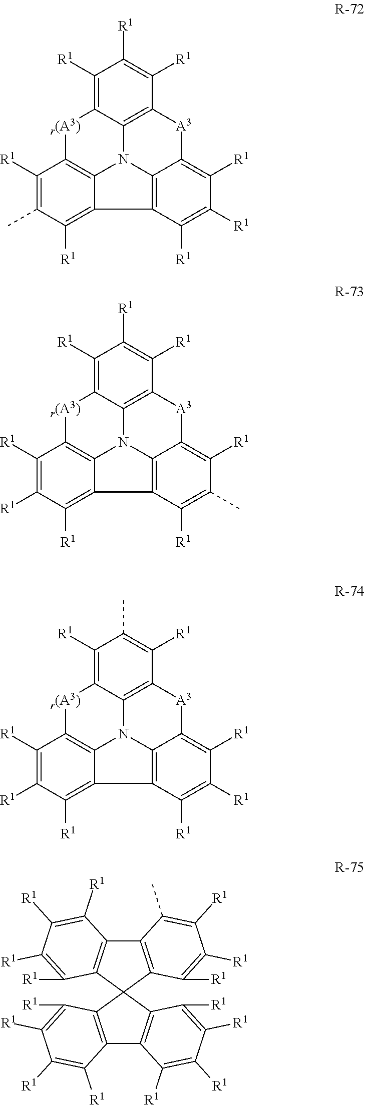

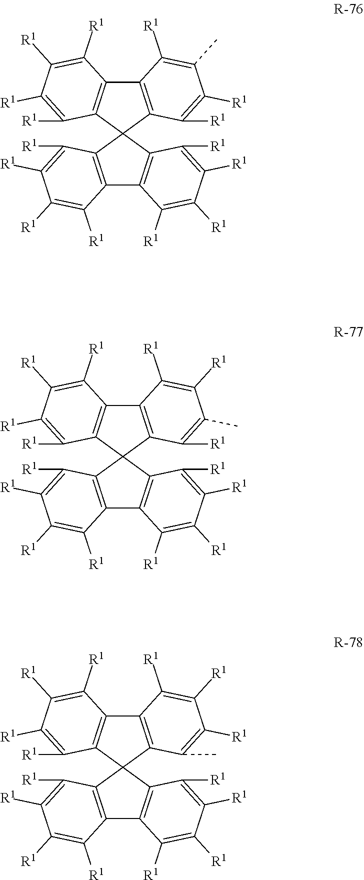

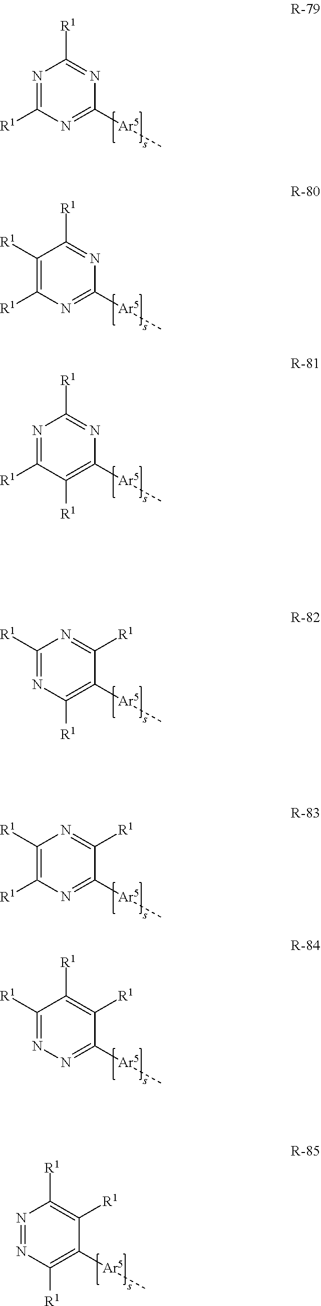

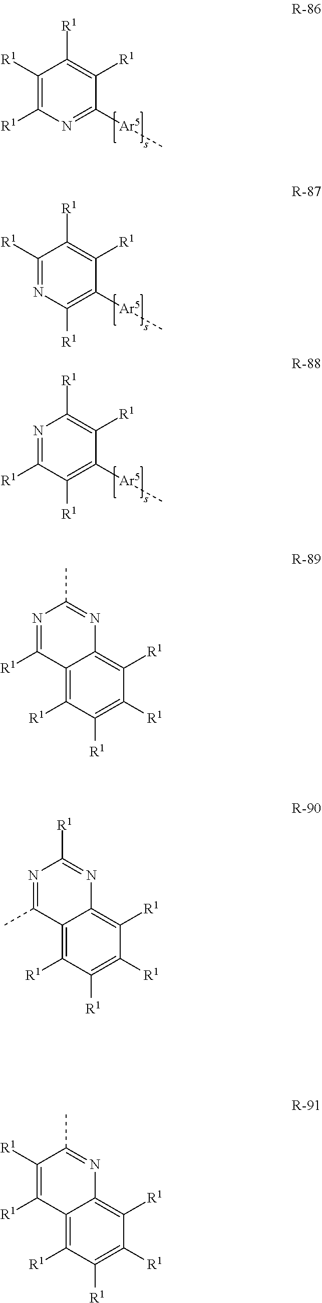

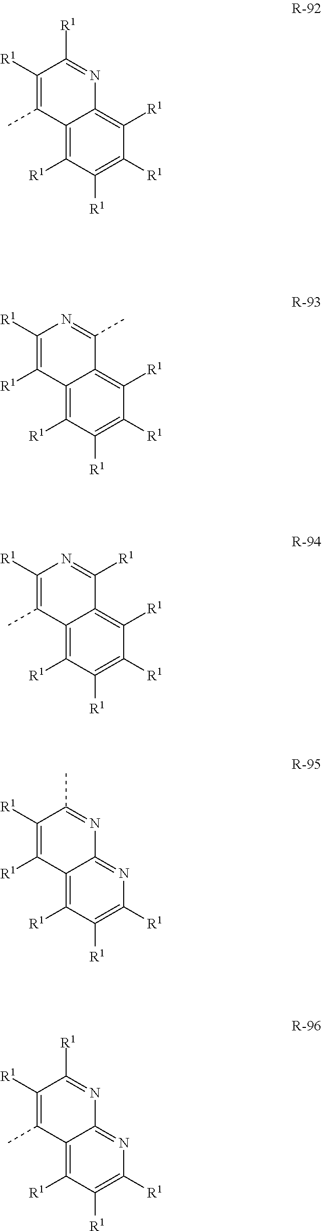

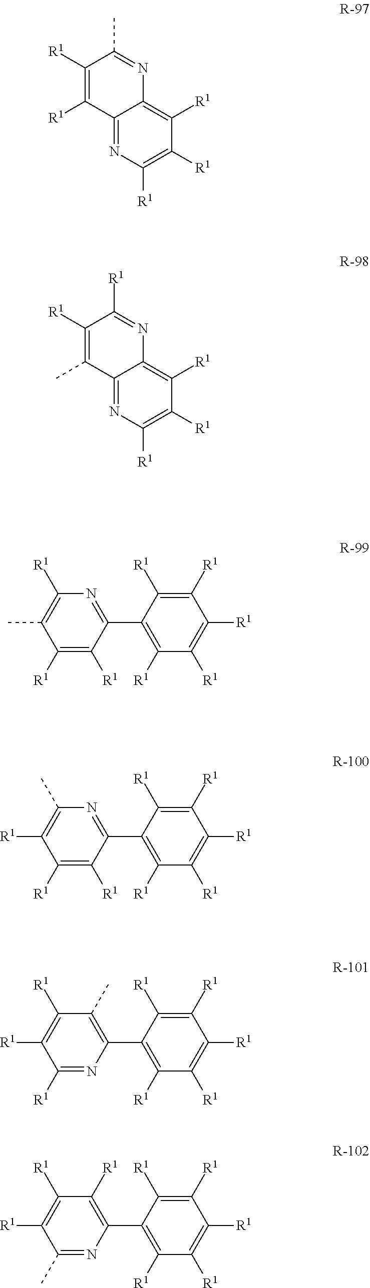

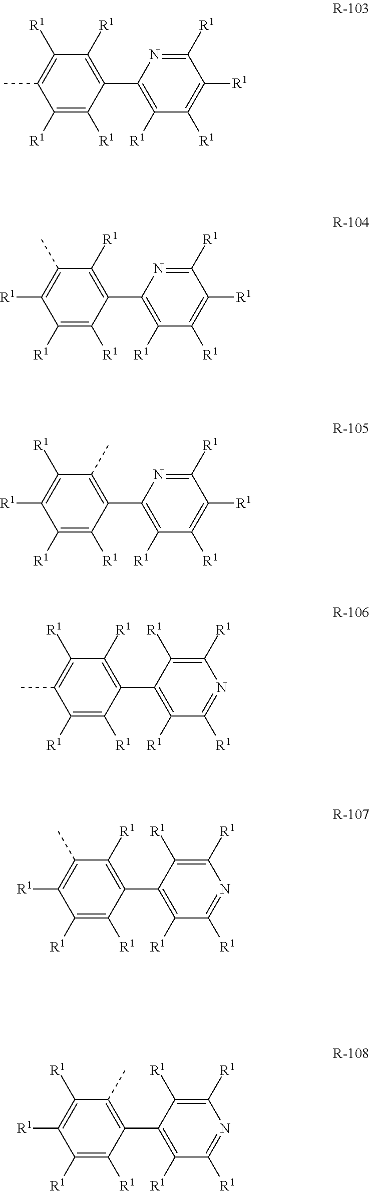

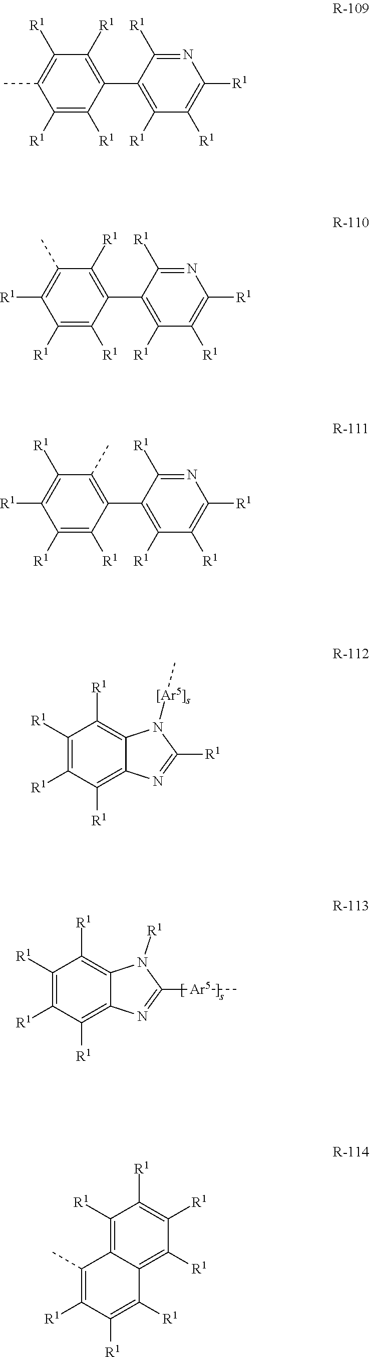

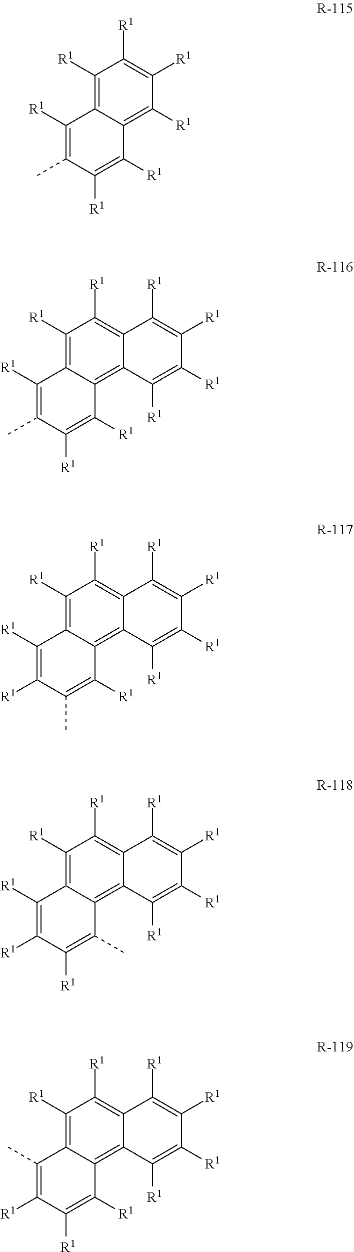

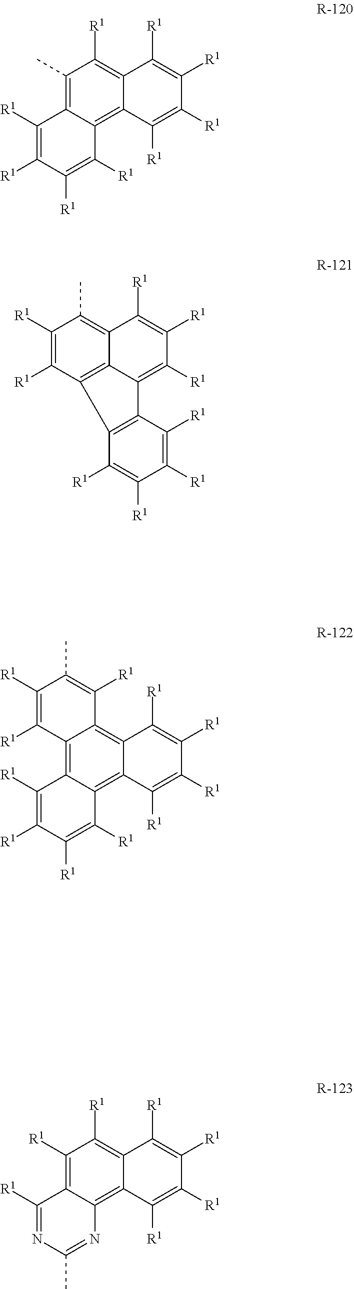

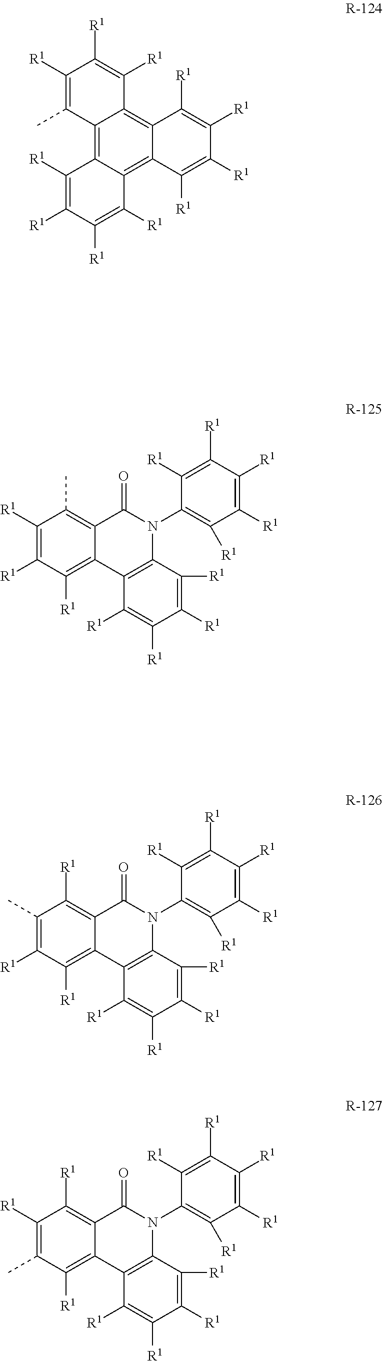

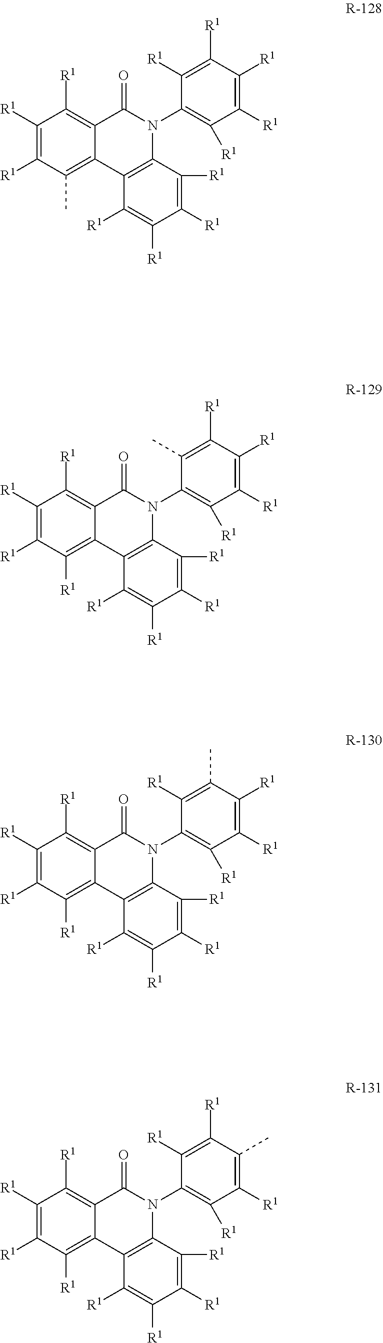

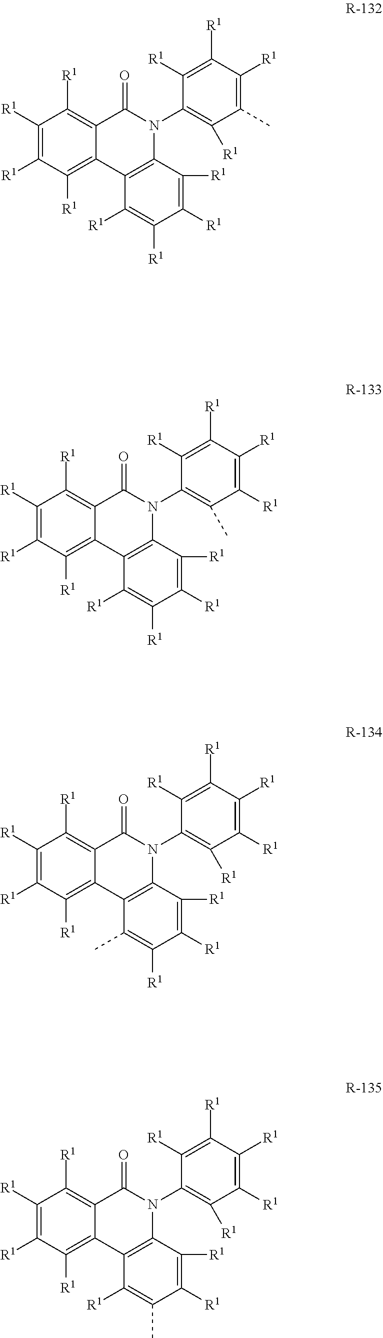

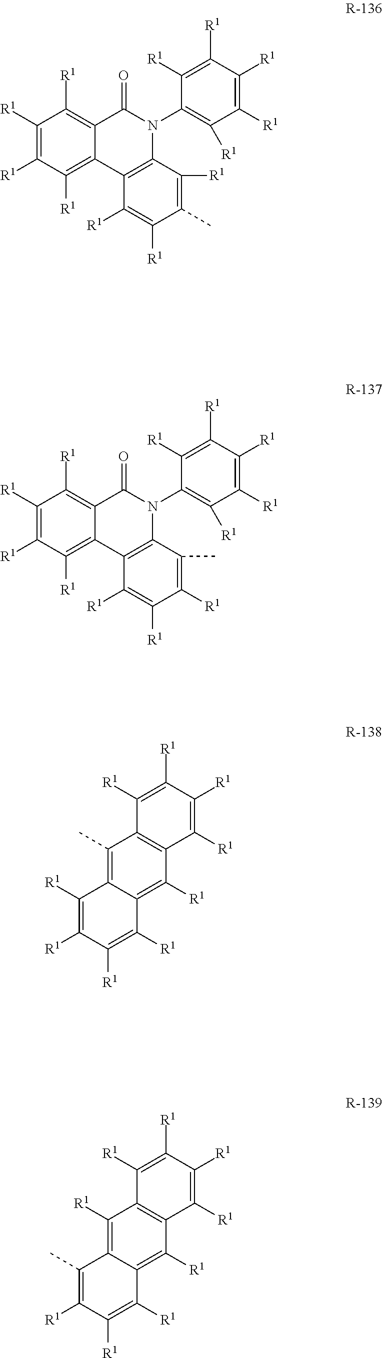

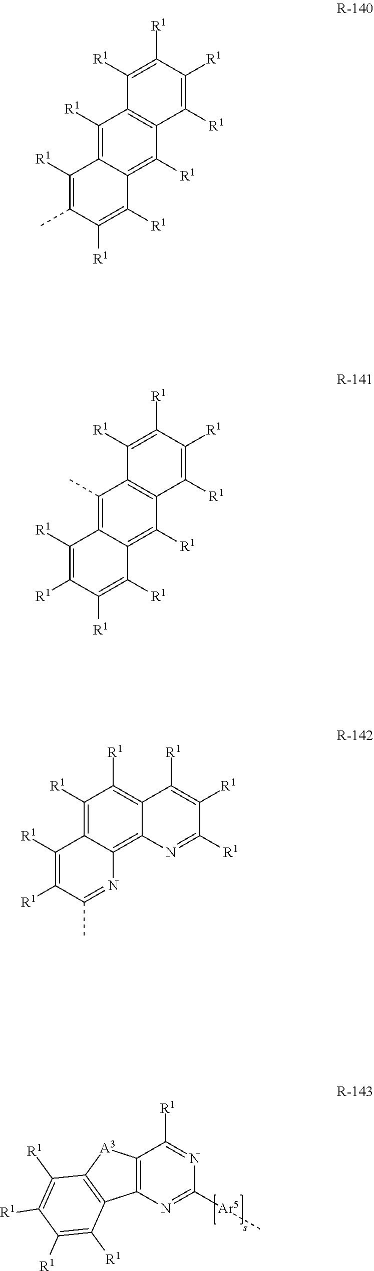

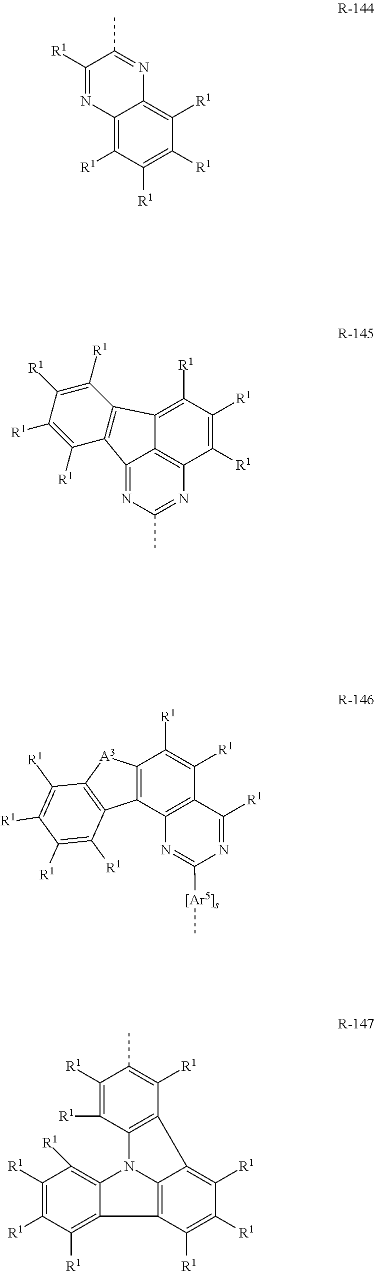

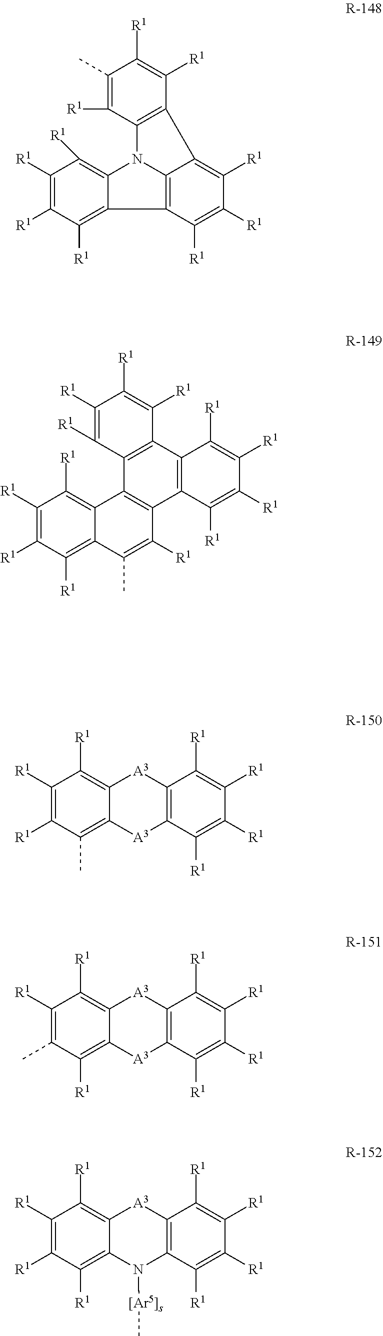

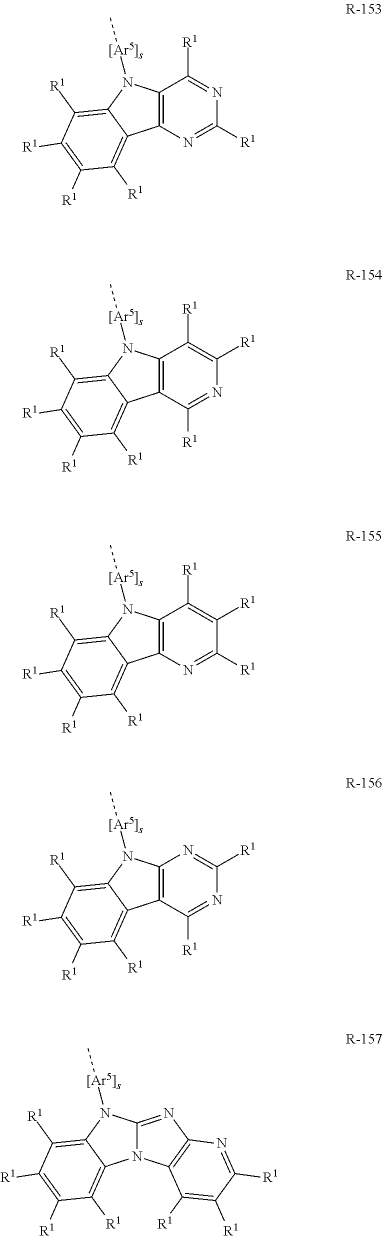

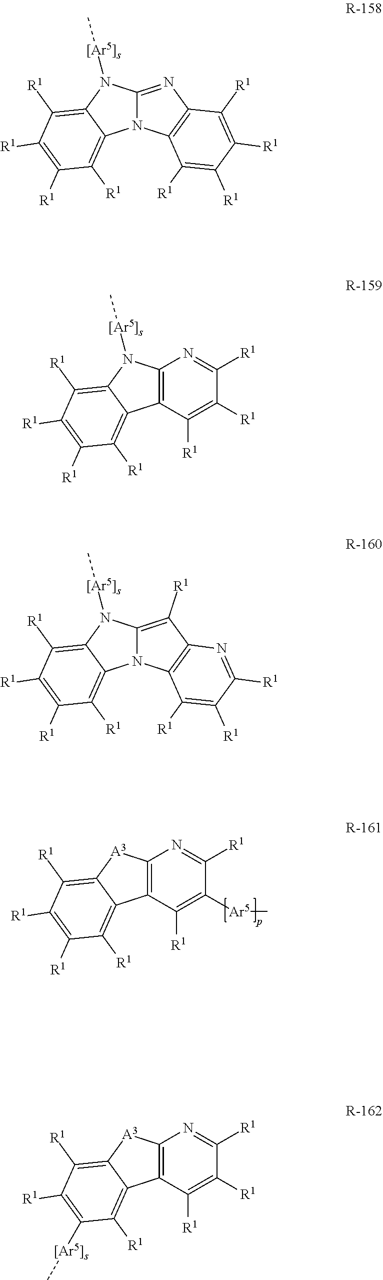

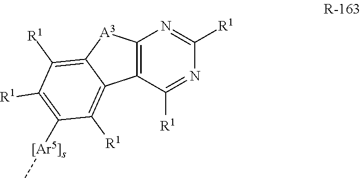

where R¹ has the definitions given above, the dotted bond represents the bond to formula (1) or formula (2), and the following definitions are additionally applicable:
Ar⁵ is the same or different at each instance and is a divalent aromatic or heteroaromatic ring system which has 6 to 18 aromatic ring atoms and may be substituted in each case by one or more R¹ radicals;
A³ is the same or different at each instance and is BR¹, C(R¹)₂, NR¹, PR¹, O or S, preferably C(R¹)₂, BR¹, NR¹, O or S;
A⁴ is the same or different at each instance and is C(R¹)₂, NR¹, O or S;
s is 0 or 1, where s=0 means that the Ar⁵ group is absent and that the corresponding aromatic or heteroaromatic group is bonded directly to a carbon atom of the base skeleton in formula (1);
r is 0 or 1, where r=0 means that no A³ group is bonded at this position and R¹ radicals are bonded to the corresponding carbon atoms instead.

In a preferred embodiment, Ar⁵ comprises divalent aromatic or heteroaromatic ring systems based on the groups of R-1 to R-163, where s=0 and the dotted bond and an R¹ represents the bond to the aromatic or heteroaromatic group according to R-1 to R-163.

When the abovementioned R-1 to R-163 groups for R have two or more A³ groups, possible options for these include all combinations from the definition of A³. Preferred embodiments in that case are those in which one A³ group is C(R¹)₂, BR¹, NR¹, O or S and the other A³ group is C(R¹)₂ or in which both A³ groups are S or O or in which both A³ groups are O or S.

When A³ is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for R-1 to R-35, where these structures may be substituted by one or more R¹ radicals, but are preferably unsubstituted.

When A³ is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

In a further preferred embodiment of the invention, R¹ is the same or different at each instance and is selected from the group consisting of H, D, F, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

In a further preferred embodiment of the invention, all R¹ radicals, if they are an aromatic or heteroaromatic ring system, or R² radicals, if they are aromatic or heteroaromatic groups, are selected from the R-1 to R-163 groups, in which case these, however, are correspondingly substituted by R², or by the groups mentioned for R².

In a preferred embodiment, the R radicals do not form any further aromatic or heteroaromatic groups fused to the base skeleton of the formula (1), unless specified explicitly in the preferred embodiments.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

| | |
|---|---|
| 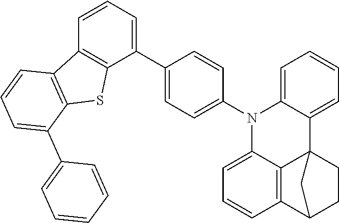 [SMILES: c1ccc(-c2cccc3c2sc2c(-c4ccc(N5c6ccccc6C67CCC(C6)c6cccc5c67)cc4)cccc23)cc1] | 1 |
| 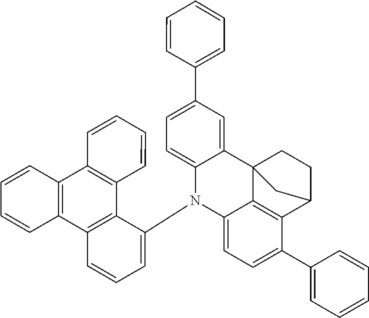 [SMILES: c1ccc(-c2ccc3c(c2)C24CCC(C2)c2c(-c5ccccc5)ccc(c24)N3c2cccc3c4ccccc4c4ccccc4c23)cc1] | 2 |
| 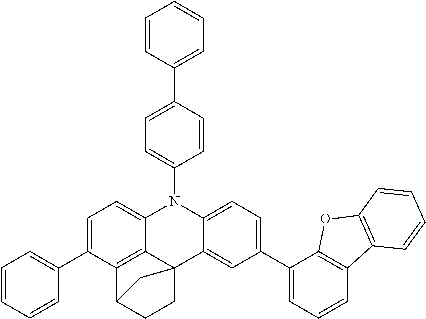 [SMILES: c1ccc(-c2ccc(N3c4ccc(-c5cccc6c5oc5ccccc56)cc4C45CCC(C4)c4c(-c6ccccc6)ccc3c45)cc2)cc1] | 3 |
| 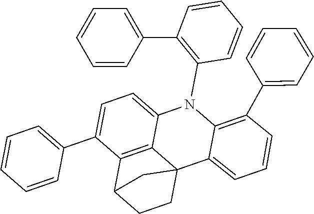 [SMILES: c1ccc(-c2ccccc2N2c3ccc(-c4ccccc4)c4c3C3(CCC4C3)c3cccc(-c4ccccc4)c32)cc1] | 4 |
| 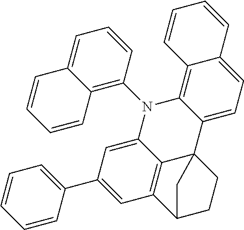 [SMILES: c1ccc(-c2cc3c4c(c2)N(c2cccc5ccccc25)c2c(ccc5ccccc25)C42CCC3C2)cc1] | 5 |
| 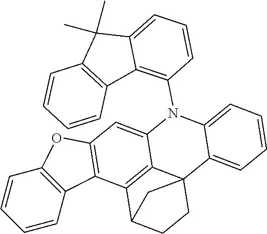 [SMILES: CC1(C)c2ccccc2-c2c(N3c4ccccc4C45CCC(C4)c4c5c3cc3oc5ccccc5c43)cccc21] | 6 |
| 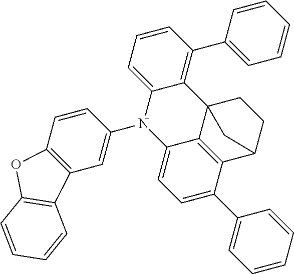 [SMILES: c1ccc(-c2ccc3c4c2C2CCC4(C2)c2c(-c4ccccc4)cccc2N3c2ccc3oc4ccccc4c3c2)cc1] | 7 |
| 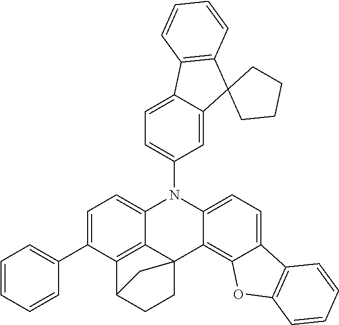 [SMILES: c1ccc(-c2ccc3c4c2C2CCC4(C2)c2c(ccc4c2oc2ccccc24)N3c2ccc3c(c2)C2(CCCC2)c2ccccc2-3)cc1] | 8 |
| 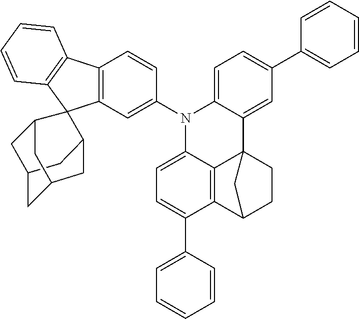 [SMILES: c1ccc(-c2ccc3c(c2)C24CCC(C2)c2c(-c5ccccc5)ccc(c24)N3c2ccc3c(c2)C2(c4ccccc4-3)C3CC4CC(C3)CC2C4)cc1] | 9 |
| 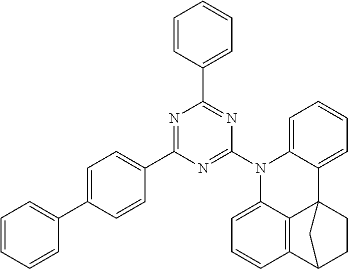 [SMILES: c1ccc(-c2ccc(-c3nc(-c4ccccc4)nc(N4c5ccccc5C56CCC(C5)c5cccc4c56)n3)cc2)cc1] | 10 |
| 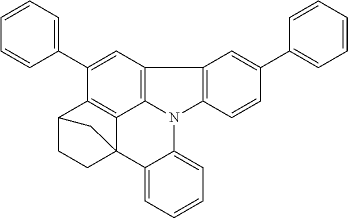 [SMILES: c1ccc(-c2ccc3c(c2)c2cc(-c4ccccc4)c4c5c2n3-c2ccccc2C52CCC4C2)cc1] | 11 |
| 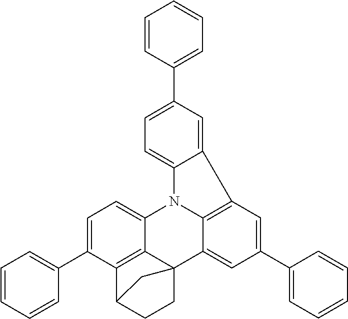 [SMILES: c1ccc(-c2ccc3c(c2)c2cc(-c4ccccc4)cc4c2n3-c2ccc(-c3ccccc3)c3c2C42CCC3C2)cc1] | 12 |
| 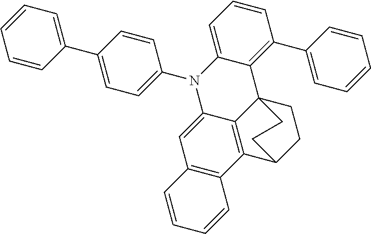 [SMILES: c1ccc(-c2ccc(N3c4cccc(-c5ccccc5)c4C45CCC(CC4)c4c5c3cc3ccccc43)cc2)cc1] | 13 |
| 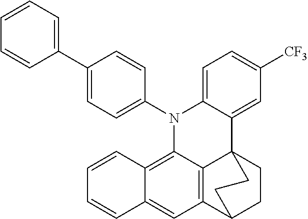 [SMILES: FC(F)(F)c1ccc2c(c1)C13CCC(CC1)c1cc4ccccc4c(c13)N2c1ccc(-c2ccccc2)cc1] | 14 |
| 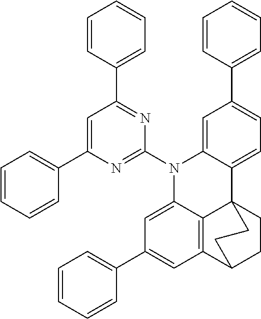 [SMILES: c1ccc(-c2ccc3c(c2)N(c2nc(-c4ccccc4)cc(-c4ccccc4)n2)c2cc(-c4ccccc4)cc4c2C32CCC4CC2)cc1] | 15 |
| 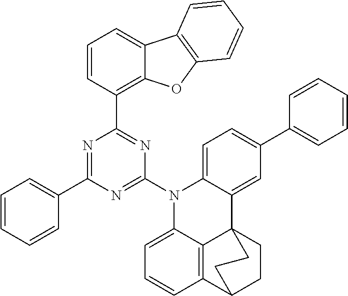 [SMILES: c1ccc(-c2ccc3c(c2)C24CCC(CC2)c2cccc(c24)N3c2nc(-c3ccccc3)nc(-c3cccc4c3oc3ccccc34)n2)cc1] | 16 |
| 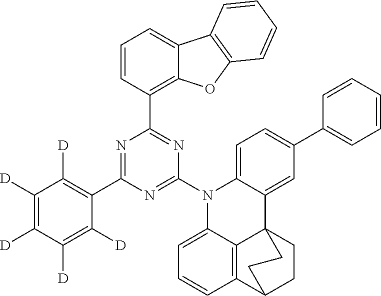 [SMILES: [2H]c1c([2H])c([2H])c(-c2nc(-c3cccc4c3oc3ccccc34)nc(N3c4ccc(-c5ccccc5)cc4C45CCC(CC4)c4cccc3c45)n2)c([2H])c1[2H]] | 17 |
| 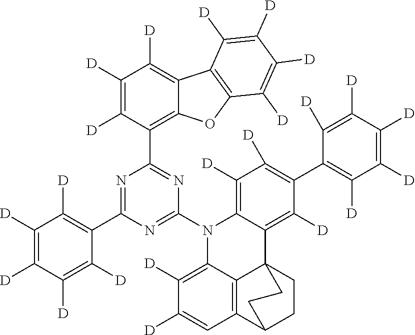 [SMILES: [2H]c1cc2c3c(c1[2H])N(c1nc(-c4c([2H])c([2H])c([2H])c([2H])c4[2H])nc(-c4c([2H])c([2H])c([2H])c5c4oc4c([2H])c([2H])c([2H])c([2H])c45)n1)c1c([2H])c([2H])c(-c4c([2H])c([2H])c([2H])c([2H])c4[2H])c([2H])c1C31CCC2CC1] | 18 |
| 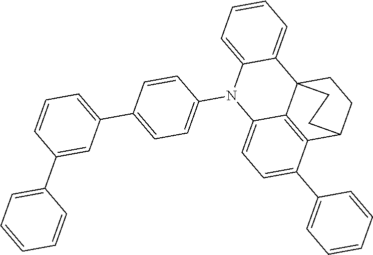 [SMILES: c1ccc(-c2cccc(-c3ccc(N4c5ccccc5C56CCC(CC5)c5c(-c7ccccc7)ccc4c56)cc3)c2)cc1] | 19 |
| 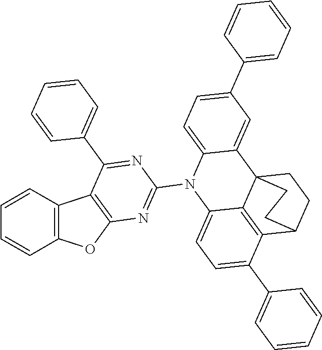 [SMILES: c1ccc(-c2ccc3c(c2)C24CCC(CC2)c2c(-c5ccccc5)ccc(c24)N3c2nc(-c3ccccc3)c3c(n2)oc2ccccc23)cc1] | 20 |
| 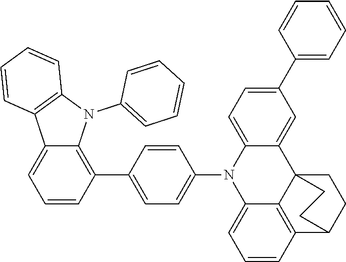 [SMILES: c1ccc(-c2ccc3c(c2)C24CCC(CC2)c2cccc(c24)N3c2ccc(-c3cccc4c5ccccc5n(-c5ccccc5)c34)cc2)cc1] | 21 |
| 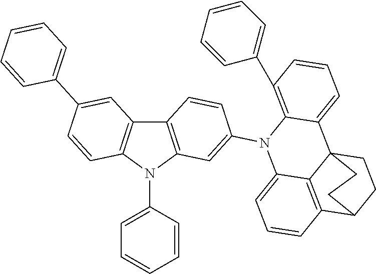 [SMILES: c1ccc(-c2ccc3c(c2)c2ccc(N4c5cccc6c5C5(CCC6CC5)c5cccc(-c6ccccc6)c54)cc2n3-c2ccccc2)cc1] | 22 |
| 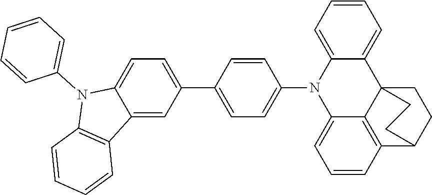 [SMILES: c1ccc(-n2c3ccccc3c3cc(-c4ccc(N5c6ccccc6C67CCC(CC6)c6cccc5c67)cc4)ccc32)cc1] | 23 |
| 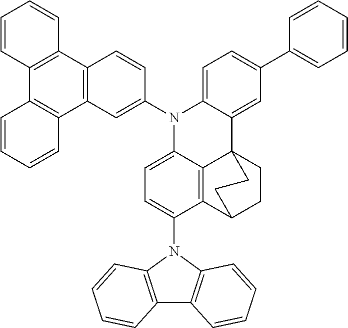 [SMILES: c1ccc(-c2ccc3c(c2)C24CCC(CC2)c2c(-n5c6ccccc6c6ccccc65)ccc(c24)N3c2ccc3c4ccccc4c4ccccc4c3c2)cc1] | 24 |
| 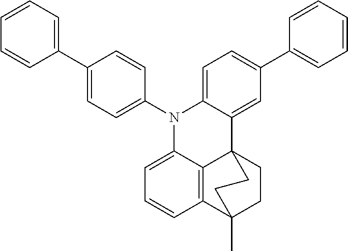 [SMILES: CC12CCC3(CC1)c1cc(-c4ccccc4)ccc1N(c1ccc(-c4ccccc4)cc1)c1cccc2c13] | 25 |
| 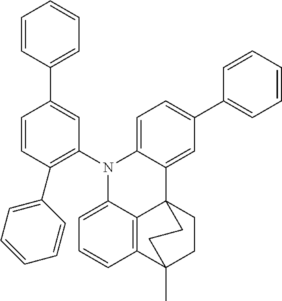 [SMILES: CC12CCC3(CC1)c1cc(-c4ccccc4)ccc1N(c1cc(-c4ccccc4)ccc1-c1ccccc1)c1cccc2c13] | 26 |
| 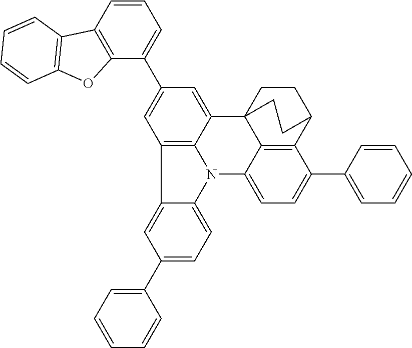 [SMILES: c1ccc(-c2ccc3c(c2)c2cc(-c4cccc5c4oc4ccccc45)cc4c2n3-c2ccc(-c3ccccc3)c3c2C42CCC3CC2)cc1] | 27 |
| 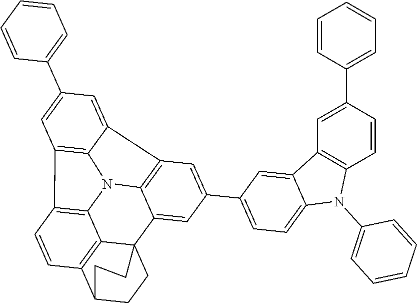 [SMILES: c1ccc(-c2ccc3c(c2)c2cc(-c4cc5c6c(c4)c4cc(-c7ccccc7)cc7c8ccc9c(c8n6c47)C54CCC9CC4)ccc2n3-c2ccccc2)cc1] | 28 |
| 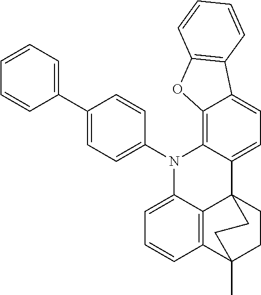 [SMILES: CC12CCC3(CC1)c1ccc4c(oc5ccccc54)c1N(c1ccc(-c4ccccc4)cc1)c1cccc2c13] | 29 |
| 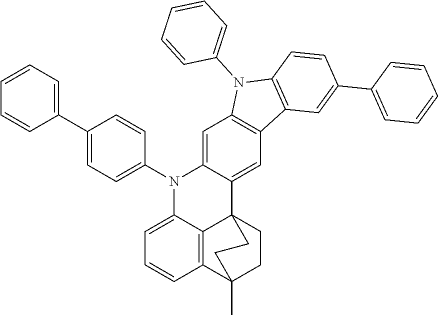 [SMILES: CC12CCC3(CC1)c1cc4c5cc(-c6ccccc6)ccc5n(-c5ccccc5)c4cc1N(c1ccc(-c4ccccc4)cc1)c1cccc2c13] | 30 |
| 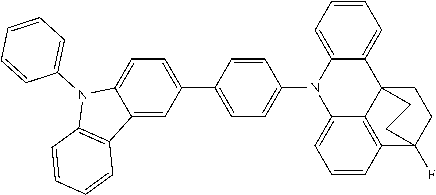 [SMILES: FC12CCC3(CC1)c1ccccc1N(c1ccc(-c4ccc5c(c4)c4ccccc4n5-c4ccccc4)cc1)c1cccc2c13] | 31 |
| 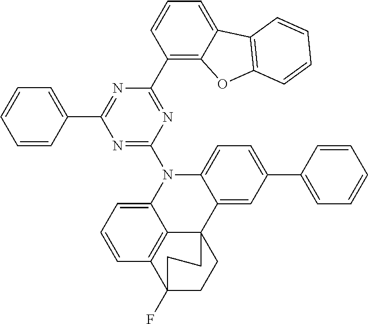 [SMILES: FC12CCC3(CC1)c1cc(-c4ccccc4)ccc1N(c1nc(-c4ccccc4)nc(-c4cccc5c4oc4ccccc45)n1)c1cccc2c13] | 32 |
| 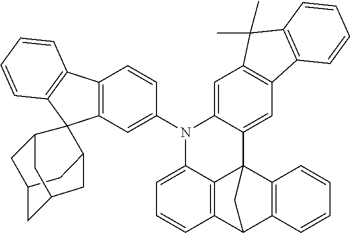 [SMILES: CC1(C)c2ccccc2-c2cc3c(cc21)N(c1ccc2c(c1)C1(c4ccccc4-2)C2CC4CC(C2)CC1C4)c1cccc2c1C31CC2c2ccccc21] | 33 |
| 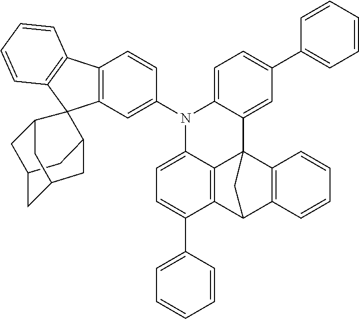 [SMILES: c1ccc(-c2ccc3c(c2)C24CC(c5ccccc52)c2c(-c5ccccc5)ccc(c24)N3c2ccc3c(c2)C2(c4ccccc4-3)C3CC4CC(C3)CC2C4)cc1] | 34 |
| 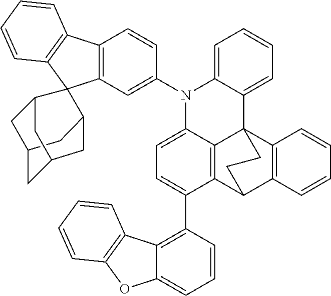 [SMILES: c1ccc2c(c1)-c1ccc(N3c4ccccc4C45CCC(c6ccccc64)c4c(-c6cccc7oc8ccccc8c67)ccc3c45)cc1C21C2CC3CC(C2)CC1C3] | 35 |
| 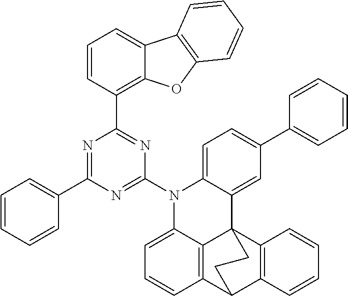 [SMILES: c1ccc(-c2ccc3c(c2)C24CCC(c5ccccc52)c2cccc(c24)N3c2nc(-c3ccccc3)nc(-c3cccc4c3oc3ccccc34)n2)cc1] | 36 |
| 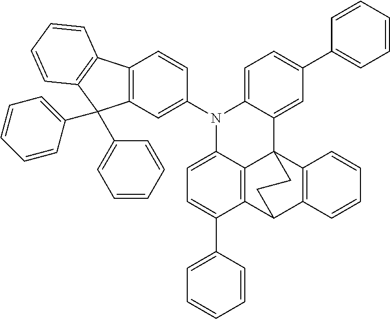 [SMILES: c1ccc(-c2ccc3c(c2)C24CCC(c5ccccc52)c2c(-c5ccccc5)ccc(c24)N3c2ccc3c(c2)C(c2ccccc2)(c2ccccc2)c2ccccc2-3)cc1] | 37 |
| 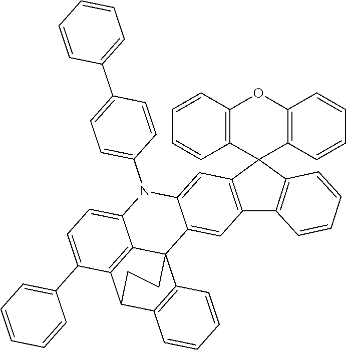 [SMILES: c1ccc(-c2ccc(N3c4cc5c(cc4C46CCC(c7ccccc74)c4c(-c7ccccc7)ccc3c46)-c3ccccc3C53c4ccccc4Oc4ccccc43)cc2)cc1] | 38 |
| 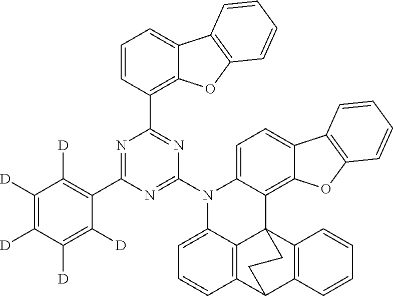 [SMILES: [2H]c1c([2H])c([2H])c(-c2nc(-c3cccc4c3oc3ccccc34)nc(N3c4cccc5c4C4(CCC5c5ccccc54)c4c3ccc3c4oc4ccccc43)n2)c([2H])c1[2H]] | 39 |
| 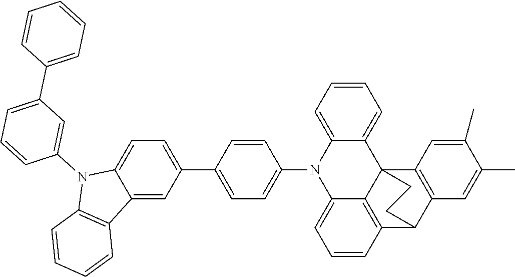 [SMILES: Cc1cc2c(cc1C)C13CCC2c2cccc(c21)N(c1ccc(-c2ccc4c(c2)c2ccccc2n4-c2cccc(-c4ccccc4)c2)cc1)c1ccccc13] | 40 |
| 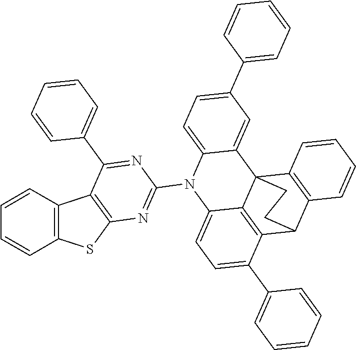 [SMILES: c1ccc(-c2ccc3c(c2)C24CCC(c5ccccc52)c2c(-c5ccccc5)ccc(c24)N3c2nc(-c3ccccc3)c3c(n2)sc2ccccc23)cc1] | 41 |
| 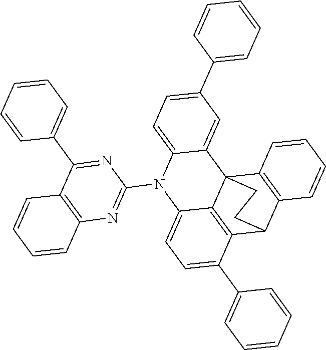 [SMILES: c1ccc(-c2ccc3c(c2)C24CCC(c5ccccc52)c2c(-c5ccccc5)ccc(c24)N3c2nc(-c3ccccc3)c3ccccc3n2)cc1] | 42 |
| 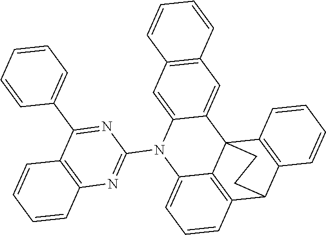 [SMILES: c1ccc(-c2nc(N3c4cc5ccccc5cc4C45CCC(c6ccccc64)c4cccc3c45)nc3ccccc23)cc1] | 43 |
| 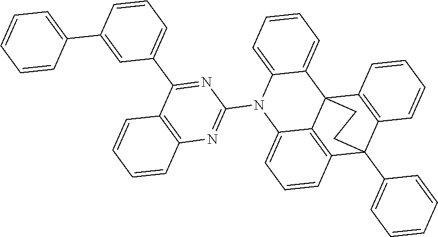 [SMILES: c1ccc(-c2cccc(-c3nc(N4c5ccccc5C56CCC(c7ccccc7)(c7ccccc75)c5cccc4c56)nc4ccccc34)c2)cc1] | 44 |
| 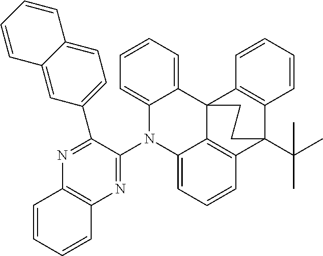 [SMILES: CC(C)(C)C12CCC3(c4ccccc4N(c4nc5ccccc5nc4-c4ccc5ccccc5c4)c4cccc1c43)c1ccccc12] | 45 |
| 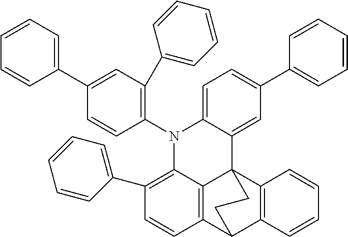 [SMILES: c1ccc(-c2ccc(N3c4ccc(-c5ccccc5)cc4C45CCC(c6ccccc64)c4ccc(-c6ccccc6)c3c45)c(-c3ccccc3)c2)cc1] | 46 |
| 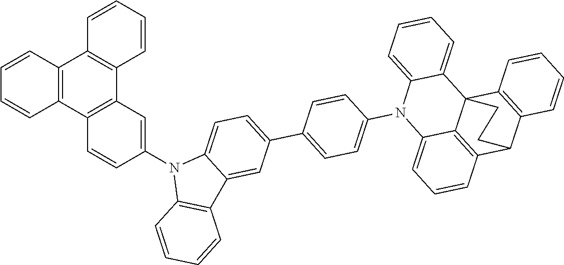 [SMILES: c1ccc2c(c1)C1CCC23c2ccccc2N(c2ccc(-c4ccc5c(c4)c4ccccc4n5-c4ccc5c6ccccc6c6ccccc6c5c4)cc2)c2cccc1c23] | 47 |
| 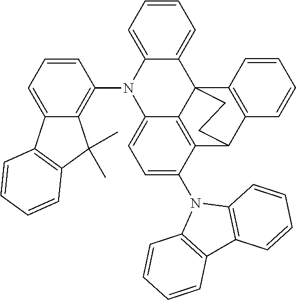 [SMILES: CC1(C)c2ccccc2-c2cccc(N3c4ccccc4C45CCC(c6ccccc64)c4c(-n6c7ccccc7c7ccccc76)ccc3c45)c21] | 48 |
| 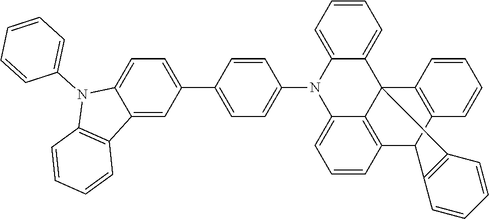 [SMILES: c1ccc(-n2c3ccccc3c3cc(-c4ccc(N5c6ccccc6C67c8ccccc8C(c8ccccc86)c6cccc5c67)cc4)ccc32)cc1] | 49 |
| 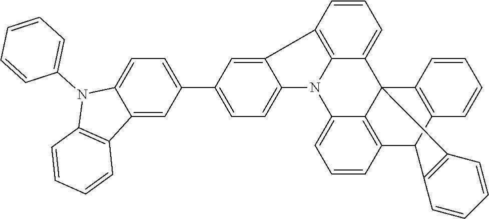 [SMILES: c1ccc(-n2c3ccccc3c3cc(-c4ccc5c(c4)c4cccc6c4n5-c4cccc5c4C64c6ccccc6C5c5ccccc54)ccc32)cc1] | 50 |
| 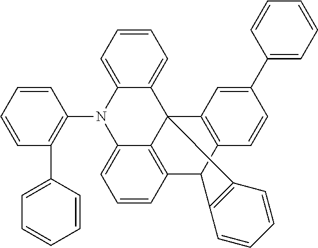 [SMILES: c1ccc(-c2ccc3c(c2)C24c5ccccc5C3c3cccc(c32)N(c2ccccc2-c2ccccc2)c2ccccc24)cc1] | 51 |
| 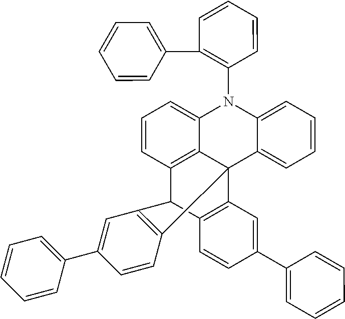 [SMILES: c1ccc(-c2ccc3c(c2)C2c4ccc(-c5ccccc5)cc4C34c3ccccc3N(c3ccccc3-c3ccccc3)c3cccc2c34)cc1] | 52 |
| 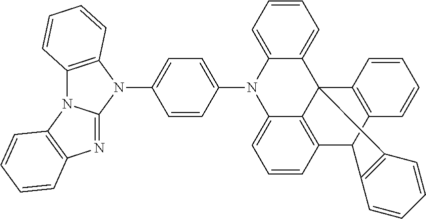 [SMILES: c1ccc2c(c1)C1c3ccccc3C23c2ccccc2N(c2ccc(-n4c5ccccc5n5c6ccccc6nc45)cc2)c2cccc1c23] | 53 |
| 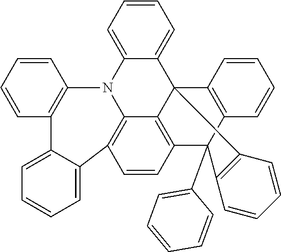 [SMILES: c1ccc(C23c4ccccc4C4(c5ccccc5N5c6ccccc6-c6ccccc6-c6ccc2c4c65)c2ccccc23)cc1] | 54 |
| 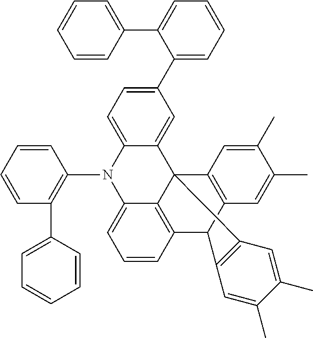 [SMILES: Cc1cc2c(cc1C)C13c4cc(C)c(C)cc4C2c2cccc(c21)N(c1ccccc1-c1ccccc1)c1ccc(-c2ccccc2-c2ccccc2)cc13] | 55 |
| 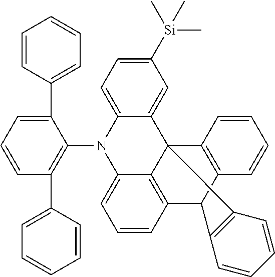 [SMILES: C[Si](C)(C)c1ccc2c(c1)C13c4ccccc4C(c4ccccc41)c1cccc(c13)N2c1c(-c2ccccc2)cccc1-c1ccccc1] | 56 |
| 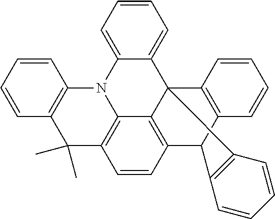 [SMILES: CC1(C)c2ccccc2N2c3ccccc3C34c5ccccc5C(c5ccccc53)c3ccc1c2c34] | 57 |
| 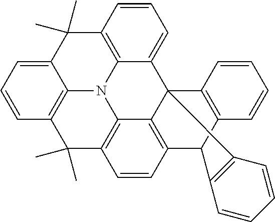 [SMILES: CC1(C)c2cccc3c2N2c4c1cccc4C14c5ccccc5C(c5ccccc51)c1ccc(c2c14)C3(C)C] | 58 |
| 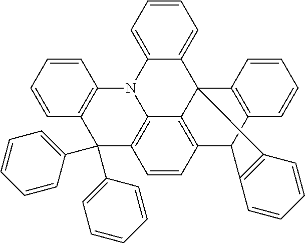 [SMILES: c1ccc(C2(c3ccccc3)c3ccccc3N3c4ccccc4C45c6ccccc6C(c6ccccc64)c4ccc2c3c45)cc1] | 59 |
| 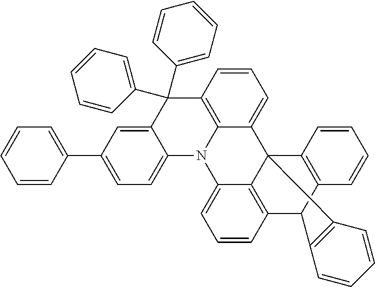 [SMILES: c1ccc(-c2ccc3c(c2)C(c2ccccc2)(c2ccccc2)c2cccc4c2N3c2cccc3c2C42c4ccccc4C3c3ccccc32)cc1] | 60 |
| 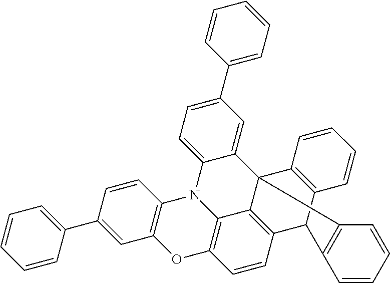 [SMILES: c1ccc(-c2ccc3c(c2)Oc2ccc4c5c2N3c2ccc(-c3ccccc3)cc2C52c3ccccc3C4c3ccccc32)cc1] | 61 |
| 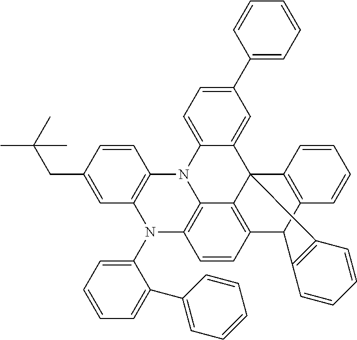 [SMILES: CC(C)(C)Cc1ccc2c(c1)N(c1ccccc1-c1ccccc1)c1ccc3c4c1N2c1ccc(-c2ccccc2)cc1C41c2ccccc2C3c2ccccc21] | 62 |
| 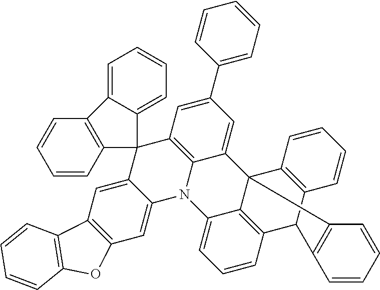 [SMILES: c1ccc(-c2cc3c4c(c2)C25c6ccccc6C(c6ccccc62)c2cccc(c25)N4c2cc4oc5ccccc5c4cc2C32c3ccccc3-c3ccccc32)cc1] | 63 |
| 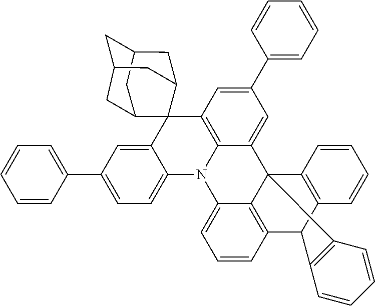 [SMILES: c1ccc(-c2ccc3c(c2)C2(c4cc(-c5ccccc5)cc5c4N3c3cccc4c3C53c5ccccc5C4c4ccccc43)C3CC4CC(C3)CC2C4)cc1] | 64 |
| 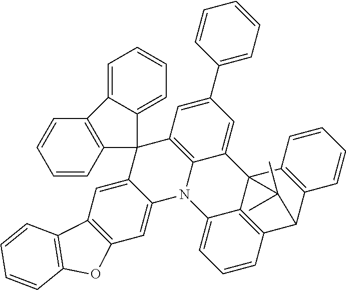 [SMILES: CC1(C)C2c3ccccc3C13c1cc(-c4ccccc4)cc4c1N(c1cc5oc6ccccc6c5cc1C41c4ccccc4-c4ccccc41)c1cccc2c13] | 65 |
| 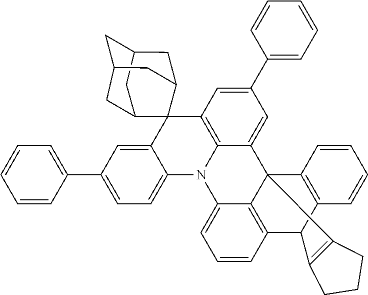 [SMILES: c1ccc(-c2ccc3c(c2)C2(c4cc(-c5ccccc5)cc5c4N3c3cccc4c3C53C5=C(CCC5)C4c4ccccc43)C3CC4CC(C3)CC2C4)cc1] | 66 |
| 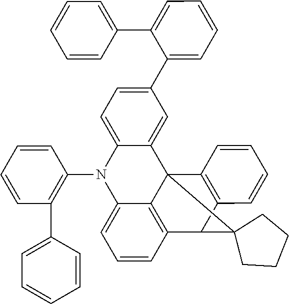 [SMILES: c1ccc(-c2ccccc2-c2ccc3c(c2)C24c5ccccc5C(c5cccc(c52)N3c2ccccc2-c2ccccc2)C42CCCC2)cc1] | 67 |
| 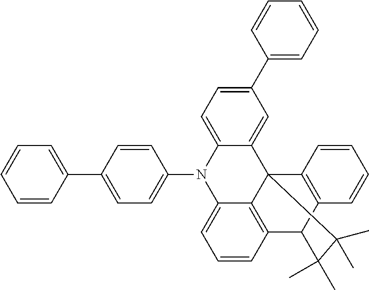 [SMILES: CC1(C)C2c3ccccc3C3(c4cc(-c5ccccc5)ccc4N(c4ccc(-c5ccccc5)cc4)c4cccc2c43)C1(C)C] | 68 |
| 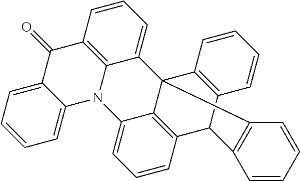 [SMILES: O=c1c2ccccc2n2c3c(cccc13)C13c4ccccc4C(c4ccccc41)c1cccc-2c13] | 69 |
| 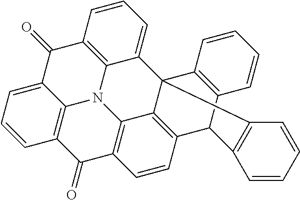 [SMILES: O=c1c2cccc3c2n2c4c5c(ccc4c(=O)c4cccc1c42)C1c2ccccc2C35c2ccccc21] | 70 |
| 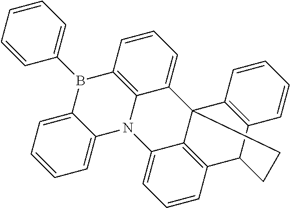 [SMILES: c1ccc(B2c3ccccc3N3c4cccc5c4C4(CCC5c5ccccc54)c4cccc2c43)cc1] | 71 |
| 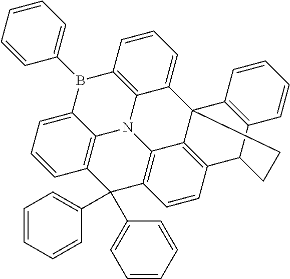 [SMILES: c1ccc(B2c3cccc4c3N3c5c2cccc5C(c2ccccc2)(c2ccccc2)c2ccc5c(c23)C42CCC5c3ccccc32)cc1] | 72 |
| 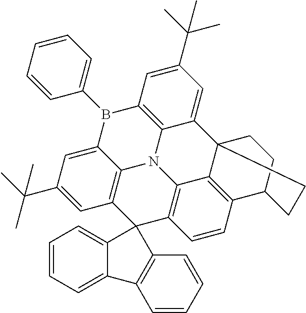 [SMILES: CC(C)(C)c1cc2c3c(c1)C14CCC(CC1)c1ccc5c(c14)N3c1c(cc(C(C)(C)C)cc1C51c3ccccc3-c3ccccc31)B2c1ccccc1] | 73 |
| 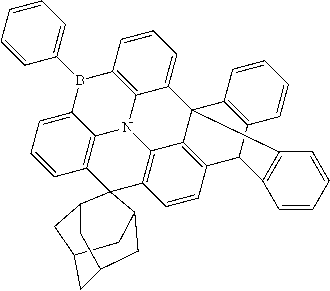 [SMILES: c1ccc(B2c3cccc4c3N3c5c2cccc5C2(c5ccc6c(c53)C43c4ccccc4C6c4ccccc43)C3CC4CC(C3)CC2C4)cc1] | 74 |
| 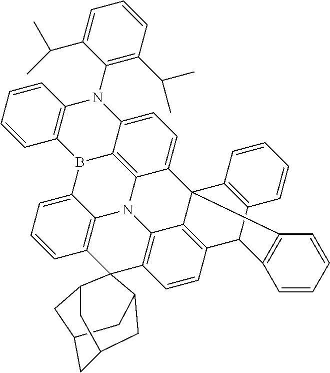 [SMILES: CC(C)c1cccc(C(C)C)c1N1c2ccccc2B2c3cccc4c3N3c5c(ccc1c52)C12c5ccccc5C(c5ccccc51)c1ccc(c3c12)C41C2CC3CC(C2)CC1C3] | 75 |
| 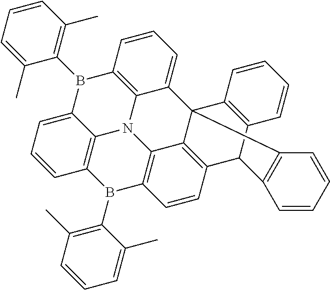 [SMILES: Cc1cccc(C)c1B1c2cccc3c2N2c4c1cccc4C14c5ccccc5C(c5ccccc51)c1ccc(c2c14)B3c1c(C)cccc1C] | 76 |
| 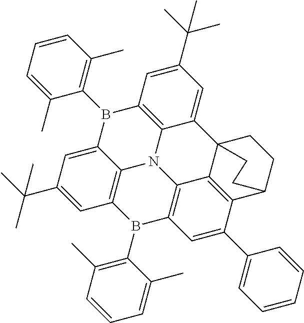 [SMILES: Cc1cccc(C)c1B1c2cc(C(C)(C)C)cc3c2N2c4c1cc(C(C)(C)C)cc4C14CCC(CC1)c1c(-c5ccccc5)cc(c2c14)B3c1c(C)cccc1C] | 77 |
| 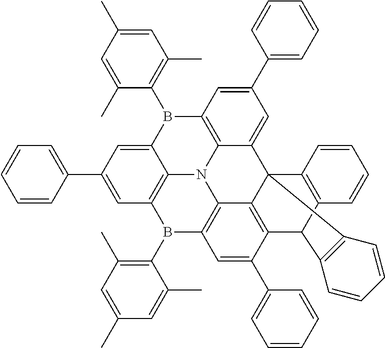 [SMILES: Cc1cc(C)c(B2c3cc(-c4ccccc4)cc4c3N3c5c2cc(-c2ccccc2)cc5C25c6ccccc6C(c6ccccc62)c2c(-c6ccccc6)cc(c3c25)B4c2c(C)cc(C)cc2C)c(C)c1] | 78 |
| 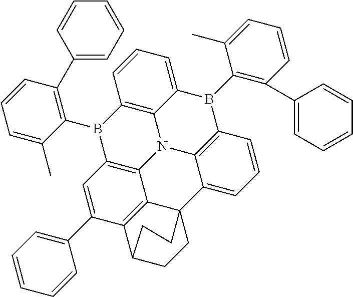 [SMILES: Cc1cccc(-c2ccccc2)c1B1c2cccc3c2N2c4c1cccc4C14CCC(CC1)c1c(-c5ccccc5)cc(c2c14)B3c1c(C)cccc1-c1ccccc1] | 79 |
| 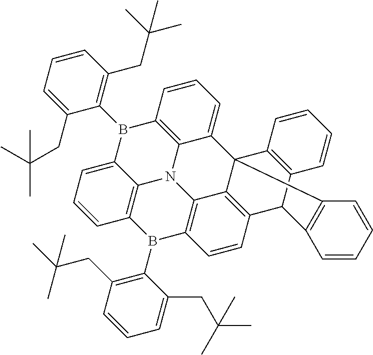 [SMILES: CC(C)(C)Cc1cccc(CC(C)(C)C)c1B1c2cccc3c2N2c4c1cccc4C14c5ccccc5C(c5ccccc51)c1ccc(c2c14)B3c1c(CC(C)(C)C)cccc1CC(C)(C)C] | 80 |
| 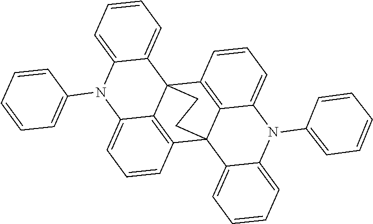 [SMILES: c1ccc(N2c3ccccc3C34CCC5(c6ccccc6N(c6ccccc6)c6cccc3c65)c3cccc2c34)cc1] | 81 |
| 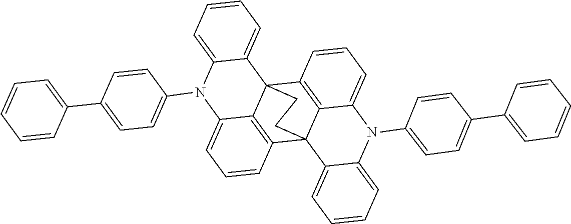 [SMILES: c1ccc(-c2ccc(N3c4ccccc4C45CCC6(c7ccccc7N(c7ccc(-c8ccccc8)cc7)c7cccc4c76)c4cccc3c45)cc2)cc1] | 82 |
| 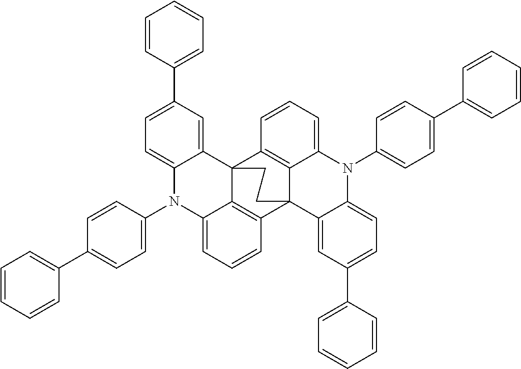 [SMILES: c1ccc(-c2ccc(N3c4ccc(-c5ccccc5)cc4C45CCC6(c7cc(-c8ccccc8)ccc7N(c7ccc(-c8ccccc8)cc7)c7cccc4c76)c4cccc3c45)cc2)cc1] | 83 |
| 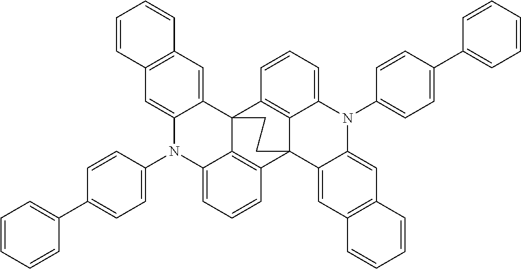 [SMILES: c1ccc(-c2ccc(N3c4cc5ccccc5cc4C45CCC6(c7cc8ccccc8cc7N(c7ccc(-c8ccccc8)cc7)c7cccc4c76)c4cccc3c45)cc2)cc1] | 84 |
| 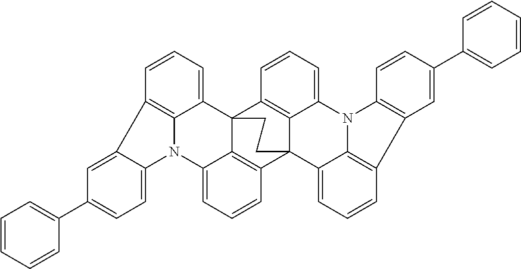 [SMILES: c1ccc(-c2ccc3c(c2)c2cccc4c2n3-c2cccc3c2C42CCC34c3c(cccc32)-n2c3ccc(-c5ccccc5)cc3c3cccc4c32)cc1] | 85 |
| 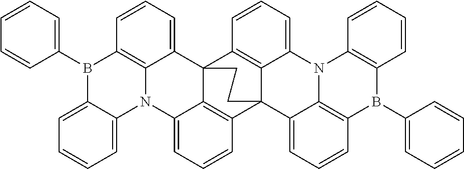 [SMILES: c1ccc(B2c3ccccc3N3c4cccc5c4C4(CCC56c5cccc7c5N(c5ccccc5B7c5ccccc5)c5cccc4c56)c4cccc2c43)cc1] | 86 |
| 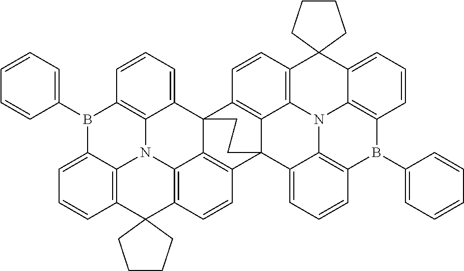 [SMILES: c1ccc(B2c3cccc4c3N3c5c2cccc5C25CCC6(c7cccc8c7N7c9c(cccc9C9(CCCC9)c9ccc2c6c97)B8c2ccccc2)c2ccc(c3c25)C42CCCC2)cc1] | 87 |
| 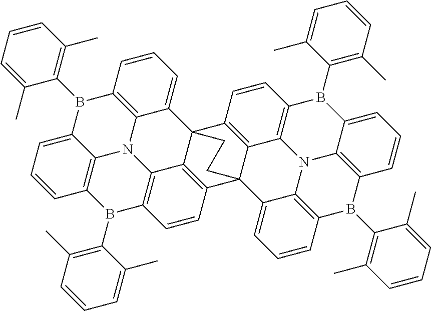 [SMILES: Cc1cccc(C)c1B1c2cccc3c2N2c4c1cccc4C14CCC5(c6cccc7c6N6c8c(cccc8B(c8c(C)cccc8C)c8ccc1c5c86)B7c1c(C)cccc1C)c1ccc(c2c14)B3c1c(C)cccc1C] | 88 |
| 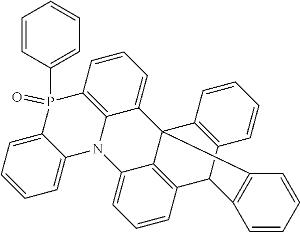 [SMILES: O=P1(c2ccccc2)c2ccccc2N2c3cccc4c3C3(c5ccccc5C4c4ccccc43)c3cccc1c32] | 89 |
| 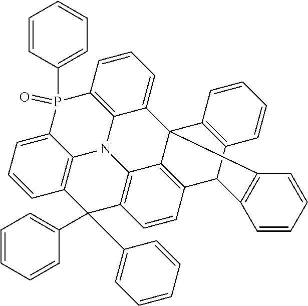 [SMILES: O=P1(c2ccccc2)c2cccc3c2N2c4c(cccc41)C14c5ccccc5C(c5ccccc51)c1ccc(c2c14)C3(c1ccccc1)c1ccccc1] | 90 |
| 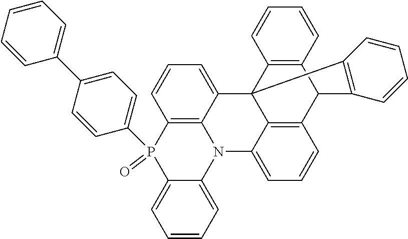 [SMILES: O=P1(c2ccc(-c3ccccc3)cc2)c2ccccc2N2c3cccc4c3C3(c5ccccc5C4c4ccccc43)c3cccc1c32] | 91 |
| 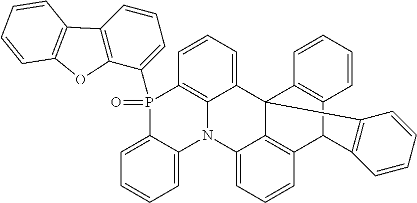 [SMILES: O=P1(c2cccc3c2oc2ccccc23)c2ccccc2N2c3cccc4c3C3(c5ccccc5C4c4ccccc43)c3cccc1c32] | 92 |
| 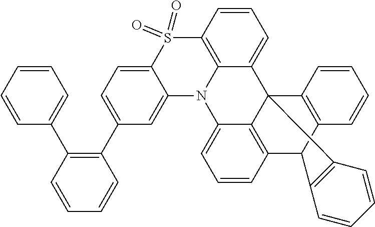 [SMILES: O=S1(=O)c2ccc(-c3ccccc3-c3ccccc3)cc2N2c3cccc4c3C3(c5ccccc5C4c4ccccc43)c3cccc1c32] | 93 |
| 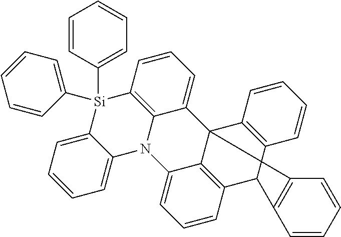 [SMILES: c1ccc([Si]2(c3ccccc3)c3ccccc3N3c4cccc5c4C4(c6ccccc6C5c5ccccc54)c4cccc2c43)cc1] | 94 |
| 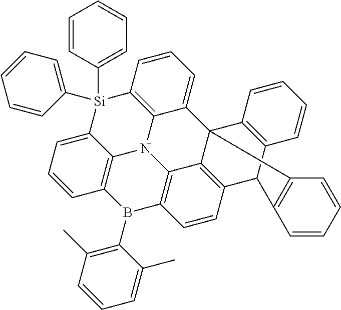 [SMILES: Cc1cccc(C)c1B1c2cccc3c2N2c4c(cccc4[Si]3(c3ccccc3)c3ccccc3)C34c5ccccc5C(c5ccccc53)c3ccc1c2c34] | 95 |
| 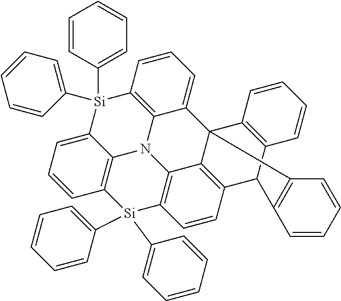 [SMILES: c1ccc([Si]2(c3ccccc3)c3cccc4c3N3c5c2cccc5[Si](c2ccccc2)(c2ccccc2)c2ccc5c(c23)C42c3ccccc3C5c3ccccc32)cc1] | 96 |
| 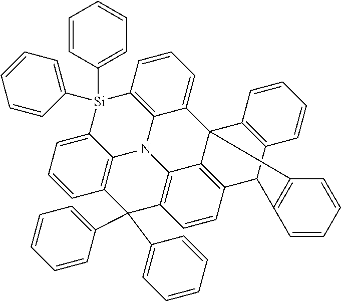 [SMILES: c1ccc(C2(c3ccccc3)c3cccc4c3N3c5c(cccc5[Si]4(c4ccccc4)c4ccccc4)C45c6ccccc6C(c6ccccc64)c4ccc2c3c45)cc1] | 97 |
| 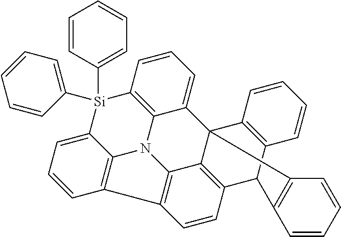 [SMILES: c1ccc([Si]2(c3ccccc3)c3cccc4c3-n3c5c2cccc5c2ccc5c(c23)C42c3ccccc3C5c3ccccc32)cc1] | 98 |

The compounds of the invention can be prepared by synthesis steps known to those skilled in the art, for example bromination, Suzuki coupling, Ullmann coupling, Heck reaction, Hartwig-Buchwald coupling, cyanations, etc.

The present invention therefore further provides a process for preparing the compounds of the invention, characterized by the following steps:
(A) synthesis of the compound of formula (1) comprising A¹A²-Ar¹ or formula (2) comprising Ar¹A²Ar¹;
(B) introducing the Ar² group in A¹A²-Ar¹ or Ar¹A²Ar¹;
(C) ring closure between Ar² and Ar¹ with introduction of NR′;
(D)optional further functionalization and/or exchange of R′.

The introduction of the Ar² group is preferably effected on the carbon atom with a covalent bond to A¹, A² and Ar¹ for formula (1), or with a covalent bond to both Ar¹ and A² for formula (2).

One example of a synthesis is shown by scheme 1. First of all, the base skeleton composed of A¹A²-Ar¹ is provided, which has a group capable of coupling in the form of X², for example Br, Cl or I. To this is coupled an Ar² group modified correspondingly with a compatible coupling group X³ and a nitrate group. Thereafter, the Ar¹ and Ar² groups are joined via a Cadogan-type ring closure. Usually, the compound of the formula (1) with R′=H is then obtained. By further coupling reactions, R′ can then be exchanged for a different group other than H, preferably Ar³. In further ring closure reactions, the Y and/or Z groups can then be introduced.

The compounds of the formula (2) can be prepared analogously.

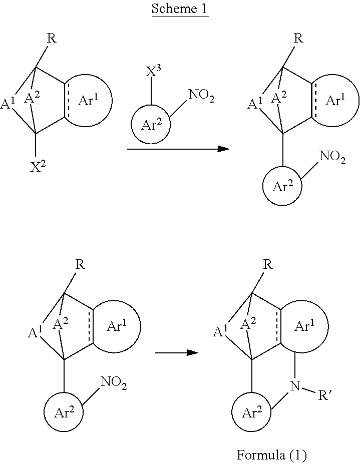

The inventive compounds can be prepared proceeding from compounds known from the literature that are brominated or iodinated at the bridgehead carbon atom according to M. Oi et al., Chem. Sci., 2019, 10, 6107. In step 1, the bridgehead carbon atom is first lithiated by reaction of the bromide with n-BuLi, followed by a transmetalation with copper(I) chloride and then palladium-catalyzed C—C coupling with a 2-nitroiodoaromatic. In step 2, reductive Cadogan-type cyclization, for example according to A. W. Freeman et al., J. Org. Chem. 2005, 70, 5014-5019 or CN110845508, leads to the 9,10-dihydroacridane system. The latter can finally be coupled in a C-N coupling of the Buchwald-Hartwig or Ullmann type to an aryl or heteroaryl halide (Ar³—X) to give further-preferred products of the invention.

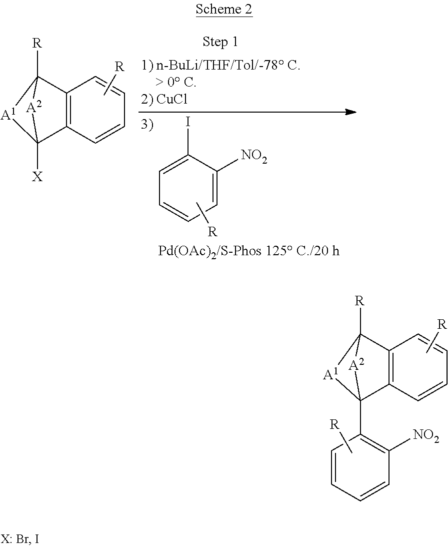

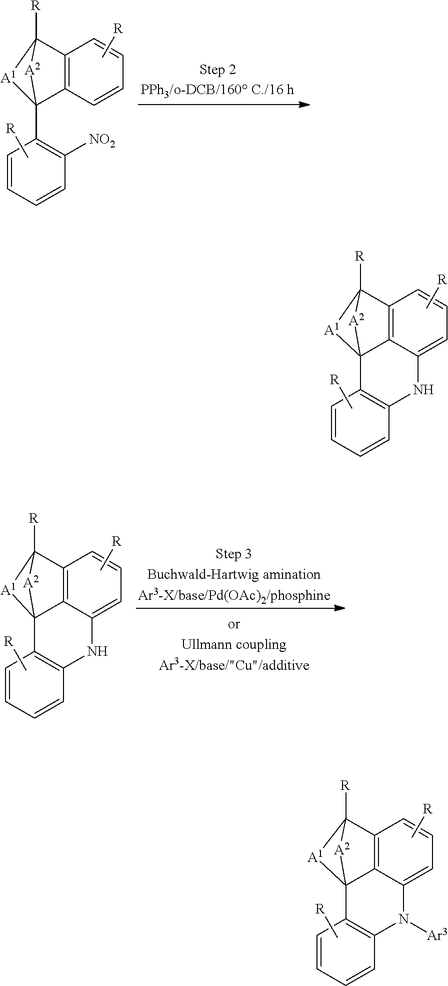

The 9,10-dihydroacridane intermediate from step 2 or the product from step 3, scheme 2, can be regioselectively halogenated in the para position(s) to the nitrogen atom with n-haloimides, for example N-chloro-, -bromo- or -iodosuccinimide; see step 1, scheme 3. The halogen functions thus introduced can be further functionalized in C-C coupling reactions of the Suzuki, Negishi, Sonogashira or Heck type, etc., or in C-N coupling reactions of the Buchwald-Hartwig or Ullmann type; see step 2, scheme 3.

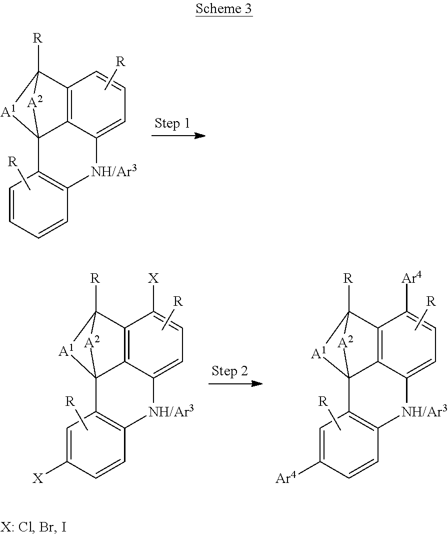

If, in step 3, scheme 2, o-halogen-substituted aromatics are introduced by use of 1,2-Cl, Br- or —Cl, I- or —Br, I-aromatics, these can be cyclized, under palladium catalysis in the presence of a base and a tertiary phosphine or of an NHC to carbazoles, for example, according to P. B. Tiruveedhula, et al., Org. & Biomol. Chem., 2015, 13(43), 10705 or F. Chen et al., RSC Adv., 2015, 5, 51512 or T. Kader et al., Chem. Europ. J., 2019, 25(17), 4412, or analogously to U.S. Pat. No. 9,000,421 B1; see scheme 4. It is possible here to control regioselectivity via steric and/or electronic effects of the substituents R. If o,o′-halogen-substituted aromatics are being converted, it is possible in this way to obtain 10H-benzo-[1,7]pyrrolizino[2,3,4,5,6-defg]acridines.

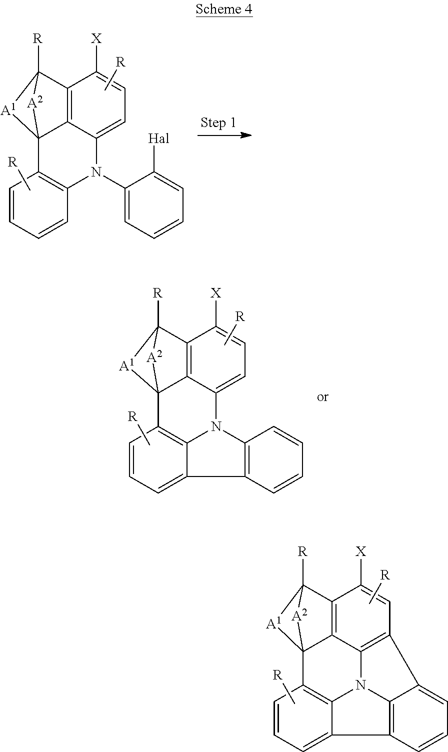

The B-N heterocycles of the invention can be prepared proceeding from the 9,10-dihydroacridane intermediates from step 2, scheme 2; see scheme 5. First of all, an o,o′-bischloroarylene function is introduced via Sₙ2-Ar reaction according to step 1a or alternatively via Buchwald-Hartwig coupling according to step 1b; see scheme 5. The latter is then converted by means of t-BuLi to the o,o′-bislithioarylene function and subsequently reacted in situ with BBr₃. Double electrophilic cyclization in the presence of the base DIPEA and final reaction of the remaining B-Br functionality with an aryllithium species gives the B-N heterocycles of the invention.

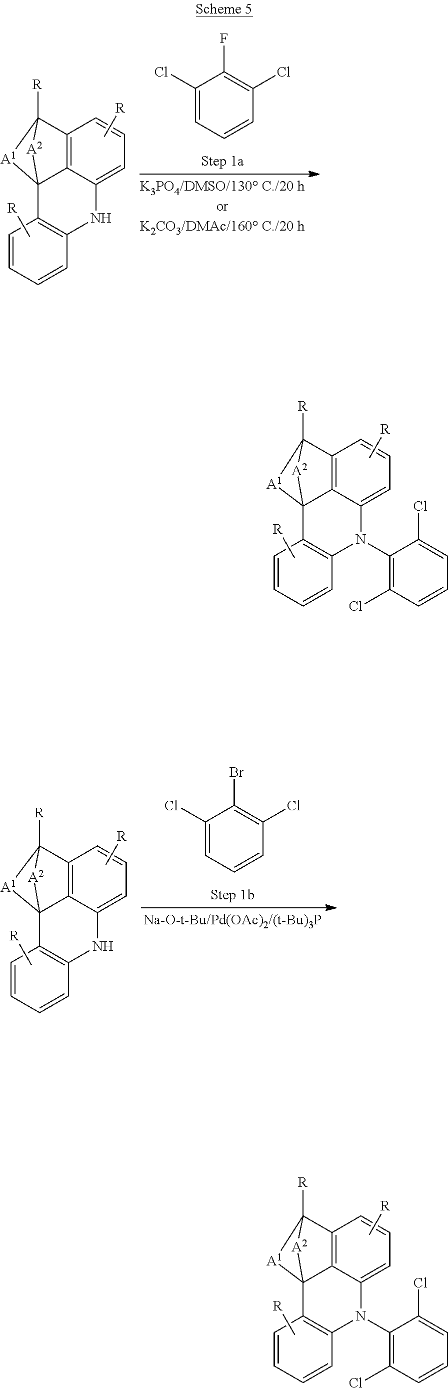

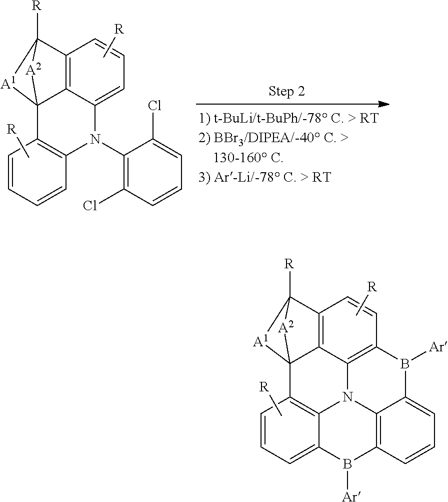

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation, especially a solution, dispersion or emulsion, comprising at least one compound of the invention and at least one further compound. The further compound may, for example, be a solvent, especially one of the abovementioned solvents or a mixture of these solvents. The production of such solutions is known to the person skilled in the art and is described, for example, in WO 2002/072714, WO 2003/019694 and the literature cited therein. The further compound may alternatively be at least one further organic or inorganic compound which is likewise used in the electronic device, for example an emitting compound and/or a matrix material. This further compound may also be polymeric.

The compounds of the invention are suitable for use in an electronic device, especially in an organic electroluminescent device (OLED). Depending on the substitution, the compounds may be used in different functions and layers.

The present invention therefore further provides for the use of a compound of the invention in an electronic device.

The present invention still further provides an electronic device comprising at least one compound of the invention.

The compounds of the invention may especially be used in the form of a racemate or of a pure enantiomer.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs).

The device is more preferably an organic electroluminescent device, comprising anode, cathode and at least one emitting layer, where at least one organic layer that may be an emitting layer, hole transport layer, electron transport layer, hole blocker layer, electron blocker layer or other functional layer comprises at least one compound of the invention. The layer is dependent on the substitution of the compound.

Apart from these layers, the organic electroluminescent device may comprise still further layers, for example in each case one or more hole injection layers, hole transport layers, hole blocker layers, electron transport layers, electron injection layers, exciton blocker layers, electron blocker layers, charge generation layers and/or organic or inorganic p/n junctions. It is likewise possible for interlayers having an exciton-blocking function, for example, to be introduced between two emitting layers. However, it should be pointed out that not necessarily every one of these layers need be present.

In this case, it is possible for the organic electroluminescent device to contain an emitting layer, or for it to contain a plurality of emitting layers. If a plurality of emission layers are present, these preferably have several emission maxima between 380 nm and 750 mm overall, such that the overall result is white emission; in other words, various emitting compounds which may fluoresce or phosphoresce are used in the emitting layers. Especially preferred are systems having three emitting layers, where the three layers show blue, green and orange or red emission (the basic construction is described, for example, in WO 2005/011013). The organic electroluminescent device of the invention may also be a tandem OLED, especially for white-emitting OLEDs.

The compound of formula (1) is preferably used in an organic electroluminescent device comprising one or more phosphorescent emitters. The compound of the invention according to the above-detailed embodiments may be used in different layers, according to the exact structure.

In this case, the organic electroluminescent device may contain an emitting layer, or it may contain a plurality of emitting layers, where at least one layer contains at least one compound of the invention. In addition, the compound of the invention can also be used in an electron transport layer and/or in a hole blocker layer and/or in a hole transport layer and/or in an exciton blocker layer.

The expression “phosphorescent compound” typically refers to compounds where light is emitted through a spin-forbidden transition, for example a transition from an excited triplet state or a state having a higher spin quantum number, for example a quintet state.

Suitable phosphorescent compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preferred phosphorescent compounds are all luminescent complexes with transition metals or lanthanides, especially when they contain copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill. It is also possible for the person skilled in the art without an inventive step to use further phosphorescent complexes in combination with the compounds of the formula (1) in organic electroluminescent devices. Further examples are adduced in a table which follows.

According to the invention, it is also possible to use the compound of the formula (1) in an electronic device containing one or more fluorescent emitting compounds.

In a preferred embodiment of the invention, the compounds of the formula (1) are used as hole-transporting material. In that case, the compounds are preferably present in a hole transport layer, an electron blocker layer or a hole injection layer. Particular preference is given to use in an electron blocker layer.

A hole transport layer in the context of the present application is a layer having a hole-transporting function between the anode and emitting layer.

Hole injection layers and electron blocker layers in the context of the present application are understood to mean particular embodiments of hole transport layers. A hole injection layer, in the case of a multitude of hole transport layers between anode and emitting layer, is a hole transport layer which directly adjoins the anode or is separated therefrom only by a single coating of the anode. An electron blocker layer, in the case of a plurality of hole transport layers between the anode and emitting layer, is that hole transport layer which directly adjoins the emitting layer on the anode side. The OLED of the invention preferably comprises, between anode and emitting layer, two, three or four hole-transporting layers, of which preferably at least one, more preferably exactly one or two, contain(s) a compound of the formula (1).

If the compound of the formula (1) is used as hole transport material in a hole transport layer, a hole injection layer or an electron blocker layer, the compound may be used as pure material, i.e. in a proportion of 100%, in the hole transport layer, or it may be used in combination with one or more further compounds. In a preferred embodiment, the organic layer containing the compound of the formula (1) then additionally contains one or more p-dopants. p-Dopants that are used according to the present invention are preferably those organic electron acceptor compounds that are capable of oxidizing one or more of the other compounds in the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600, WO 2012/095143 and DE 102012209523.

Particularly preferred p-dopants are quinodimethane compounds, azaindenofluorenediones, azaphenylenes, azatriphenylenes, 12, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃.

The p-dopants are preferably distributed essentially homogeneously in the p-doped layers. This can be achieved, for example, by coevaporation of the p-dopant and the hole transport material matrix.

Preferred p-dopants are especially the following compounds:

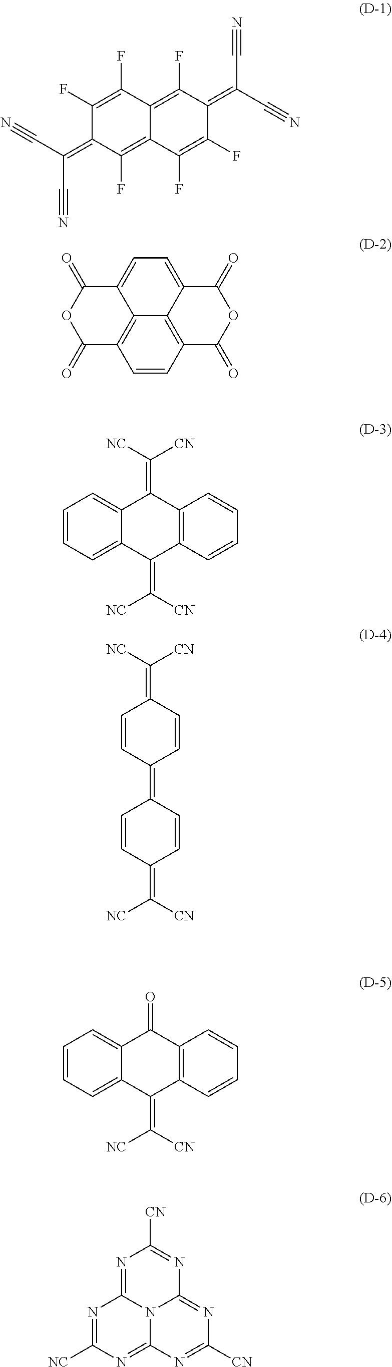

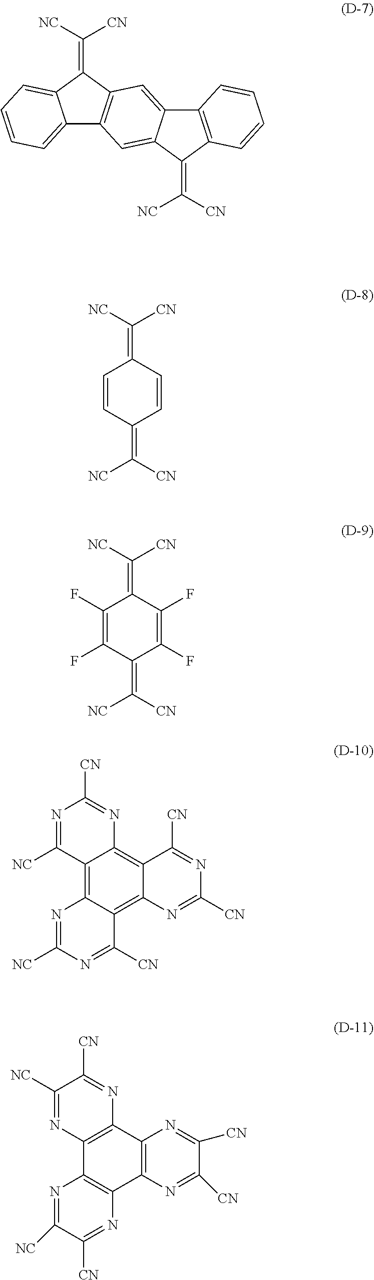 [SMILES: N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21 ; N#Cc1nc2c3nc(C#N)c(C#N)nc3c3nc(C#N)c(C#N)nc3c2nc1C#N]

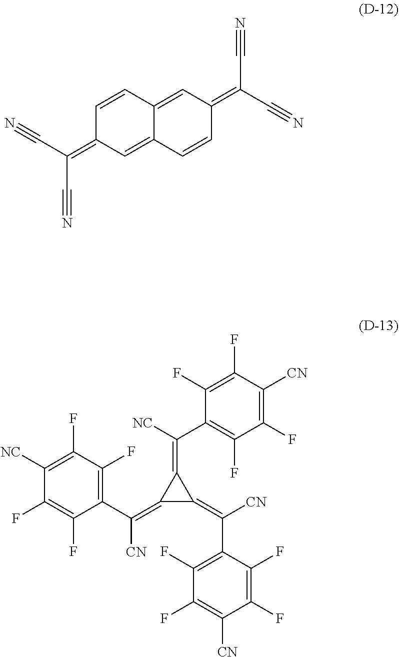 [SMILES: N#CC(C#N)=c1ccc2cc(=C(C#N)C#N)ccc2c1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

In a further preferred embodiment of the invention, the compound of the formula (1) is used as hole transport material in combination with a hexaazatriphenylene derivative as described in US 2007/0092755. Particular preference is given here to using the hexaazatriphenylene derivative in a separate layer.

In a further embodiment of the present invention, the compound of the formula (1) is used in an emitting layer as matrix material in combination with one or more emitting compounds, preferably phosphorescent compounds.

The proportion of the matrix material in the emitting layer in this case is between 50.0% and 99.9% by volume, preferably between 80.0% and 99.5% by volume, more preferably between 92.0% and 99.5% by volume, for fluorescent emitting layers and between 85.0% and 97.0% by volume for phosphorescent emitting layers.

Correspondingly, the proportion of the emitting compound is between 0.1% and 50.0% by volume, preferably between 0.5% and 20.0% by volume, more preferably between 0.5% and 8.0% by volume, for fluorescent emitting layers and between 3.0% and 15.0% by volume for phosphorescent emitting layers.

An emitting layer of an organic electroluminescent device may also comprise systems containing a multitude of matrix materials (mixed matrix systems) and/or a multitude of emitting compounds. In that case too, in general, the emitting compounds are those which have the smaller proportion in the system, and the matrix materials those which have the greater proportion in the system. In individual cases, however, the proportion of a single matrix material in the system may be smaller than the proportion of a single emitting compound.

The compounds of the formula (1) are preferably used as a constituent of mixed matrix systems. The mixed matrix systems preferably consist of two or three different matrix materials, more preferably of two different matrix materials. Preferably, in this case, one of the two materials is a material having hole-transporting properties and the other material is a material having electron-transporting properties. The compound of the formula (1) is preferably the matrix material having hole-transporting properties. The desired electron-transporting and hole-transporting properties of the mixed matrix components may, however, also be combined predominantly or completely in a single mixed matrix component, in which case the further mixed matrix component(s) fulfill(s) other functions. The two different matrix materials may be present in a ratio of 1:50 to 1:1, preferably 1:20 to 1:1, more preferably 1:10 to 1:1 and most preferably 1:4 to 1:1. Preference is given to using mixed matrix systems in phosphorescent organic electroluminescent devices. A source of more detailed information about mixed matrix systems is application WO 2010/108579.

The mixed matrix systems may contain one or more emitting compounds, preferably one or more phosphorescent compounds. In general, preference is given to using mixed matrix systems in phosphorescent organic electroluminescent devices.

Particularly suitable matrix materials which can be used in combination with the compounds of the invention as matrix components of a mixed matrix system are selected from the preferred matrix materials specified below for phosphorescent compounds or the preferred matrix materials for fluorescent compounds, according to what type of emitting compound is used in the mixed matrix system.

Preferred phosphorescent compounds for use in mixed matrix systems are the same as described further up as generally preferred phosphorescent emitter materials.

Preferred embodiments of the different functional materials in the electronic device are adduced hereinafter.

Examples of phosphorescent compounds are adduced below.

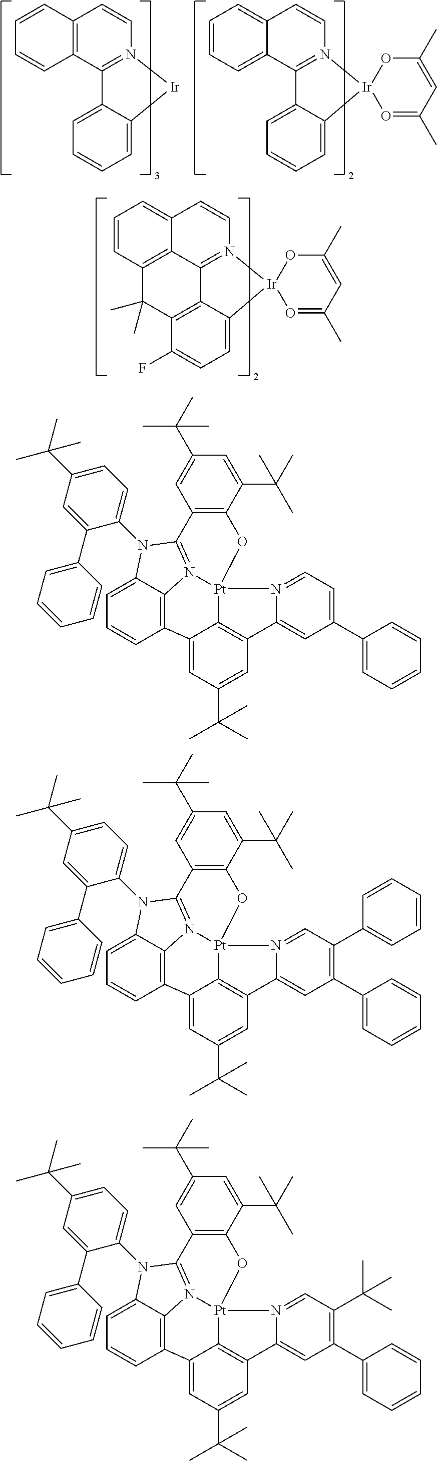

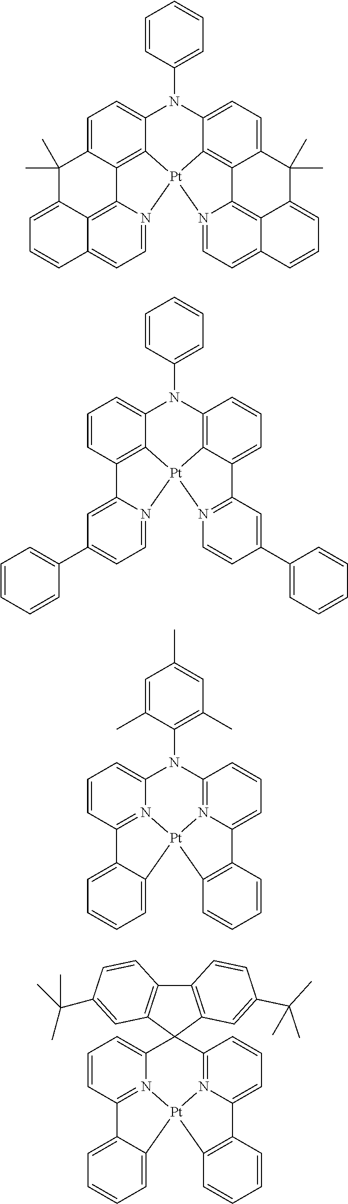

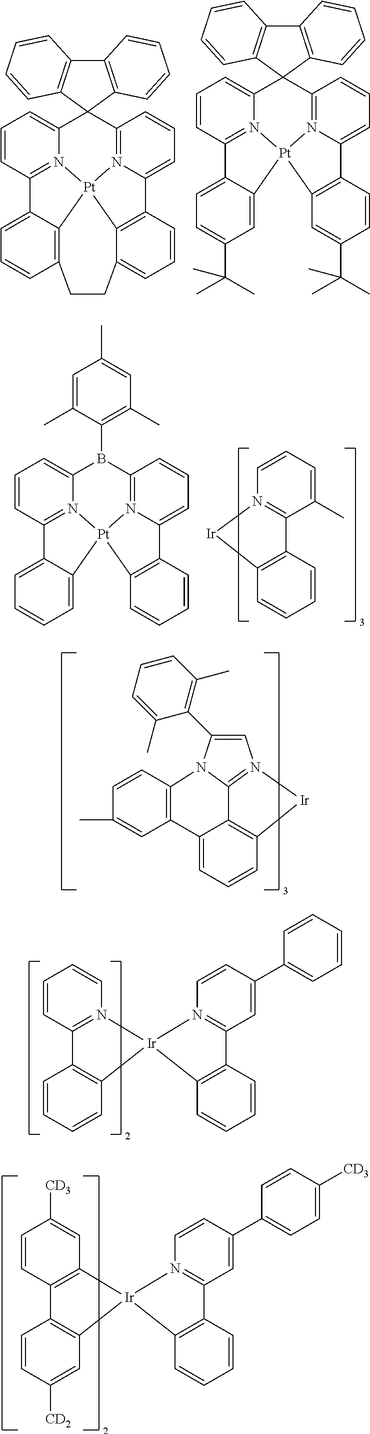

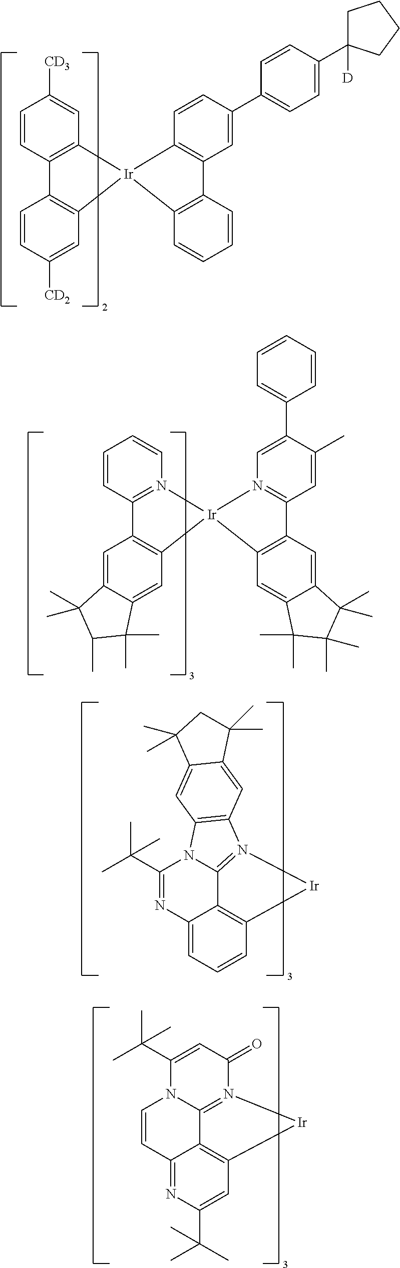

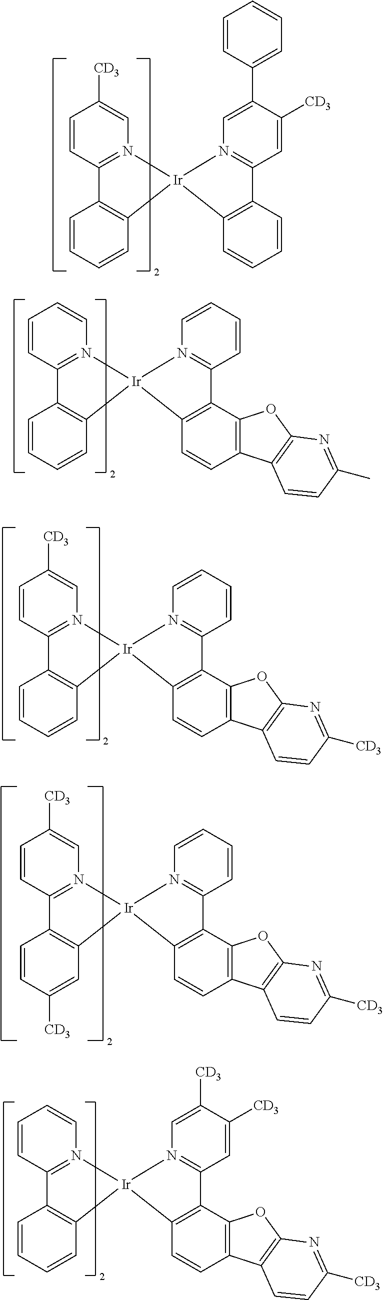

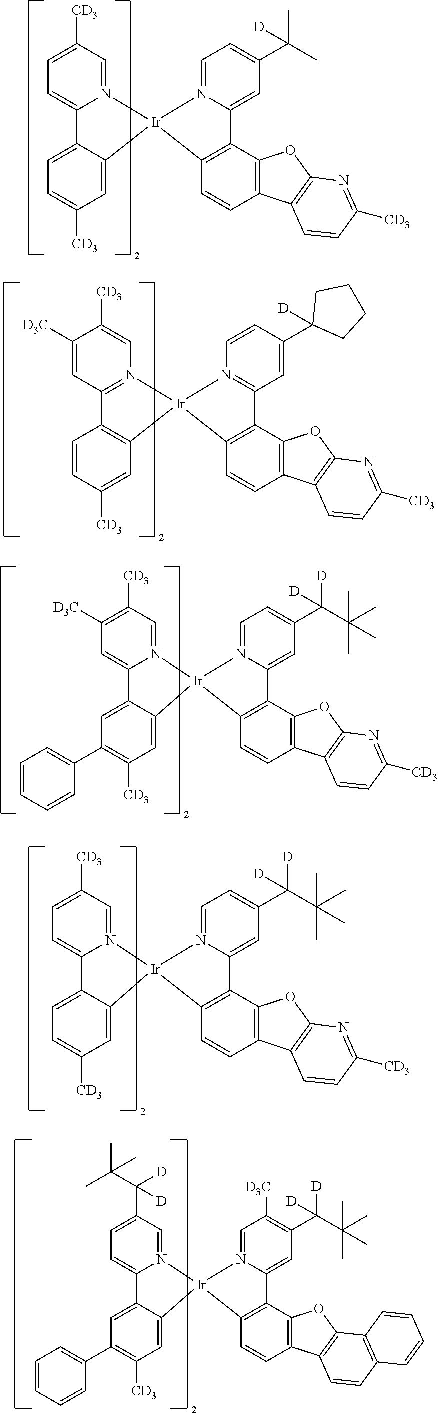

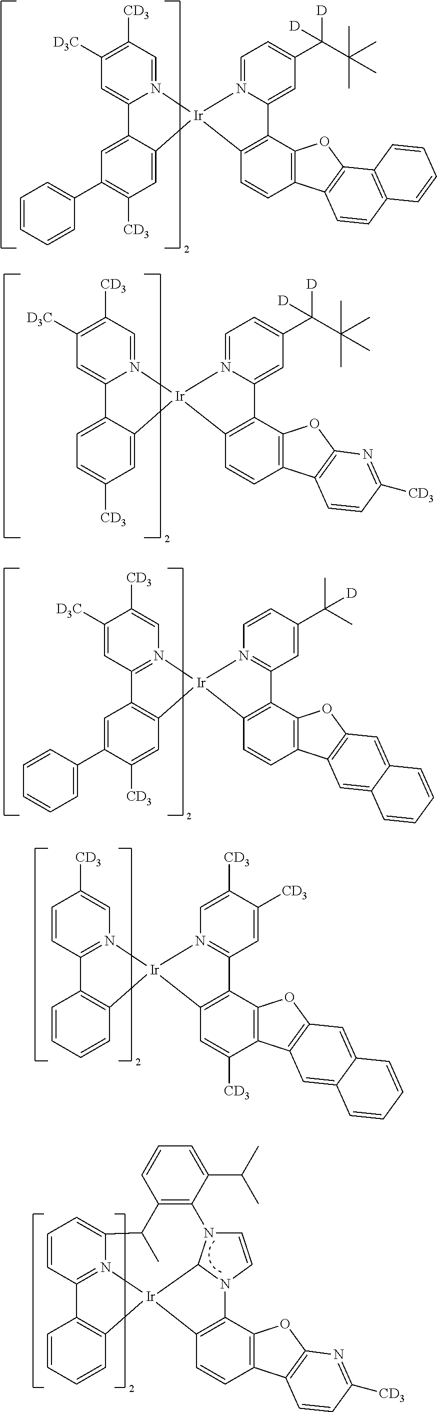

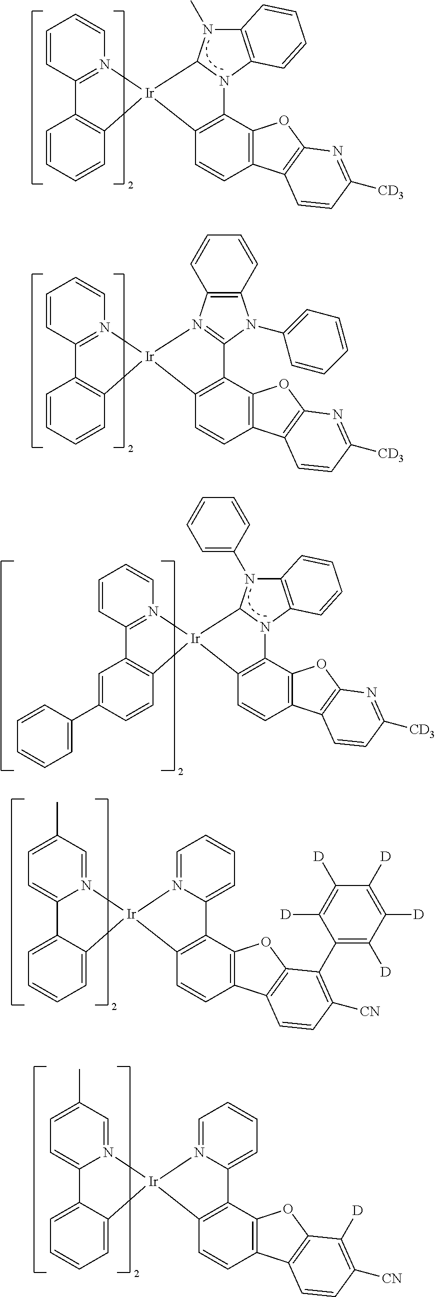

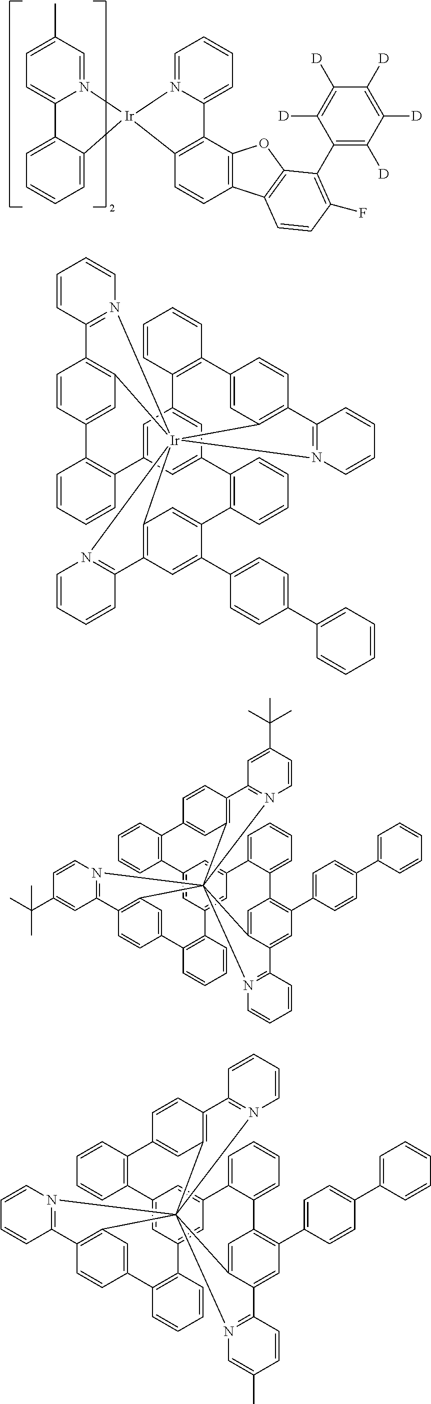

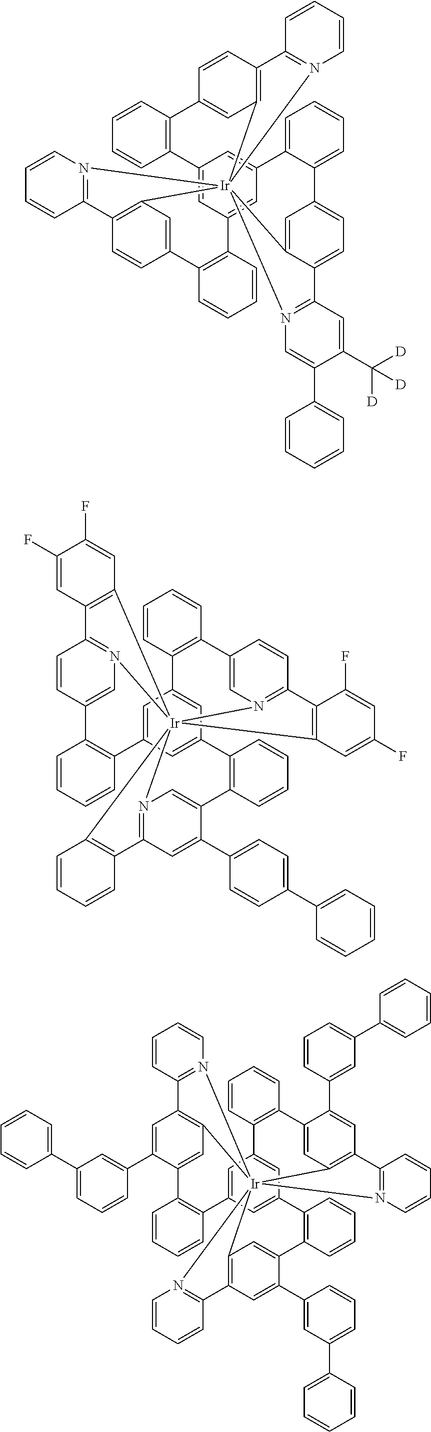

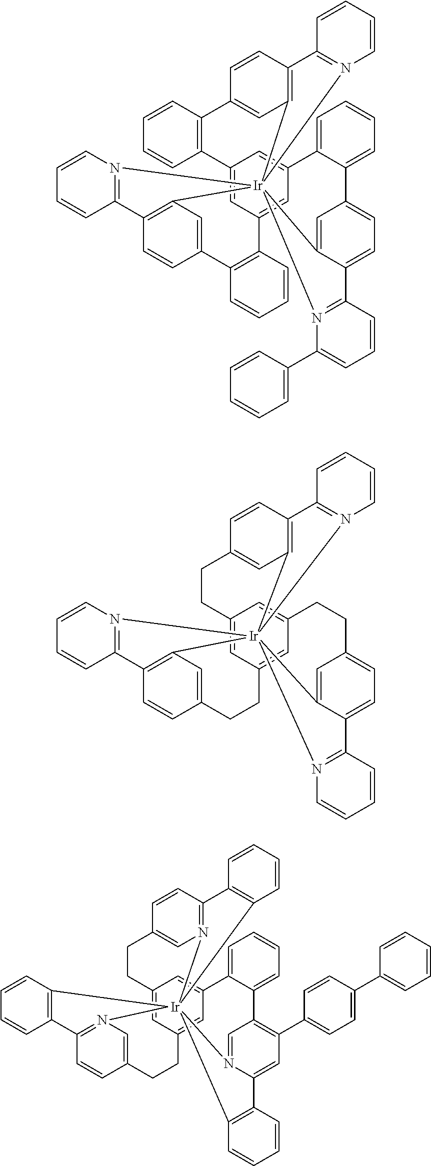

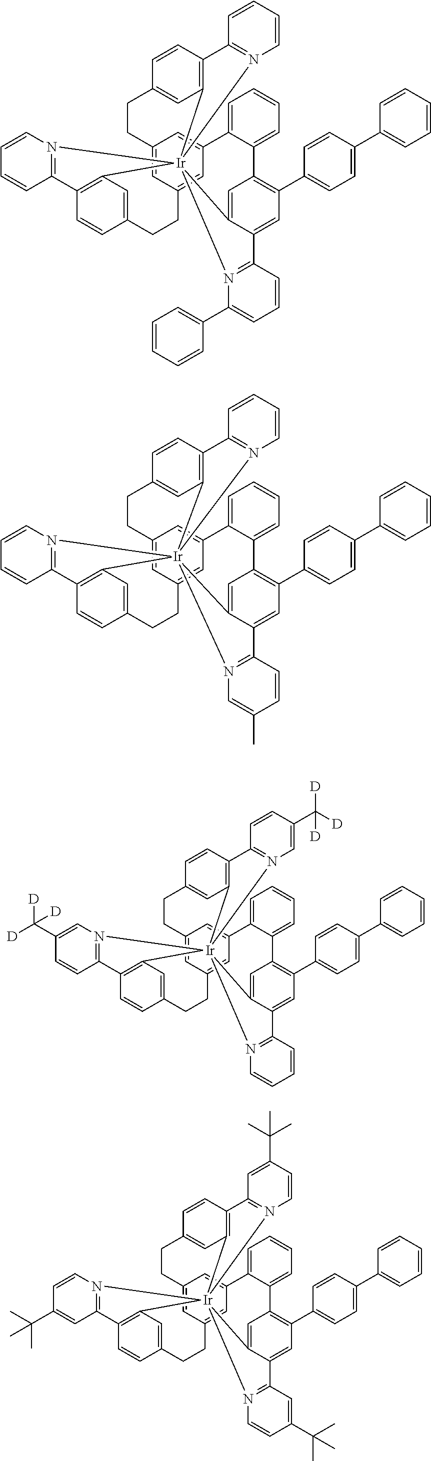

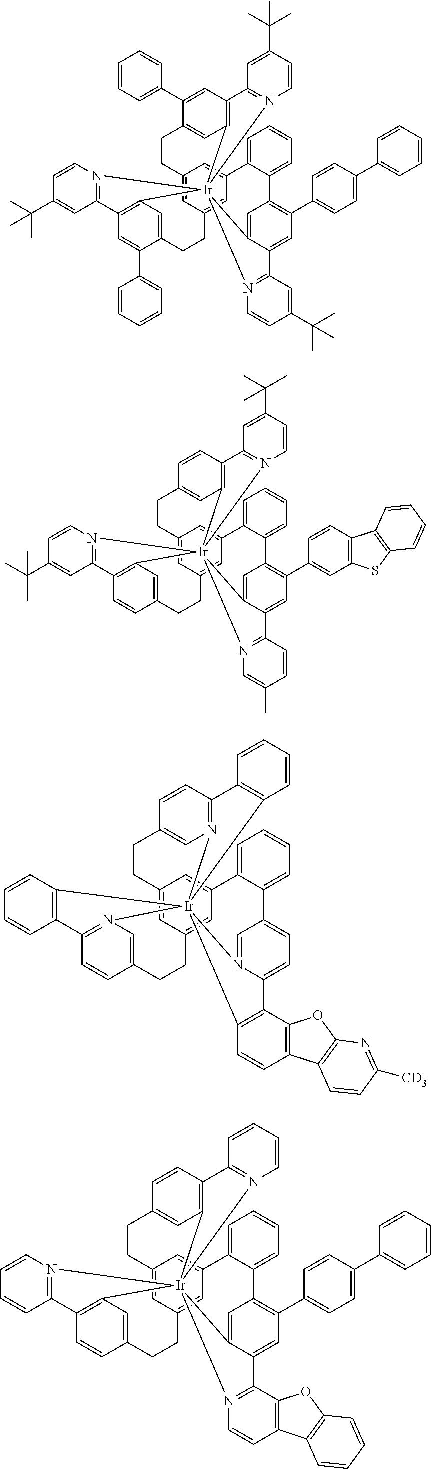

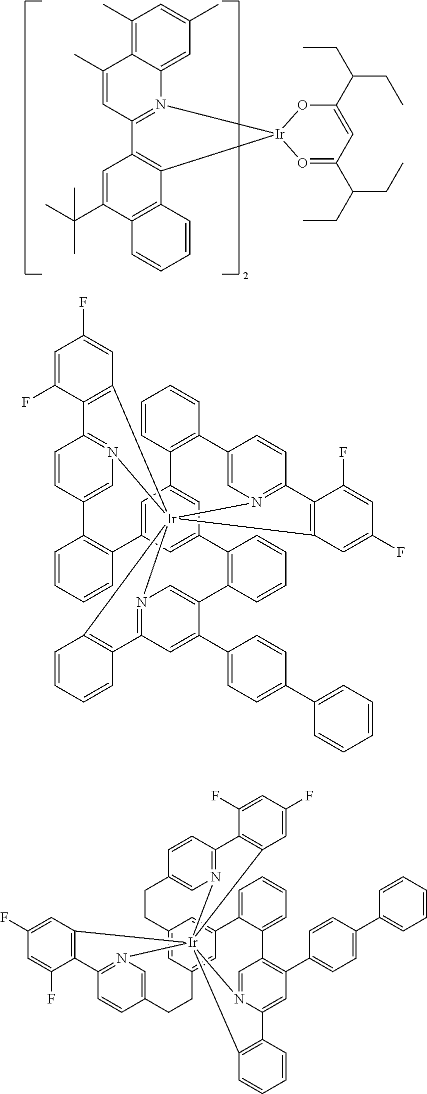

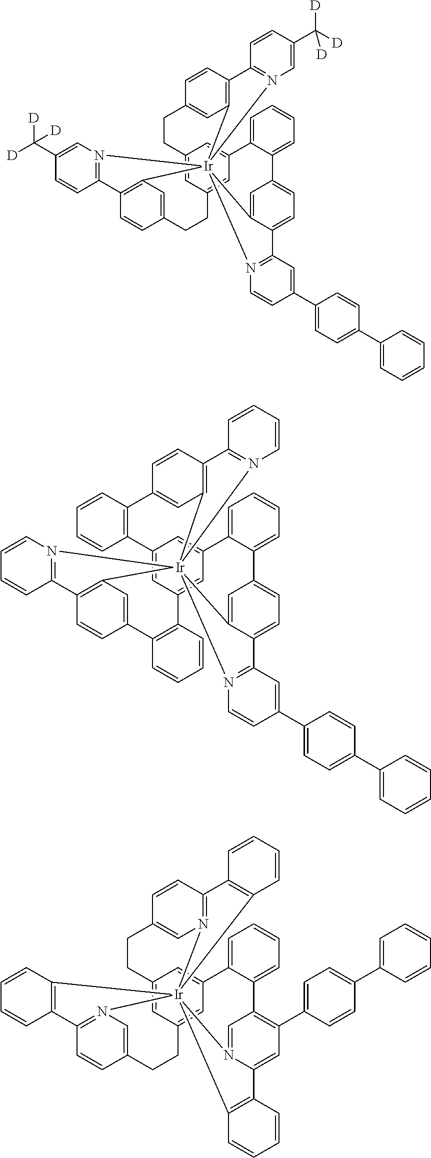

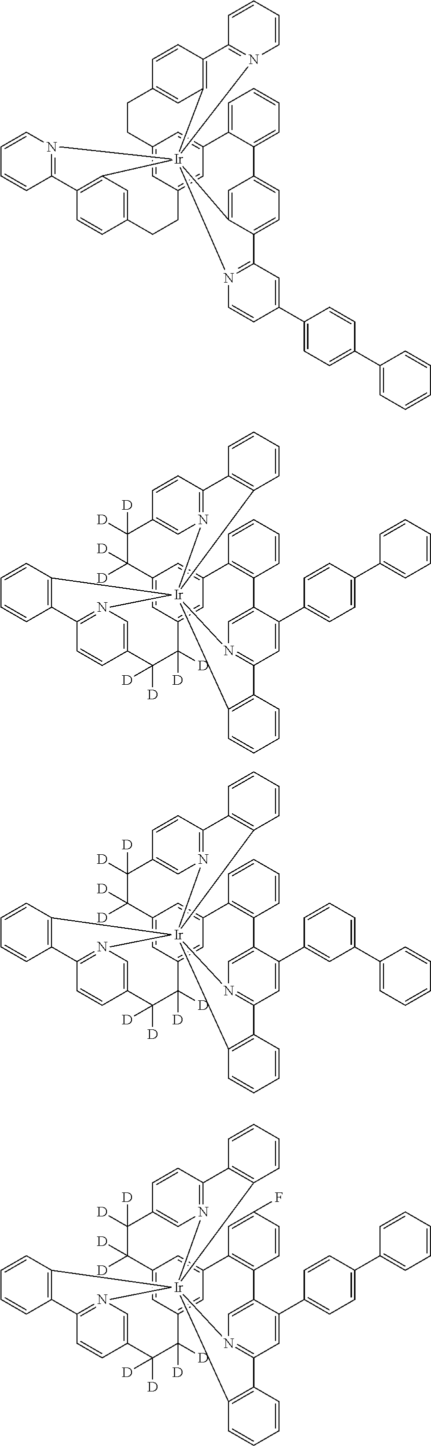

Preferred fluorescent emitting compounds are selected from the class of the arylamines. An arylamine or an aromatic amine in the context of the present invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, in which the diarylamino groups are preferably bonded to the pyrene in the 1 position or 1,6 positions. Further preferred emitting compounds are indenofluoreneamines or fluorenediamines, for example according to WO 2006/108497 or WO 2006/122630, benzoindenofluoreneamines or -fluorenediamines, for example according to WO 2008/006449, and dibenzoindenofluoreneamines or -diamines, for example according to WO 2007/140847, and the indenofluorene derivatives having fused aryl groups disclosed in WO 2010/012328. Likewise preferred are the pyrenearylamines disclosed in WO 2012/048780 and in WO 2013/185871. Likewise preferred are the benzoindenofluoreneamines disclosed in WO 2014/037077, the benzofluoreneamines disclosed in WO 2014/106522, the extended benzoindenofluorenes disclosed in WO 2014/111269 and in WO 2017/036574, the phenoxazines disclosed in WO 2017/028940 and in WO 2017/028941, and the fluorine derivatives bonded to furan units or to thiophene units that are disclosed in WO 2016/150544.

Useful matrix materials, preferably for fluorescent compounds, include materials from different substance classes. Preferred matrix materials are selected from the classes of the oligoaryls (e.g. 2,2′,7,7′-tetraphenylspirobifluorene according to EP 676461 or dinaphthylanthracene), especially of the oligoaryls having fused aromatic groups, the oligoarylenevinylenes (e.g. DPVBi or spiro-DPVBi according to EP 676461), the polypodal metal complexes (for example according to WO 2004/081017), the hole-conducting compounds (for example according to WO 2004/058911), the electron-conducting compounds, especially ketones, phosphine oxides, sulfoxides, etc. (for example according to WO 2005/084081 and WO 2005/084082), the atropisomers (for example according to WO 2006/048268), the boronic acid derivatives (for example according to WO 2006/117052) or the benzanthracenes (for example according to WO 2008/145239). Particularly preferred matrix materials are selected from the classes of the oligoarylenes with naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoaryl in the context of the present invention is understood to mean a compound in which at least three aryl or arylene groups are bonded to one another. Further preferred are the anthracene derivatives disclosed in WO 2006/097208, WO 2006/131192, WO 2007/065550, WO 2007/110129, WO 2007/065678, WO 2008/145239, WO 2009/100925, WO 2011/054442 and EP 1553154, the pyrene compounds disclosed in EP 1749809, EP 1905754 and US 2012/0187826, the benzanthracenylanthracene compounds disclosed in WO 2015/158409, the indenobenzofurans disclosed in WO 2017/025165, and the phenanthrylanthracenes disclosed in WO 2017/036573.

Preferred matrix materials for phosphorescent compounds, just like compounds of formula (1), are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, lactams, for example according to WO 2011/116865 or WO 2011/137951, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

Suitable charge transport materials as usable in the hole injection layer or hole transport layer or in the electron blocker layer or in the electron transport layer of the electronic component of the invention are, as well as the compounds of the formula (1), for example, the compounds disclosed in Y. Shirota et al., Chem. Rev. 2007, 107(4), 953-1010, or other materials as used in these layers according to the prior art.

The OLED of the invention preferably comprises two or more different hole-transporting layers. The compound of the formula (1) may be used here in one or more of or in all the hole-transporting layers. In a preferred embodiment, the compound of the formula (1) is used in exactly one or exactly two hole-transporting layers, and other compounds, preferably aromatic amine compounds, are used in the further hole-transporting layers present. Further compounds which are used alongside the compounds of the formula (1), preferably in hole-transporting layers of the OLEDs of the invention, are especially indenofluoreneamine derivatives (for example according to WO 06/122630 or WO 06/100896), the amine derivatives disclosed in EP 1661888, hexaazatriphenylene derivatives (for example according to WO 01/049806), amine derivatives with fused aromatics (for example according to U.S. Pat. No. 5,061,569), the amine derivatives disclosed in WO 95/09147, monobenzoindenofluoreneamines (for example according to WO 08/006449), dibenzoindenofluoreneamines (for example according to WO 07/140847), spirobifluoreneamines (for example according to WO 2012/034627 or WO 2013/120577), fluoreneamines (for example according to WO 2014/015937, WO 2014/015938, WO 2014/015935 and WO 2015/082056), spirodibenzopyranamines (for example according to WO 2013/083216), dihydroacridine derivatives (for example according to WO 2012/150001), spirodibenzofurans and spirodibenzothiophenes (for example according to WO 2015/022051, WO 2016/102048 and WO 2016/131521), phenanthrenediarylamines (for example according to WO 2015/131976), spirotribenzotropolones (for example according to WO 2016/087017), spirobifluorenes with meta-phenyldiamine groups (for example according to WO 2016/078738), spirobisacridines (for example according to WO 2015/158411), xanthenediarylamines (for example according to WO 2014/072017), and 9,10-dihydroanthracene spiro compounds with diarylamino groups according to WO 2015/086108.

Very particular preference is given to the use of spirobifluorenes substituted in the 4 position by diarylamino groups as hole-transporting compounds, especially to the use of those compounds that are claimed and disclosed in WO 2013/120577, and to the use of spirobifluorenes substituted in the 2 position by diarylamino groups as hole-transporting compounds, especially to the use of those compounds that are claimed and disclosed in WO 2012/034627.

Materials used for the electron transport layer may be any materials that are used as electron transport materials in the electron transport layer according to the prior art. Particularly suitable are aluminum complexes, e.g. Alq3, zirconium complexes, e.g. Zrq4, lithium complexes, e.g. Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the aforementioned compounds as disclosed in JP 2000/053957, WO 2003/060956, WO 2004/028217, WO 2004/080975 and WO 2010/072300.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

According to the invention, the electronic devices containing one or more compounds of the formula (1) can be used in displays, as light sources in lighting applications and as light sources in medical and/or cosmetic applications (e.g. light therapy).

The compounds of the invention and the organic electroluminescent devices of the invention are notable for one or more of the following surprising properties:
1. The compounds of the invention lead to long lifetimes.
2. The compounds of the invention lead to high efficiencies, especially to a high EQE.
3. The compounds of the invention lead to low operating voltages.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature. In the case of compounds that can have multiple enantiomeric, diastereomeric or tautomeric forms, one form is shown in a representative manner.

1) Synthons LS Known from the Literature:

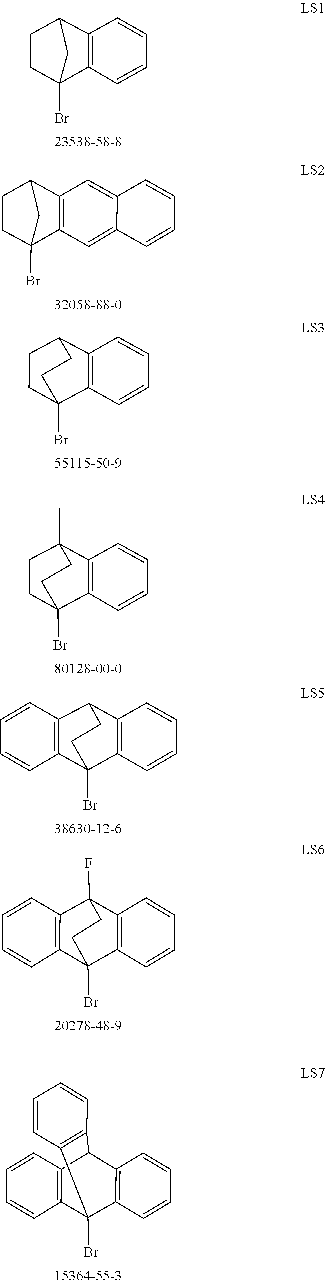

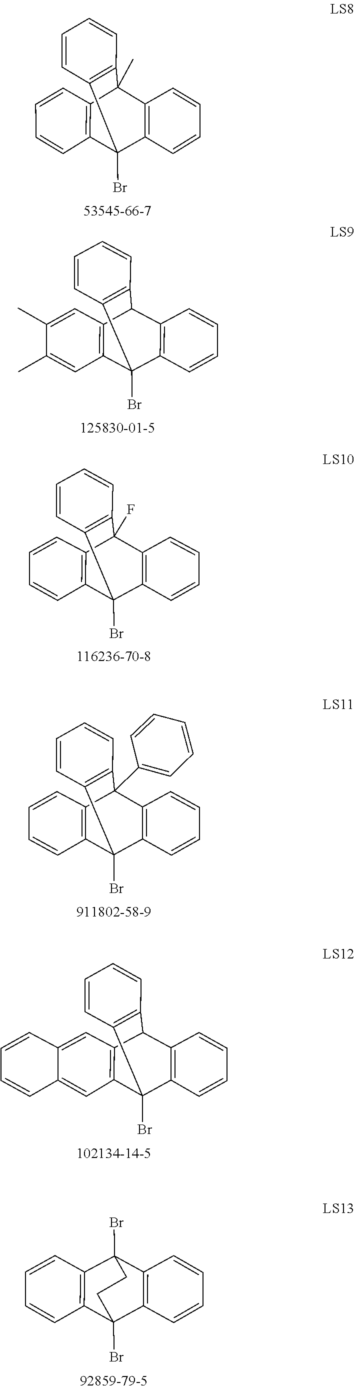 [SMILES: CC12c3ccccc3C(Br)(c3ccccc31)c1ccccc12 ; Cc1cc2c(cc1C)C1(Br)c3ccccc3C2c2ccccc21 ; FC12c3ccccc3C(Br)(c3ccccc31)c1ccccc12 ; BrC12c3ccccc3C(c3ccccc3)(c3ccccc31)c1ccccc12 ; BrC12c3ccccc3C(c3ccccc31)c1cc3ccccc3cc12 ; BrC12CCC(Br)(c3ccccc31)c1ccccc12]

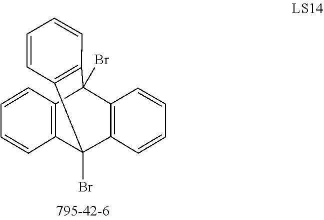 [SMILES: BrC12c3ccccc3C(Br)(c3ccccc31)c1ccccc12]

### 2) Synthesis of Synthons S:

### Example S1

### Stage 1:

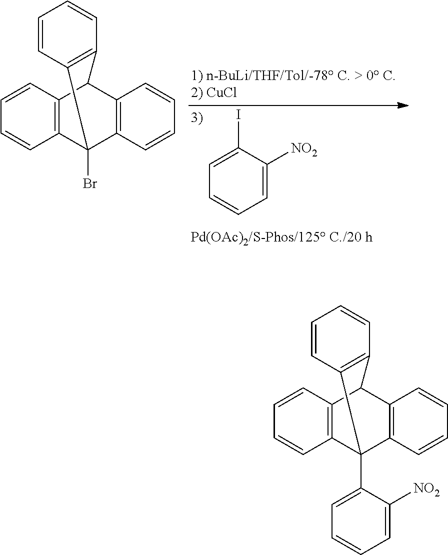 [SMILES: BrC12c3ccccc3C(c3ccccc31)c1ccccc12 ; O=[N+]([O-])c1ccccc1I ; O=[N+]([O-])c1ccccc1C12c3ccccc3C(c3ccccc31)c1ccccc12]

Preparation analogous to M. Oi et al., Chem. Sci., 2019, 10, 6107, Example 9. Starting materials: 33.3 g (100 mmol) of 9-bromotriptycene; instead of methyl 2-iodobenzoate, 27.4 g (110 mmol) of 2-iodonitrobenzene is used. Purification by flash chromatography (automated column system from A. Semrau, silica gel, n-heptane:ethyl acetate eluent, gradient). Yield: 23.0 g (61 mmol), 61%; purity: 97% by ¹H-NMR.

### Stage 2:

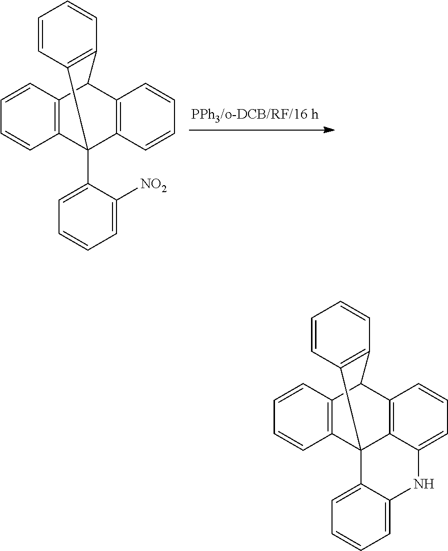 [SMILES: O=[N+]([O-])c1ccccc1C12c3ccccc3C(c3ccccc31)c1ccccc12 ; c1ccc2c(c1)Nc1cccc3c1C21c2ccccc2C3c2ccccc21]

Preparation according to A. W. Freeman et al., J. Org. Chem. 2005, 70, 5014.

Starting materials: 37.5 g (100 mmol) of S1, stage 1, 65.6 g (250 mmol) of PPh₃, 250 ml of o-dichlorobenzene (o-DCB), RF, 16 h. Workup: draw off o-DCB under reduced pressure, precipitate OPPh₃ by means of cyclohexane, filter off with suction, concentrate the filtrate. Purification by flash chromatography (automated column system from A. Semrau, silica gel, n-heptane:DCM eluent, gradient). Yield: 29.6 g (87 mmol), 87%; purity: 97% by ¹H-NMR.

The following compounds can be obtained analogously:

| Ex. | Reactants | Product | Yield |
|---|---|---|---|
| S2 | LS1 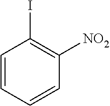 609-73-4 | 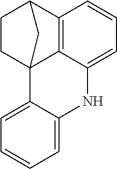 [SMILES: c1ccc2c(c1)Nc1cccc3c1C21CCC3C1] | 57% |
| S3 | LS4 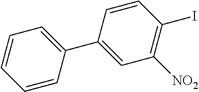 [SMILES: O=[N+]([O-])c1cc(-c2ccccc2)ccc1I] 2499-68-5 | 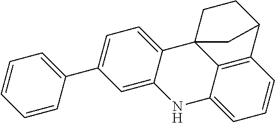 [SMILES: c1ccc(-c2ccc3c(c2)Nc2cccc4c2C32CCC4C2)cc1] | 55% |
| S4 | LS2 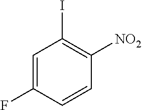 [SMILES: O=[N+]([O-])c1ccc(F)cc1I] 41860-64-4 | 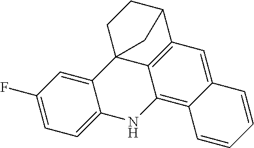 [SMILES: Fc1ccc2c(c1)C13CCC(C1)c1cc4ccccc4c(c13)N2] | 48% |
| S5 | LS3 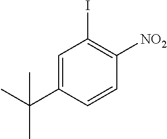 942204-75-3 | 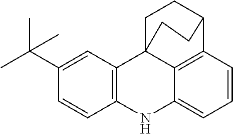 [SMILES: CC(C)(C)c1ccc2c(c1)C13CCC(CC1)c1cccc(c13)N2] | 56% |
| S6 | LS3 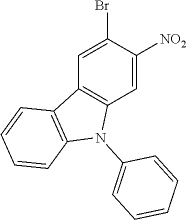 [SMILES: O=[N+]([O-])c1cc2c(cc1Br)c1ccccc1n2-c1ccccc1] 2101959-49-1 | 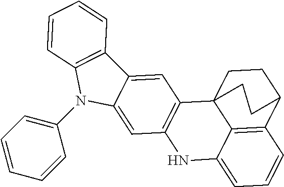 [SMILES: c1ccc(-n2c3ccccc3c3cc4c(cc32)Nc2cccc3c2C42CCC3CC2)cc1] | 50% |
| S7 | LS4 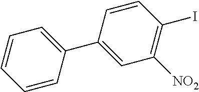 [SMILES: O=[N+]([O-])c1cc(-c2ccccc2)ccc1I] 2499-68-5 | 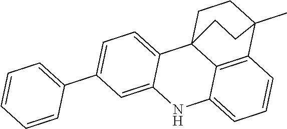 [SMILES: CC12CCC3(CC1)c1ccc(-c4ccccc4)cc1Nc1cccc2c13] | 57% |
| S8 | LS5 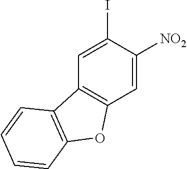 [SMILES: O=[N+]([O-])c1cc2oc3ccccc3c2cc1I] 2222130-29-0 | 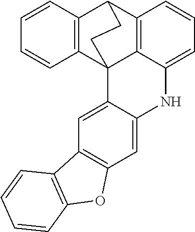 [SMILES: c1ccc2c(c1)C1CCC23c2cc4c(cc2Nc2cccc1c23)oc1ccccc14] | 54% |
| S9 | LS5 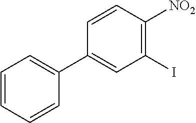 [SMILES: O=[N+]([O-])c1ccc(-c2ccccc2)cc1I] 905920-49-2 | 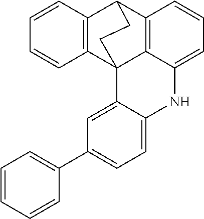 [SMILES: c1ccc(-c2ccc3c(c2)C24CCC(c5ccccc52)c2cccc(c24)N3)cc1] | 61% |
| S10 | LS6 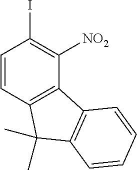 [SMILES: CC1(C)c2ccccc2-c2c1ccc(I)c2[N+](=O)[O-]] 2222315-64-0 | 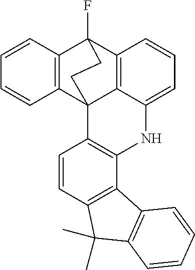 [SMILES: CC1(C)c2ccccc2-c2c1ccc1c2Nc2cccc3c2C12CCC3(F)c1ccccc12] | 47% |
| S11 | LS7 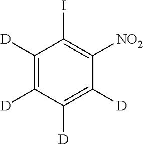 [SMILES: [2H]c1c([2H])c([2H])c([N+](=O)[O-])c(I)c1[2H]] 1946843-41-9 | 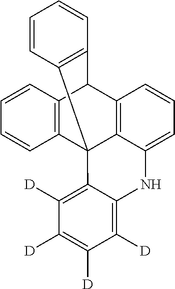 [SMILES: [2H]c1c([2H])c([2H])c2c(c1[2H])Nc1cccc3c1C21c2ccccc2C3c2ccccc21] | 58% |
| S12 | LS7 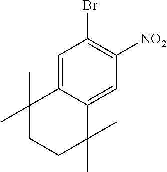 [SMILES: CC1(C)CCC(C)(C)c2cc([N+](=O)[O-])c(Br)cc21] 116233-18-2 | 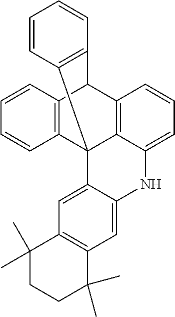 [SMILES: CC1(C)CCC(C)(C)c2cc3c(cc21)Nc1cccc2c1C31c3ccccc3C2c2ccccc21] | 55% |
| S13 | LS7 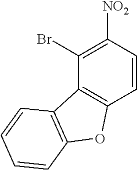 [SMILES: O=[N+]([O-])c1ccc2oc3ccccc3c2c1Br] 2101959-46-8 | 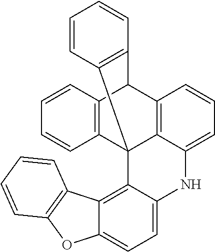 [SMILES: c1ccc2c(c1)C1c3ccccc3C23c2c(cccc21)Nc1ccc2oc4ccccc4c2c13] | 21% |
| S14 | LS7 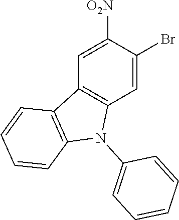 [SMILES: O=[N+]([O-])c1cc2c3ccccc3n(-c3ccccc3)c2cc1Br] 2101959-48-0 | 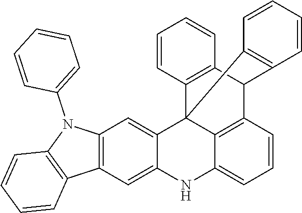 [SMILES: c1ccc(-n2c3ccccc3c3cc4c(cc32)C23c5ccccc5C(c5ccccc52)c2cccc(c23)N4)cc1] | 45% |
| S15 | LS7 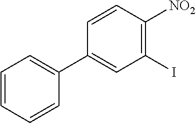 [SMILES: O=[N+]([O-])c1ccc(-c2ccccc2)cc1I] 905920-49-2 | 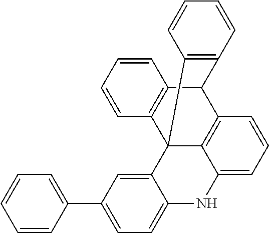 [SMILES: c1ccc(-c2ccc3c(c2)C24c5ccccc5C(c5ccccc52)c2cccc(c24)N3)cc1] | 60% |
| S16 | LS8 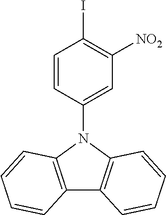 [SMILES: O=[N+]([O-])c1cc(-n2c3ccccc3c3ccccc32)ccc1I] 1974246-32-6 | 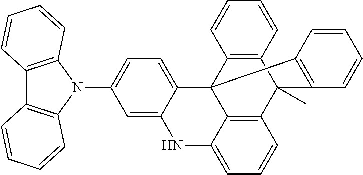 [SMILES: CC12c3ccccc3C3(c4ccc(-n5c6ccccc6c6ccccc65)cc4Nc4cccc1c43)c1ccccc12] | 48% |
| S17 | LS9 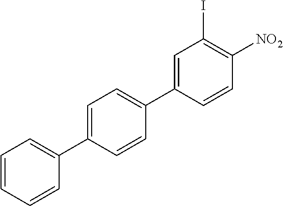 [SMILES: O=[N+]([O-])c1ccc(-c2ccc(-c3ccccc3)cc2)cc1I] 1946843-09-9 | 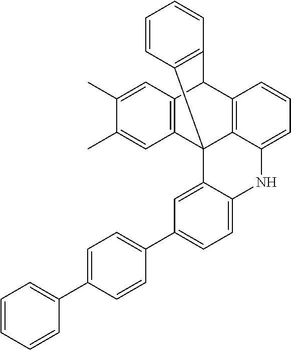 [SMILES: Cc1cc2c(cc1C)C13c4cc(-c5ccc(-c6ccccc6)cc5)ccc4Nc4cccc(c41)C2c1ccccc13] | 57% |
| S18 | LS10 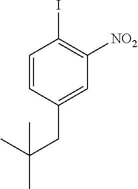 144146-99-6 | 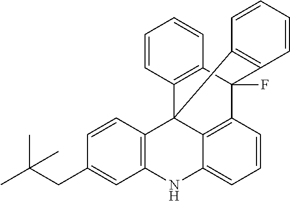 [SMILES: CC(C)(C)Cc1ccc2c(c1)Nc1cccc3c1C21c2ccccc2C3(F)c2ccccc21] | 53% |
| S19 | LS11 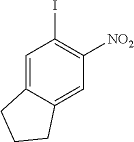 [SMILES: O=[N+]([O-])c1cc2c(cc1I)CCC2] 1156468-88-6 | 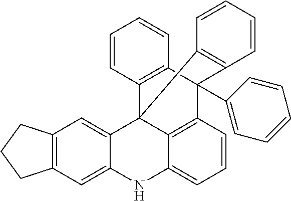 [SMILES: c1ccc(C23c4ccccc4C4(c5cc6c(cc5Nc5cccc2c54)CCC6)c2ccccc23)cc1] | 57% |
| S20 | LS11 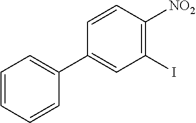 [SMILES: O=[N+]([O-])c1ccc(-c2ccccc2)cc1I] 905920-49-2 | 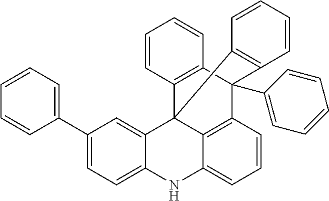 [SMILES: c1ccc(-c2ccc3c(c2)C24c5ccccc5C(c5ccccc5)(c5ccccc52)c2cccc(c24)N3)cc1] | 59% |
| S21 | LS12 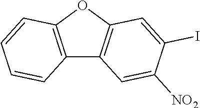 [SMILES: O=[N+]([O-])c1cc2c(cc1I)oc1ccccc12] 854396-49-9 | 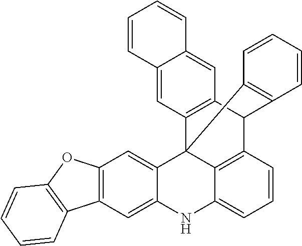 [SMILES: c1ccc2c(c1)C1c3cc4ccccc4cc3C23c2cc4oc5ccccc5c4cc2Nc2cccc1c23] | 58% |
| S22 | LS13 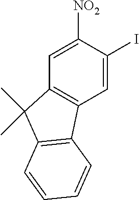 [SMILES: CC1(C)c2ccccc2-c2cc(I)c([N+](=O)[O-])cc21] 2241120-20-5 | 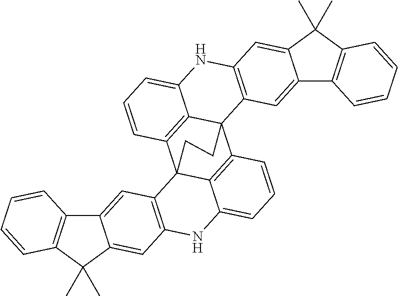 [SMILES: CC1(C)c2ccccc2-c2cc3c(cc21)Nc1cccc2c1C31CCC23c2cc4c(cc2Nc2cccc1c23)C(C)(C)c1ccccc1-4] | 23% |
| S23 | LS14 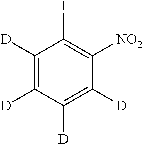 [SMILES: [2H]c1c([2H])c([2H])c([N+](=O)[O-])c(I)c1[2H]] 1946843-41-9 | 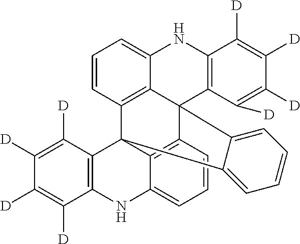 [SMILES: [2H]c1c([2H])c([2H])c2c(c1[2H])Nc1cccc3c1C21c2ccccc2C32c3c(cccc31)Nc1c([2H])c([2H])c([2H])c([2H])c12] | 26% |

### Example S100

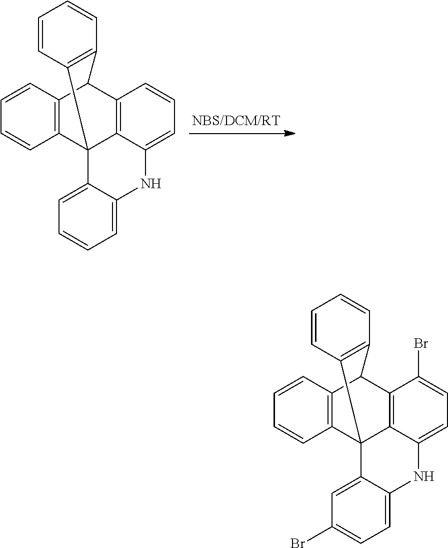 [SMILES: c1ccc2c(c1)Nc1cccc3c1C21c2ccccc2C3c2ccccc21 ; Brc1ccc2c(c1)C13c4ccccc4C(c4ccccc41)c1c(Br)ccc(c13)N2]

Preparation analogous to Y. Hu et al., ACS Appl. Polymer Mat. 2019, 1(2), 221. For every CH function para to NH, 1.05 eq of NBS is used. Starting materials: 34.4 g (100 mmol) of S1. Yield: 44.8 g (89 mmol) 89%. Purity: 97% by ¹H-NMR.

The following compounds can be obtained analogously:

| Ex. | Reactant | Product | Yield |
|---|---|---|---|
| S101 | S2 | 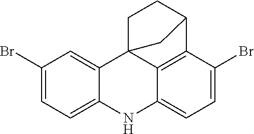 [SMILES: Brc1ccc2c(c1)C13CCC(C1)c1c(Br)ccc(c13)N2] | 93% |
| S102 | S5 | 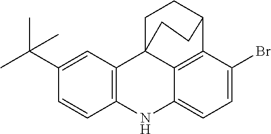 [SMILES: CC(C)(C)c1ccc2c(c1)C13CCC(CC1)c1c(Br)ccc(c13)N2] | 95% |
| S103 | S8 | 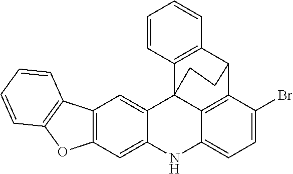 [SMILES: Brc1ccc2c3c1C1CCC3(c3cc4c(cc3N2)oc2ccccc24)c2ccccc21] | 87% |
| S104 | S15 | 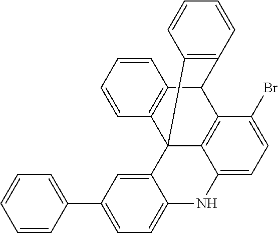 [SMILES: Brc1ccc2c3c1C1c4ccccc4C3(c3cc(-c4ccccc4)ccc3N2)c2ccccc21] | 92% |

### Example S200

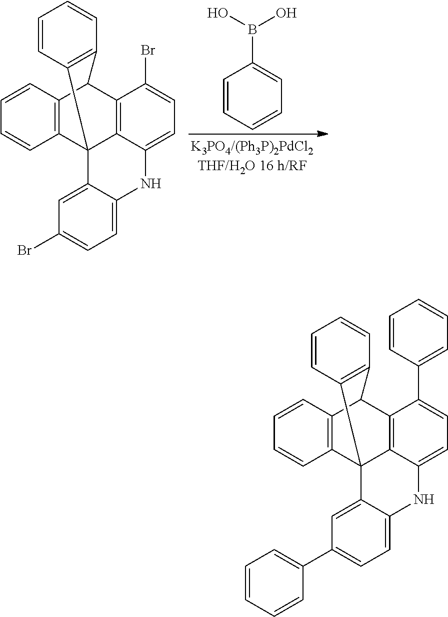 [SMILES: Brc1ccc2c(c1)C13c4ccccc4C(c4ccccc41)c1c(Br)ccc(c13)N2 ; OB(O)c1ccccc1 ; c1ccc(-c2ccc3c(c2)C24c5ccccc5C(c5ccccc52)c2c(-c5ccccc5)ccc(c24)N3)cc1]

Preparation analogous to B. van Veller et al., J. Am. Chem. Soc., 2012, 134(17), 7282. Starting materials: 50.1 g (100 mmol) of S100. Yield: 43.4 g (88 mmol) 88%. Purity: 97% by ¹H-NMR.

The following compounds can be obtained analogously:

| Ex. | Reactant | Product | Yield |
|---|---|---|---|
| S201 | S100 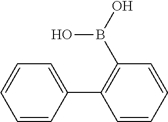 [SMILES: OB(O)c1ccccc1-c1ccccc1] 914675-52-8 | 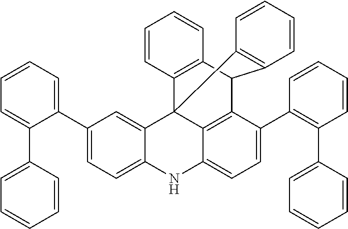 [SMILES: c1ccc(-c2ccccc2-c2ccc3c(c2)C24c5ccccc5C(c5ccccc52)c2c(-c5ccccc5-c5ccccc5)ccc(c24)N3)cc1] | 83% |
| S202 | S100 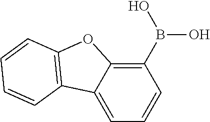 [SMILES: OB(O)c1cccc2c1oc1ccccc12] 100124-06-9 | 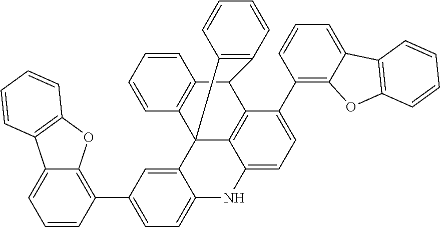 [SMILES: c1ccc2c(c1)C1c3ccccc3C23c2cc(-c4cccc5c4oc4ccccc45)ccc2Nc2ccc(-c4cccc5c4oc4ccccc45)c1c23] | 86% |
| S203 | S101 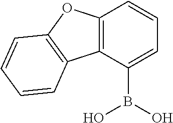 [SMILES: OB(O)c1cccc2oc3ccccc3c12] 162608-19-4 | 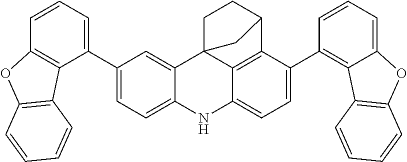 [SMILES: c1ccc2c(c1)oc1cccc(-c3ccc4c(c3)C35CCC(C3)c3c(-c6cccc7oc8ccccc8c67)ccc(c35)N4)c12] | 79% |
| S204 | S102 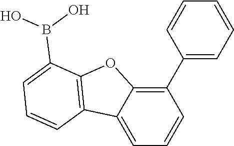 [SMILES: OB(O)c1cccc2c1oc1c(-c3ccccc3)cccc12] 1010068-85-5 | 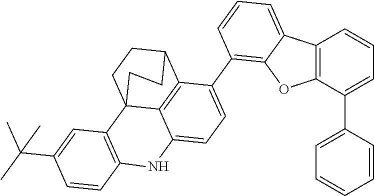 [SMILES: CC(C)(C)c1ccc2c(c1)C13CCC(CC1)c1c(-c4cccc5c4oc4c(-c6ccccc6)cccc45)ccc(c13)N2] | 90% |
| S205 | S103 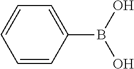 98-80-6 | 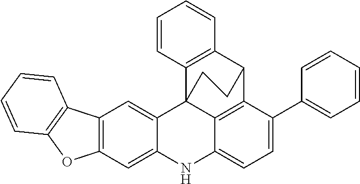 [SMILES: c1ccc(-c2ccc3c4c2C2CCC4(c4cc5c(cc4N3)oc3ccccc35)c3ccccc32)cc1] | 87% |
| S206 | S104 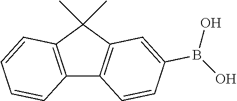 [SMILES: CC1(C)c2ccccc2-c2ccc(B(O)O)cc21] 333432-28-3 | 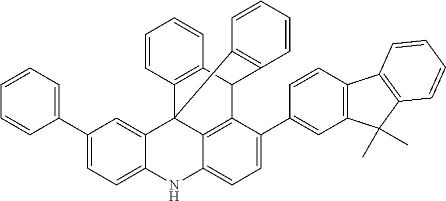 [SMILES: CC1(C)c2ccccc2-c2ccc(-c3ccc4c5c3C3c6ccccc6C5(c5cc(-c6ccccc6)ccc5N4)c4ccccc43)cc21] | 91% |
| S207 | S101 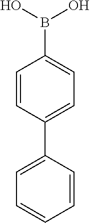 [SMILES: OB(O)c1ccc(-c2ccccc2)cc1] 5122-94-1 | 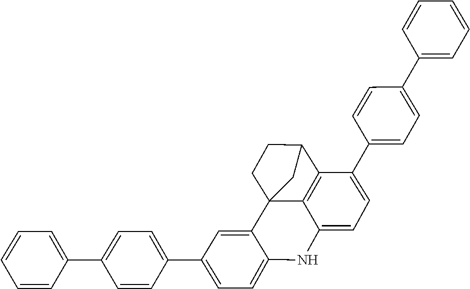 [SMILES: c1ccc(-c2ccc(-c3ccc4c(c3)C35CCC(C3)c3c(-c6ccc(-c7ccccc7)cc6)ccc(c35)N4)cc2)cc1] | 89% |
| S208 | S104 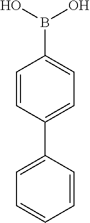 [SMILES: OB(O)c1ccc(-c2ccccc2)cc1] 5122-94-1 | 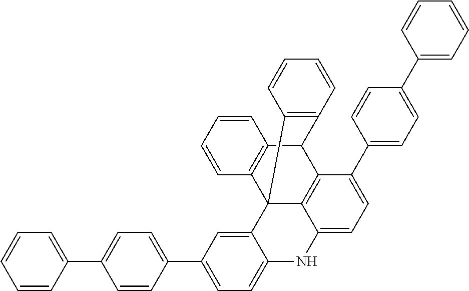 [SMILES: c1ccc(-c2ccc(-c3ccc4c(c3)C35c6ccccc6C(c6ccccc63)c3c(-c6ccc(-c7ccccc7)cc6)ccc(c35)N4)cc2)cc1] | 87% |

### Example S300

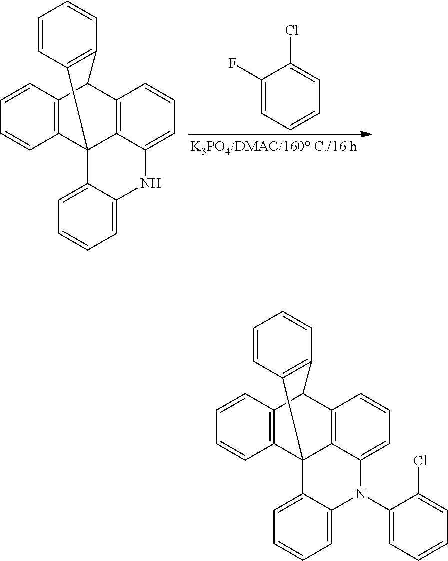 [SMILES: c1ccc2c(c1)Nc1cccc3c1C21c2ccccc2C3c2ccccc21 ; Fc1ccccc1Cl ; Clc1ccccc1N1c2ccccc2C23c4ccccc4C(c4ccccc42)c2cccc1c23]

Preparation analogous to C. M. Tonge et al., J. Am. Chem. Soc., 2019, 141, 35, 13970. Starting materials: 34.3 g (100 mmol) of S1. Yield: 29.1 g (64 mmol) 64%. Purity: 97% by ¹H-NMR.

The following compounds can be obtained analogously:

| Ex. | Reactants | Product | Yield |
|---|---|---|---|
| S301 | S1 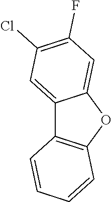 [SMILES: Fc1cc2oc3ccccc3c2cc1Cl] 2457274-69-8 | 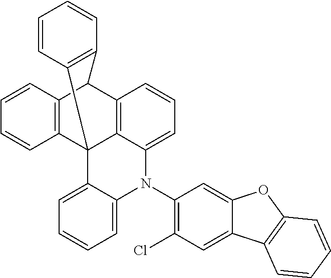 [SMILES: Clc1cc2c(cc1N1c3ccccc3C34c5ccccc5C(c5ccccc53)c3cccc1c34)oc1ccccc12] | 48% |
| S302 | S12 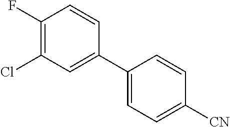 [SMILES: N#Cc1ccc(-c2ccc(F)c(Cl)c2)cc1] 869854-49-9 | 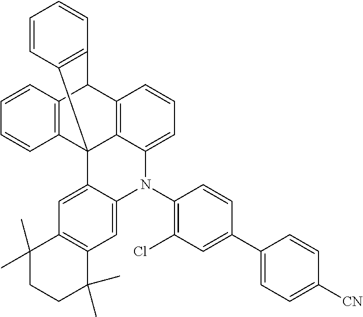 [SMILES: CC1(C)CCC(C)(C)c2cc3c(cc21)N(c1ccc(-c2ccc(C#N)cc2)cc1Cl)c1cccc2c1C31c3ccccc3C2c2ccccc21] | 67% |
| S303 | S21 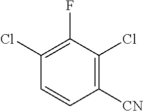 [SMILES: N#Cc1ccc(Cl)c(F)c1Cl] 161612-68-6 | 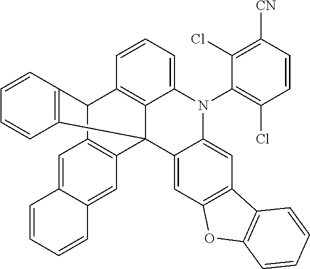 [SMILES: N#Cc1ccc(Cl)c(N2c3cc4c(cc3C35c6ccccc6C(c6cc7ccccc7cc63)c3cccc2c35)oc2ccccc24)c1Cl] | 45% |
| S304 | S1 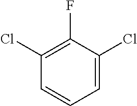 2268-05-5 | 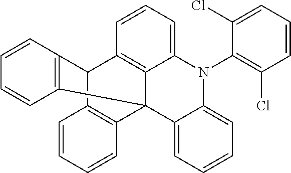 [SMILES: Clc1cccc(Cl)c1N1c2ccccc2C23c4ccccc4C(c4ccccc42)c2cccc1c23] | 49% |
| S305 | S5 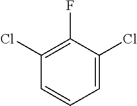 2268-05-5 | 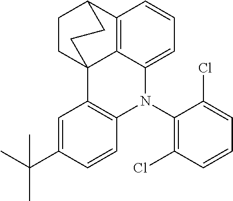 [SMILES: CC(C)(C)c1ccc2c(c1)C13CCC(CC1)c1cccc(c13)N2c1c(Cl)cccc1Cl] | 51% |

### 3) Synthesis of the Compounds of the Invention:

### Example B1

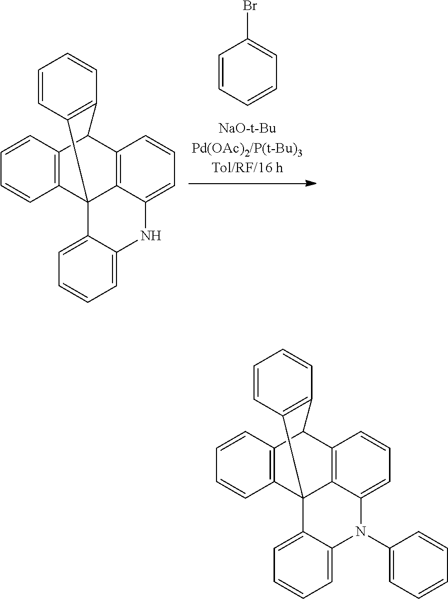 [SMILES: c1ccc2c(c1)Nc1cccc3c1C21c2ccccc2C3c2ccccc21 ; Brc1ccccc1 ; c1ccc(N2c3ccccc3C34c5ccccc5C(c5ccccc53)c3cccc2c34)cc1]

Preparation analogous to a) S. S. Reddy et al., Dyes and Pigments, (2016), 134, 315, or b) X. Liu et al., Angew. Chem. IE, 2021, 60(5), 2455 or c) W.-L. Tsai et al., Chem. Commun, 2015, 51(71), 13662. Route a), starting materials: 34.4 g (100 mmol) of S1. Purification is effected in each case by repeated hot extraction crystallization (customary organic solvents or combinations thereof, preferably acetonitrile-DCM, 1:3 to 3:1 vv) or chromatography and fractional sublimation or heat treatment under high vacuum. Yield: 38.2 g (91 mmol), 91%; purity: >99.9% by HPLC.

The following compounds can be obtained analogously:

| Ex. | Reactants | Product | Yield |
|---|---|---|---|
| B2 | S2 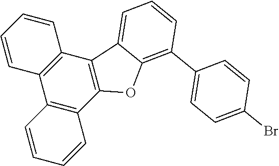 [SMILES: Brc1ccc(-c2cccc3c2oc2c4ccccc4c4ccccc4c32)cc1] 1846602-83-2 | 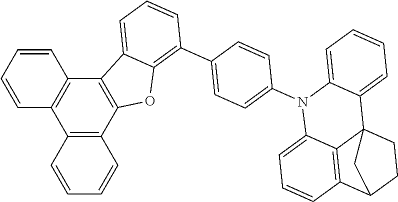 [SMILES: c1ccc2c(c1)N(c1ccc(-c3cccc4c3oc3c5ccccc5c5ccccc5c43)cc1)c1cccc3c1C21CCC3C1] | 88% |
| B3 | S2 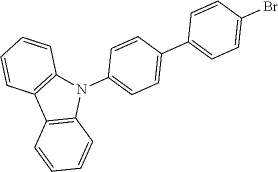 [SMILES: Brc1ccc(-c2ccc(-n3c4ccccc4c4ccccc43)cc2)cc1] | 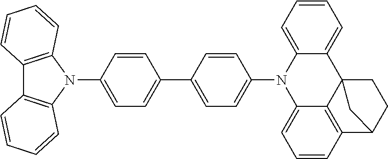 [SMILES: c1ccc2c(c1)N(c1ccc(-c3ccc(-n4c5ccccc5c5ccccc54)cc3)cc1)c1cccc3c1C21CCC3C1] | 79% |
| B4 | S3 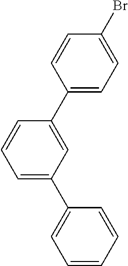 [SMILES: Brc1ccc(-c2cccc(-c3ccccc3)c2)cc1] 54590-38-3 | 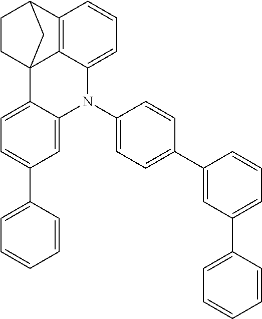 [SMILES: c1ccc(-c2cccc(-c3ccc(N4c5cc(-c6ccccc6)ccc5C56CCC(C5)c5cccc4c56)cc3)c2)cc1] | 90% |
| B5 | S3 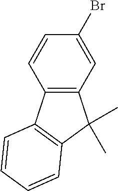 [SMILES: CC1(C)c2ccccc2-c2ccc(Br)cc21] 28320-31-2 | 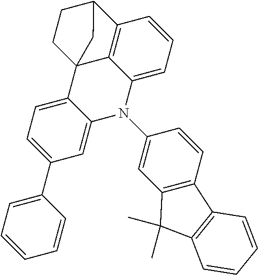 [SMILES: CC1(C)c2ccccc2-c2ccc(N3c4cc(-c5ccccc5)ccc4C45CCC(C4)c4cccc3c45)cc21] | 77% |
| B6 | S4 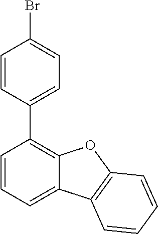 [SMILES: Brc1ccc(-c2cccc3c2oc2ccccc23)cc1] 955959-84-9 | 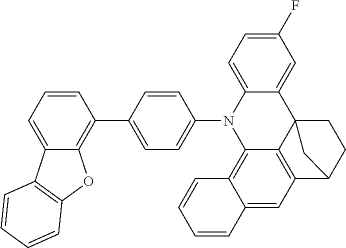 [SMILES: Fc1ccc2c(c1)C13CCC(C1)c1cc4ccccc4c(c13)N2c1ccc(-c2cccc3c2oc2ccccc23)cc1] | 80% |
| B7 | S5 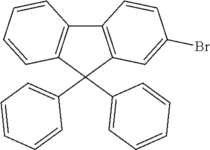 [SMILES: Brc1ccc2c(c1)C(c1ccccc1)(c1ccccc1)c1ccccc1-2] 474918-32-6 | 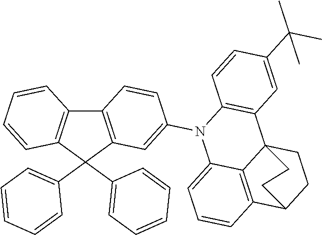 [SMILES: CC(C)(C)c1ccc2c(c1)C13CCC(CC1)c1cccc(c13)N2c1ccc2c(c1)C(c1ccccc1)(c1ccccc1)c1ccccc1-2] | 75% |
| B8 | S5 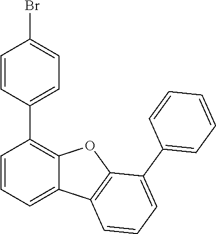 [SMILES: Brc1ccc(-c2cccc3c2oc2c(-c4ccccc4)cccc23)cc1] 1556069-46-5 | 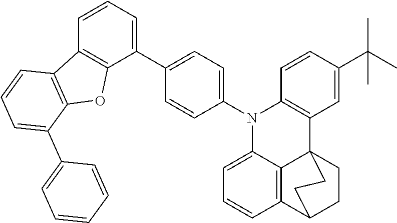 [SMILES: CC(C)(C)c1ccc2c(c1)C13CCC(CC1)c1cccc(c13)N2c1ccc(-c2cccc3c2oc2c(-c4ccccc4)cccc23)cc1] | 79% |
| B9 | S6 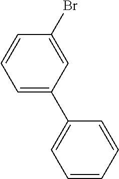 [SMILES: Brc1cccc(-c2ccccc2)c1] 2113-58-7 | 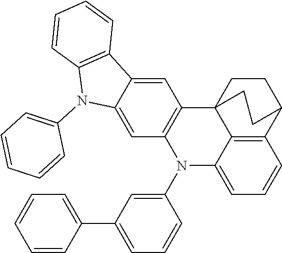 [SMILES: c1ccc(-c2cccc(N3c4cc5c(cc4C46CCC(CC4)c4cccc3c46)c3ccccc3n5-c3ccccc3)c2)cc1] | 73% |
| B10 | S6 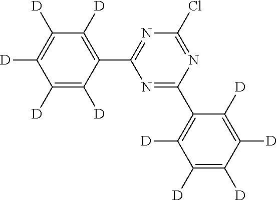 [SMILES: [2H]c1c([2H])c([2H])c(-c2nc(Cl)nc(-c3c([2H])c([2H])c([2H])c([2H])c3[2H])n2)c([2H])c1[2H]] 1300115-09-6 | 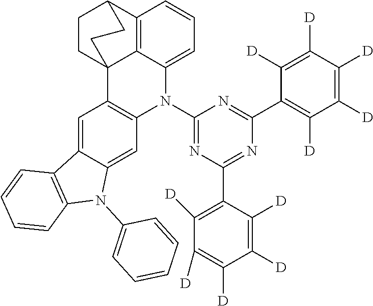 [SMILES: [2H]c1c([2H])c([2H])c(-c2nc(-c3c([2H])c([2H])c([2H])c([2H])c3[2H])nc(N3c4cc5c(cc4C46CCC(CC4)c4cccc3c46)c3ccccc3n5-c3ccccc3)n2)c([2H])c1[2H]] | 80% |
| B11 | S7 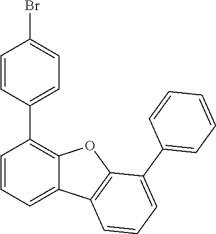 [SMILES: Brc1ccc(-c2cccc3c2oc2c(-c4ccccc4)cccc23)cc1] 1556069-46-5 | 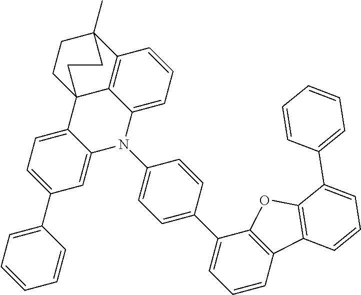 [SMILES: CC12CCC3(CC1)c1ccc(-c4ccccc4)cc1N(c1ccc(-c4cccc5c4oc4c(-c6ccccc6)cccc45)cc1)c1cccc2c13] | 78% |
| B12 | S8 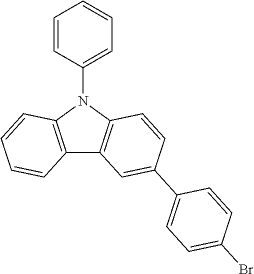 [SMILES: Brc1ccc(-c2ccc3c(c2)c2ccccc2n3-c2ccccc2)cc1] 1028648-93-9 | 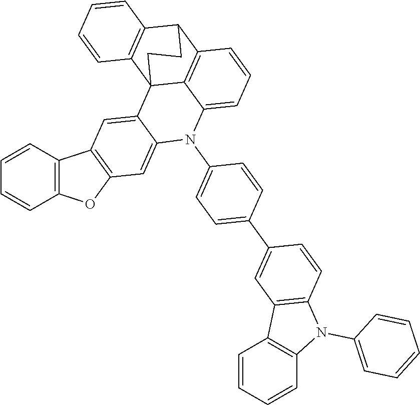 [SMILES: c1ccc(-n2c3ccccc3c3cc(-c4ccc(N5c6cc7oc8ccccc8c7cc6C67CCC(c8ccccc86)c6cccc5c67)cc4)ccc32)cc1] | 81% |
| B13 | S8 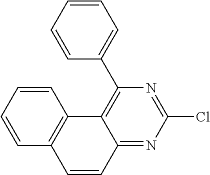 [SMILES: Clc1nc(-c2ccccc2)c2c(ccc3ccccc32)n1] 1528708-72-3 | 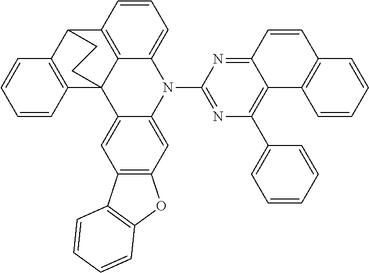 [SMILES: c1ccc(-c2nc(N3c4cc5oc6ccccc6c5cc4C45CCC(c6ccccc64)c4cccc3c45)nc3ccc4ccccc4c23)cc1] | 69% |
| B14 | S9 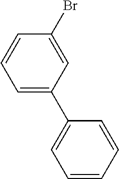 [SMILES: Brc1cccc(-c2ccccc2)c1] 2113-58-7 | 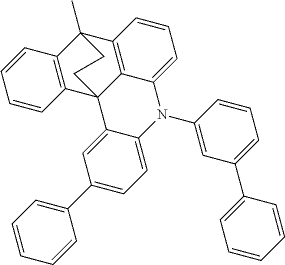 [SMILES: CC12CCC3(c4cc(-c5ccccc5)ccc4N(c4cccc(-c5ccccc5)c4)c4cccc1c43)c1ccccc12] | 80% |
| B15 | S9 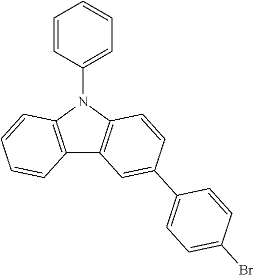 [SMILES: Brc1ccc(-c2ccc3c(c2)c2ccccc2n3-c2ccccc2)cc1] 1028648-93-9 | 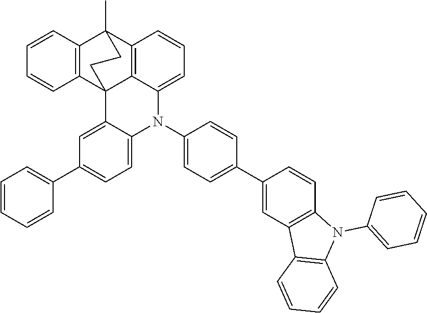 [SMILES: CC12CCC3(c4cc(-c5ccccc5)ccc4N(c4ccc(-c5ccc6c(c5)c5ccccc5n6-c5ccccc5)cc4)c4cccc1c43)c1ccccc12] | 77% |
| B16 | S10 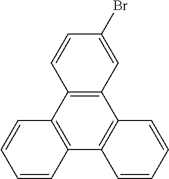 [SMILES: Brc1ccc2c3ccccc3c3ccccc3c2c1] 19111-88-6 | 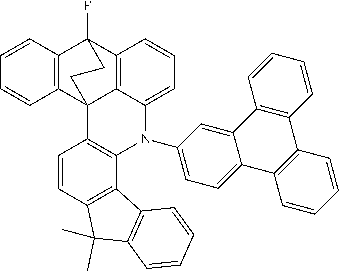 [SMILES: CC1(C)c2ccccc2-c2c1ccc1c2N(c2ccc3c4ccccc4c4ccccc4c3c2)c2cccc3c2C12CCC3(F)c1ccccc12] | 69% |
| B17 | S11 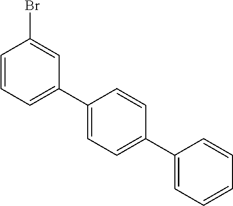 [SMILES: Brc1cccc(-c2ccc(-c3ccccc3)cc2)c1] 1762-88-4 | 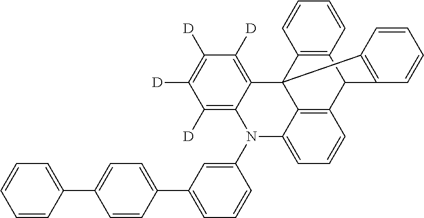 [SMILES: [2H]c1c([2H])c([2H])c2c(c1[2H])N(c1cccc(-c3ccc(-c4ccccc4)cc3)c1)c1cccc3c1C21c2ccccc2C3c2ccccc21] | 84% |
| B18 | S11 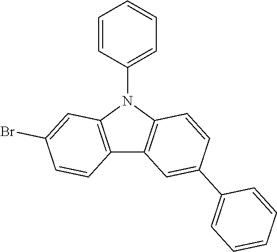 [SMILES: Brc1ccc2c3cc(-c4ccccc4)ccc3n(-c3ccccc3)c2c1] 1644466-58-9 | 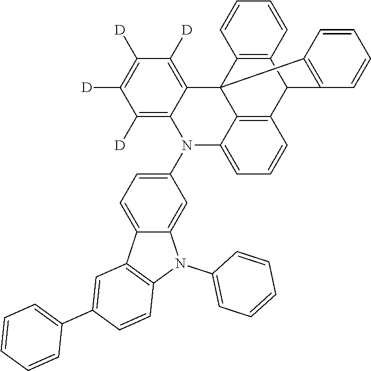 [SMILES: [2H]c1c([2H])c([2H])c2c(c1[2H])N(c1ccc3c4cc(-c5ccccc5)ccc4n(-c4ccccc4)c3c1)c1cccc3c1C21c2ccccc2C3c2ccccc21] | 82% |
| B19 | S11 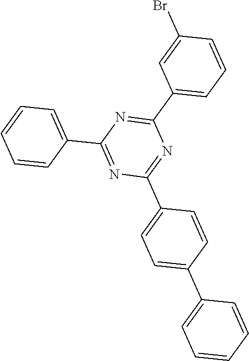 [SMILES: Brc1cccc(-c2nc(-c3ccccc3)nc(-c3ccc(-c4ccccc4)cc3)n2)c1] 1644466-58-9 | 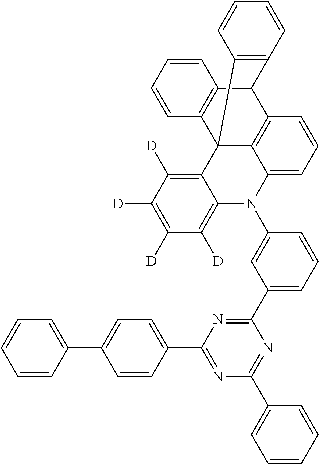 [SMILES: [2H]c1c([2H])c([2H])c2c(c1[2H])N(c1cccc(-c3nc(-c4ccccc4)nc(-c4ccc(-c5ccccc5)cc4)n3)c1)c1cccc3c1C21c2ccccc2C3c2ccccc21] | 68% |
| B20 | S11 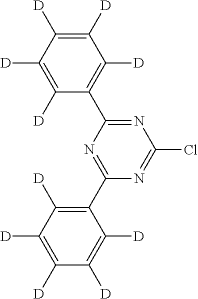 [SMILES: [2H]c1c([2H])c([2H])c(-c2nc(Cl)nc(-c3c([2H])c([2H])c([2H])c([2H])c3[2H])n2)c([2H])c1[2H]] 1300115-09-6 | 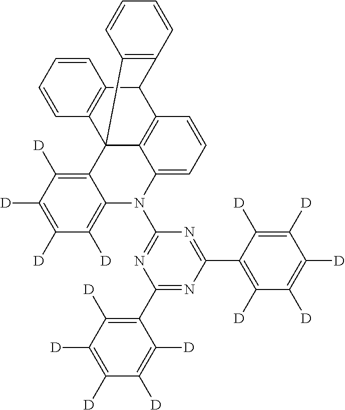 [SMILES: [2H]c1c([2H])c([2H])c(-c2nc(-c3c([2H])c([2H])c([2H])c([2H])c3[2H])nc(N3c4cccc5c4C4(c6ccccc6C5c5ccccc54)c4c([2H])c([2H])c([2H])c([2H])c43)n2)c([2H])c1[2H]] | 79% |
| B21 | S12 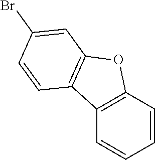 [SMILES: Brc1ccc2c(c1)oc1ccccc12] 26608-06-0 | 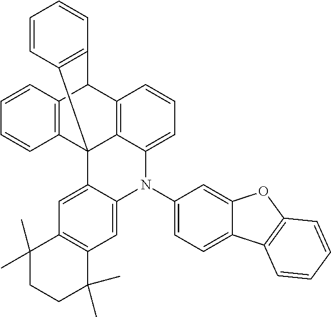 [SMILES: CC1(C)CCC(C)(C)c2cc3c(cc21)N(c1ccc2c(c1)oc1ccccc12)c1cccc2c1C31c3ccccc3C2c2ccccc21] | 75% |
| B22 | S13 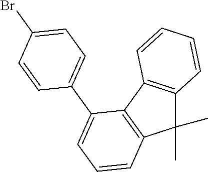 [SMILES: CC1(C)c2ccccc2-c2c(-c3ccc(Br)cc3)cccc21] 1860896-38-3 | 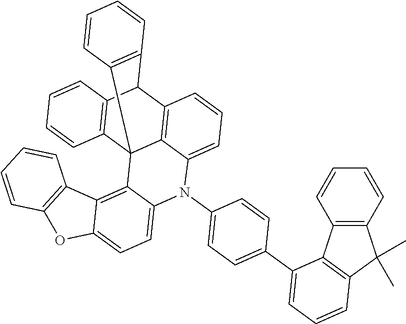 [SMILES: CC1(C)c2ccccc2-c2c(-c3ccc(N4c5cccc6c5C5(c7ccccc7C6c6ccccc65)c5c4ccc4oc6ccccc6c54)cc3)cccc21] | 82% |
| B23 | S14 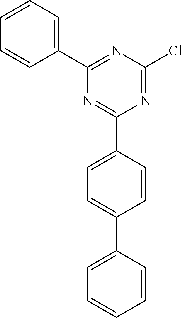 [SMILES: Clc1nc(-c2ccccc2)nc(-c2ccc(-c3ccccc3)cc2)n1] 1472062-94-4 | 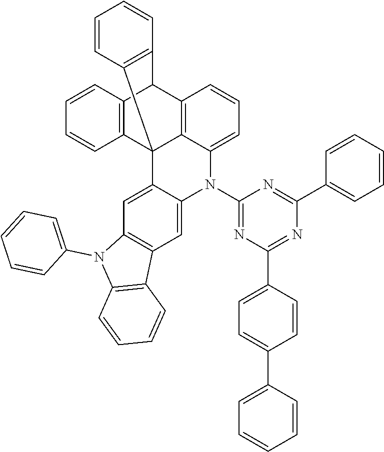 [SMILES: c1ccc(-c2ccc(-c3nc(-c4ccccc4)nc(N4c5cc6c7ccccc7n(-c7ccccc7)c6cc5C56c7ccccc7C(c7ccccc75)c5cccc4c56)n3)cc2)cc1] | 75% |
| B24 | S15 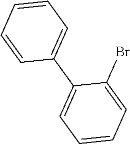 [SMILES: Brc1ccccc1-c1ccccc1] 2052-08-5 | 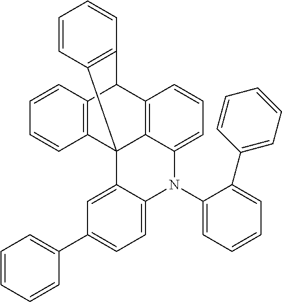 [SMILES: c1ccc(-c2ccc3c(c2)C24c5ccccc5C(c5ccccc52)c2cccc(c24)N3c2ccccc2-c2ccccc2)cc1] | 70% |
| B25 | S15 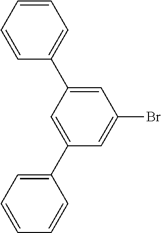 [SMILES: Brc1cc(-c2ccccc2)cc(-c2ccccc2)c1] 103068-20-8 | 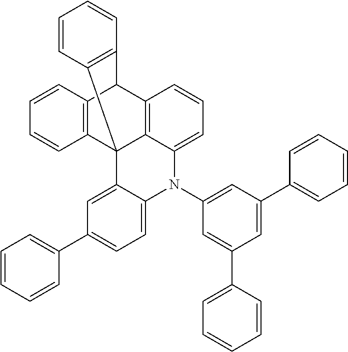 [SMILES: c1ccc(-c2cc(-c3ccccc3)cc(N3c4ccc(-c5ccccc5)cc4C45c6ccccc6C(c6ccccc64)c4cccc3c45)c2)cc1] | 79% |
| B26 | S15 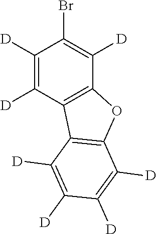 [SMILES: [2H]c1c([2H])c([2H])c2c(oc3c([2H])c(Br)c([2H])c([2H])c32)c1[2H]] 2727880-09-1 | 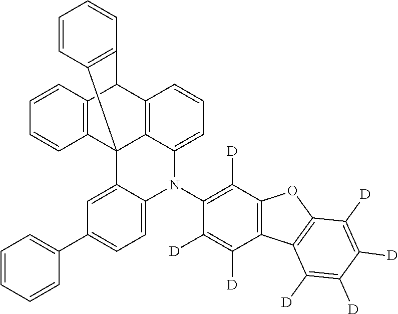 [SMILES: [2H]c1c([2H])c([2H])c2c(oc3c([2H])c(N4c5ccc(-c6ccccc6)cc5C56c7ccccc7C(c7ccccc75)c5cccc4c56)c([2H])c([2H])c32)c1[2H]] | 74% |
| B27 | S15 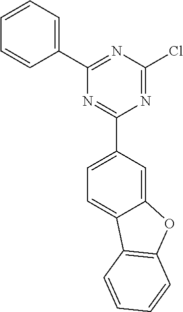 [SMILES: Clc1nc(-c2ccccc2)nc(-c2ccc3c(c2)oc2ccccc23)n1] 2142681-84-1 | 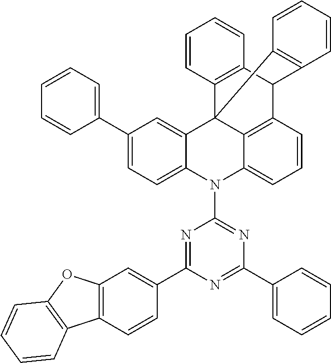 [SMILES: c1ccc(-c2ccc3c(c2)C24c5ccccc5C(c5ccccc52)c2cccc(c24)N3c2nc(-c3ccccc3)nc(-c3ccc4c(c3)oc3ccccc34)n2)cc1] | 86% |
| B28 | S16 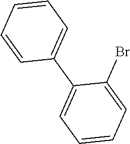 [SMILES: Brc1ccccc1-c1ccccc1] 2052-08-5 | 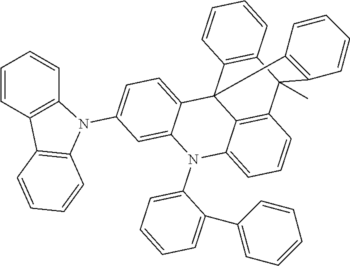 [SMILES: CC12c3ccccc3C3(c4ccc(-n5c6ccccc6c6ccccc65)cc4N(c4ccccc4-c4ccccc4)c4cccc1c43)c1ccccc12] | 85% |
| B29 | S16 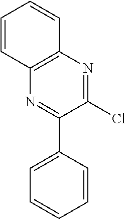 [SMILES: Clc1nc2ccccc2nc1-c1ccccc1] 7065-92-1 | 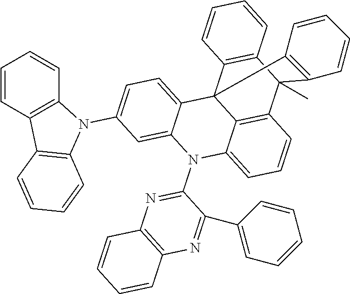 [SMILES: CC12c3ccccc3C3(c4ccc(-n5c6ccccc6c6ccccc65)cc4N(c4nc5ccccc5nc4-c4ccccc4)c4cccc1c43)c1ccccc12] | 64% |
| B30 | S17 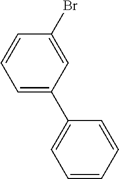 [SMILES: Brc1cccc(-c2ccccc2)c1] 2113-58-7 | 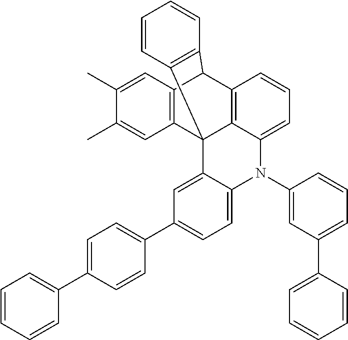 [SMILES: Cc1cc2c(cc1C)C13c4ccccc4C2c2cccc(c21)N(c1cccc(-c2ccccc2)c1)c1ccc(-c2ccc(-c4ccccc4)cc2)cc13] | 76% |
| B31 | S18 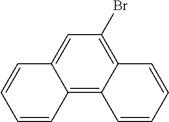 [SMILES: Brc1cc2ccccc2c2ccccc12] 573-18-1 | 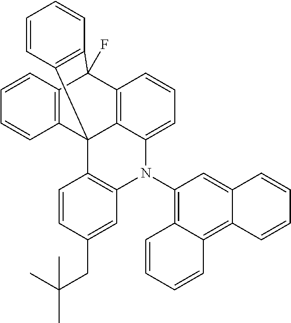 [SMILES: CC(C)(C)Cc1ccc2c(c1)N(c1cc3ccccc3c3ccccc13)c1cccc3c1C21c2ccccc2C3(F)c2ccccc21] | 68% |
| B32 | S19 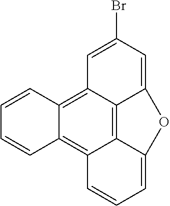 [SMILES: Brc1cc2oc3cccc4c5ccccc5c(c1)c2c34] 2653325-64-3 | 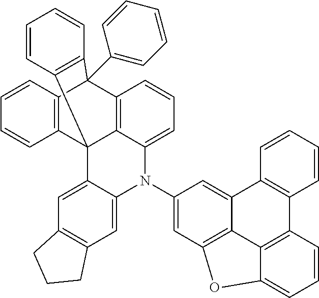 [SMILES: c1ccc(C23c4ccccc4C4(c5cc6c(cc5N(c5cc7oc8cccc9c%10ccccc%10c(c5)c7c89)c5cccc2c54)CCC6)c2ccccc23)cc1] | 73% |
| B33 | S20 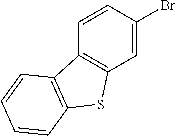 [SMILES: Brc1ccc2c(c1)sc1ccccc12] 97511-04-1 | 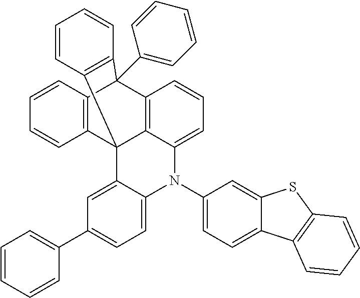 [SMILES: c1ccc(-c2ccc3c(c2)C24c5ccccc5C(c5ccccc5)(c5ccccc52)c2cccc(c24)N3c2ccc3c(c2)sc2ccccc23)cc1] | 80% |
| B34 | S20 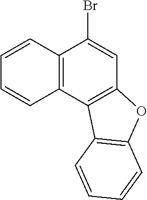 [SMILES: Brc1cc2oc3ccccc3c2c2ccccc12] 1622452-00-9 | 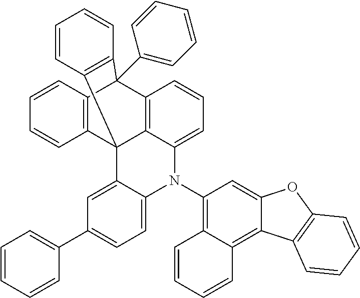 [SMILES: c1ccc(-c2ccc3c(c2)C24c5ccccc5C(c5ccccc5)(c5ccccc52)c2cccc(c24)N3c2cc3oc4ccccc4c3c3ccccc23)cc1] | 70% |
| B35 | S20 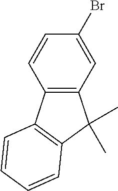 [SMILES: CC1(C)c2ccccc2-c2ccc(Br)cc21] 28320-31-2 | 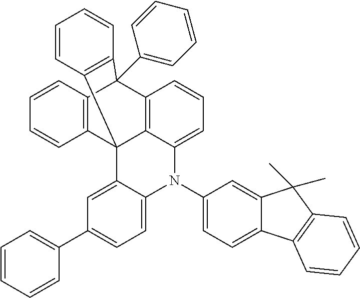 [SMILES: CC1(C)c2ccccc2-c2ccc(N3c4ccc(-c5ccccc5)cc4C45c6ccccc6C(c6ccccc6)(c6ccccc64)c4cccc3c45)cc21] | 83% |
| B36 | S21 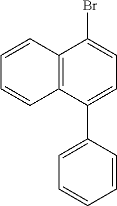 [SMILES: Brc1ccc(-c2ccccc2)c2ccccc12] 59951-65-4 | 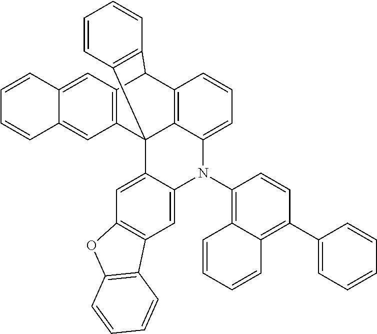 [SMILES: c1ccc(-c2ccc(N3c4cc5c(cc4C46c7ccccc7C(c7cc8ccccc8cc74)c4cccc3c46)oc3ccccc35)c3ccccc23)cc1] | 70% |
| B37 | S22 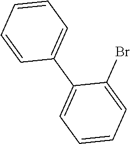 [SMILES: Brc1ccccc1-c1ccccc1] 2052-08-5 | 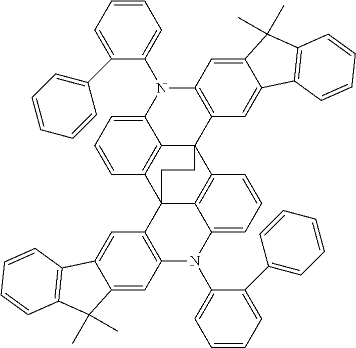 [SMILES: CC1(C)c2ccccc2-c2cc3c(cc21)N(c1ccccc1-c1ccccc1)c1cccc2c1C31CCC23c2cc4c(cc2N(c2ccccc2-c2ccccc2)c2cccc1c23)C(C)(C)c1ccccc1-4] | 69% |
| B38 | S23 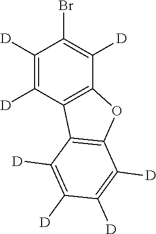 [SMILES: [2H]c1c([2H])c([2H])c2c(oc3c([2H])c(Br)c([2H])c([2H])c32)c1[2H]] 2727880-09-1 | 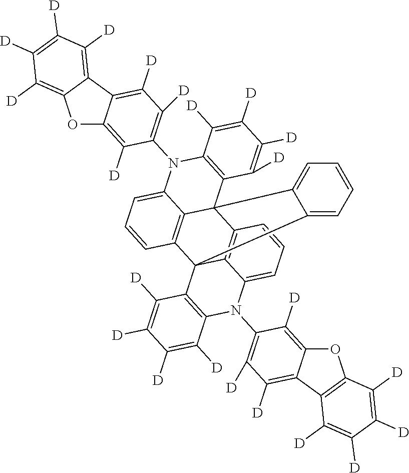 [SMILES: [2H]c1c([2H])c([2H])c2c(c1[2H])N(c1c([2H])c([2H])c3c(oc4c([2H])c([2H])c([2H])c([2H])c43)c1[2H])c1cccc3c1C21c2ccccc2C32c3c(cccc31)N(c1c([2H])c([2H])c3c(oc4c([2H])c([2H])c([2H])c([2H])c43)c1[2H])c1c([2H])c([2H])c([2H])c([2H])c12] | 56% |
| B39 | S200 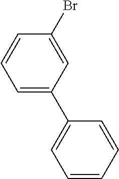 [SMILES: Brc1cccc(-c2ccccc2)c1] 2113-58-7 | 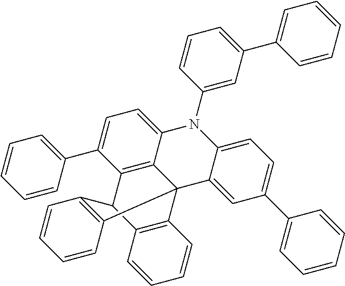 [SMILES: c1ccc(-c2cccc(N3c4ccc(-c5ccccc5)cc4C45c6ccccc6C(c6ccccc64)c4c(-c6ccccc6)ccc3c45)c2)cc1] | 81% |
| B40 | S200 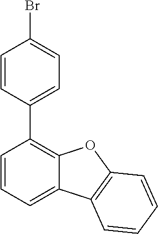 [SMILES: Brc1ccc(-c2cccc3c2oc2ccccc23)cc1] 955959-84-9 | 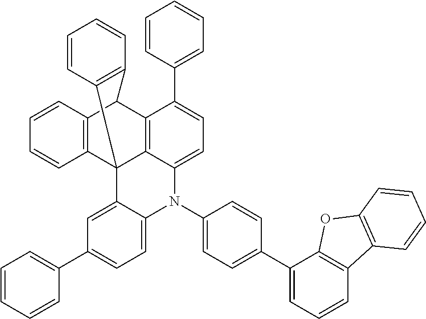 [SMILES: c1ccc(-c2ccc3c(c2)C24c5ccccc5C(c5ccccc52)c2c(-c5ccccc5)ccc(c24)N3c2ccc(-c3cccc4c3oc3ccccc34)cc2)cc1] | 84% |
| B41 | S200 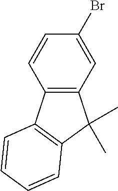 [SMILES: CC1(C)c2ccccc2-c2ccc(Br)cc21] 28320-31-2 | 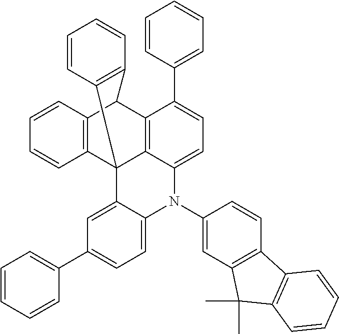 [SMILES: CC1(C)c2ccccc2-c2ccc(N3c4ccc(-c5ccccc5)cc4C45c6ccccc6C(c6ccccc64)c4c(-c6ccccc6)ccc3c45)cc21] | 79% |
| B42 | S201 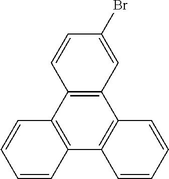 [SMILES: Brc1ccc2c3ccccc3c3ccccc3c2c1] 19111-88-6 | 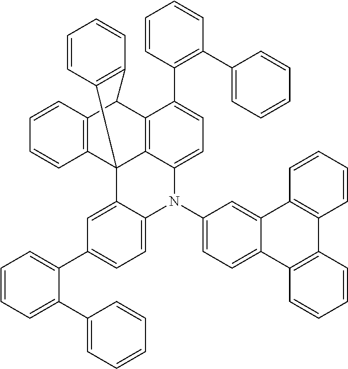 [SMILES: c1ccc(-c2ccccc2-c2ccc3c(c2)C24c5ccccc5C(c5ccccc52)c2c(-c5ccccc5-c5ccccc5)ccc(c24)N3c2ccc3c4ccccc4c4ccccc4c3c2)cc1] | 75% |
| B43 | S202 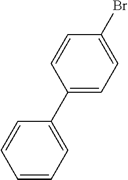 [SMILES: Brc1ccc(-c2ccccc2)cc1] 92-66-0 | 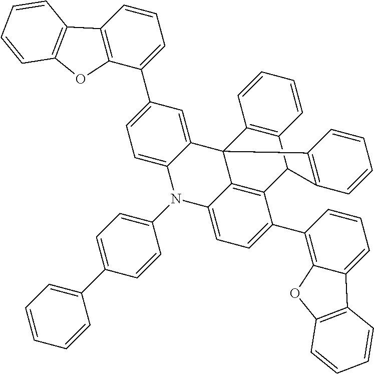 [SMILES: c1ccc(-c2ccc(N3c4ccc(-c5cccc6c5oc5ccccc56)cc4C45c6ccccc6C(c6ccccc64)c4c(-c6cccc7c6oc6ccccc67)ccc3c45)cc2)cc1] | 84% |
| B44 | S203 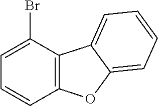 [SMILES: Brc1cccc2oc3ccccc3c12] 50548-45-3 | 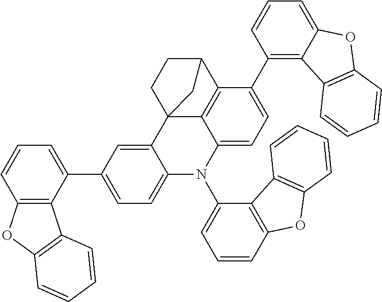 [SMILES: c1ccc2c(c1)oc1cccc(-c3ccc4c(c3)C35CCC(C3)c3c(-c6cccc7oc8ccccc8c67)ccc(c35)N4c3cccc4oc5ccccc5c34)c12] | 67% |
| B45 | S204 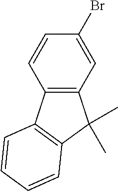 [SMILES: CC1(C)c2ccccc2-c2ccc(Br)cc21] 28320-31-2 | 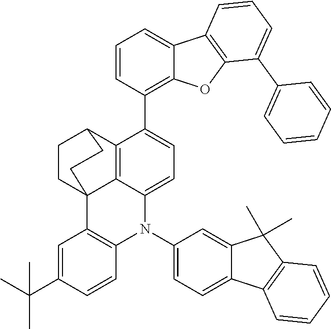 [SMILES: CC(C)(C)c1ccc2c(c1)C13CCC(CC1)c1c(-c4cccc5c4oc4c(-c6ccccc6)cccc45)ccc(c13)N2c1ccc2c(c1)C(C)(C)c1ccccc1-2] | 80% |
| B46 | S205 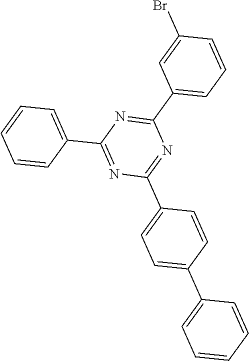 [SMILES: Brc1cccc(-c2nc(-c3ccccc3)nc(-c3ccc(-c4ccccc4)cc3)n2)c1] 1644466-58-9 | 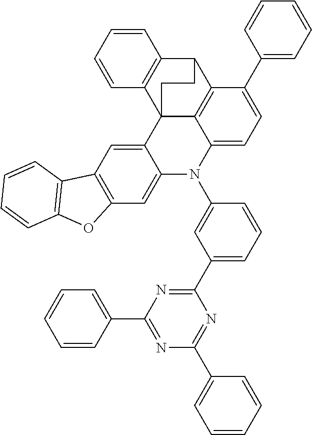 [SMILES: c1ccc(-c2nc(-c3ccccc3)nc(-c3cccc(N4c5cc6oc7ccccc7c6cc5C56CCC(c7ccccc75)c5c(-c7ccccc7)ccc4c56)c3)n2)cc1] | 75% |
| B47 | S206 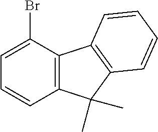 [SMILES: CC1(C)c2ccccc2-c2c(Br)cccc21] 942615-32-9 | 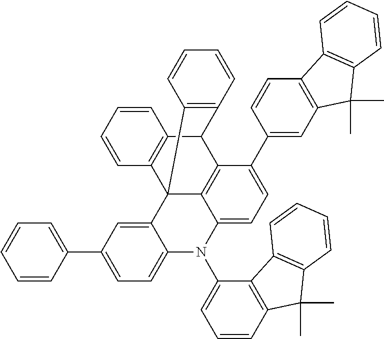 [SMILES: CC1(C)c2ccccc2-c2ccc(-c3ccc4c5c3C3c6ccccc6C5(c5ccccc53)c3cc(-c5ccccc5)ccc3N4c3cccc4c3-c3ccccc3C4(C)C)cc21] | 69% |
| B48 | S207 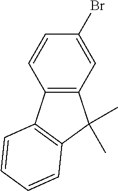 [SMILES: CC1(C)c2ccccc2-c2ccc(Br)cc21] 28320-31-2 | 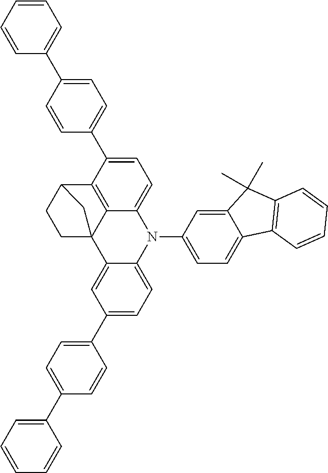 [SMILES: CC1(C)c2ccccc2-c2ccc(N3c4ccc(-c5ccc(-c6ccccc6)cc5)cc4C45CCC(C4)c4c(-c6ccc(-c7ccccc7)cc6)ccc3c45)cc21] | 83% |
| B49 | S208 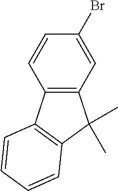 [SMILES: CC1(C)c2ccccc2-c2ccc(Br)cc21] 28320-31-2 | 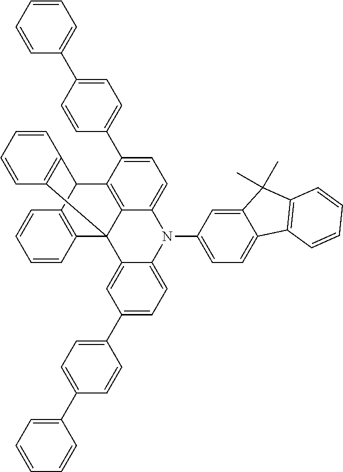 [SMILES: CC1(C)c2ccccc2-c2ccc(N3c4ccc(-c5ccc(-c6ccccc6)cc5)cc4C45c6ccccc6C(c6ccccc64)c4c(-c6ccc(-c7ccccc7)cc6)ccc3c45)cc21] | 80% |

### Example B100A & B100B

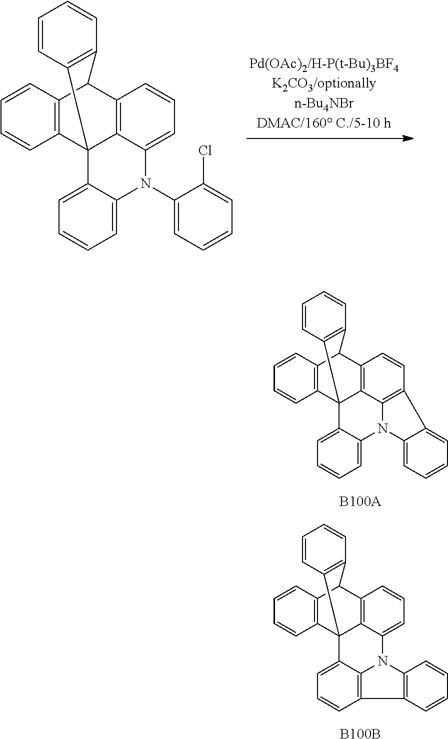 [SMILES: Clc1ccccc1N1c2ccccc2C23c4ccccc4C(c4ccccc42)c2cccc1c23 ; c1ccc2c(c1)C1c3ccccc3C23c2ccccc2-n2c4ccccc4c4ccc1c3c42 ; "B100B" ; c1ccc2c(c1)C1c3ccccc3C23c2c1cccc2-n1c2ccccc2c2cccc3c21]

Procedure analogous to a) T. Kader et al., Chem. Europ. J., 2019, 25(17), 4412 or analogous to b) U.S. Pat. No. 9,000,421 B1, using tricyclohexylphosphonium tetrafluoroborate or with c) NHC-Pd complexes, for example allyl[1,3-bis(2,6-diisopropylphenyl)imidazol-2-ylidene]chloropalladium(II).

Route a), starting materials: 45.4 g (100 mmol) of S300. The regioisomers are separated by flash chromatography (Torrent automated column system, from A. Semrau). Purification is effected in each case by repeated hot extraction crystallization (customary organic solvents or combinations thereof, preferably acetonitrile-DCM, 1:3 to 3:1 vv) or chromatography and fractional sublimation or heat treatment under high vacuum. Yield: B100A 11.3 g (27 mmol) 27%; B100B 12.8 g (31 mmol) 31%. Purity: >99.9% by HPLC.

The following compounds can be obtained analogously:

| Ex. | Reactants | Product | Yield |
|---|---|---|---|
| B101A B101B | S301 | 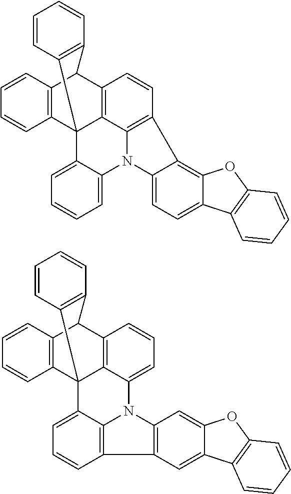 | 21% 24% |
| B102 | S302 | 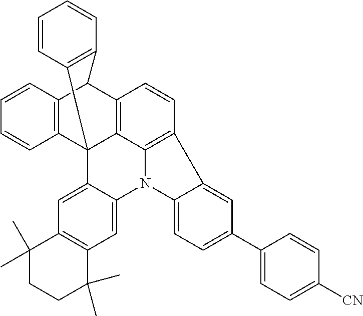 [SMILES: CC1(C)CCC(C)(C)c2cc3c(cc21)-n1c2ccc(-c4ccc(C#N)cc4)cc2c2ccc4c(c21)C31c2ccccc2C4c2ccccc21] | 37% |
| B103 | S303 | 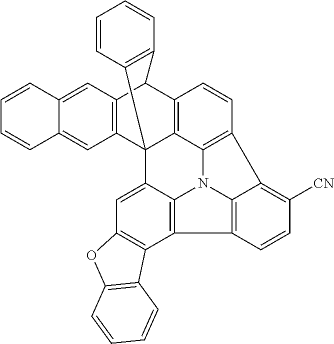 [SMILES: N#Cc1ccc2c3c4c(cc5c3n3c6c7c(ccc6c1c23)C1c2ccccc2C75c2cc3ccccc3cc21)oc1ccccc14] | 17% |

### Example, Dopants D1A and D1B

Steps 1 to 3 of the sequence that follows are conducted as a three-stage one-pot reaction. The workup in step 3 is effected under protective gas.

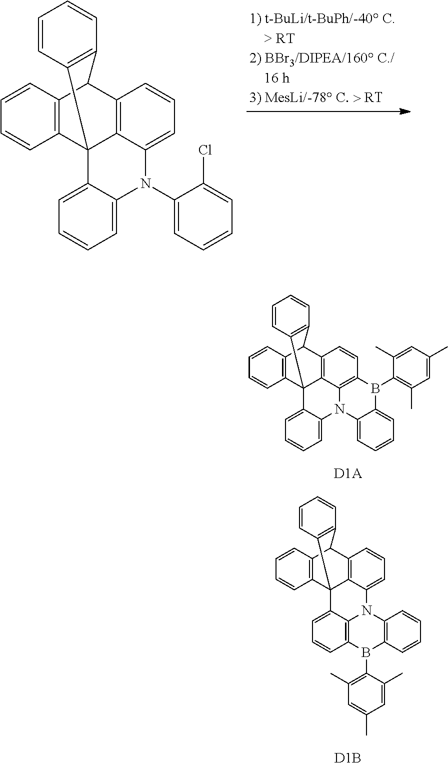

### Step 1: Lithiation of S300

A baked-out, argon-inertized four-neck flask with magnetic stirrer bar, dropping funnel, water separator, reflux condenser and argon blanketing is charged with 45.4 g (100 mmol) of S300 in 1400 ml of tert-butylbenzene. The reaction mixture is cooled down to −40° C., and then 110.5 ml (210 mmol) of tert-butyllithium, 1.9 M in n-pentane, is added dropwise over the course of 30 min. The mixture is stirred at −40° C. for a further 30 min, allowed to warm up to room temperature, then heated 70° C., in the course of which the n-pentane is distilled off via the water separator over about 1 h.

Step 2: Transmetalation and Cyclization The reaction mixture is cooled back down to −40° C. 10.4 ml (110 mmol) of boron tribromide is added dropwise over a period of about 10 min. On completion of addition, the reaction mixture is stirred at RT for 1 h. Then the reaction mixture is cooled down to 0° C., and 19.2 ml (110 mmol) of di-iso-propylethylamine is added dropwise over a period of about 30 min. Then the reaction mixture is stirred at 160° C. for 16 h. After cooling, di-iso-propylethylammmonium hydrobromide is filtered off using a double-ended frit, and the filtrate is cooled down to −78° C.

### Step 3: Arylation

A second baked-out, argon-inertized Schlenk flask with magnetic stirrer bar is charged with 29.9 g (150 mmol) of 2-bromo-1,3,5-trimethylbenzene [576-83-0] in 1000 ml of diethyl ether and cooled down to −78° C. Then 60.0 ml (150 mmol) of n-butyllithium, 2.5 M in n-hexane, is added dropwise thereto and the mixture is stirred for a further 30 min. The reaction mixture is allowed to warm up to RT and stirred for a further 1 h, and the solvent is removed completely under reduced pressure. The lithium organyl is suspended in 300 ml of toluene and transferred into the cryogenic reaction mixture from step 2. The mixture is stirred for a further 1 h, and the reaction mixture is left to warm up to RT overnight. 15 ml of acetone is added cautiously to the reaction mixture, which is concentrated to dryness. The oily residue is absorbed with ECM onto ISOLUTE© and hot-filtered through a silica gel bed with an n-pentane-DCM mixture (10:1). The filtrate is concentrated to dryness. The regioisomers are separated by flash chromatography (silica gel, n-heptane/ethyl acetate, Torrent automated column system, from A. Semrau). Purification is effected in each case by repeated hot extraction crystallization (customary organic solvents or combinations thereof, preferably acetonitrile-DCM, 1:3 to 3:1 vv) or chromatography and fractional sublimation or heat treatment under high vacuum. Yield: D1A 9.4 g (17 mmol) 17%; D1B 8.0 g (15 mmol) 15%. Purity: >99.9% by HPLC.

The following compounds can be obtained analogously:

| Ex. | Reactants | Product | Yield |
|---|---|---|---|
| D2 | S304 50 mmol 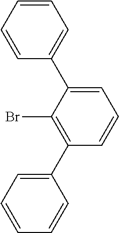 [SMILES: Brc1c(-c2ccccc2)cccc1-c1ccccc1] 126866-29-3 | 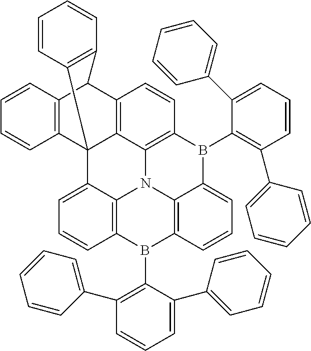 [SMILES: c1ccc(-c2cccc(-c3ccccc3)c2B2c3cccc4c3N3c5c2cccc5C25c6ccccc6C(c6ccccc62)c2ccc(c3c25)B4c2c(-c3ccccc3)cccc2-c2ccccc2)cc1] | 16% |
| D3 | S304 50 mmol 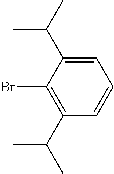 57190-18-7 | 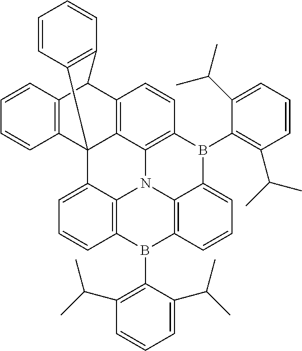 [SMILES: CC(C)c1cccc(C(C)C)c1B1c2cccc3c2N2c4c1cccc4C14c5ccccc5C(c5ccccc51)c1ccc(c2c14)B3c1c(C(C)C)cccc1C(C)C] | 21% |
| D4 | S304 50 mmol 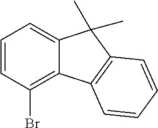 [SMILES: CC1(C)c2ccccc2-c2c(Br)cccc21] 942615-32-9 | 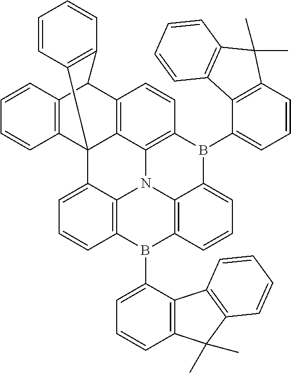 [SMILES: CC1(C)c2ccccc2-c2c(B3c4cccc5c4N4c6c3cccc6C36c7ccccc7C(c7ccccc73)c3ccc(c4c36)B5c3cccc4c3-c3ccccc3C4(C)C)cccc21] | 18% |
| D5 | S305 50 mmol 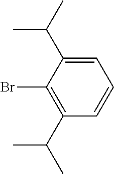 57190-18-7 | 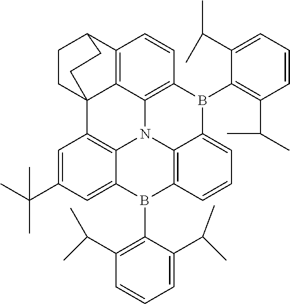 [SMILES: CC(C)c1cccc(C(C)C)c1B1c2cccc3c2N2c4c1cc(C(C)(C)C)cc4C14CCC(CC1)c1ccc(c2c14)B3c1c(C(C)C)cccc1C(C)C] | 14% |

### Example: Production of the OLEDs

### 1) Vacuum-Processed Devices:

OLEDs of the invention and OLEDs according to the prior art are produced by a general method according to WO 2004/058911, which is adapted to the circumstances described here (variation in layer thickness, materials used).

In the examples which follow, the results for various OLEDs are presented. Cleaned glass plates (cleaning in Miele laboratory glass washer, Merck Extran detergent) coated with structured ITO (indium tin oxide) of thickness 50 nm are pretreated with UV ozone for 25 minutes (UVP PR-100 UV ozone generator). These coated glass plates form the substrates to which the OLEDs are applied.

### 1a) Blue Fluorescent OLED Components—BF:

The compounds of the invention can be used in the hole injection layer (HIL), hole transport layer (HTL) and the electron blocker layer (EBL). All materials are applied by thermal vapor deposition in a vacuum chamber. The emission layer (EML) here always consists of at least one matrix material (host material) SMB (see table 1) and an emitting dopant (dopant, emitter) D, which is added to the matrix material(s) in a particular proportion by volume by co-evaporation. Details given in such a form as SMB:D (97:3%) mean here that the material SMB is present in the layer in a proportion by volume of 97% and the dopant D in a proportion of 3%.

Analogously, the electron transport layer may also consist of a mixture of two materials; see table 1. The materials used for production of the OLEDs are shown in table 5.

The OLEDs are characterized in a standard manner. For this purpose, the electroluminescence spectra, current efficiency (measured in cd/A), power efficiency (measured in lm/W) and external quantum efficiency (EQE, measured in percent) as a function of luminance, calculated from current-voltage-luminance characteristics (IUL characteristics) assuming Lambertian emission characteristics, and also lifetime are determined. EQE in (%) and voltage in (V) are reported at a luminance of 1000 cd/m². Lifetime is determined at a starting luminance of 10 000 cd/m². The measured period of time within which the brightness of the reference has dropped to 80% of initial brightness is set at 100%. The lifetime of the OLED components containing the compounds of the invention is reported in percent relative to the reference.

The OLEDs have the Following Layer Structure:

### Substrate

Hole injection layer (HIL) composed of HTM1 doped with 5% NDP-9 (commercially available from Novaled), 20 nm
Hole transport layer (HTL), see table 1
Electron blocker layer (EBL), see table 1
Emission layer (EML), see table 1
Electron transport layer (ETL), composed of ETM1:ETM2 (50%:50%), 30 nm
Electron injection layer (EIL) composed of ETM2, 1 nm
Cathode composed of aluminum, 100 nm

TABLE 1
Structure of blue fluorescent OLED components

| | HTL | EBL | EML |
|---|---|---|---|
| Ex. | thickness | thickness | thickness |
| BF-Ref1 | HTM1 | Ref-EBM1 | SMB1:Ref-D1 |
| | 180 nm | 10 nm | (95%:5%) |
| | | | 20 nm |
| BF1 | B8 | EBM1 | SMB1:Ref-D1 |
| | 180 nm | 10 nm | (95%:5%) |
| | | | 20 nm |
| BF2 | HTM1 | B5 | SMB1:Ref-D1 |
| | 180 nm | 10 nm | (95%:5%) |
| | | | 20 nm |
| BF3 | HTM1 | B11 | SMB3:Ref-D1 |
| | 180 nm | 10 nm | (95%:5%) |
| | | | 20 nm |
| BF4 | HTM1 | B14 | SMB1:Ref-D1 |
| | 180 nm | 10 nm | (95%:5%) |
| | | | 20 nm |
| BF5 | HTM1 | B25 | SMB1:Ref-D1 |
| | 180 nm | 10 nm | (95%:5%) |
| | | | 20 nm |
| BF6 | HTM1 | B35 | SMB1:Ref-D1 |
| | 180 nm | 10 nm | (95%:5%) |
| | | | 20 nm |
| BF7 | HTM1 | B39 | SMB1:Ref-D1 |
| | 180 nm | 10 nm | (95%:5%) |
| | | | 20 nm |
| BF8 | HTM1 | B47 | SMB1:Ref-D1 |
| | 180 nm | 10 nm | (95%:5%) |
| | | | 20 nm |
| BF9 | HTM1 | B48 | SMB1:Ref-D1 |
| | 180 nm | 10 nm | (95%:5%) |
| | | | 20 nm |
| BF10 | HTM1 | B49 | SMB1:Ref-D1 |
| | 180 nm | 10 nm | (95%:5%) |
| | | | 20 nm |
| BF11 | HTM1 | B49 | SMB2:D2 (95%:5%) |
| | 180 nm | 10 nm | 20 nm |

TABLE 2
Results for blue fluorescence OLED components

| | EQE (%) | Voltage | LT80 [%] |
|---|---|---|---|
| | 1000 | (V) 1000 | 10000 |
| Ex. | cd/m² | cd/m² | cd/m² |
| BF-Ref1 | 8.8 | 3.9 | 100 |
| BF1 | 9.4 | 3.8 | 160 |
| BF2 | 9.5 | 3.8 | 140 |
| BF3 | 10.0 | 3.9 | 160 |
| BF4 | 9.4 | 3.7 | 130 |
| BF5 | 9.6 | 3.8 | 140 |
| BF6 | 9.3 | 3.8 | 150 |
| BF7 | 9.2 | 3.7 | 130 |
| BF8 | 9.7 | 3.8 | 120 |
| BF9 | 92 | 3.7 | 130 |
| BF10 | 9.3 | 3.8 | 150 |
| BF11* | 9.9 | 3.7 | 80 |

*deep blue component

### 1b) Phosphorescent OILED Components:

The inventive compounds B can be used in the hole injection layer (HIL), the hole transport layer (HTL), the electron blocker layer (EBL) and the emission layer (EML) as matrix material (host material). For this purpose, all the materials are applied by thermal vapor deposition in a vacuum chamber. The emission layer here always consists of at least one or more than one matrix material M and a phosphorescent dopant Ir which is added to the matrix material(s) in a particular proportion by volume by co-evaporation. Details given in such a form as M1:M2:Ir (55%:35%:10%) mean here that the material M1 is present in the layer in a proportion by volume of 55%, M2 in a proportion by volume of 35% and Ir in a proportion by volume of 10%. Analogously, the electron transport layer may also consist of a mixture of two materials. The exact structure of the OLEDs can be found in table 3. The materials used for production of the OLEDs are shown in table 5.

The OLEDs are characterized in a standard manner. For this purpose, the electroluminescence spectra, current efficiency (measured in cd/A), power efficiency (measured in lm/W) and external quantum efficiency (EQE, measured in percent) as a function of luminance, calculated from current-voltage-luminance characteristics (IUL characteristics) assuming Lambertian emission characteristics, and also lifetime are determined. EQE in (%) and voltage in (V) are reported at a luminance of 1000 cd/m². Lifetime is determined at a starting luminance of 1000 cd/m² (blue, red) or 10 000 cd/m² (green, yellow). The measured period of time within which the brightness of the reference has dropped to 80% of initial brightness is set at 100%. The lifetime of the OLED components containing the compounds of the invention is reported in percent relative to the reference.

The OLEDs have the Following Layer Structure:

### Substrate

Hole injection layer (HIL) composed of HTM1 doped with 5% NDP-9 (commercially available from Novaled), 20 nm
Hole transport layer (HTL), see table 3
Electron blocker layer (EBL), see table 3
Emission layer (EML), see table 3
Hole blocker layer (HBL), see table 3
Electron transport layer (ETL), composed of ETM1:ETM2 (50%:50%), 30 nm
Electron injection layer (EIL) composed of ETM2, 1 nm
Cathode composed of aluminum, 100 nm

TABLE 3
Structure of phosphorescence OLED components

| | | | | |
|---|---|---|---|---|
| | HTL | EBL | EML | HBL |
| Ex. | thickness | thickness | thickness | thickness |

Blue

| | | | | |
|---|---|---|---|---|
| BP-Ref1 | HTM1 | Ref-EBM2 | M3:M4:IrB1 | HBM2 |
| | 180 nm | 20 nm | (30%:65%:5%) | 5 nm |
| | | | 25 nm | |
| BP1 | HTM1 | B1 | M3:M4:IrB1 | HBM2 |
| | 180 nm | 20 nm | (30%:65%:5%) | 5 nm |
| | | | 25 nm | |
| BP2 | HTM1 | B18 | M3:M4:IrB1 | HBM2 |
| | 180 nm | 20 nm | (30%:65%:5%) | 5 nm |
| | | | 25 nm | |
| BP3 | HTM1 | B18 | M3:B100A:IrB1 | HBM2 |
| | 180 nm | 20 nm | (30%:65%:5%) | 5 nm |
| | | | 25 nm | |

Green

| | | | | |
|---|---|---|---|---|
| GP- | HTM1 | Ref-EBM1 | M1:M2:IrG1 | HBM1 |
| Ref1 | 50 nm | 20 nm | (30%:60%:10%) | 5 nm |
| | | | 40 nm | |
| GP1 | B7 | EBM1 | M1:M2:IrG1 | HBM1 |
| | 50 nm | 20 nm | (30%:60%:10%) | 5 nm |
| | | | 40 nm | |
| GP2 | B8 | EBM1 | M1:M2:IrG1 | HBM1 |
| | 50 nm | 20 nm | (30%:60%:10%) | 5 nm |
| | | | 40 nm | |
| GP3 | HTM1 | B17 | M1:M2:IrG1 | HBM1 |
| | 50 nm | 20 nm | (30%:60%:10%) | 5 nm |
| | | | 40 nm | |
| GP4 | HTM1 | B21 | M1:M2:IrG1 | HBM1 |
| | 50 nm | 20 nm | (30%:60%:10%) | 5 nm |
| | | | 40 nm | |
| GP5 | HTM1 | B22 | M1:M2:IrG1 | HBM1 |
| | 50 nm | 20 nm | (30%:60%:10%) | 5 nm |
| | | | 40 nm | |
| GP6 | HTM1 | B40 | M1:M2:IrG1 | HBM1 |
| | 50 nm | 20 nm | (30%:60%:10%) | 5 nm |
| | | | 40 nm | |
| GP7 | HTM1 | B41 | M1:M2:IrG1 | HBM1 |
| | 50 nm | 20 nm | (30%:60%:10%) | 5 nm |
| | | | 40 nm | |
| GP8 | HTM1 | EBM1 | B20:M2:IrG1 | HBM1 |
| | 50 nm | 20 nm | (40%:50%:10%) | 5 nm |
| | | | 40 nm | |

Yellow

| | | | | |
|---|---|---|---|---|
| GP- | HTM1 | Ref-EBM1 | M1:M2:IrG2 | HBM1 |
| Ref4 | 40 nm | 20 nm | (30%:70%:10%) | 5 nm |
| | | | 40 nm | |
| GP50 | B37 | EBM1 | M1:M2:IrG2 | HBM1 |
| | 40 nm | 20 nm | (22%:66%:12%) | 5 nm |
| | | | 40 nm | |
| GP51 | HTM1 | B4 | M1:M2:IrG2 | HBM1 |
| | 40 nm | 20 nm | (22%:66%:12%) | 5 nm |
| | | | 40 nm | |
| GP52 | HTM1 | B26 | M1:M2:IrG2 | HBM1 |
| | 40 nm | 20 nm | (22%:66%:12%) | 5 nm |
| | | | 40 nm | |
| GP53 | HTM1 | B33 | M1:M2:IrG2 | HBM1 |
| | 40 nm | 20 nm | (22%:66%:12%) | 5 nm |
| | | | 40 nm | |
| GP54 | HTM1 | B43 | M1:M2:IrG2 | HBM1 |
| | 40 nm | 20 nm | (22%:66%:12%) | 5 nm |
| | | | 40 nm | |
| GP55 | HTM1 | EBM1 | B15:M2:IrG2 | HBM1 |
| | 40 nm | 20 nm | (25%:60%:15%) | 5 nm |
| | | | 40 nm | |
| GP56 | HTM1 | EBM1 | B27:M2:IrG2 | HBM1 |
| | 40 nm | 20 nm | (25%:60%:15%) | 5 nm |
| | | | 40 nm | |

Red

| | | | | |
|---|---|---|---|---|
| RP-Ref1 | HTM1 | Ref-EBM1 | M5:IrR1 | HBM1 |
| | 90 nm | 10 nm | (95%:5%) | 10 nm |
| | | | 35 nm | |
| RP1 | B6 | EBM1 | M5:IrR1 | HBM1 |
| | 90 nm | 10 nm | (95%:5%) | 10 nm |
| | | | 35 nm | |
| RP2 | HTM1 | B3 | M5:IrR1 | HBM1 |
| | 90 nm | 10 nm | (95%:5%) | 10 nm |
| | | | 35 nm | |
| RP3 | HTM1 | B12 | M5:IrR1 | HBM1 |
| | 90 nm | 10 nm | (95%:5%) | 10 nm |
| | | | 35 nm | |
| RP4 | HTM1 | B34 | M5:IrR1 | HBM1 |
| | 90 nm | 10 nm | (95%:5%) | 10 nm |
| | | | 35 nm | |
| RP5 | HTM1 | B32 | M5:IrR1 | HBM1 |
| | 90 nm | 10 nm | (95%:5%) | 10 nm |
| | | | 35 nm | |
| RP6 | HTM1 | B36 | M5:IrR1 | HBM1 |
| | 90 nm | 10 nm | (95%:5%) | 10 nm |
| | | | 35 nm | |
| RP7 | HTM1 | EBM1 | B10:IrR1 | HBM1 |
| | 90 nm | 10 nm | (95%:3%) | 10 nm |
| | | | 35 nm | |
| RP8 | HTM1 | EBM1 | B29:IrR1 | HBM1 |
| | 90 nm | 10 nm | (95%:3%) | 10 nm |
| | | | 35 nm | |
| RP9 | HTM1 | EBM1 | B46:IrR1 | HBM1 |
| | 90 nm | 10 nm | (95%:3%) | 10 nm |
| | | | 35 nm | |

TABLE 4
Results for phosphorescence OLED components

| | | | |
|---|---|---|---|
| | EQE (%) | Voltage (V) | LT80 (%) |
| Ex. | 1000 cd/m² | 1000 cd/m² | 1000 cd/m² |

Blue

| | | | |
|---|---|---|---|
| BP-Ref1 | 15.3 | 4.8 | 100 |
| BP1 | 22.6 | 4.3 | 550 |
| BP2 | 21.9 | 4.2 | 750 |
| BP3 | 22.0 | 4.3 | 650 |

Green

| | | | |
|---|---|---|---|
| GP-Ref1 | 22.4 | 3.4 | 100 |
| GP1 | 22.7 | 3.1 | 120 |
| GP2 | 23.0 | 3.2 | 110 |
| GP3 | 22.9 | 3.2 | 130 |
| GP4 | 23.3 | 3.1 | 160 |
| GP5 | 23.2 | 3.1 | 190 |
| GP6 | 23.4 | 3.1 | 200 |
| GP7 | 23.2 | 3.0 | 180 |
| GP8 | 23.7 | 3.2 | 170 |

Yellow

| | | | |
|---|---|---|---|
| GP-Ref4 | 29.1 | 3.2 | 100 |
| GP50 | 31.2 | 2.9 | 150 |
| GP51 | 30.6 | 3.2 | 170 |
| GP52 | 30.4 | 3.2 | 190 |
| GP53 | 31.0 | 3.2 | 120 |
| GP54 | 30.7 | 3.1 | 210 |
| GP55 | 31.3 | 3.3 | 200 |
| GP56 | 30.9 | 3.1 | 190 |

Red

| | | | |
|---|---|---|---|
| RP-Ref1 | 16.2 | 3.6 | 100 |
| RP1 | 17.4 | 3.1 | 120 |
| RP2 | 16.9 | 3.4 | 210 |
| RP3 | 17.3 | 3.3 | 230 |
| RP4 | 16.7 | 3.5 | 160 |
| RP5 | 17.8 | 3.4 | 180 |
| RP6 | 17.5 | 3.2 | 190 |
| RP7 | 17.6 | 3.2 | 130 |
| RP8 | 16.5 | 3.4 | 220 |
| RP9 | 17.0 | 3.3 | 160 |

TABLE 5
Structural formulae of the materials used

| | |
|---|---|
| 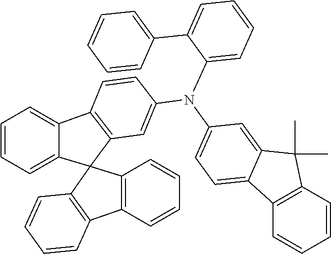 [SMILES: CC1(C)c2ccccc2-c2ccc(N(c3ccc4c(c3)C3(c5ccccc5-c5ccccc53)c3ccccc3-4)c3ccccc3-c3ccccc3)cc21] HTM1 136463-08-5 | 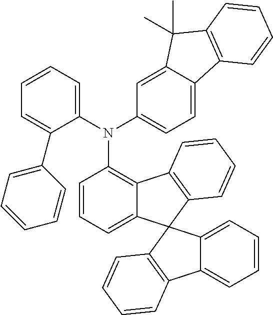 [SMILES: CC1(C)c2ccccc2-c2ccc(N(c3ccccc3-c3ccccc3)c3cccc4c3-c3ccccc3C43c4ccccc4-c4ccccc43)cc21] EBM1 1450933-44-4 |
| 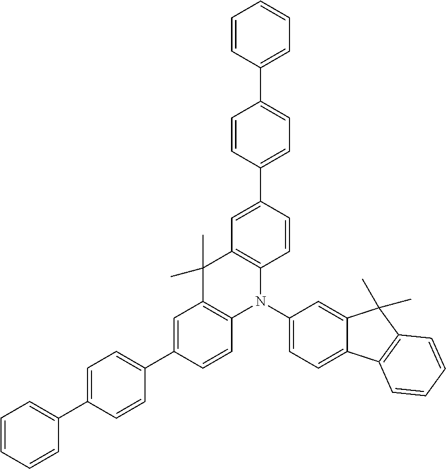 [SMILES: CC1(C)c2ccccc2-c2ccc(N3c4ccc(-c5ccc(-c6ccccc6)cc5)cc4C(C)(C)c4cc(-c5ccc(-c6ccccc6)cc5)ccc43)cc21] 1403980-09-5 Ref-EBM1 | 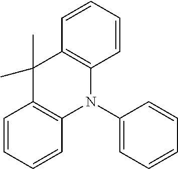 [SMILES: CC1(C)c2ccccc2N(c2ccccc2)c2ccccc21] 717880-39-2 Ref-EBM2 |
| 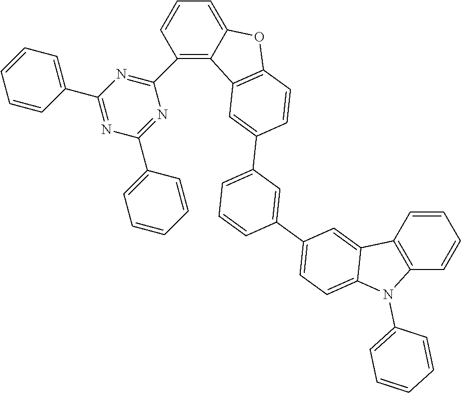 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1] M1 1822310-86-0 | 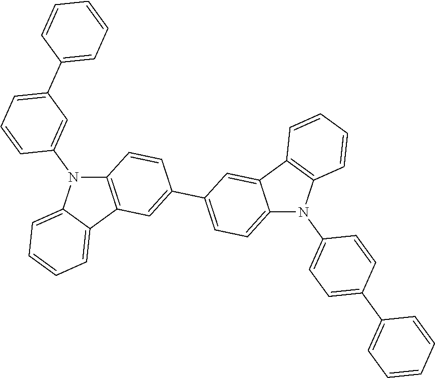 [SMILES: c1ccc(-c2ccc(-n3c4ccccc4c4cc(-c5ccc6c(c5)c5ccccc5n6-c5cccc(-c6ccccc6)c5)ccc43)cc2)cc1] M2 1643479-48-3 |
| 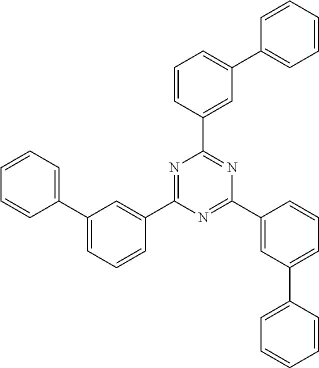 [SMILES: c1ccc(-c2cccc(-c3nc(-c4cccc(-c5ccccc5)c4)nc(-c4cccc(-c5ccccc5)c4)n3)c2)cc1] M3 = HBM2 1201800-83-0 | 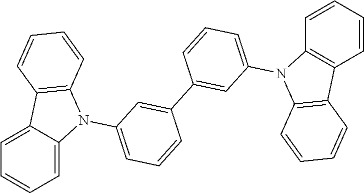 [SMILES: c1cc(-c2cccc(-n3c4ccccc4c4ccccc43)c2)cc(-n2c3ccccc3c3ccccc32)c1] M4 342638-54-4 |
| 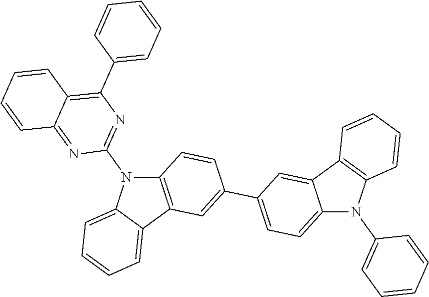 [SMILES: c1ccc(-c2nc(-n3c4ccccc4c4cc(-c5ccc6c(c5)c5ccccc5n6-c5ccccc5)ccc43)nc3ccccc23)cc1] M5 1398395-92-0 | 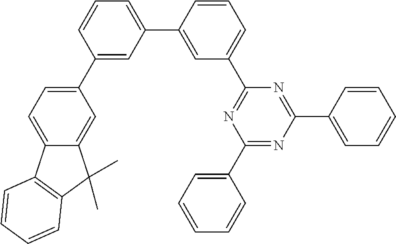 [SMILES: CC1(C)c2ccccc2-c2ccc(-c3cccc(-c4cccc(-c5nc(-c6ccccc6)nc(-c6ccccc6)n5)c4)c3)cc21] HBM1 1955543-58-3 |
| 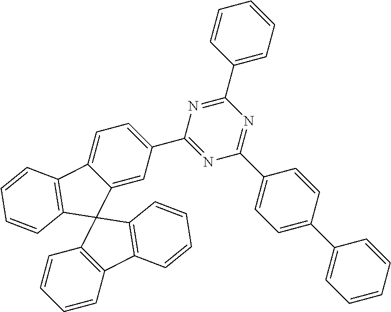 [SMILES: c1ccc(-c2ccc(-c3nc(-c4ccccc4)nc(-c4ccc5c(c4)C4(c6ccccc6-c6ccccc64)c4ccccc4-5)n3)cc2)cc1] ETM1 1819335-36-8 | 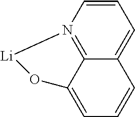 ETM2 25388-93-3 |
| 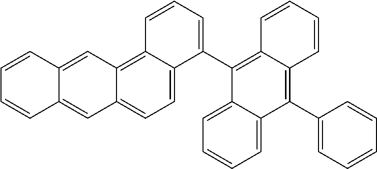 [SMILES: c1ccc(-c2c3ccccc3c(-c3cccc4c3ccc3cc5ccccc5cc34)c3ccccc23)cc1] SMB1 1087346-88-0 | 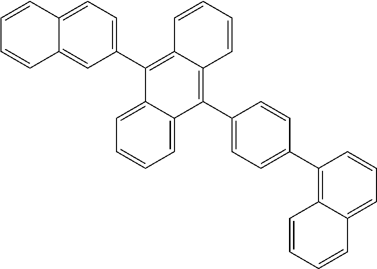 [SMILES: c1ccc2cc(-c3c4ccccc4c(-c4ccc(-c5cccc6ccccc56)cc4)c4ccccc34)ccc2c1] SMB2 667940-34-3 |
| 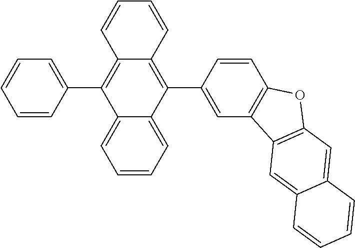 [SMILES: c1ccc(-c2c3ccccc3c(-c3ccc4oc5cc6ccccc6cc5c4c3)c3ccccc23)cc1] SMB3 1627916-48-6 | Fluorescent blue 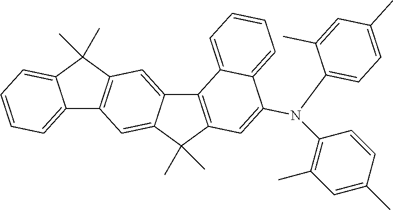 [SMILES: Cc1ccc(N(c2ccc(C)cc2C)c2cc3c(c4ccccc24)-c2cc4c(cc2C3(C)C)-c2ccccc2C4(C)C)c(C)c1] Ref-D1 1182175-28-4 |
| Phosphorescent blue 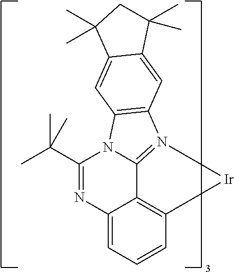 IrB1 1541114-98-0 | Phosphorescent green 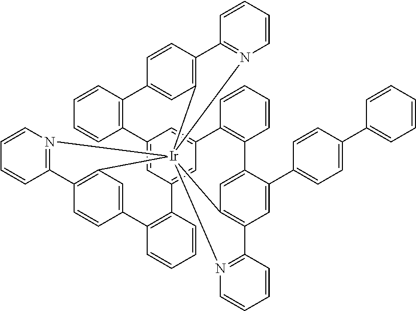 IrG1 2245866-06-0 |
| Phosphorescent yellow 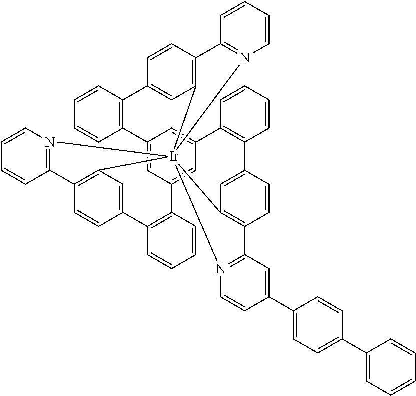 IrG2 2245945-28-0 | Phosphorescent deep red 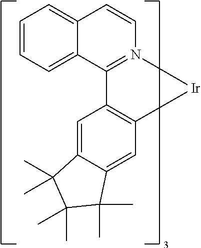 IrR1 1562420-79-4 |

1.-10. (canceled)

11. A compound of one of the formulae (1) and (2): 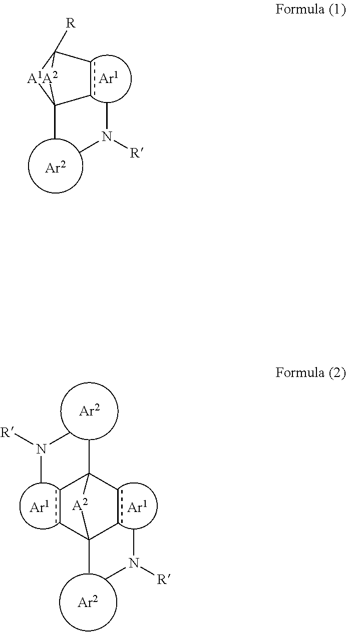
where the symbols used are as follows:
Ar¹, Ar² are the same or different at each instance and are an aromatic or heteroaromatic ring system which has 5 to 60 aromatic ring atoms and may be substituted by one or more R radicals, where Ar¹ and/or Ar² may each be joined to R′ via an R radical or a single bond;
A¹, A² are the same or different at each instance and are a divalent alkylene group having 1 to 4 carbon atoms, a divalent alkenylene group having 2 to 4 carbon atoms or a divalent arylene or heteroarylene group having 5 to 60 ring atoms, where the alkylene, alkenylene, arylene and heteroarylene groups may each be substituted by one or more R groups;
R′ is the same or different at each instance and is H, D, F, Cl, Br, I, OAr′, SAr′, B(R¹)₂, B(OR¹)₂, CHO, C(═O)R, CR═C(R)₂, CN, C(═O)OR, C(═O)NR, Si(R)₃, NO₂, P(═O)(R)₂, OSO₂R, OR, S(═O)R, S(═O)₂R, SR, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R radicals, where one or more nonadjacent CH₂ groups may be replaced by —RC═CR—, —C≡C—, Si(R)₂, NR, CONR, C═O, C═S, —C(═O)O—, P(═O)(R), —O—, —S—, SO or SO₂, or an aromatic or heteroaromatic ring system which has 5 to 60 aromatic ring atoms, and may be substituted in each case by one or more R radicals, where two or more R radicals may together form an aliphatic, heteroaliphatic, aromatic or heteroaromatic ring system which may be substituted by one or more R¹ radicals;
R is the same or different at each instance and is H, D, F, Cl, Br, I, N(Ar′)₂, N(R¹)₂, OAr′, SAr′, B(R¹)₂, B(OR¹)₂, CHO, C(═O)R¹, CR¹=C(R¹)₂, CN, C(═O)OR¹, C(═O)NR¹, Si(R¹)₃, NO₂, P(R¹)₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C≡C—, Si(R¹)₂, NR¹, CONR¹, C═O, C═S, —C(═O)O—, P(═O)(R¹), —O—, —S—, SO or SO₂, or an aromatic or heteroaromatic ring system which has 5 to 60 aromatic ring atoms, and may be substituted in each case by one or more R¹ radicals, where two or more R radicals preferably bonded to the same cycle or the same carbon atom may together and/or with R′ form an aliphatic, heteroaliphatic, aromatic or heteroaromatic ring system which may be substituted by one or more R¹ radicals;
Ar¹ is the same or different at each instance and is an aromatic or heteroaromatic ring system which has 5 to 40 aromatic ring atoms and may be substituted by one or more R¹ radicals;
R¹ is the same or different at each instance and is H, D, F, I, B(R²)₂, B(OR²)₂, N(R²)₂, CHO, C(═O)R², CR²=C(R²)₂, CN, C(═O)OR², Si(R²)₃, NO₂, P(R²)₂, P(═O)(R²)₂, OSO₂R², SR², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—Si(R²)₂, C═O, C═S, —C(═O)O—, NR², CONR², P(═O)(R²), —O—, —S—, SO or SO₂, and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R¹ radicals together may form an aliphatic, heteroaliphatic, aromatic or heteroaromatic ring system;
R² is the same or different at each instance and is H, D, F, CN or an aliphatic, aromatic or heteroaromatic organic radical having 1 to 20 carbon atoms, in which one or more hydrogen atoms may also be replaced by D or F; at the same time, two or more R² substituents may be joined to one another and may form a ring.

12. A compound as claimed in claim 11, selected from one of the compounds of the formulae (3) and (4): 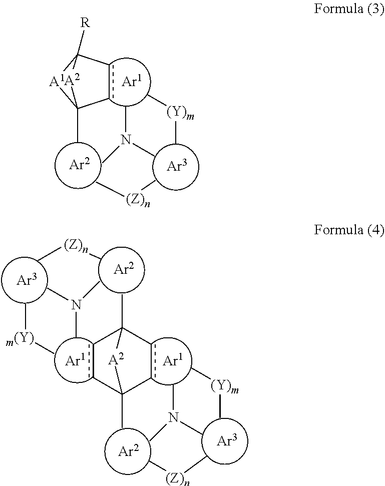
where the symbols used have the definitions given in claim 11, and in addition:
Ar³ is the same or different at each instance and is an aromatic or heteroaromatic ring system which has 5 to 60 aromatic ring atoms and may be substituted by one or more R radicals;
m, n are the same or different at each instance and are 0 or 1;
Y, Z are the same or different at each instance and are a single bond, BR, BAr′, C═O, C(R)₂, —RC═CR—, o-arylene, NR, NAr′, PR, SO₂, SiR₂, SiAr′₂, P(O)R, P(O)Ar′, O or S.

13. A compound as claimed in claim 12, selected from the compounds of the formulae (5) and (6): 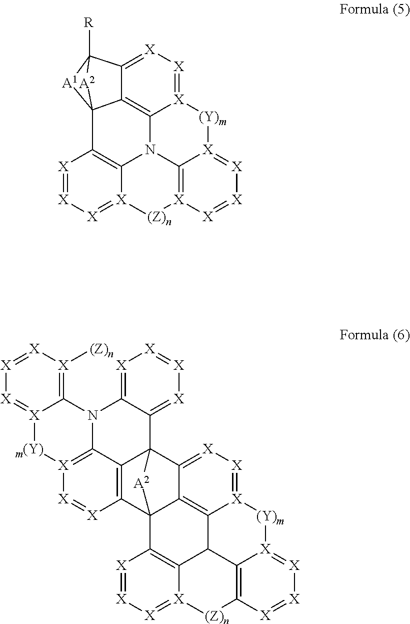
where:
X is the same or different at each instance and is C when Z or Y is bonded there, CR, CH, CD or N, with the proviso that not more than three X groups per cycle are N.

14. A compound as claimed in claim 13, selected from the compounds of the formulae (5-1) to (5-4) and (6-1) to (6-4):

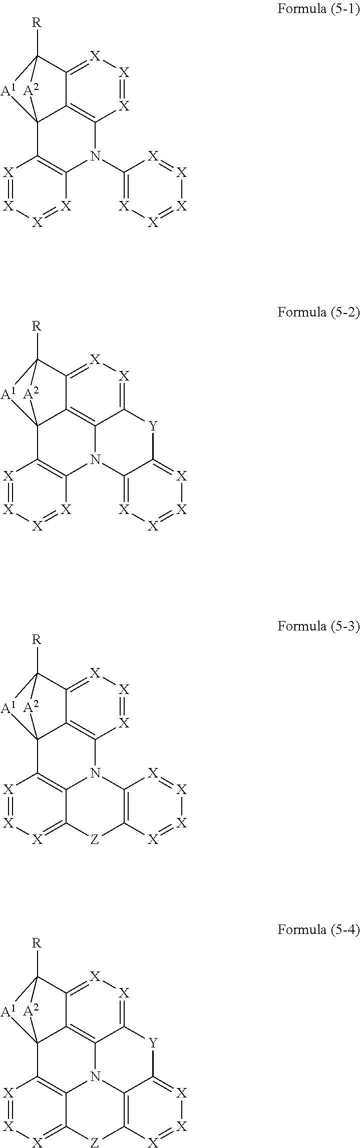

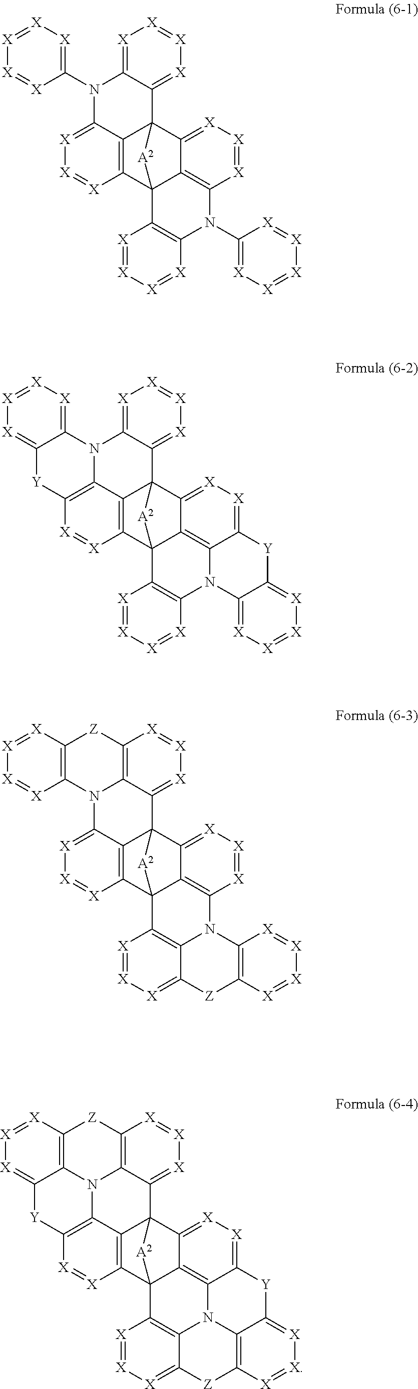

15. A process for preparing a compound as claimed in claim 11, characterized by the following steps:
(A) synthesis of the compound of formula (1) comprising A¹A²-Ar¹ or formula (2) comprising Ar¹A²Ar¹;
(B) introducing the Ar² group in A¹A²-Ar¹ or Ar¹A²Ar¹;
(C) ring closure between Ar² and Ar¹ with introduction of NR′;
(D) optional further functionalization and/or exchange of R′.

16. An oligomer, polymer or dendrimer comprising one or more compounds of formula (1) as claimed in claim 11, wherein the bond(s) to the polymer, oligomer or dendrimer may be at any desired positions in formula (1).

17. A formulation comprising at least one compound as claimed in claim 11 and at least one further compound and/or at least one solvent.

18. An electronic device comprising at least one oligomer, polymer or dendrimer as claimed in claim 16.

19. An electronic device comprising at least one compound as claimed in claim 11.

20. The electronic device as claimed in claim 19, which is an organic electroluminescent device, characterized in that the device comprises anode, cathode and at least one emitting layer, where at least one organic layer that may be an emitting layer, hole transport layer, electron transport layer, hole blocker layer, electron blocker layer or other functional layer comprises the at least one compound.
